(19) United States
(12) Patent Application Publication
(10) Pub. No.: US 2011/0230465 A1
(43) Pub. Date: Sep. 22, 2011

(54) VIRAL POLYMERASE INHIBITORS

(75) Inventors: Timothy STAMMERS, Rosemere (CA); Xavier BARBEAU, St-Augustin de Desmaures (CA); Pierre BEAULIEU, Rosemere (CA); Megan BERTRAND-LAPERLE, Laval (CA); Christian BROCHU, Blainville (CA); Paul J. EDWARDS, Blainville (CA); Pasquale FORGIONE, Montreal (CA); Cédrickx GODBOUT, Mascouche (CA); Oliver HUCKE, Montreal (CA); Marc-André JOLY, Terrebonne (CA); Serge LANDRY, St-Jerome (CA); Olivier LEPAGE, Laval (CA); Julie NAUD, Blainville (CA); Marc PESANT, Saint-Colomban (CA); Martin POIRIER, Blainville (CA); Maude POIRIER, Lavel (CA); Bounkham THAVONEKHAM, Longueuil (CA)

(73) Assignee: BOEHRINGER INGLEHEIM INTERNATIONAL GMBH, Ingelheim (DE)

(21) Appl. No.: 12/883,677

(22) Filed: Sep. 16, 2010

Related U.S. Application Data
(60) Provisional application No. 61/354,820, filed on Jun. 15, 2010.
(60) Provisional application No. 61/243,783, filed on Sep. 18, 2009.

(51) Int. Cl. A61K 31/517; C07D 401/12; C07D 401/14; C07D 239/88; C07D 413/14; C07D 405/12; C07D 417/14; C07D 487/04; C07C 251/48; C07C 233/13; C07C 211/29; C07C 255/58; C07C 255/59; C07C 237/44; C07F 5/04; C07D 213/74; C07D 239/30; A61K 31/5377; A61K 31/541; A61P 31/14

(52) U.S. Cl. 514/210.21; 544/284; 544/287; 544/116; 544/230; 544/58.2; 544/281; 564/229; 564/219; 564/384; 558/415; 558/422; 564/165; 544/229; 546/304; 544/322; 514/266.21; 514/266.2; 514/266.3; 514/266.23; 514/255.05; 514/234.5; 514/228.2

(57) ABSTRACT

Compounds of formula I:

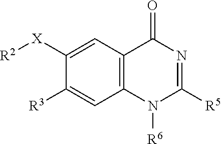

wherein X, R², R³, R⁵ and R⁶ are defined herein, are useful as inhibitors of the hepatitis C virus NS5B polymerase.

### RELATED APPLICATIONS

This application claims benefit of U.S. Ser. No. 61/243,783, filed Sep. 18, 2009, and U.S. Ser. No. 61/354,820 filed Jun. 15, 2010, both of which are herein incorporated by reference.

### FIELD OF THE INVENTION

The invention relates to compounds, compositions and methods for the treatment of hepatitis C virus (HCV) infection. In particular, the present invention provides novel inhibitors of the hepatitis C virus NS5B polymerase, pharmaceutical compositions containing such compounds and methods for using these compounds in the treatment of HCV infection.

### BACKGROUND OF THE INVENTION

It is estimated that at least 170 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma. The development of new and specific anti-HCV treatments is a high priority, and virus-specific functions essential for replication are the most attractive targets for drug development.

### SUMMARY OF THE INVENTION

The present invention provides a novel series of compounds having inhibitory activity against the HCV polymerase enzyme. In particular compounds according to this invention inhibit RNA synthesis by inhibiting the RNA dependent RNA polymerase of HCV, specifically, the enzyme NS5B encoded by HCV.

One aspect of the invention provides compounds of formula (I):

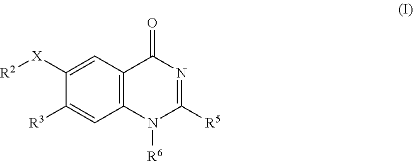

wherein:
X is selected from O, CH₂ and S;
R² is (C₃₋₆)cycloalkyl, aryl or Het, all of which being optionally substituted with 1 to 5 R²⁰ substituents, wherein R²⁰ in each case is independently selected from:
   a) halo, cyano, oxo or nitro;
   b) R⁷, —C(═O)—R⁷, —C(═O)OR⁷, —SR⁷, —SOR⁷, —SO₂R⁷, —(C₁₋₆)alkylene-R⁷, —(C₁₋₆)alkylene-C(═O)R⁷, —(C₁₋₆)alkylene-C(═O)OR⁷, —(C₁₋₆)alkylene-OR⁷, —(C₁₋₆)alkylene-SR⁷, —(C₁₋₆)alkylene-SOR⁷ or —(C₁₋₆)alkylene-SO₂R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, (C₃₋₇)spirocycloalkyl optionally containing 1 to 3 heteroatom selected from N, O and S, aryl and Het;
      wherein the (C₁₋₆)alkyl, (C₂₋₆)alkenyl, (C₂₋₆)alkynyl, (C₁₋₆)haloalkyl and (C₃₋₇)cycloalkyl are optionally substituted with 1 to 5 substituents each independently selected from —OH, oxo, —(C₁₋₆)alkyl (optionally substituted with —O—(C₁₋₆)alkyl), halo, —(C₁₋₆)haloalkyl, (C₃₋₇)cycloalkyl, —O—(C₁₋₆)alkyl, cyano, COOH, —N(R⁸)R⁹, —C(═O)N(R⁸)R⁹, (C₃₋₇)spirocycloalkyl optionally containing 1 to 3 heteroatoms selected from N, O and S, aryl, —(C₁₋₆)alkyl-aryl, Het and —(C₁₋₆)alkyl-Het; and
      wherein each of the aryl and Het is optionally substituted with 1 to 3 substituents each independently selected from:
      i) halo, cyano, oxo, thioxo, imino, —OH, —COOH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, SO₂NH₂, —SO₂—NH(C₁₋₆)alkyl, —SO₂—N((C₁₋₆)alkyl)₂, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, OH, (C₁₋₆)alkyl or —O(C₁₋₆)alkyl; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, —(C₁₋₆)alkylene-SO₂—N(R⁸)R⁹ or —(C₁₋₆)alkylene-NR⁹—SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl, (C₃₋₇)cycloalkyl, —C(═O)R⁷ and —C(═O)OR⁷; and
      R⁹ is in each instance independently selected from halo, cyano, R⁷, OR⁷, —(C₁₋₆)alkylene-R⁷, —SO₂R⁷, —C(═O)R⁷, —OC(═O)R⁷, —C(═O)OR⁷ and —C(═O)N(R⁸)R⁷; wherein R⁷ and R⁸ are as defined above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₆)alkyl optionally substituted with OH, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R³ is selected from H, halo, (C₁₋₆)alkyl, (C₁₋₆)haloalkyl, —O—(C₁₋₆)alkyl, —S—(C₁₋₆)alkyl, cyano, —NH₂, —NH(C₁₋₆)alkyl and —N((C₁₋₆)alkyl)₂;
R⁵ is selected from H, (C₁₋₆)alkyl, (C₃₋₇)cycloalkyl, —(C₁₋₆)alkyl-(C₃₋₇)cycloalkyl, —O—(C₁₋₆)alkyl, —S—(C₁₋₆)alkyl, cyano, —NH₂, —NH(C₁₋₆)alkyl, —N((C₁₋₆)alkyl)₂, —NHC(═O)—(C₁₋₃)alkyl, aryl, —(C₁₋₆)alkyl-aryl, Het or —(C₁₋₆)alkyl-Het; wherein the (C₁₋₆)alkyl, aryl, —(C₁₋₆)alkyl-aryl, Het or —(C₁₋₆)alkyl-Het are optionally substituted with 1 to 4 substituents each independently selected from (C₁₋₆)alkyl, halo, —OH, —COOH, —O(C₁₋₆)alkyl, —C(═O)—(C₁₋₆)alkyl, —C(═O)—O—(C₁₋₆)alkyl, cyano, —NH₂, —NH(C₁₋₆)alkyl, and —N((C₁₋₆)alkyl)₂;
R⁶ is selected from (C₁₋₈)alkyl, (C₂₋₈)alkenyl, (C₂₋₈)alkynyl, (C₃₋₇)cycloalkyl, aryl and Het,
   wherein said R⁶ can be optionally substituted with 1 to 6 R²¹ substituents,
   wherein R²¹ in each case is independently selected from:
   a) halo, NH₂, NO₂, cyano, azido or oxo;
   b) R²¹⁰, OR²¹⁰, NR²¹⁰R²¹¹, SR²¹⁰, SOR²¹⁰, SO₂R²¹⁰, C(═O)R²¹⁰, C(═O)OR²¹⁰, C(═O)NR²¹⁰R²¹¹, NR²¹¹C(═O)R²¹², NR²¹¹C(═O)OR²¹², NR²¹¹C(═O)NR²¹¹R²¹², NR²¹¹SO₂R²¹⁰, NR²¹¹SO₂NR²¹⁰R²¹² and SO₂NR²¹⁰R²¹¹;
      wherein R²¹⁰ is selected from H, (C₁₋₈)alkyl, (C₁₋₈)haloalkyl, (C₂₋₈)alkenyl, (C₂₋₈)alkynyl, (C₃₋₇)cycloalkyl, (C₅₋₇)cycloalkenyl, (C₃₋₇)spirocycloalkyl optionally containing 1 to 3 heteroatom selected from N, O and S, C(═O)R²¹¹, C(═O)OR²¹¹, aryl and Het, all of which can be optionally substituted with 1 to 6 substituents selected from OH, NH₂, cyano, oxo, NO₂, halo, R²¹², OR²¹¹, SR²¹¹, NR²¹¹R²¹², NR²¹¹C(═O)R²¹², NR²¹¹C(═O)OR²¹², NR²¹¹C(═O)NR²¹¹R²¹², NR²¹¹SO₂R²¹⁰, NR²¹¹SO₂NR²¹⁰R²¹², C(═O)R²¹¹, C(═O)OR²¹¹, C(═O)NR²¹¹R²¹², and wherein R²¹¹ is selected from H, (C₁₋₆)alkyl, and (C₃₋₇)cycloalkyl; and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl and Het, all of which being optionally substituted with 1 to 6 substituents selected from OH, NH₂, cyano, oxo, NO₂, halo, (C₁₋₆)alkyl, (C₃₋₇)cycloalkyl, (C₁₋₆)haloalkyl, O—(C₁₋₆)alkyl, S—(C₁₋₆)alkyl, NH(C₁₋₆)alkyl, N((C₁₋₆)alkyl)₂, aryl and Het, wherein aryl and Het can be optionally substituted with 1 to 3 substituents selected from OH, halo, (C₁₋₃)alkyl and —O(C₁₋₃)alkyl;
         or R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
            or a salt thereof.

Another aspect of this invention provides a compound of formula (I), or a pharmaceutically acceptable salt thereof, as a medicament.

Still another aspect of this invention provides a pharmaceutical composition comprising a therapeutically effective amount of a compound of formula (I) or a pharmaceutically acceptable salt thereof; and one or more pharmaceutically acceptable carriers.

According to an embodiment of this aspect, the pharmaceutical composition according to this invention additionally comprises at least one other antiviral agent.

The invention also provides the use of a pharmaceutical composition as described hereinabove for the treatment of a hepatitis C viral infection in a human being having or at risk of having the infection.

A further aspect of the invention involves a method of treating a hepatitis C viral infection in a human being having or at risk of having the infection, the method comprising administering to the human being a therapeutically effective amount of a compound of formula (I), a pharmaceutically acceptable salt thereof, or a composition thereof as described hereinabove.

Another aspect of the invention involves a method of treating a hepatitis C viral infection in a human being having or at risk of having the infection, the method comprising administering to the human being a therapeutically effective amount of a combination of a compound of formula (I) or a pharmaceutically acceptable salt thereof, and at least one other antiviral agent; or a composition thereof.

Also within the scope of this invention is the use of a compound of formula (I) as described herein, or a pharmaceutically acceptable salt thereof, for the treatment of a hepatitis C viral infection in a human being having or at risk of having the infection.

Another aspect of this invention provides the use of a compound of formula (I) as described herein, or a pharmaceutically acceptable salt thereof, for the manufacture of a medicament for the treatment of a hepatitis C viral infection in a human being having or at risk of having the infection.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound of formula (I) according to this invention or a pharmaceutically acceptable salt thereof.

Still another aspect of this invention relates to a method of inhibiting the replication of hepatitis C virus comprising exposing the virus to an effective amount of the compound of formula (I), or a salt thereof, under conditions where replication of hepatitis C virus is inhibited.

Further included in the scope of the invention is the use of a compound of formula (I), or a salt thereof, to inhibit the replication of hepatitis C virus.

In another aspect the invention provides novel intermediates useful in the production of compounds of Formula (I). In particular, the novel intermediates comprise one or more of the intermediates selected from the group consisting of intermediates designated 154a1, 154a2, 154a3, 154a4, 154a5, 154a6, 154a7, 154a8, 154a9, 154b1 and 154c1, as disclosed in the Examples.

### DETAILED DESCRIPTION OF THE INVENTION

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general, for groups comprising two or more subgroups, the first named subgroup is the radical attachment point, for example, the substituent “—C₁₋₃-alkyl-aryl” means an aryl group which is bound to a C₁₋₃-alkyl group, wherein the C₁₋₃-alkyl group is bound to the core. It is understood that substituents may be attached to any one of the subgroups, unless specified otherwise. In the previous example of “—C₁₋₃-alkyl-aryl”, substituents may be attached to either the C₁₋₃-alkyl or aryl portion thereof or both.

In case a compound of the present invention is depicted in the form of a chemical name and as a formula in case of any discrepancy the formula shall prevail.

The following designation

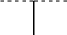

is used in sub-formulas to indicate the bond which is connected to the rest of the molecule as defined.

The term “C₁₋ₙ-alkyl”, wherein n is an integer from 2 to n, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)—, H₃C—CH₂—CH(CH₂CH₃)— and H₃C—CH(CH₂CH₃)—CH₂—.

The term “C₁₋ₙ-alkylene” wherein n is an integer 2 to n, either alone or in combination with another radical, denotes an acyclic, straight or branched chain divalent alkyl radical containing from 1 to n carbon atoms. For example the term C₁₋₄-alkylene includes —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))— and —(CHCH(CH₃)₂)—.

The term “C₂₋ₙ-alkenyl”, is used for a group as defined in the definition for “C₁₋ₙ-alkyl” with at least two carbon atoms, if at least two of those carbon atoms of said group are bonded to each other by a double bond.

The term “C₂₋ₙ-alkynyl”, is used for a group as defined in the definition for “C₁₋ₙ-alkyl” with at least two carbon atoms, if at least two of those carbon atoms of said group are bonded to each other by a triple bond.

The term “C₃₋ₙ-cycloalkyl”, wherein n is an integer 4 to n, either alone or in combination with another radical denotes a cyclic, saturated, unbranched hydrocarbon radical with 3 to n C atoms. For example the term C₃₋₇-cycloalkyl includes cyclopropyl, cyclobutyl, cyclopentyl, cyclohexyl and cycloheptyl.

The term “C₃₋ₙ-cycloalkenyl”, wherein n is an integer 4 to n, either alone or in combination with another radical, denotes an cyclic, unsaturated but nonaromatic, unbranched hydrocarbon radical with 3 to n C atoms, at least two of which are bonded to each other by a double bond. For example the term C₃₋₇-cycloalkenyl includes cyclopropenyl, cyclobutenyl, cyclopentenyl, cyclopentadienyl, cyclohexenyl, cyclohexadienyl, cycloheptenyl cycloheptadienyl and cycloheptatrienyl.

The term “aryl” as used herein, either alone or in combination with another radical, denotes a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, naphthyl, anthracenyl, phenanthrenyl, tetrahydronaphthyl and dihydronaphthyl.

The term “Het” as used herein, either alone or in combination with another radical, is intended to mean a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S; wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂, unless specified otherwise. When a Het group is substituted, it is understood that substituents may be attached to any carbon atom or heteroatom thereof which would otherwise bear a hydrogen atom, unless specified otherwise.

The term “heteroatom” as used herein is intended to mean O, S or N.

The term “heterocycle” as used herein and unless specified otherwise, either alone or in combination with another radical, is intended to mean a 4- to 7-membered saturated, unsaturated or aromatic heterocycle containing from 1 to 4 heteroatoms each independently selected from O, N and S; or a monovalent radical derived by removal of a hydrogen atom therefrom. Examples of such heterocycles include, but are not limited to, azetidine, pyrrolidine, tetrahydrofuran, tetrahydrothiophene, thiazolidine, oxazolidine, pyrrole, thiophene, furan, pyrazole, imidazole, isoxazole, oxazole, isothiazole, thiazole, triazole, tetrazole, piperidine, piperazine, azepine, diazepine, pyran, 1,4-dioxane, 4-morpholine, 4-thiomorpholine, pyridine, pyridine-N-oxide, pyridazine, pyrazine, pyrimidine, and the following heterocycles:

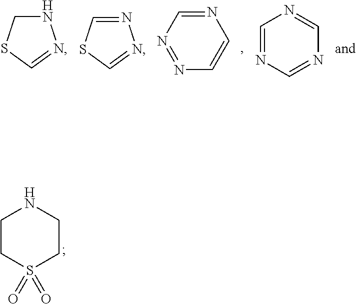

and saturated, unsaturated and aromatic derivatives thereof.

The term “heteropolycycle” as used herein and unless specified otherwise, either alone or in combination with another radical, is intended to mean a heterocycle as defined above fused to one or more other cycle, including a carbocycle, a heterocycle or any other cycle; or a monovalent radical derived by removal of a hydrogen atom therefrom. Examples of such heteropolycycles include, but are not limited to, indole, isoindole, benzimidazole, benzothiophene, benzofuran, benzodioxole, benzothiazole, quinoline, isoquinoline, naphthyridine, and the following heteropolycycles:

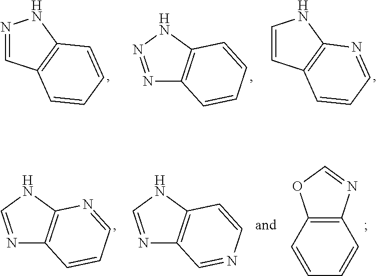

and saturated, unsaturated and aromatic derivatives thereof.

The term “halo” as used herein is intended to mean a halogen substituent selected from fluoro, chloro, bromo or iodo.

The term “oxo” as used herein is intended to mean an oxygen atom attached to a carbon atom as a substituent by a double bond (═O).

The term “thioxo” as used herein is intended to mean a sulfur atom attached to a carbon atom as a substituent by a double bond (═S).

The term “imino” as used herein is intended to mean a NH group attached to a carbon atom as a substituent by a double bond (═NH).

The term “cyano” or “CN” as used herein is intended to mean a nitrogen atom attached to a carbon atom by a triple bond (CEN).

The term “salt thereof” as used herein is intended to mean any acid and/or base addition salt of a compound according to the invention, including but not limited to a pharmaceutically acceptable salt thereof. Salts of other acids than those mentioned above which for example are useful for purifying or isolating the compounds of the present invention (e.g. trifluoro acetate salts) also comprise a part of the invention.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, benzenesulfonates, benzoates, besylates, bicarbonates, bitartrates, bromides/hydrobromides, Ca-edetates/edetates, camsylates, carbonates, chlorides/hydrochlorides, citrates, edisylates, ethane disulfonates, estolates esylates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates subacetates, succinates, sulfamides, sulfates, tannates, tartrates, teoclates, toluenesulfonates, triethiodides, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

The term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectible levels in the blood.

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The present invention also provides all pharmaceutically-acceptable isotopically labeled compounds of the present invention wherein one or more atoms are replaced by atoms having the same atomic number, but an atomic mass or mass number different from the atomic mass or mass number predominantly found in nature.

### Preferred Embodiments

In the following preferred embodiments, groups and substituents of the compounds of formula (I):

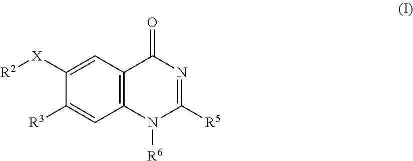

are described in detail.

Any and each individual definition as set out herein may be combined with any and each individual definition as set out herein.

### X:

X-A: In another embodiment, X is O, S or CH₂.
X-B: In another embodiment, X is O or S.
X-C: In one embodiment, X is O.

### R²:

R²-A: In one embodiment, R² is selected from (C₃₋₆)cycloalkyl, aryl or Het optionally substituted with 1 to 5 R²⁰ substituents, wherein R²⁰ is as defined herein.
R²-B: In another embodiment, R² is selected from (C₄₋₆)cycloalkyl, aryl or Het optionally substituted with 1 to 3 R²⁰ substituents, wherein R²⁰ is as defined herein.
R²-C: In another embodiment, R² is selected from aryl or Het optionally substituted with 1 to 3 R²⁰ substituents, wherein R²⁰ is as defined herein.
R²-D: In another embodiment, R² is selected from phenyl or Het wherein Het is a 5- or 6 membered heterocycle containing 1 to 3 heteroatoms each independently selected from O, N and S, or a 9- or 10-membered bicyclic heteropolycycle containing 1 to 3 heteroatoms each independently selected from O, N and S; wherein phenyl and Het are optionally substituted with 1 to 3 R²⁰ substituents, wherein R²⁰ is as defined herein.
R²-E: In another embodiment, R² is phenyl or Het wherein Het is a 5- or 6 membered aromatic heterocycle containing 1 or 2 N heteroatoms or a 9- to 10-membered bicyclic heteropolycycle containing 1 or 2 N heteroatoms; wherein phenyl and Het are optionally substituted with 1 to 3 R²⁰ substituents, wherein R²⁰ is as defined herein.
R²-F: In another embodiment, R² is selected from the following formulas:

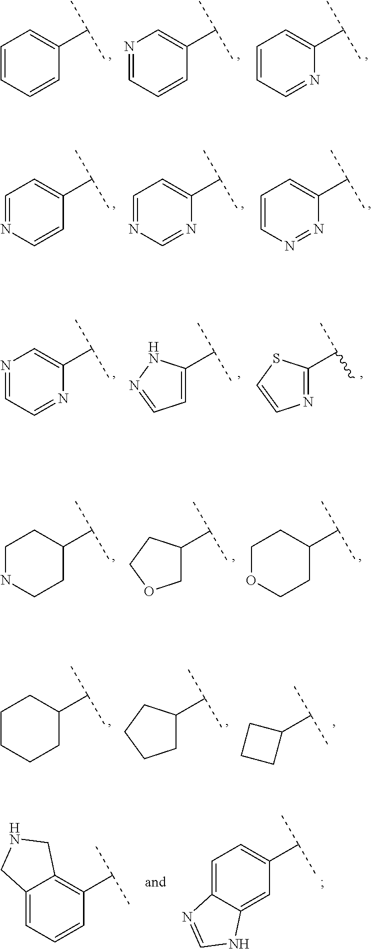

wherein R² is optionally substituted with 1 to 3 R²⁰ substituents, wherein R²⁰ is as defined herein.
R²-G: In another embodiment, R² is selected from the formulas:

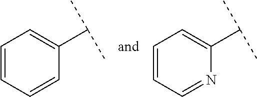

wherein R² is optionally substituted with 1 to 3 R²⁰ substituents, wherein R²⁰ is as defined herein.
R²-H: In another embodiment, R² is selected from the following formulas:

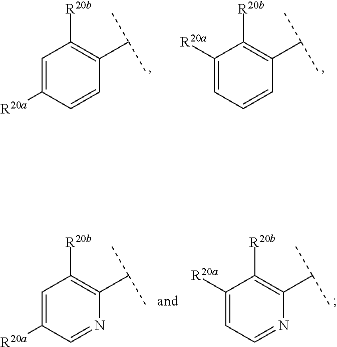

wherein R^{20b} is as defined:
   R^{20b}-A: In this embodiment, R^{20b} is selected from H, halo, (C₁₋₆)alkyl, (C₁₋₆)haloalkyl, (C₃₋₇)cycloalkyl and —O—(C₁₋₆)haloalkyl.
   R^{20b}-B: In this embodiment, R^{20b} is selected from H, Cl, Br, CH₃, CHF₂, CF₃, cyclopropyl, cyclobutyl and —OCF₃.
   R^{20b}-C: In this embodiment, R^{20b} is H, CHF₂ or CF₃.
   R^{20b}-D: In this embodiment, R^{20b} is H or CF₃.
   R^{20b}-E: In this embodiment, R^{20b} is CF₃;
   and R^{20a} is R²⁰ wherein R²⁰ is as defined herein.

### R²⁰:

R²⁰-A: In one embodiment, R²⁰ is selected from:
   a) halo, cyano, oxo or nitro;
   b) R⁷, —C(═O)—R⁷, —C(═O)OR⁷, —SR⁷, —SOR⁷, —SO₂R⁷, —(C₁₋₆)alkylene-R⁷, —(C₁₋₆)alkylene-C(═O)R⁷, —(C₁₋₆)alkylene-C(═O)OR⁷, —(C₁₋₆)alkylene-OR⁷, —(C₁₋₆)alkylene-SR⁷, —(C₁₋₆)alkylene-SOR⁷ or —(C₁₋₆)alkylene-SO₂R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, (C₃₋₇)spirocycloalkyl optionally containing 1 to 3 heteroatom selected from N, O and S, aryl and Het;
      wherein the (C₁₋₆)alkyl, (C₂₋₆)alkenyl, (C₂₋₆)alkynyl, (C₁₋₆)haloalkyl and (C₃₋₇)cycloalkyl are optionally substituted with 1 to 5 substituents each independently selected from —OH, oxo, —(C₁₋₆)alkyl (optionally substituted with —O—(C₁₋₆)alkyl), halo, —(C₁₋₆)haloalkyl, (C₃₋₇)cycloalkyl, —O—(C₁₋₆)alkyl, cyano, COOH, —N(R⁸)R⁹, —C(═O)N(R⁸)R⁹, (C₃₋₇)spirocycloalkyl optionally containing 1 to 3 heteroatoms selected from N, O and S, aryl, —(C₁₋₆)alkyl-aryl, Het and —(C₁₋₆)alkyl-Het; and
      wherein each of the aryl and Het is optionally substituted with 1 to 3 substituents each independently selected from:
      i) halo, cyano, oxo, thioxo, imino, —OH, —COOH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, SO₂NH₂, —SO₂—NH(C₁₋₆)alkyl, —SO₂—N((C₁₋₆)alkyl)₂, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, OH, (C₁₋₆)alkyl or —O(C₁₋₆)alkyl; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, —(C₁₋₆)alkylene-SO₂—N(R⁸)R⁹ or —(C₁₋₆)alkylene-NR⁹—SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl, (C₃₋₇)cycloalkyl, —C(═O)R⁷ and —C(═O)OR⁷; and
      R⁹ is in each instance independently selected from halo, cyano, R⁷, OR⁷, —(C₁₋₆)alkylene-R⁷, —SO₂R⁷, —C(═O)R⁷, —OC(═O)R⁷, —C(═O)OR⁷ and —C(═O)N(R⁸)R⁷; wherein R⁷ and R⁸ are as defined above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₆)alkyl optionally substituted with OH, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.
R²⁰-B: In one embodiment, R²⁰ is selected from:
   a) halo, cyano, oxo;
   b) R⁷, —C(═O)—R⁷, —C(═O)OR⁷, —SO₂R⁷, —(C₁₋₆)alkylene-R⁷, —(C₁₋₆)alkylene-C(═O)R⁷, —(C₁₋₆)alkylene-C(═O)OR⁷, —(C₁₋₆)alkylene-OR′;
      wherein R⁷ is in each instance independently selected from H, (C₁₋₆)alkyl, (C₂₋₆)alkenyl, (C₁₋₆)haloalkyl, (C₃₋₇)cycloalkyl, (C₃₋₇)spirocycloalkyl optionally containing 1 to 3 heteroatom selected from N, O and S, aryl and Het;
      wherein the (C₁₋₆)alkyl, (C₂₋₆)alkenyl, (C₁₋₆)haloalkyl and (C₃₋₇)cycloalkyl are optionally substituted with 1 to 4 substituents each independently selected from —OH, oxo, —(C₁₋₆)alkyl, halo, —(C₁₋₆)haloalkyl, (C₃₋₇)cycloalkyl, —O—(C₁₋₆)alkyl, cyano, COOH, —N(R⁸)R⁹, —C(═O)N(R⁸)R⁹, (C₃₋₇)spirocycloalkyl optionally containing 1 to 3 heteroatoms selected from N, O and S, aryl and Het; and
      wherein each of the aryl and Het is optionally substituted with 1 to 3 substituents each independently selected from:
      i) halo, cyano, oxo, —OH, —COOH, —O—(C₁₋₆)alkyl, SO₂NH₂, —SO₂—NH(C₁₋₄)alkyl, —SO₂—N((C₁₋₄)alkyl)₂, —SO₂(C₁₋₄)alkyl, —NH₂, —NH(C₁₋₄)alkyl, —N((C₁₋₄)alkyl)₂;
      ii) (C₁₋₄)alkyl optionally substituted with —OH or —O—(C₁₋₃)alkyl; and
      iii) aryl or Het, wherein each of the aryl and Het is optionally substituted with halo, OH, (C₁₋₃)alkyl or —O(C₁₋₃)alkyl; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, —(C₁₋₆)alkylene-SO₂—N(R⁸)R⁹ or —(C₁₋₆)alkylene-NR⁹—SO₂—N(R⁸)R⁹; wherein the (C₁₋₆)alkylene is optionally substituted with 1 or 2 substituents each independently selected from —OH, halo, —O—(C₁₋₃)alkyl, cyano, COOH, —NH₂, —NH(C₁₋₄)alkyl and —N((C₁₋₄)alkyl)₂;
      R⁸ is in each instance independently selected from H, (C₁₋₆)alkyl, (C₃₋₇)cycloalkyl, —C(═O)R⁷ and —C(═O)OR⁷; and
      R⁹ is in each instance independently selected from halo, cyano, R⁷, OR⁷, —SO₂R⁷, —C(═O)R⁷, —OC(═O)R⁷ and —C(═O)OR⁷; wherein R⁷ is as defined above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₆)alkyl optionally substituted with —OH, (C₁₋₆)haloalkyl, halo, —O(C₁₋₆)alkyl, —NH₂, —NH(C₁₋₆)alkyl and —N((C₁₋₆)alkyl)₂.
R²⁰-C: In one embodiment, R²⁰ is selected from:
   a) halo or cyano;
   b) R⁷, —C(═O)—R⁷, —C(═O)OR⁷, —SO₂R⁷, —(C₁₋₆)alkylene-C(═O)R⁷, or —(C₁₋₆)alkylene-C(═O)OR⁷;
      wherein R⁷ is in each instance independently selected from H, (C₁₋₄)alkyl, (C₂₋₄)alkenyl, (C₁₋₄haloalkyl, (C₃₋₇)cycloalkyl, aryl and Het;
      wherein the (C₁₋₄)alkyl, (C₂₋₄alkenyl, and (C₁₋₄)haloalkyl are optionally substituted with 1 to 3 substituents each independently selected from —OH, —(C₁₋₆)alkyl, halo, (C₃₋₇)cycloalkyl, —O—(C₁₋₃)alkyl, cyano, COOH, —N(R⁸)R⁹, —C(═O)N(R⁸)R⁹, aryl and Het; and
      wherein each of the aryl and Het is optionally substituted with 1 to 3 substituents each independently selected from:
      i) halo, cyano, oxo, —OH, —COOH, —O—(C₁₋₆)alkyl, SO₂NH₂, —SO₂—NH(C₁₋₄)alkyl, —SO₂—N((C₁₋₄)alkyl)₂, —SO₂(C₁₋₄)alkyl, —NH₂, —NH(C₁₋₄)alkyl, —N((C₁₋₄)alkyl)₂;
      ii) (C₁₋₄)alkyl optionally substituted with —OH or —O—(C₁₋₃)alkyl; and
      iii) aryl or Het, wherein each of the aryl and Het is optionally substituted with halo, OH, (C₁₋₃)alkyl or —O(C₁₋₃)alkyl;
      wherein Het is a 5- or 6-membered heterocycle containing 1 to 4 heteroatoms, each independently selected from N, O and S, or Het is a 9- or 10-membered heteropolycycle containing 1 to 4 heteroatoms, each independently selected from N, O and S; wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸) R⁹, —(C₁₋₆)alkylene-SO₂—N(R⁸)R⁹ or —(C₁₋₆)alkylene-NR⁹—SO₂—N(R⁸)R⁹; wherein the (C₁₋₆)alkylene is optionally substituted with 1 or 2 substituents each independently selected from —OH, halo, —O—(C₁₋₃)alkyl, cyano, COOH, —NH₂, —NH(C₁₋₄)alkyl and —N((C₁₋₄)alkyl)₂;
      R⁸ is in each instance independently selected from H and (C₁₋₄)alkyl; and
      R⁹ is in each instance independently selected from halo, cyano, R⁷, OR⁷, —SO₂R⁷, —C(═O)R⁷ and —C(═O)OR⁷; wherein R⁷ is as defined above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₃)alkyl optionally substituted with —OH, (C₁₋₃)haloalkyl, halo, —O(C₁₋₃)alkyl, —NH₂, —NH(C₁₋₃)alkyl and —N((C₁₋₃)alkyl)₂.
R²⁰-D: In one embodiment, R²⁰ is selected from:
   a) halo or cyano;
   b) R⁷, —C(═O)—R⁷, —C(═O)OR⁷, —SO₂R⁷, or —(C₁₋₆)alkylene-C(═O)OR⁷;
      wherein R⁷ is in each instance independently selected from H, (C₁₋₄)alkyl, (C₂₋₄)alkenyl, (C₁₋₄)haloalkyl, (C₃₋₇)cycloalkyl, aryl and Het;
      wherein the (C₁₋₄)alkyl, (C₂₋₄)alkenyl, and (C₁₋₄)haloalkyl are optionally substituted with 1 to 3 substituents each independently selected from —OH, —(C₁₋₄)alkyl, halo, (C₃₋₇)cycloalkyl, —O—(C₁₋₃)alkyl, cyano, COOH, —N(R⁸)R⁹, —C(═O)N(R⁸)R⁹, aryl and Het; and
      wherein each of the aryl and Het is optionally substituted with 1 to 3 substituents each independently selected from:
      i) halo, cyano, oxo, —OH, —COOH, —O—(C₁₋₆)alkyl, SO₂NH₂, —SO₂—NH(C₁₋₃)alkyl, —SO₂—N((C₁₋₃)alkyl)₂, —SO₂(C₁₋₃)alkyl, —NH₂, —NH(C₁₋₃)alkyl, —N((C₁₋₃)alkyl)₂;
      ii) (C₁₋₄)alkyl optionally substituted with —OH or —O—(C₁₋₃)alkyl; and
      iii) phenyl or Het, wherein each of the phenyl and Het is optionally substituted with halo, OH, (C₁₋₃)alkyl or —O(C₁₋₃)alkyl;
      wherein each Het is selected from:

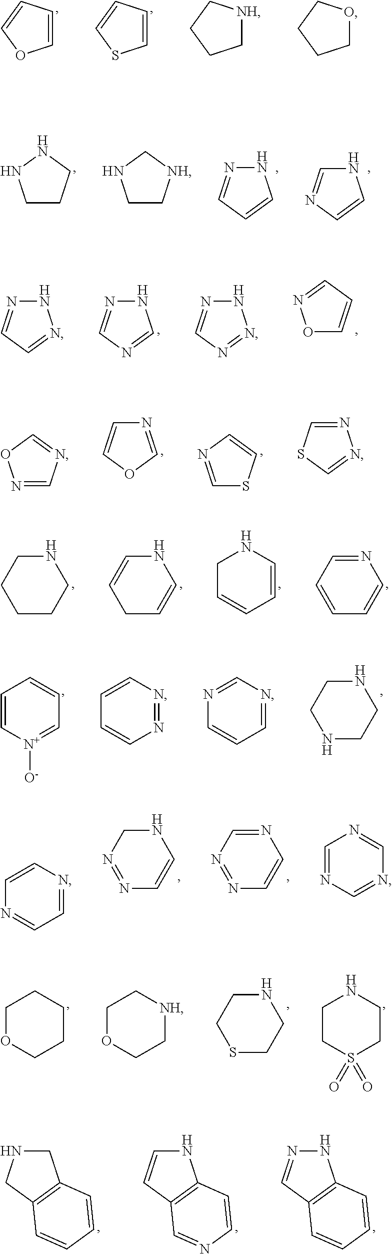

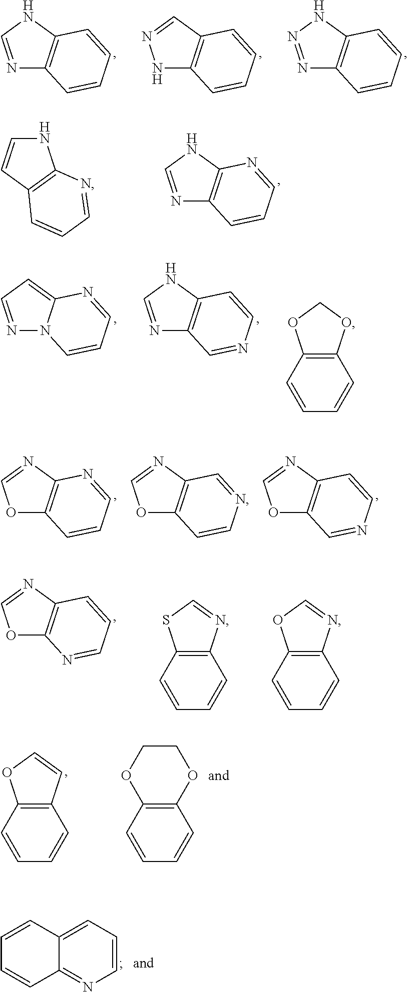

c) —N(R⁸)R⁹, —C(═O)—N(R⁸)R⁹, —SO₂—N(R⁸)R⁹, —(C₁₋₃)alkylene-N(R⁸)R⁹ or —(C₁₋₃)alkylene-C(═O)—N(R⁸)R⁹; wherein the (C₁₋₃)alkylene is optionally substituted with 1 or 2 substituents each independently selected from —OH and —O—(C₁₋₃)alkyl;
   R⁸ is in each instance independently selected from H and (C₁₋₃)alkyl; and
   R⁹ is in each instance independently selected from halo, cyano, R⁷, OR⁷, —SO₂R⁷, —C(═O)R⁷ and —C(═O)OR⁷; wherein R⁷ is as defined above;
      or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
      wherein the heterocycle is optionally substituted with 1 to 3 substituents each independently selected from (C₁₋₃)alkyl optionally substituted with —OH, —O(C₁₋₃)alkyl, —NH₂, —NH(C₁₋₃)alkyl and —N((C₁₋₃)alkyl)₂.
R²⁰-E: In one embodiment, R²⁰ is selected from:
   a) halo or cyano;
   b) R⁷, —C(═O)—R⁷, —C(═O)OR⁷, —OR⁷ or —(C₁₋₆)alkylene-C(═O)OR⁷;
      wherein R⁷ is in each instance independently selected from H, (C₁₋₄)alkyl, phenyl and Het;
      wherein the (C₁₋₄)alkyl is optionally substituted with 1 to 3 substituents each independently selected from —OH, halo, (C₃₋₇)cycloalkyl, —O—(C₁₋₃)alkyl, cyano, COOH, —N(R⁸)R⁹, —C(═O)N(R⁸)R⁹, aryl and Het; and
      wherein each of the phenyl and Het is optionally substituted with 1 to 3 substituents each independently selected from:
      i) halo, cyano, oxo, —OH, —COOH, —O—(C₁₋₆)alkyl, SO₂NH₂, —SO₂—NH(C₁₋₃)alkyl, —SO₂—N((C₁₋₃)alkyl)₂, —NH₂, —NH(C₁₋₃)alkyl, —N((C₁₋₃)alkyl)₂;
      ii) (C₁₋₄)alkyl optionally substituted with —OH or —O—(C₁)alkyl; and
      iii) phenyl or Het, wherein each of the phenyl and Het is optionally substituted with halo, OH or —O(C₁)alkyl;
      wherein each Het is selected from:

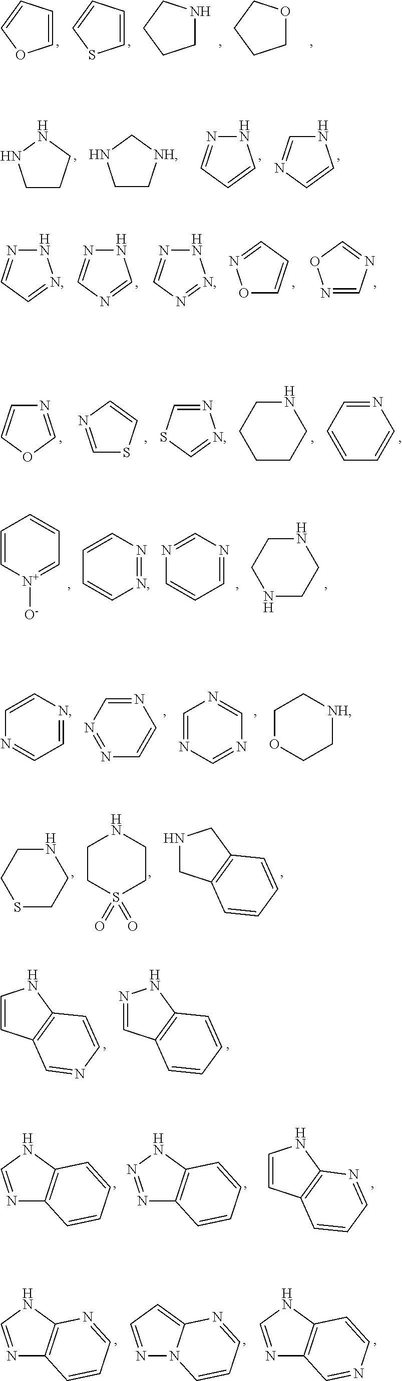

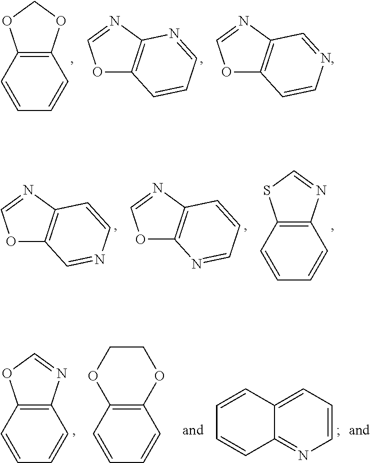

c) —N(R⁸)R⁹, —C(═O)—N(R⁸)R⁹, —SO₂—N(R⁸)R⁹, —(C₁₋₃)alkylene-N(R⁸)R⁹ or —(C₁₋₃)alkylene-C(═O)—N(R⁸)R⁹; wherein the (C₁₋₃)alkylene is optionally substituted with 1 or 2 substituents each independently selected from —OH and —O—(C₁₋₃)alkyl;
   R⁸ is in each instance independently selected from H and (C₁₋₃)alkyl; and
   R⁹ is in each instance independently selected from halo, cyano, R⁷, OR⁷, —SO₂R⁷, —C(═O)R⁷ and —C(═O)OR⁷; wherein R⁷ is as defined above;
      or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
      wherein the heterocycle is optionally substituted with 1 to 3 substituents each independently selected from (C₁₋₃)alkyl optionally substituted with —OH, —O(C₁₋₃)alkyl, —NH₂, —NH(C₁₋₃)alkyl and —N((C₁₋₃)alkyl)₂.
R²⁰-F: In another embodiment, R²⁹ is selected from H, F, Cl, Br, OH, CF₃, (C₁₋₃)alkyl, O—(C₁₋₃)alkyl, (C₁₋₃)alkyl-COOH, (C₁₋₃)alkyl-CONH₂, NH₂, NH(C₁₋₃)alkyl, N((C₁₋₃)alkyl)₂, phenyl or Het, wherein the phenyl and Het are optionally substituted with 1 to 2 substituents independently selected from halo, OH, (C₁₋₃)alkyl, —NH₂, —NH(C₁₋₃)alkyl, —N((C₁₋₃)alkyl)₂, O—(C₁₋₃)alkyl, phenyl or Het, wherein each Het is selected from:

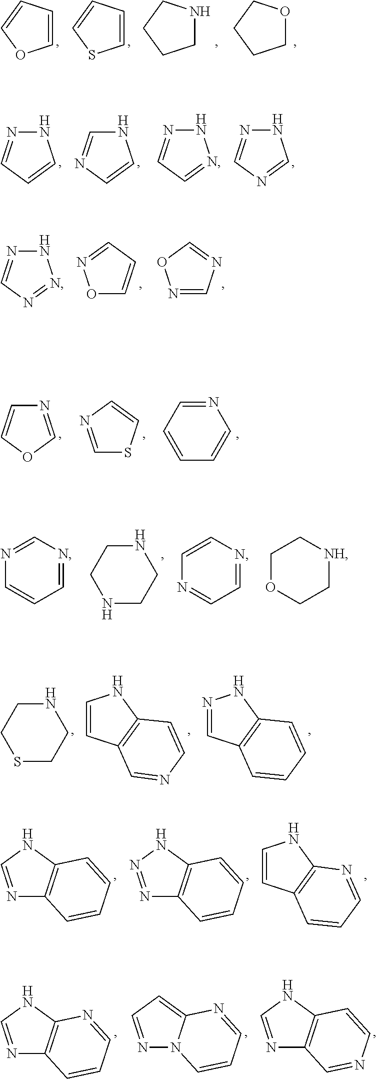

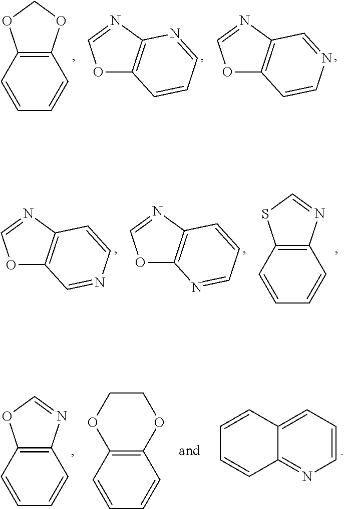

### R³:

R³-A: In one embodiment, R³ is selected from H, halo, (C₁₋₆)alkyl, (C₁₋₆)haloalkyl, —O—(C₁₋₆)alkyl, —S—(C₁₋₆)alkyl, cyano, —NH₂, —NH(C₁₋₆)alkyl and —N((C₁₋₆)alkyl)₂;
R³-B: In one embodiment, R³ is selected from H, halo, (C₁₋₆)alkyl, —O—(C₁₋₆)alkyl, cyano, —NH₂, —NH(C₁₋₆)alkyl and —N((C₁₋₆)alkyl)₂.
R³-C: In another embodiment, R³ is selected from H, F, Br, Cl, (C₁₋₆)alkyl, —O—(C₁₋₆)alkyl, and —N((C₁₋₆)alkyl)₂.
R³-D: In another embodiment, R³ is selected from H, F, Br, Cl, —OCH₃ and —N(CH₃)₂.
R³-E: In another embodiment, R³ is H or F.
R³-F: In another embodiment, R³ is H.

### R⁵:

R⁵-A: In one embodiment, R⁵ is selected from H, (C₁₋₆)alkyl, (C₃₋₇)cycloalkyl, —(C₁₋₆)alkyl-(C₃₋₇)cycloalkyl, —O—(C₁₋₆)alkyl, —S—(C₁₋₆)alkyl, cyano, —NH₂, —NH(C₁₋₆)alkyl, —N((C₁₋₆)alkyl)₂, —NHC(═O)—(C₁₋₃)alkyl, aryl, —(C₁₋₆)alkyl-aryl, Het or —(C₁₋₆)alkyl-Het; wherein the (C₁₋₆)alkyl, aryl, —(C₁₋₆)alkyl-aryl, Het or —(C₁₋₆)alkyl-Het are optionally substituted with 1 to 4 substituents each independently selected from (C₁₋₆)alkyl, halo, —OH, —COOH, —O(C₁₋₆)alkyl, —C(═O)—(C₁₋₆)alkyl, —C(═O)—O—(C₁₋₆)alkyl, cyano, —NH₂, —NH(C₁₋₆)alkyl, and —N((C₁₋₆)alkyl)₂.
R⁵-B: In one embodiment, R⁵ is selected from H, (C₁₋₆)alkyl, —O—(C₁₋₆)alkyl, —S—(C₁₋₆)alkyl, —NH₂, —NH(C₁₋₆)alkyl, —N((C₁₋₆)alkyl)₂, —NHC(═O)—(C₁₋₃)alkyl, —(C₁₋₆)alkyl-aryl or —(C₁₋₆)alkyl-Het, wherein the (C₁₋₆)alkyl, —(C₁₋₆)alkyl-aryl or —(C₁₋₆)alkyl-Het are optionally substituted with 1 to 4 substituents each independently selected from (C₁₋₆)alkyl, halo, —OH, —COOH, —O(C₁₋₆)alkyl, cyano, —NH₂, —NH(C₁₋₆)alkyl, and —N((C₁₋₆)alkyl)₂.
R⁵-C: In another embodiment, R⁵ is selected from H, (C₁₋₆)alkyl, —O—(C₁₋₆)alkyl, NH₂, —NHC(═O)—(C₁₋₃)alkyl, —(C₁₋₆)alkyl-aryl or —(C₁₋₆)alkyl-Het.
R⁵-D: In another embodiment, R⁵ is H, CH₃, —OCH₂CH₃, NH₂, —NHC(═O)—CH₃, —(CH₂)₂-aryl.
R⁵-E: In another embodiment, R⁵ is H or CH₃.
R⁵-F: In one embodiment, R⁵ is H.

### R⁶:

R⁶-A: In one embodiment, R⁶ is selected from (C₁₋₈)alkyl, (C₂₋₈)alkenyl, (C₂₋₈)alkynyl, (C₃₋₇)cycloalkyl, aryl and Het, wherein R⁶ is optionally substituted with 1 to 6 R²¹ substituents, wherein R²¹ is as defined herein.
R⁶-B: In one embodiment, R⁶ is selected from (C₁₋₆)alkyl, aryl and Het, wherein R⁶ is optionally substituted with 1 to 3 R²¹ substituents, wherein R²¹ is as defined herein.
R⁶-C: In one embodiment, R⁶ is selected from (C₁₋₆)alkyl, phenyl and Het, wherein R⁶ is optionally substituted with 1 to 3 R²¹ substituents, wherein Het is 5- or 6 membered aromatic heterocycle containing 1 or 2 N heteroatoms, and wherein R²¹ is as defined herein.
R⁶-D: In one embodiment, R⁶ is selected from (C₁₋₆)alkyl, wherein R⁶ is optionally substituted with 1 to 3 R²¹ substituents, wherein R²¹ is as defined herein.
R⁶-E: In still another embodiment, R⁶ is selected from:

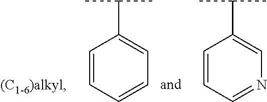

wherein R⁶ is optionally substituted with 1 to 3 R²¹ substituents, wherein R²¹ is as defined herein.
R⁶-F: In still another embodiment, R⁶ is selected from:

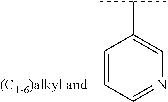

wherein R⁶ is optionally substituted with 1 to 3 R²¹ substituents, wherein R²¹ is as defined herein.

### R²¹:

R²¹-A: In another embodiment, R²¹ is selected from:
   a) halo, NH₂, NO₂, cyano, azido or oxo;
   b) R²¹⁰, OR²¹⁰, NR²¹⁰R²¹¹, SR²¹⁰, SOR²¹⁰, SO₂R²¹⁰, C(═O)R²¹⁰, C(′O)OR²¹⁰, C(═O)NR²¹⁰R²¹¹, NR²¹¹C(═O)R²¹², NR²¹¹C(═O)OR²¹², NR²¹¹C(═O)NR²¹¹R²¹², NR²¹¹SO₂NR²¹⁰, NR²¹¹SO₂NR²¹⁰R²¹² and SO₂NR²¹⁰R²¹¹;
      wherein R²¹⁰ is selected from H, (C₁₋₈)alkyl, (C₁₋₈)haloalkyl, (C₂₋₈)alkenyl, (C₂₋₈)alkynyl, (C₃₋₇)cycloalkyl, (C₅₋₇)cycloalkenyl, (C₃₋₇)spirocycloalkyl optionally containing 1 to 3 heteroatom selected from N, O and S, C(═O)R²¹¹, C(═O)OR²¹¹, aryl and Het, all of which can be optionally substituted with 1 to 6 substituents selected from OH, NH₂, cyano, oxo, NO₂, halo, R²¹², OR²¹¹, SR²¹¹, NR²¹¹R²¹², NR²¹¹C(═O)R²¹², NR²¹¹C(═O)OR²¹², NR²¹¹C(═O)NR²¹¹R²¹², NR²¹¹SO₂R²¹⁰, NR²¹¹SO₂NR²¹⁰R²¹², C(═O)R²¹¹, C(═O)OR²¹¹, C(═O)NR²¹¹R²¹²,
      and wherein R²″ is selected from H, (C₁₋₆)alkyl, and (C₃₋₇)cycloalkyl;
      and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl, Het, all of which being optionally substituted with 1 to 6 substituents selected from OH, NH₂, cyano, oxo, NO₂, halo, (C₁₋₆)alkyl, (C₃₋₇)cycloalkyl, (C₁₋₆)haloalkyl, O—(C₁₋₆)alkyl, S—(C₁₋₆)alkyl, NH(C₁₋₆)alkyl, N((C₁₋₆)alkyl)₂, aryl and Het,
      wherein aryl and Het can be optionally substituted with 1 to 3 substituents selected from OH, halo, (C₁₋₃)alkyl and —O(C₁₋₃)alkyl;
         or R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.
R²¹-B: In another embodiment, R²¹ is selected from:
   a) halo, NH₂, cyano, azido or oxo;
   b) R²¹⁰, OR²¹⁰, NR²¹⁰R²¹¹, C(═O)R²¹⁰, C(═O)OR²¹⁰, —C(═O)NR²¹⁰R²¹¹, NR²¹¹C(═O)R²¹², NR²¹¹C(═O)OR²¹², NR²¹¹C(═O)NR²¹¹R²¹² and NR²¹¹SO₂R²¹⁰;
      wherein R²¹⁰ is selected from H, (C₁₋₆)alkyl, (C₂₋₆)alkenyl, (C₃₋₆)cycloalkyl, (C₆₋₇)cycloalkenyl, (C₃₋₇)spirocycloalkyl, aryl and Het, all of which can be optionally substituted with 1 to 6 substituents selected from OH, NH₂, cyano, oxo, halo, R²¹², OR²¹¹, SR²¹¹, NR²¹¹R²¹², C(═O)R²¹¹, C(═O)OR²¹¹ and C(═O)NR²¹¹R²¹²,
      and wherein R²¹¹ is selected from H and (C₁₋₆)alkyl;
      and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, (C₃₋₇)cycloalkyl, O—(C₁₋₆)alkyl, S—(C₁₋₆)alkyl, NH(C₁₋₆)alkyl, N((C₁₋₆)alkyl)₂, aryl and Het, wherein aryl and Het can be optionally substituted with 1 to 3 substituents selected from OH, halo, (C₁₋₃)alkyl and —O(C₁₋₃)alkyl;
         or R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, OH, —O(C₁₋₆)alkyl and —NH₂.
R²¹-C: In another embodiment, R²¹ is selected from:
   a) Halo;
   b) R²¹⁰, OR²¹⁰, —C(═O)NR²¹⁰R²¹¹, NR²¹¹C(═O)R²¹² and NR²¹¹C(═O)OR²¹²;
      wherein R²¹⁰ is selected from H, (C₁₋₆)alkyl, (C₂₋₆)alkenyl, (C₃₋₆)cycloalkyl, (C₅₋₇)cycloalkenyl, (C₃₋₇)spirocycloalkyl, aryl and Het, all of which can be optionally substituted with 1 to 3 substituents selected from OH, NH₂, cyano, oxo, halo, R²¹², OR²¹¹, NR²¹¹R²¹², C(═O)R²¹¹, C(═O)OR²¹¹ and C(═O)NR²¹¹R²¹²,
      and wherein R²¹¹ is selected from H and (C₁₋₆)alkyl;
      and wherein R²¹² is selected from H, (C₁₋₆)alkyl, (C₂₋₆)alkenyl, (C₂₋₆)alkynyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, O—(C₁₋₆)alkyl, aryl and Het;
         or R²¹⁰ and R²¹¹, or R²¹¹ and R²¹² together with the N to which they are attached, are linked to form a 4- to 7-membered heterocycle optionally further containing 1 to 2 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with (C₁₋₆)alkyl, oxo or —O(C₁₋₆)alkyl.
R²¹-D: In another embodiment, R²¹ is selected from:
   a) Halo;
   b) R²¹⁰, OR²¹⁰, —C(═O)NR²¹⁰R²¹¹ and NR²¹¹C(═O)R²¹²;
      wherein R²¹⁰ is selected from H, (C₁₋₆)alkyl, (C₂₋₆)alkenyl, (C₃₋₆)cycloalkyl, (C₅₋₇)cycloalkenyl, (C₃₋₇)spirocycloalkyl, aryl and Het, all of which can be optionally substituted with 1 to 3 substituents selected from OH, NH₂, cyano, halo, R²¹², OR²¹¹ and C(═O)NR²¹¹R²¹²,
      and wherein R²¹¹ is selected from H and (C₁₋₆)alkyl;
      and wherein R²¹² is selected from H, (C₁₋₄)alkyl, (C₂₋₄)alkenyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, O—(C₁₋₆)alkyl, aryl and Het;
         wherein Het is a 5 to 7 membered heterocycle having 1 to 2 N atoms and 0 to 2 heteroatoms each independently selected from O and S
            or R²¹⁰ and R²¹¹, or R²¹¹ and R²¹² together with the N to which they are attached, are linked to form a 4- to 7-membered heterocycle optionally further containing 1 to 2 heteroatoms each independently selected from N, O and S, wherein each S heteroatom may, independently and where possible, exist in an oxidized state such that it is further bonded to one or two oxygen atoms to form the groups SO or SO₂.
R²¹-E: In another embodiment, R²¹ is selected from F, Cl, Br; OH, NH₂, (C₁₋₃)alkyl, (C₂₋₄)alkenyl, aryl or Het, wherein (C₁₋₃)alkyl, (C₂₋₄)alkenyl, aryl and Het are optionally substituted with halo, OH, (C₁₋₃)alkyl, (C₃₋₆)cycloalkyl, O—(C₁₋₃)alkyl, C(═O)N((C₁₋₃)alkyl)₂, NHC(═O)(C₁₋₃)alkyl, NHC(═O)NH(C₁₋₃)alkyl, phenyl or Het wherein Het is a 5 to 7 membered heterocycle having 1 to 2 N atoms and 0 to 2 heteroatoms each independently selected from O and S.
R²¹-F: In another embodiment, R²¹ is selected from F, Cl, Br, OH, (C₁₋₃)alkyl, phenyl or Het, wherein (C₁₋₃)alkyl, phenyl and Het are optionally substituted with halo, OH, (C₁₋₃)alkyl, O—(C₁₋₃)alkyl, phenyl or Het, wherein Het is a 5 to 7 membered heterocycle having 1 to 2 N atoms and 0 to 2 heteroatoms each independently selected from O and S.
R²¹-G: In another embodiment, R²¹ is selected from:
   a) halo, cyano, azido or oxo;
   b) R²¹⁰, OR²¹⁰, C(═O)R²¹⁰, C(═O)OR²¹⁰, —C(═O)NR²¹⁰R²¹¹, NR²¹¹C(═O)R²¹², NR²¹¹C(═O)OR²¹², NR²¹¹C(═O)NR²¹¹R²¹² and NR²¹¹SO₂R²¹⁰;
      wherein R²¹⁰ is selected from H (with the proviso that when R⁶ is (C₁₋₈)alkyl and R²¹ is OR²¹⁰, then R²¹⁰ cannot be H), (C₁₋₆)alkyl, (C₂₋₆)alkenyl, (C₃₋₆)cycloalkyl, (C₅₋₇)cycloalkenyl, (C₃₋₇)spirocycloalkyl, aryl and Het, all of which can be optionally substituted with 1 to 6 substituents selected from OH, NH₂, cyano, oxo, halo, R²¹², OR²¹¹, SR²¹¹, NR²¹¹R²¹², C(═O)R²¹¹, C(═O)OR²¹¹ and C(═O)NR²¹¹R²¹²,
      and wherein R²¹¹ is selected from H and (C₁₋₆)alkyl;
      and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, (C₃₋₇)cycloalkyl, O—(C₁₋₆)alkyl, S—(C₁₋₆)alkyl, NH(C₁₋₆)alkyl, N((C₁₋₆)alkyl)₂, aryl and Het (with the proviso that Het cannot be triazole or tetrazole); or
      R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, OH, —O(C₁₋₆)alkyl and —NH₂.
R²¹-H: In another embodiment, R²¹ is selected from:
   a) halo;
   b) R²¹⁰, OR²¹⁰, C(═O)NR²¹⁰R²¹¹, NR²¹¹C(═O)R²¹² and NR²¹¹C(═O)OR²¹²;
      wherein R²¹⁰ is selected from H (with the proviso that when R⁶ is (C₁₋₈)alkyl and R²¹ is OR²¹⁰, then R²¹⁰ cannot be H), (C₁₋₆)alkyl, (C₂₋₆)alkenyl, (C₃₋₆)cycloalkyl, (C₅₋₇)cycloalkenyl, (C₃₋₇)spirocycloalkyl, aryl and Het, all of which can be optionally substituted with 1 to 3 substituents selected from OH, NH₂, cyano, oxo, halo, R²¹², OR²¹¹, NR²¹¹R²¹², C(═O)R²¹¹, C(═O)OR²¹¹, and C(═O)NR²¹¹R²¹²,
      and wherein R²¹¹ is selected from H and (C₁₋₆)alkyl;
      and wherein R²¹² is selected from H, (C₁₋₆)alkyl, (C₂₋₆)alkenyl, (C₂₋₆)alkynyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, O—(C₁₋₆)alkyl, aryl and Het (with the proviso that Het cannot be triazole or tetrazole);
         or R²¹⁰ and R²¹¹, or R²¹¹ and R²¹² together with the N to which they are attached, are linked to form a 4- to 7-membered heterocycle optionally further containing 1 to 2 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with (C₁₋₆)alkyl, oxo or —O(C₁₋₆)alkyl.
R²¹-I: In another embodiment, R²¹ is selected from:
   a) Halo;
      b) R²¹⁰, OR²¹⁰, —C(═O)NR²¹⁰R²¹¹ and NR²¹¹C(═O)R²¹²;
      wherein R²¹⁰ is selected from H (with the proviso that when R⁶ is (C₁₋₈)alkyl and R²¹ is OR²¹⁰, then R²¹⁰ cannot be H), (C₁₋₆)alkyl, (C₂₋₆)alkenyl, (C₃₋₆)cycloalkyl, (C₅₋₇)cycloalkenyl, (C₃₋₇)spirocycloalkyl, aryl and Het, all of which can be optionally substituted with 1 to 3 substituents selected from OH, NH₂, cyano, halo, R²¹², OR²¹¹ and C(═O)NR²¹¹R²¹²,
      and wherein R²¹¹ is selected from H and (C₁₋₆)alkyl;
      and wherein R²¹² is selected from H, (C₁₋₄)alkyl, (C₂₋₄)alkenyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, O—(C₁₋₆)alkyl, aryl and Het;
         wherein Het is a 5 to 7 membered heterocycle having 1 to 2 N atoms and 0 to 2 heteroatoms each independently selected from O and S;
            or R²¹⁰ and R²¹¹, or R²¹¹ and R²¹² together with the N to which they are attached, are linked to form a 4- to 7-membered heterocycle optionally further containing 1 to 2 heteroatoms each independently selected from N, O and S, wherein each S heteroatom may, independently and where possible, exist in an oxidized state such that it is further bonded to one or two oxygen atoms to form the groups SO or SO₂.

Examples of preferred subgeneric embodiments of the present invention are set forth in the following table, wherein each substituent group of each embodiment is defined according to the definitions set forth above:

| Embodiment | X | R² | R²⁰ | R³ | R⁵ | R⁶ | R²¹ |
|---|---|---|---|---|---|---|---|
| E-1 | X-C | R²-A | R²⁰-E | R³-E | R⁵-E | R⁶-C | R²¹-F |
| E-2 | X-C | R²-A | R²⁰-E | R³-D | R⁵-D | R⁶-C | R²¹-E |
| E-3 | X-B | R²-B | R²⁰-E | R³-F | R⁵-F | R⁶-E | R²¹-F |
| E-4 | X-C | R²-C | R²⁰-E | R³-E | R⁵-E | R⁶-D | R²¹-I |
| E-5 | X-B | R²-C | R²⁰-D | R³-D | R⁵-D | R⁶-F | R²¹-D |
| E-6 | X-B | R²-C | R²⁰-D | R³-F | R⁵-F | R⁶-C | R²¹-F |
| E-7 | X-C | R²-C | R²⁰-C | R³-F | R⁵-B | R⁶-E | R²¹-F |
| E-8 | X-B | R²-D | R²⁰-C | R³-F | R⁵-F | R⁶-E | R²¹-E |
| E-9 | X-B | R²-E | R²⁰-F | R³-C | R⁵-C | R⁶-E | R²¹-D |
| E-10 | X-C | R²-E | R²⁰-D | R³-E | R⁵-E | R⁶-F | R²¹-E |
| E-11 | X-B | R²-E | R²⁰-B | R³-B | R⁵-C | R⁶-B | R²¹-D |
| E-12 | X-C | R²-F | R²⁰-F | R³-F | R⁵-F | R⁶-F | R²¹-F |
| E-13 | X-C | R²-F | R²⁰-E | R³-F | R⁵-F | R⁶-C | R²¹-B |
| E-14 | X-C | R²-F | R²⁰-D | R³-D | R⁵-D | R⁶-A | R²¹-F |
| E-15 | X-C | R²-G | R²⁰-E | R³-F | R⁵-F | R⁶-E | R²¹-I |
| E-16 | X-C | R²-G | R²⁰-E | R³-E | R⁵-E | R⁶-E | R²¹-G |
| E-17 | X-C | R²-G | R²⁰-F | R³-F | R⁵-E | R⁶-D | R²¹-H |
| E-18 | X-C | R²-H | R²⁰-F | R³-F | R⁵-F | R⁶-F | R²¹-F |
| E-19 | X-C | R²-H | R²⁰-F | R³-F | R⁵-F | R⁶-E | R²¹-H |
| E-20 | X-C | R²-H | R²⁰-F | R³-D | R⁵-D | R⁶-C | R²¹-F |
| E-21 | X-B | R²-H | R²⁰-D | R³-E | R⁵-E | R⁶-C | R²¹-F |
| E-22 | X-C | R²-H | R²⁰-D | R³-E | R⁵-E | R⁶-E | R²¹-D |
| E-23 | X-C | R²-H | R²⁰-E | R³-D | R⁵-D | R⁶-B | R²¹-C |
| E-24 | X-B | R²-H | R²⁰-C | R³-F | R⁵-F | R⁶-E | R²¹-C |
| E-25 | X-A | R²-H | R²⁰-F | R³-E | R⁵-E | R⁶-D | R²¹-I |

Examples of most preferred compounds according to this invention are each single compound listed in the following Tables 1 to 9.

Unless specifically indicated, throughout the specification and the appended claims, a given chemical formula or name shall encompass tautomers and all stereo, optical and geometrical isomers (e.g. enantiomers, diastereomers, E/Z isomers, atropisomers) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

### Pharmaceutical Composition

Suitable preparations for administering the compounds of formula (I) will be apparent to those with ordinary skill in the art and include for example tablets, pills, capsules, suppositories, lozenges, troches, solutions, syrups, elixirs, sachets, injectables, inhalatives and powders etc. The content of the pharmaceutically active compound(s) should be in the range from 0.05 to 90 wt.-%, preferably 0.1 to 50 wt.-% of the composition as a whole.

Suitable tablets may be obtained, for example, by mixing one or more compounds according to formula (I) with known excipients, for example inert diluents, carriers, disintegrants, adjuvants, surfactants, binders and/or lubricants. The tablets may also consist of several layers.

The dose range of the compounds of general formula (applicable per day is usually from 0.01 to 200 mg/kg of body weight, preferably from 0.1 to 100 mg/kg of body weight, more preferably from 0.1 to 50 mg/kg of body weight. Each dosage unit may conveniently contain from 5% to 95% active compound (w/w). Preferably such preparations contain from 20% to 80% active compound.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

### Combination Therapy

Combination therapy is contemplated wherein a compound according to the invention, or a pharmaceutically acceptable salt thereof, is co-administered with at least one additional antiviral agent. The additional agents may be combined with compounds of this invention to create a single dosage form. Alternatively these additional agents may be separately administered, concurrently or sequentially, as part of a multiple dosage form.

When the pharmaceutical composition of this invention comprises a combination of a compound according to the invention, or a pharmaceutically acceptable salt thereof, and one or more additional antiviral agent, both the compound and the additional agent should be present at dosage levels of between about 10 to 100%, and more preferably between about 10 and 80% of the dosage normally administered in a monotherapy regimen. In the case of a synergistic interaction between the compound of the invention and the additional antiviral agent or agents, the dosage of any or all of the active agents in the combination may be reduced compared to the dosage normally administered in a monotherapy regimen.

Antiviral agents contemplated for use in such combination therapy include agents (compounds or biologicals) that are effective to inhibit the formation and/or replication of a virus in a human being, including but not limited to agents that interfere with either host or viral mechanisms necessary for the formation and/or replication of a virus in a human being. Such agents can be selected from another anti-HCV agent, an HIV inhibitor, an HAV inhibitor, and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, other inhibitors of HCV polymerase, inhibitors of another target in the HCV life cycle and other anti-HCV agents, including but not limited to ribavirin, amantadine, levovirin and viramidine.

Immunomodulatory agents include those agents (compounds or biologicals) that are effective to enhance or potentiate the immune system response in a human being. Immunomodulatory agents include, but are not limited to, TLRs (Toll-like receptor antagonists), such as ANA773(TLR-7) and IMO-2125(TLR-9), inosine monophosphate dehydrogenase inhibitors such as VX-497 (merimepodib, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons. In one preferred aspect, the other anti-HCV agent is an interferon. Preferably, the interferon is selected from the group consisting of interferon alpha 2B, pegylated interferon alpha, consensus interferon, interferon alpha 2A and lymphoblastoid interferon. In one preferred aspect, the composition comprises a compound of the invention, an interferon and ribavirin.

Inhibitors of HCV NS3 protease include agents (compounds or biologicals) that are effective to inhibit the function of HCV NS3 protease in a human being. Inhibitors of HCV NS3 protease include, for example, the candidates BI1335 (Boehringer Ingelheim), VX-813 and VX-950 (Vertex), SCH-503034 and SCH-900518 (Schering-Plough), ABT-450 (Abbott/Enanta), VBY376 (Virobay), PHY1766 (Phenomix), ITMN-191 (InterMune/Roche), TMC 435350 (Medivir/Tibotec) and MK7009 (Merck).

Inhibitors of HCV polymerase include agents (compounds or biologicals) that are effective to inhibit the function of an HCV polymerase. Such inhibitors include, but are not limited to, non-nucleoside and nucleoside inhibitors of NS4A, NS5A, NS5B polymerase. Examples of inhibitors of HCV polymerase include but are not limited to those compounds described in: WO 03/007945, WO 03/010140, WO 03/010141, U.S. Pat. No. 6,448,281, WO 02/04425, WO 2008/019477, WO 2007/087717, WO 2006/007693, WO 2005/080388, WO 2004/099241, WO 2004/065367, WO 2004/064925 (all by Boehringer Ingelheim), (all of which are herein incorporated by reference) and the candidates R-7128 (Roche/Pharmasset), PSI-7851 (Pharmasset), IDX184 (Idenix), VX-759, VX-916 and VX-222 (Vertex), MK-3281 (Merck), ABT-333 and ABT-072 (Abbott), ANA598 (Anadys) and PF868554 (Pfizer).

The term “inhibitor of another target in the HCV life cycle” as used herein means an agent (compound or biological) that is effective to inhibit the formation and/or replication of HCV in a human being other than by inhibiting the function HCV polymerase. This includes agents that interfere with either host or HCV viral targets necessary for the HCV life cycle or agents which specifically inhibit in HCV cell culture assays through an undefined or incompletely defined mechanism. Inhibitors of another target in the HCV life cycle include, for example, agents that inhibit viral targets such as Core, E1, E2, p7, NS2/3 protease, NS3 helicase, internal ribosome entry site (IRES), HCV entry and HCV assembly or host targets such as cyclophilin B, phosphatidylinositol 4-kinase IIIα, CD81, SR-B1, Claudin 1, VAP-A, VAP-B. Specific examples of inhibitors of another target in the HCV life cycle include ISIS-14803 (ISIS Pharmaceuticals), GS9190 (Gilead), GS9132 (Gilead), A-831 (AstraZeneca), NM-811 (Novartis), BMS-790052 (BMS) and DEBIO-025 (Debio Pharma).

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

HIV inhibitors include agents (compounds or biologicals) that are effective to inhibit the formation and/or replication of HIV. This includes but is not limited to agents that interfere with either host or viral mechanisms necessary for the formation and/or replication of HIV in a human being. HIV inhibitors include, but are not limited to:
NRTIs (nucleoside or nucleotide reverse transcriptase inhibitors) including but not limited to zidovudine (AZT), didanosine (ddI), zalcitabine (ddC), stavudine (d4T), lamivudine (3TC), emtricitabine, abacavir succinate, elvucitabine, adefovir dipivoxil, lobucavir (BMS-180194) lodenosine (FddA) and tenofovir including tenofovir disoproxil and tenofovir disoproxil fumarate salt, COMBIVIR™ (contains 3TC and AZT), TRIZIVIR™ (contains abacavir, 3TC and AZT), TRUVADA™ (contains tenofovir and emtricitabine), EPZICOM™ (contains abacavir and 3TC),
NNRTIs (non-nucleoside reverse transcriptase inhibitors) including but not limited to nevirapine, delaviradine, efavirenz, etravirine and rilpivirine,
protease inhibitors including but not limited to ritonavir, tipranavir, saquinavir, nelfinavir, indinavir, amprenavir, fosamprenavir, atazanavir, lopinavir, darunavir, lasinavir, brecanavir, VX-385 and TMC-114,
entry inhibitors including but not limited to
   CCR5 antagonists (including but not limited to maraviroc, vicriviroc, INCB9471 and TAK-652),
   CXCR4 antagonists (including but not limited to AMD-11070),
   fusion inhibitors (including but not limited to enfuvirtide (T-20), TR1-1144 and TR1-999) and
   others (including but not limited to BMS-488043),
integrase inhibitors (including but not limited to raltegravir (MK-0518), BMS-707035 and elvitegravir (GS 9137)),
TAT inhibitors,
maturation inhibitors (including but not limited to berivimat (PA-457)),
immunomodulating agents (including but not limited to levamisole), and
other antiviral agents including hydroxyurea, ribavirin, IL-2, IL-12 and pensafuside.

HAV inhibitors include agents (compounds or biologicals) that are effective to inhibit the formation and/or replication of HAV. This includes but is not limited to agents that interfere with either host or viral mechanisms necessary for the formation and/or replication of HAV in a human being. HAV inhibitors include but are not limited to Hepatitis A vaccines.

HBV inhibitors include agents (compounds or biologicals) that are effective to inhibit the formation and/or replication of HBV in a human being. This includes but is not limited to agents that interfere with either host or viral mechanisms necessary for the formation and/or replication of HBV in a human being. HBV inhibitors include, but are not limited to, agents that inhibit the HBV viral DNA polymerase and HBV vaccines.

Therefore, according to one embodiment, the pharmaceutical composition of this invention additionally comprises a therapeutically effective amount of one or more antiviral agents.

A further embodiment provides the pharmaceutical composition of this invention wherein the one or more antiviral agent comprises at least one other anti-HCV agent.

According to a more specific embodiment of the pharmaceutical composition of this invention, at least one other anti-HCV agent comprises at least one immunomodulatory agent.

According to another more specific embodiment of the pharmaceutical composition of this invention, at least one other anti-HCV agent comprises at least one other inhibitor of HCV polymerase.

According to yet another more specific embodiment of the pharmaceutical composition of this invention, at least one other anti-HCV agent comprises at least one inhibitor of HCV NS3 protease.

According to still another more specific embodiment of the pharmaceutical composition of this invention, the at least one other anti-HCV agent comprises at least one inhibitor of another target in the HCV life cycle.

### EXAMPLES

Other features of the present invention will become apparent from the following non-limiting examples which illustrate, by way of example, the principles of the invention. As is well known to a person skilled in the art, reactions are performed in an inert atmosphere (including but not limited to nitrogen or Ar) where necessary to protect reaction components from air or moisture. Preparation of compounds of the invention can involve the protection and deprotection of various chemical groups. The need for protection and deprotection, and the selection of appropriate protecting groups and reaction conditions can be readily determined by one skilled in the art. The chemistry of protecting groups can be found, for example, in Greene, “Protective Groups in Organic Chemistry”, John Wiley & Sons, New York (1981), and more recent editions thereof, herein incorporated by reference. Temperatures are given in degrees Celsius (° C.). Solution percentages and ratios express a volume to volume relationship, unless stated otherwise. Flash chromatography is carried out on silica gel (SiO₂) according to the procedure of W. C. Still et al., J. Org. Chem., (1978), 43, 2923. Mass spectral analyses are recorded using electrospray mass spectrometry. Purification on a combiflash is performed using an Isco Combiflash (column cartridge SiO₂). Unless otherwise specified, preparative HPLC is the purification method. Preparative HPLC is carried out under standard conditions either using a XBridge™ Prep C18 OBD 5 μM reverse phase column, 19×50 mm and gradient employing MeOH and 10 mM aqueous ammonium carbonate; or SunFire™ Prep C18 OBD 5 μM reverse phase column, 19×50 mm and gradient employing MeOH and 10 mM aqueous ammonium formate; or using a SunFire™ Prep C18 OBD 5 μM reverse phase column, 19×50 mm and gradient employing 0.1% TFA/acetonitrile and 0.1% TFA/water as solvents. Compounds are isolated as TFA salts when applicable. Analytical HPLC is carried out under standard conditions either using a Waters Sunfire™ C18 3.5 μM reverse phase column, 4.8×50 mm i.d., 120 Å at 220 nM, eluting in a linear gradient with MeOH and 10 mM aqueous ammonium formate or using a XBridge™ C18 3.5 μM reverse phase column, 4.8×50 mm i.d., 120 Å at 220 nM, eluting in a linear gradient with MeOH and 10 mM aqueous ammonium carbonate; or a Combiscreen™ ODS-AQ C18 reverse phase column, YMC, 50×4.6 mm i.d., 5 μM, 120 Å at 220 nM, elution with a linear gradient employing 0.1% TFA in H₂O and 0.1% TFA in MeCN.

Analytical HPLC, which is carried out under standard conditions using a HSS™ C18 reverse phase column, 30×2.1 mm i.d., 1.8 μM, elution with a linear gradient as described in the following table (Solvent A is 0.1% TFA in H₂O; solvent B is 0.1% TFA in MeCN):

| Time (min) | Flow (mL/min) | Solvent A (%) | Solvent B (%) |
|---|---|---|---|
| 0 | 1.0 | 98 | 2 |
| 5.2 | 1.0 | 0 | 100 |
| 5.8 | 1.0 | 0 | 100 |

Abbreviations or symbols used herein include:

Ac: acetyl; AcCI: acetyl chloride; AcOH: acetic acid; Ac₂O: acetic anhydride; 9-BBN: 9-borabicyclo[3.3.1]nonane; BINAP: 2,2′-bis(diphphenylphosphino)-1,1′-binaphthyl; Bn: benzyl (phenylmethyl); BOC or Boc: tert-butyloxycarbonyl; BOP: benzotriazol-1-yloxy-tris(dimethylamino)phosphonium; hexafluorophosphate; Bu: butyl; CDI: 1,1-carbonyldiimidazole; Cy: cyclohexyl; DBU: 1,8-diazabicyclo[5.4.0]undec-7-ene; DCE: dichloroethane; DCM: dichloromethane; DEAD: diethylazodicarboxylate; (DHQ)₂PHAL: hydroquinidine-1,4-phthalazinediyldiether; DIAD: diisopropylazodicarboxylate; DiBAI-H: di-1-butylaluminum hydride; DIPEA: diisopropylethylamine; DMA or DMAc: dimethylacetamide; DMAP: 4-dimethylaminopyridine; DMF: N,N-dimethylformamide; DMSO: dimethylsulfoxide; BnOH: benzyl alcohol; DPPA: diphenylphosphoryl azide; dppf: 1,1′-diphenylphosphinylferrocene; EC₅₀: 50% effective concentration; Et: ethyl; Et₂O: diethyl ether; EtOAc: ethyl acetate; EtOH: ethanol; HATU: O-(7-azabenzotriazol-1-yl)-N,N,N′,N′-tetramethyluronium hexafluorophosphate; Hex: hexane; HPLC: high performance liquid chromatography; IC₅₀: 50% inhibitory concentration; ⁱPr or i-Pr: 1-methylethyl (iso-propyl); i-PrOH: isopropanol; (i-Pr)₂O: diisopropylether; LC-MS: liquid chromatography-mass spectrometry; LiHMDS: lithium hexamethyl disilazide; mCPBA: meta-chloroperbenzoic acid; Me: methyl; MeCN: acetonitrile; MeI: iodomethane; MeOH: methanol; MS: mass spectrometry (ES: electrospray); MsCl: Methanesulfonyl chloride; NaHB(OAc)₃: sodium triacetoxyborohydride; NaHMDS: sodium-1,1,1,3,3,3-hexamethyldisilazane; NMO: N-morpholine oxide; NMP: N-methylpyrrolidinone; Ph: phenyl; PhN(Tf)₂: N-phenyltrifluoromethanesulfonimide; PPh₃: triphenylphosphine; Pr: n-propyl; Prep: preparative; Psi: pounds per square inch; Rpm: rotations per minute; RT: room temperature (approximately 18° C. to 25° C.); S_{N}Ar: nucleophilic aromatic substitution; t-BME: tert-butlymethylether; tert-butyl or t-butyl: 1,1-dimethylethyl; tert-BuOH or t-BuOH: tert-butanol; TBAB: tetrabutylammonium bromide; TBAF: tetrabutylammonium fluoride; TBTU: 2-(1H-benzotriazole-1-yl)-1,1,3,3-tetramethyl uronium tetrafluoroborate; TEA or Et₃N: triethylamine; TEMPO: 2,2,6,6-tetramethyl-1-piperidinyloxy free radical; TFA: trifluoroacetic acid; THF: tetrahydrofuran; TLC: thin layer chromatography; TMS: trimethylsilyl; TMSCN: trimethylsilylcyanide; UHP: urea hydroperoxide; HPLC: ultra performance liquid chromatography.

### Example 1A

### Preparation of Compound 1001

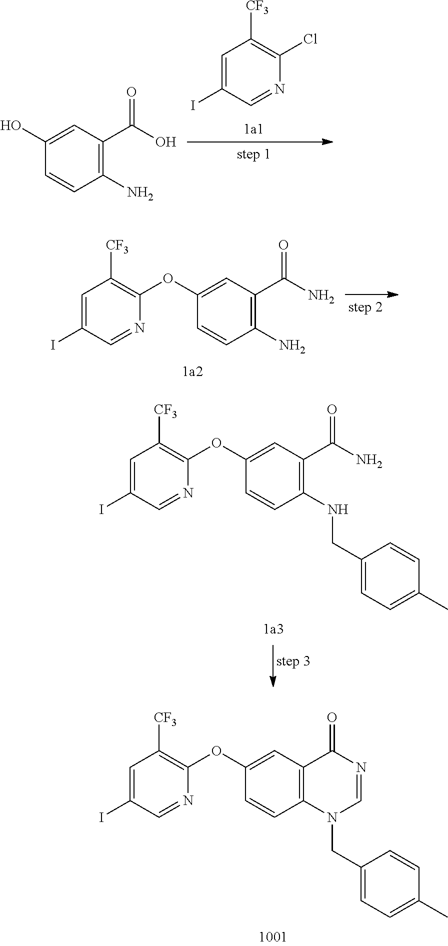

Intermediate 1a1 is prepared as described in WO 2008/01947, herein incorporated by reference.

### Step 1:

2-Amino-5-hydroxybenzoic acid (0.50 g, 3.27 mmol) is combined with pyridyl chloride 1a1 (1.00 g, 3.25 mmol) and Cs₂CO₃ (2.20 g, 6.77 mmol) in DMSO (5 mL). The mixture is heated to 80° C. and is stirred for 2 h. The mixture is allowed to cool to RT before HATU (1.33 g, 3.50 mmol), (NH₄)HCO₃ (0.63 g, 8.00 mmol) and TEA (1.25 mL, 9.00 mmol) are added. The mixture stirs 1 h before being diluted in EtOAc and washed with sat. aq. NaHCO₃ (2×) and brine. The organic phase is dried over MgSO₄, filtered and concentrated. The crude material is purified by flash chromatography (6:4 to 4:6 Hex/EtOAc) to afford intermediate 1a2.

### Step 2:

Reductive amination methodology described in the following publication: Abdel-Magid, A. F.; Carson, K. G.; Harris, B. D.; Maryanoff, C. A.; Shah, R. D. J. Org. Chem. 1996, 61, 3849, herein incorporated by reference.

Aniline 1a2 (380 mg, 0.90 mmol) is added to DCE (10 mL) followed by 4-methylbenzaldehyde (0.11 mL, 0.95 mmol), AcOH (86 μL, 1.5 mmol) and NaHB(OAc)₃ (300 mg, 1.40 mmol). The mixture is stirred for 20 h at RT. The mixture is then diluted in EtOAc and washed with sat. aq. NaHCO₃ and brine. The organic phase is dried over MgSO₄, filtered and concentrated. The crude material is purified by flash chromatography (7:3 to 6:4 Hex/EtOAc) to afford intermediate 1a3.

### Step 3:

Intermediate 1a3 (62.3 mg, 0.12 mmol) is added to (MeO)₃CH (0.50 mL) followed by TFA (25 μL). The mixture is stirred for 2 h at RT. The mixture is injected directly onto a prep. HPLC to isolate compound 1001.

### Example 2A

### Preparation of Compound 1002

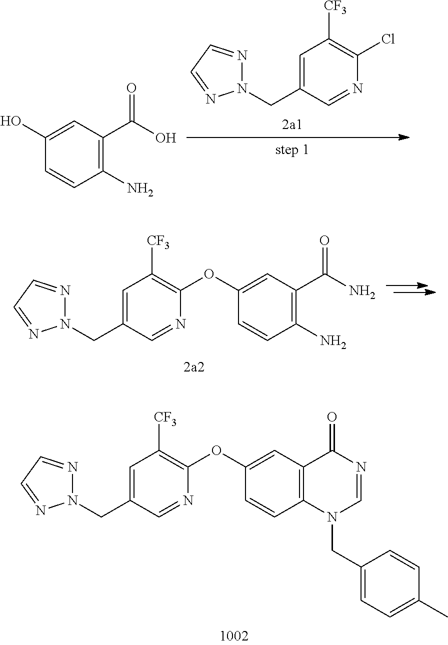

Intermediate 2a1 is prepared as described in WO 2009/018656, herein incorporated by reference. Compound 1002 is prepared in an analogous fashion to compound 1001.

### Example 3A

### Preparation of Compound 1003

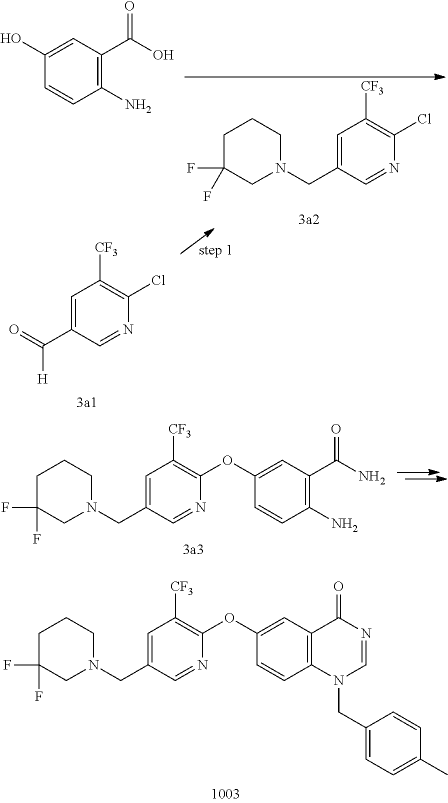

Aldehyde 3a1 is prepared as described in WO 2009/018656, herein incorporated by reference.

### Step 1:

To aldehyde 3a1 (2.0 g, 9.5 mmol) in 45 mL of DCE is added 3,3-difluoropiperidine-HCl salt (1.6 g, 10.5 mmol) and Na(AcO)₃BH (2.8 g, 13.4 mmol). The mixture is stirred overnight at RT. The mixture is diluted with EtOAc (300 mL) and washed with water (100 mL) and brine (100 mL). The organic phase is then dried over MgSO₄, filtered and concentrated. The residue is purified by flash chromatography (Combiflash, 15-40% EtOAc/Hex) to afford pyridyl chloride 3a2.

Pyridyl chloride 3a2 is coupled to 2-amino-5-hydroxybenzoic acid then elaborated to 1003 as shown in example 1A.

### Example 4A

### Preparation of Intermediate 4A4

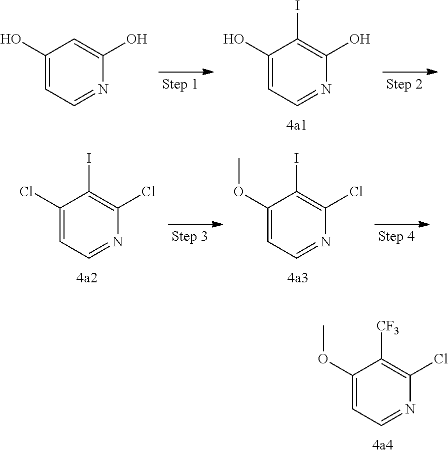

### Step 1:

A mixture of pyridine-2,4-diol (97 g, 873 mmol), K₂CO₃ (121 g, 873 mmol) and water (1 L) is heated to 100° C. Iodine (222 g, 873 mmol) is added portionwise. When the iodine is consumed, the reaction is quenched with KHSO₄ (0.873 L, 873 mmol). The solid is collected by filtration and washed with Et₂O/MeCN (1:1) (2×), dried on a stream of air overnight and co-evaporated with toluene (3×) to yield iodide 4a1.

### Step 2:

A mixture of 4a1 (207 g, 873 mmol), DMF (0.7 mL, 8.7 mmol) and POCl₃ (1 L, 11 mol) is heated at 90° C. overnight with stirring. The solution is concentrated, quenched with sat. aq. NaHCO₃, extracted with DCM, dried over MgSO₄, filtered and concentrated under vacuum. Co-evaporation with toluene affords dichloride 4a2.

### Step 3:

A mixture of 4a2 (201 g, 734 mmol) and NaOMe (51.5 g, 954 mmol) in MeOH (2 L) is stirred at RT overnight then heated at 45° C. for 3 h. The reaction is diluted with EtOAc, washed with water, brine, dried over MgSO₄, filtered and concentrated under vacuum. Upon standing crystals are formed. These are collected, washed with a small amount of (i-Pr)₂O followed by heptane and dried on a stream of air to afford 4a3.

### Step 4:

A solution of 4a3 (33.1 g, 123 mmol), KF (7.14 g, 123 mmol) and CuI (23.39 g, 123 mmol) in NMP (165 mL) is heated at 80° C. Methyl-2-chloro-2,2-difluoroacetate (51.8 mL, 491 mmol) is added and the mixture is heated 1.5 h at 120° C. The reaction mixture is poured into brine and Et₂O is added. The solid is filtered and the layers separated. The organic layer is washed with water and the aqueous layer is extracted with Et₂O. The combined organic layers are washed with brine, dried over Na₂SO₄, filtered and concentrated in vacuum followed by Kugelrohr distillation to afford the trifluoromethyl derivative 4a4.

### Example 4B

### (Synthetic Method A): Preparation of Compound 1004

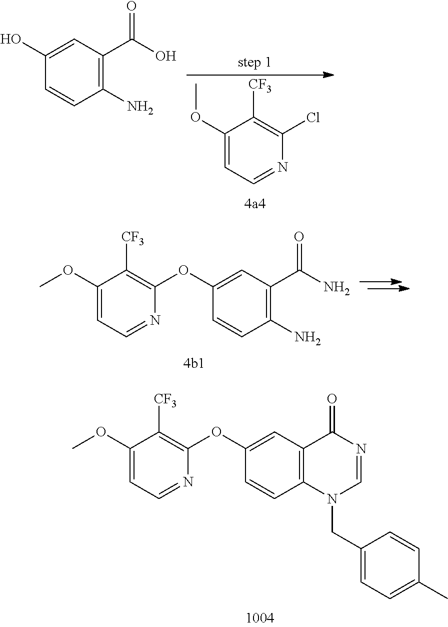

Pyridyl chloride 4a4 is added to 2-amino-5-hydroxybenzoic acid then elaborated to 1004 as shown in example 1A.

### Example 5A

### Preparation of Intermediate 5A1

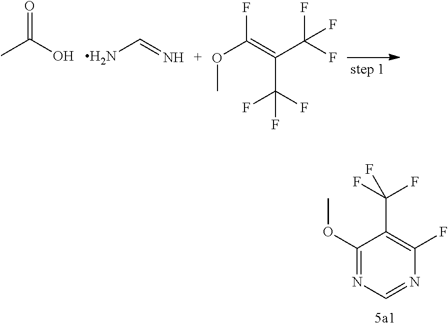

### Step 1:

Formamidine acetate (15.3 g, 147 mmol) and 1,3,3,3-tetrafluoro-1-methoxy-2-(trifluoromethyl)prop-1-ene (20.8 g, 98 mmol) are mixed in DCM (100 mL) and water (100 mL) at 0° C. The reaction mixture is stirred vigourously and NaOH (6 M, 70.7 mL) solution is added dropwise over 30 min and stirred for 35 min. The layers are then separated and the organic phase is concentrated under vacuum. The residue is purified by kugelrohr distillation (80° C., 3 mm Hg), then distilled with a vigreux column to afford the desired product 5a1.

### Example 5B

### Preparation of Compound 5B3

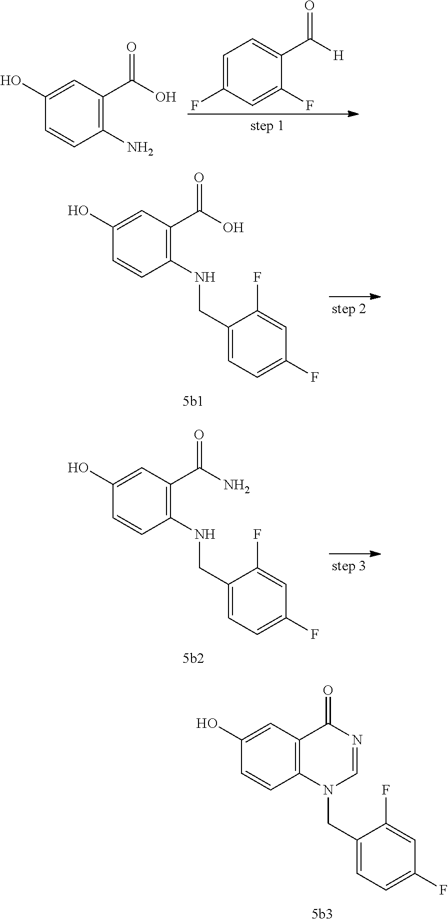

### Step 1:

2-amino-5-hydroxybenzoic acid (21.22 g, 139 mmol) is suspended in AcOH (140 mL) under Ar. To this mixture is added 2,4-difluorobenzaldehyde (23.39 g, 165 mmol) and the mixture is stirred 30 min at RT. To this mixture is added NaCNBH₃ (17.78 g, 278 mmol) in portions while cooling with a water bath and the reaction mixture is stirred overnight at RT. The reaction is quenched with H₂O (500 mL), the solid is collected by filtration and washed with H₂O. The solid is dried under vacuum at 50° C. to give compound 5b1.

### Step 2:

Compound 5b1 (22.80 g, 81 mmol) is dissolved in DMF (200 mL) under Ar and HATU (37.25 g, 97 mmol), (NH₄)HCO₃ (12.91 g, 162 mmol) and Et₃N (34.14 mL, 243 mmol) are added to this solution. The reaction mixture is stirred overnight at RT. DMF is removed under high vacuum and the residue is diluted with H₂O (400 mL), and extracted with EtOAc (3×). The combined EtOAc extracts are washed with sat. aq. NaHCO₃ and brine, dried over Na₂SO₄, filtered and concentrated under vacuum to dryness. The crude product is triturated in Hex/EtOAc (1:1) then in DCM and collected by filtration to afford 5b2.

### Step 3:

Compound 5b2 (19.58 g, 70 mmol) is suspended in (MeO)₃CH (150 mL) under Ar and TFA (7.5 mL) is added. The mixture is stirred 60 min at RT. Solvent is removed under vacuum. The residue is treated with H₂O (˜250 mL) and vigorously stirred. The solid is collected by filtration, rinsed with H₂O and hexanes and dried under high vacuum at <50° C. to give the desired product 5b3.

### Example 5C

### (Synthetic Method B): Preparation of Compound 1006

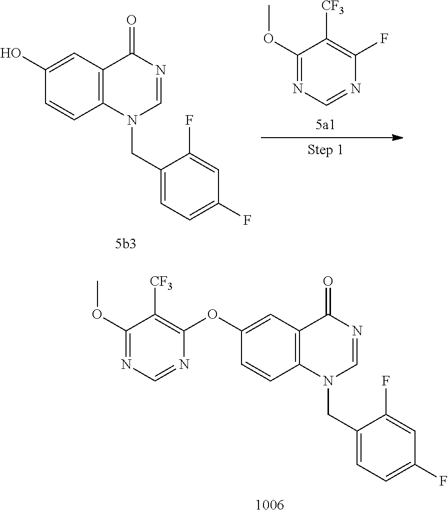

### Step 1:

To a solution of phenol 5b3 (30 mg, 0.10 mmol) in DMSO (1 mL) at RT is added K₂CO₃ (41 mg, 0.30 mmol) and the 2-fluoropyrimidine 5a1 (20 mg, 0.10 mmol). The reaction mixture is stirred 1 h at 80° C. AcOH (500 μL) is added and the resulting solution is injected onto a prep. HPLC to isolate, after lyophilisation, the desired product 1006.

### Example 6A

### Preparation of Compound 1008

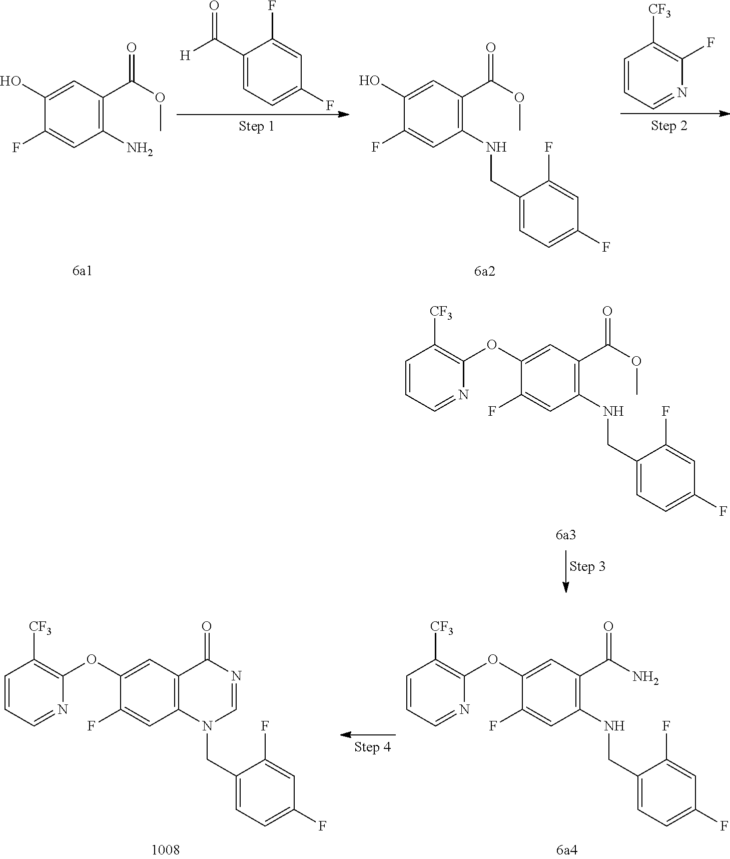

Aniline 6a1 is prepared as described in WO 2009/018656, herein incorporated by reference.

### Step 1:

The reductive amination to form 6a2 is performed as described in example 1A step 2.

### Step 2:

To the phenol 6a2 (1.4 g, 4.53 mmol) in MeCN (10 mL) at RT is added Cs₂CO₃ (2.2 g, 6.79 mmol) and 2-fluoro-3-trifluoromethylpyridine (1.5 g, 9.06 mmol). This solution is stirred at reflux for 3 h and then concentrated and purified by flash chromatography (20-50% AcOEt/Hex) to afford 6a3.

### Step 3:

i) To ester 6a3 (0.88 g, 1.9 mmol) in DMSO (10 mL) is added 10N NaOH (0.58 mL, 5.8 mmol) and this mixture is stirred 4 h at RT. THF (2 mL) and MgSO₄ are added and the solution is filtered.

ii) Ammonium bicarbonate (0.46 g, 5.8 mmol) and HATU (0.88 g, 2.3 mmol) are added to the filtrate and the mixture is stirred at RT for 2 h. The resulting mixture is diluted in water then extracted with EtOAc (3×). The organic extracts are combined, washed with brine, dried over MgSO₄, filtered then concentrated. Carboxamide 6a4 is utilized without further purification.

### Step 4:

Compound 1008 is prepared from 6a4 using the protocol described in example 1A step 3.

### Example 7A

### Preparation of Compound 1009

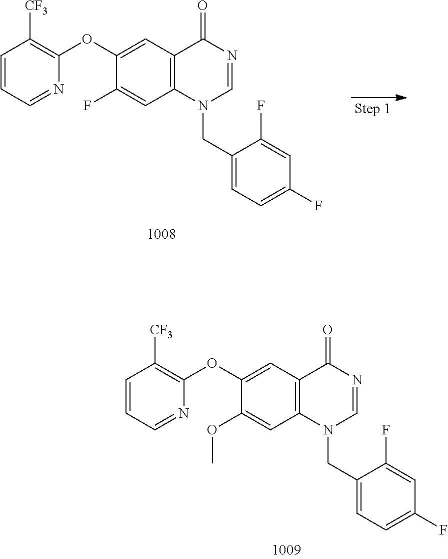

### Step 1:

To 1008 (24 mg, 0.05 mmol) in MeOH (1 mL) is added 10N NaOH (9 μL, 0.09 mmol) and this reaction is stirred 20 h at RT. The solution is then diluted with AcOH (2 mL) and purified by prep. HPLC to afford, after lyophilisation, compound 1009.

### Example 8A

### (Synthetic Method C): Preparation of Compound 1012

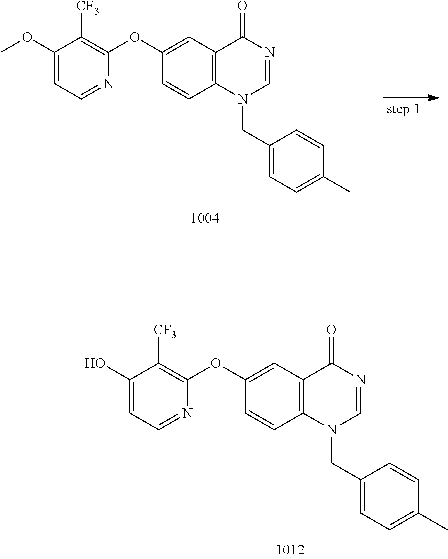

### Step 1:

To 1004 (220 mg, 0.5 mmol) in DCM (1 mL) is added HBr (48% in AcOH, 3.5 mL). The reaction is stirred 9 h at 80° C. A mixture of Et₂O/EtOAc (20/30 mL) is added and washed with a sat. aq. NaHCO₃ and brine, dried over MgSO₄, filtered and concentrated under vacuum. Trituration with Et₂O followed by filtration to isolate 1012.

### Example 9A

### (Synthetic Method D): Preparation of Compound 1010

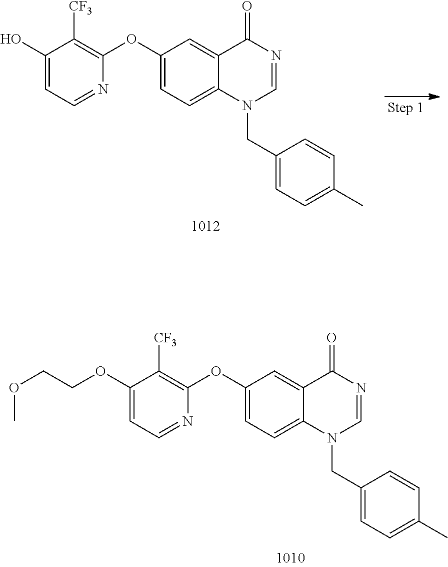

### Step 1:

To 1012 (35 mg, 0.08 mmol) in DMSO (2 mL) are added K₂CO₃ (33 mg, 0.24 mmol) and 2-bromoethyl methylether (36 μL, 0.37 mmol). The reaction is stirred 1.5 h at 80° C. then diluted with EtOAc, washed with 1N HCl, water, sat. aq. NaHCO₃ and brine. The organic phase is dried over MgSO₄, filtered and concentrated under vacuum, followed by purification with prep. HPLC to afford 1010.

### Example 10A

### (Synthetic Method E): Preparation of Compound 1011

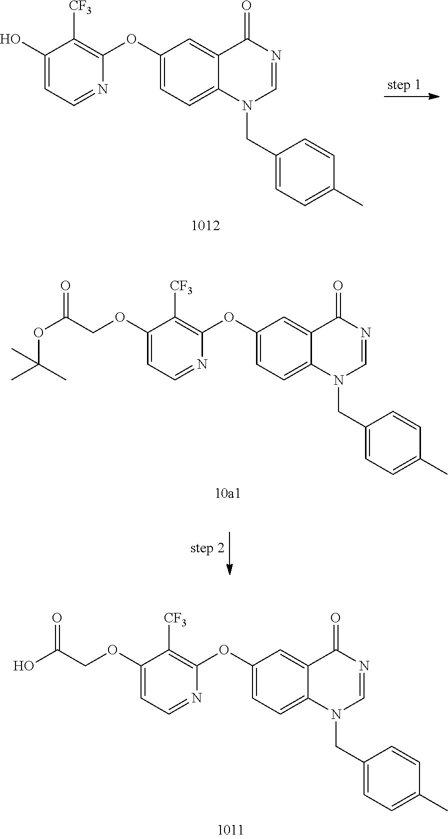

### Step 1:

Compound 10a1 is prepared from 1012 and tert-butyl bromoacetate using the protocol described in example 9A step 1.

### Step 2:

To 10a1 (44 mg, 0.08 mmol) in DCM (1 mL) is added TFA (1 mL) at RT. The reaction is stirred overnight then purified by prep. HPLC to afford 1011.

### Example 11A

### Preparation of Compound 1013

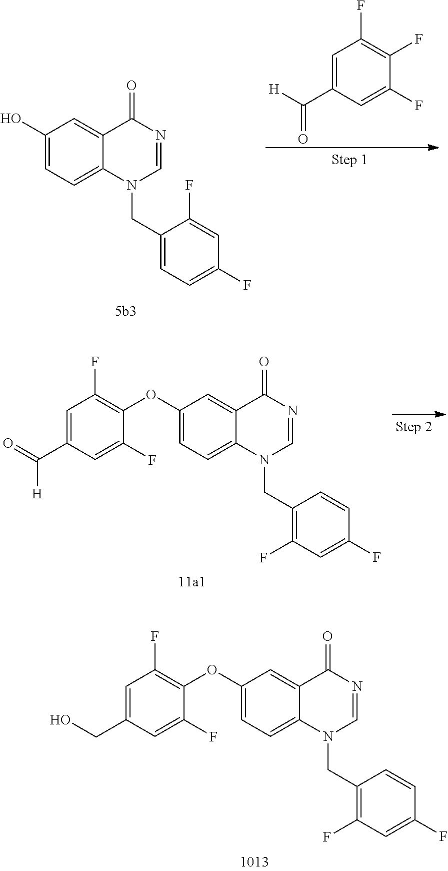

### Step 1:

The S_{N}Ar between phenol 5b3 and 3,4,5-trifluorobenzaldehyde is performed as described in example 5C step 1 with purification by flash chromatography.

### Step 2:

To a mixture of aldehyde 11a1 (66 mg, 0.15 mmol) in THF (2 mL) is added NaBH₄ (2 mg, 0.05 mmol). This mixture is stirred 2 h at RT before being diluted with AcOH (1 mL) and directly injected onto a prep. HPLC to isolate 1013.

### Example 12A

### Preparation of Intermediate 12A1

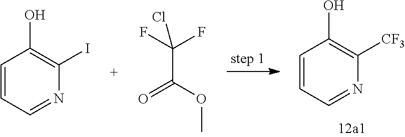

Reference: Su, D.-B.; Duan, J.-X.; Chen, Q.-Y Tet. Lett. 1991, 32, 7689, herein incorporated by reference.

### Step 1:

NMP (145 mL) is degassed 30 min with Ar and is then heated to 80° C. KF (7.62 g, 131 mmol), CuI (1) (24.99 g, 131 mmol) and 2-iodopyridin-3-ol (29 g, 131 mmol) are added in one portion, followed by methyl 2-chloro-2,2-difluoroacetate (55.4 mL, 525 mmol). The resulting mixture is heated at 120° C. under N₂ for 3 nights. The mixture is allowed to cool to RT and is then poured gently into a slowly stirring mixture of 50% concentrated NaCl (1 L) solution and Et₂O (4 L). The organic layer is decanted carefully and the aqueous layer is extracted with Et₂O (3×). The combined organic layers are concentrated, washed with brine (4×), dried with MgSO₄, filtered and concentrated. Flash chromatography on silica (DCM:MeOH 1.2%-10) then with (1-Pr₂O:EtOAc 9:1) followed by trituration in heptane provides 12a1.

### Example 12B

### (Synthetic Method F): Preparation of Compound 1014

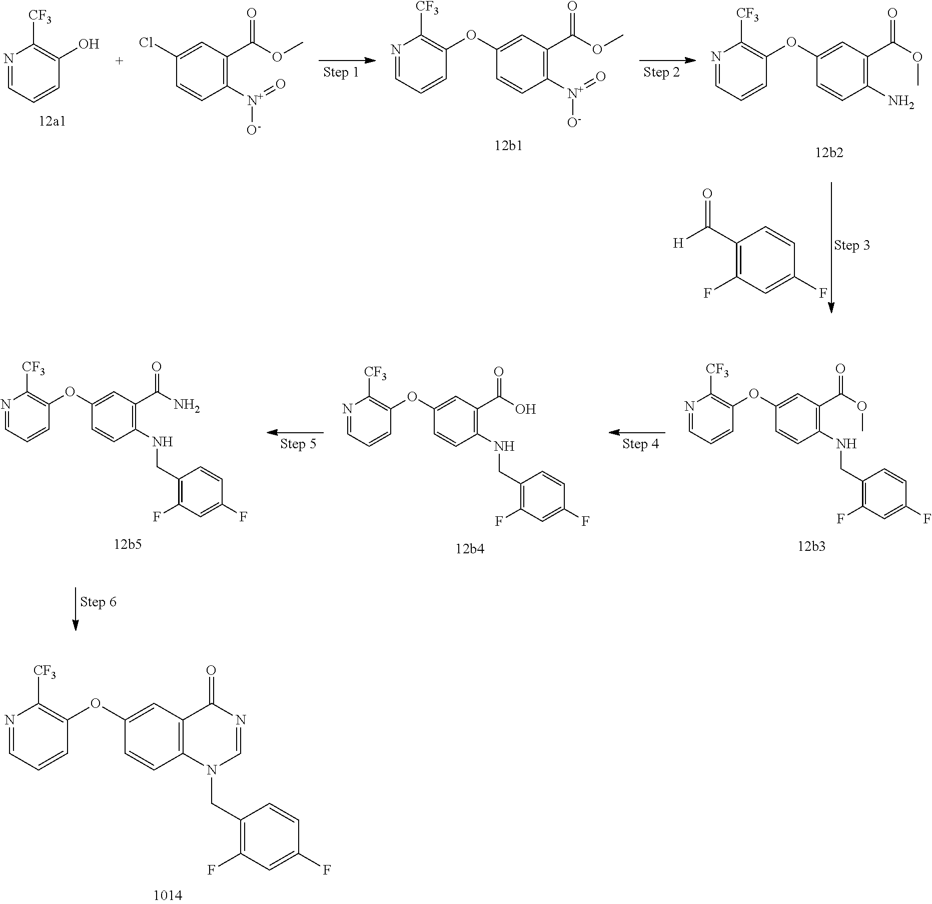

### Step 1:

To a solution of 12a1 (300 mg, 1.83 mmol) in DMSO (5 mL) at RT is added Cs₂CO₃ (894 mg, 2.76 mmol) and the methyl-5-chloro-2-nitrobenzoate (397 mg, 1.84 mmol). The reaction mixture is stirred 2 h at 70° C. The resulting mixture is diluted with EtOAc and washed with water and brine. The organic phase is dried over MgSO₄, filtered and concentrated under vacuum followed by combiflash purification (20-50% EtOAc/Hex) to afford 12b1.

### Step 2:

To a solution of 12b1 (325 mg, 0.95 mmol) in MeOH (5 mL) under nitrogen atmosphere, is added Pd/C (10% w/w, 32 mg) at RT. The reaction mixture is purged with H₂ atmosphere and then stirred under H₂ for 4 h. The resulting mixture is filtered through a Millex™ filter and concentrated under vacuum to afford 12b2.

### Step 3:

To a solution of aniline 12b2 (190 mg, 0.59 mmol) in DCE (3 mL) at RT is added 2,4-difluorobenzaldehyde (93 mg, 0.65 mmol) followed by Na(OAc)₃BH (150 mg, 0.71 mmol). The reaction mixture is stirred overnight at RT. More aldehyde (210 mg, 1.3 mmol) and Na(OAc)₃BH (300 mg, 1.4 mmol) are added after 12 h. AcOH (1 mL) and Na(CN)BH₃ (74 mg, 1.2 mmol) are also added to bring the reaction to completion after stirring 2 h at RT. The resulting mixture is diluted with EtOAc and sat. aq. NaHCO₃ and the organic phase is separated. The aqueous phase is reextracted with EtOAc and the combined organic phases are washed with water and brine, dried over MgSO₄, filtered and concentrated under vacuum, followed by combiflash purification (5-40% EtOAc/Hex) to afford 12b3.

### Step 4:

To a solution of ester 12b3 (210 mg, 0.47 mmol) in a mixture of THF/MeOH (2:1, 4.4 mL) at RT is added 1N NaOH (2.4 mL, 2.4 mmol). The reaction mixture is stirred overnight at RT. The resulting solution pH is adjusted to 5.5 with the addition of 1N HCl and the aqueous phase is extracted with EtOAc (3×). The combined organic phases are washed with brine, dried over MgSO₄, filtered and concentrated under vacuum to afford acid 12b4.

### Step 5:

Amide 12b5 is prepared from acid 12b4 using the protocol described in example 5B step 2.

### Step 6:

Compound 1014 is prepared from 12b5 using the protocol described in example 1A step 3.

### Example 13A

### (Synthetic Method G): Preparation of Compound 1015

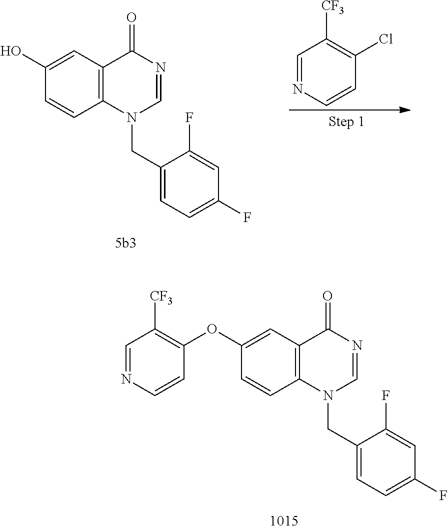

### Step 1:

To a solution of phenol 5b3 (75 mg, 0.26 mmol) in DMSO (2 mL) at RT is added K₂CO₃ (130 mg, 0.91 mmol) and the 4-chloro-3-trifluoromethylpyridine hydrochloride (85 mg, 0.39 mmol). The mixture is heated in a microwave with stirring at 150° C. for 12 min. The mixture is diluted with water then extracted with DCM (3×). The crude product is taken up in DMSO and purified by prep. HPLC to isolate, after lyophilisation, the desired product 1015.

### Example 14A

### Preparation of Compound 1016

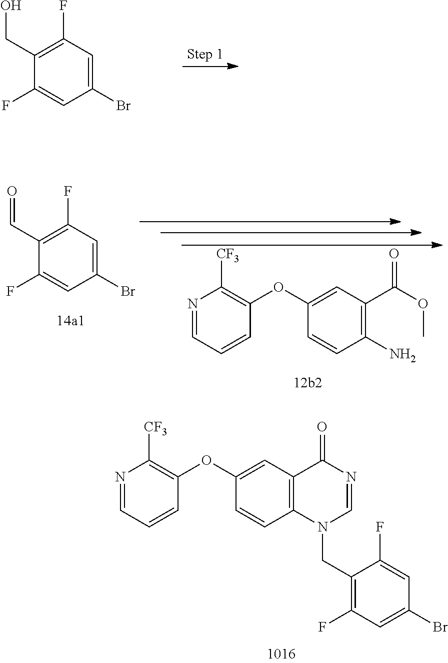

### Step 1:

To a mixture of 4-bromo-2,6-difluorobenzyl alcohol (1.00 g, 4.48 mmol) in DCM (20 mL) is added Dess-Martin periodinane (2.12 g, 5.00 mmol). This mixture is stirred 2 h at RT. The mixture is then partitioned between EtOAc and sat. aq. NaHCO₃. The organic phase is washed with brine, dried over MgSO₄, filtered and concentrated. The crude product is purified by flash chromatography to afford aldehyde 14a1.

Compound 1016 is prepared from 14a1 using the sequence described in example 12B.

### Example 15A

### Preparation of Compound 15A3

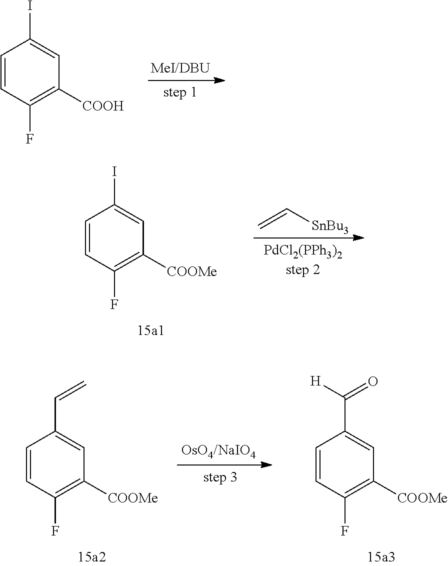

### Step 1:

To 2-fluoro-5-iodobenzoic acid (5.12 g, 19 mmol) in MeCN (100 mL) and DMF (10 mL) at 0° C., is added DBU (3.20 mL, 20.9 mmol) and MeI (1.80 mL, 28.5 mmol). The reaction is stirred overnight at RT and then is poured into water (300 mL), extracted with EtOAc (2×), washed with brine, dried with MgSO₄, filtered and concentrated under vacuum to afford 15a1.

### Step 2:

Aryliodide 15a1 (5.4 g, 19.3 mmol) is dissolved in dry dioxane (50 mL) and tributylvinylstannane (6.2 mL, 21.2 mmol) is added. The solution is degassed and Pd(PPh₃)₂Cl₂ (1.35 g, 0.10 mmol) is added. The reaction is heated 2 h at reflux. The mixture is allowed to cool to RT before being concentrated and directly subjected to flash chromatography (0-5% EtOAc/Hex) to isolate alkene 15a2.

### Step 3:

To alkene 15a2 (3.49 g, 19.4 mmol) dissolved in THF (130 mL) and water (100 mL) is added 2.5% OsO₄ in t-BuOH (1.4 mL) and NaIO₄ (11.8 g, 56.6 mmol). The mixture is stirred overnight at RT. The volatiles are removed under reduced pressure before the mixture is extracted with EtOAc (2×). The combined organic extracts are washed with brine, dried with MgSO₄, filtered and concentrated under vacuum. Purification by flash chromatography (0-5% EtOAc/Hex) affords aldehyde 15a3.

### Example 15B

### Preparation of Intermediate 15b1

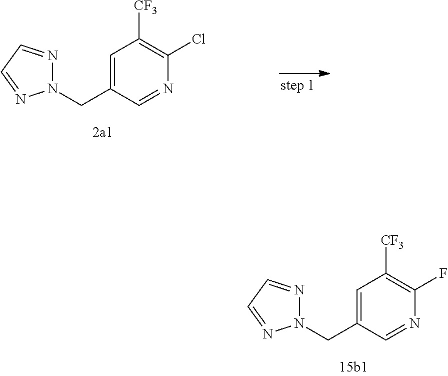

### Step 1:

To a mixture of chloropyridine 2a1 (3.00 g, 11.4 mmol) in anhydrous DMSO (15 mL) at RT is added dibenzo-18-crown-6 (2.06 g, 5.7 mmol) followed by KF (3.31 g, 57 mmol). The mixture is stirred at RT for 5 min, sonicated for 1 min, then heated in microwave (pre-stirring 30 sec) at 170° C. for 30 min. The mixture is diluted in EtOAc (600 mL) and ether (100 mL). The solid biproducts are removed by filtration. The organic filtrate is washed with water and brine. The organic phase is dried over MgSO₄, filtered and partially concentrated. The mixture is diluted in ether and hexanes ( 50/300 mL), sonicated and filtered off (medium frit to remove crown-6). The filtrate is concentrated to dryness. The crude product is purified by combiflash (5% to 50% EtOAc/Hex) to isolate the fluoropyridine 15b1.

### Example 15C

### Preparation of Compound 1017

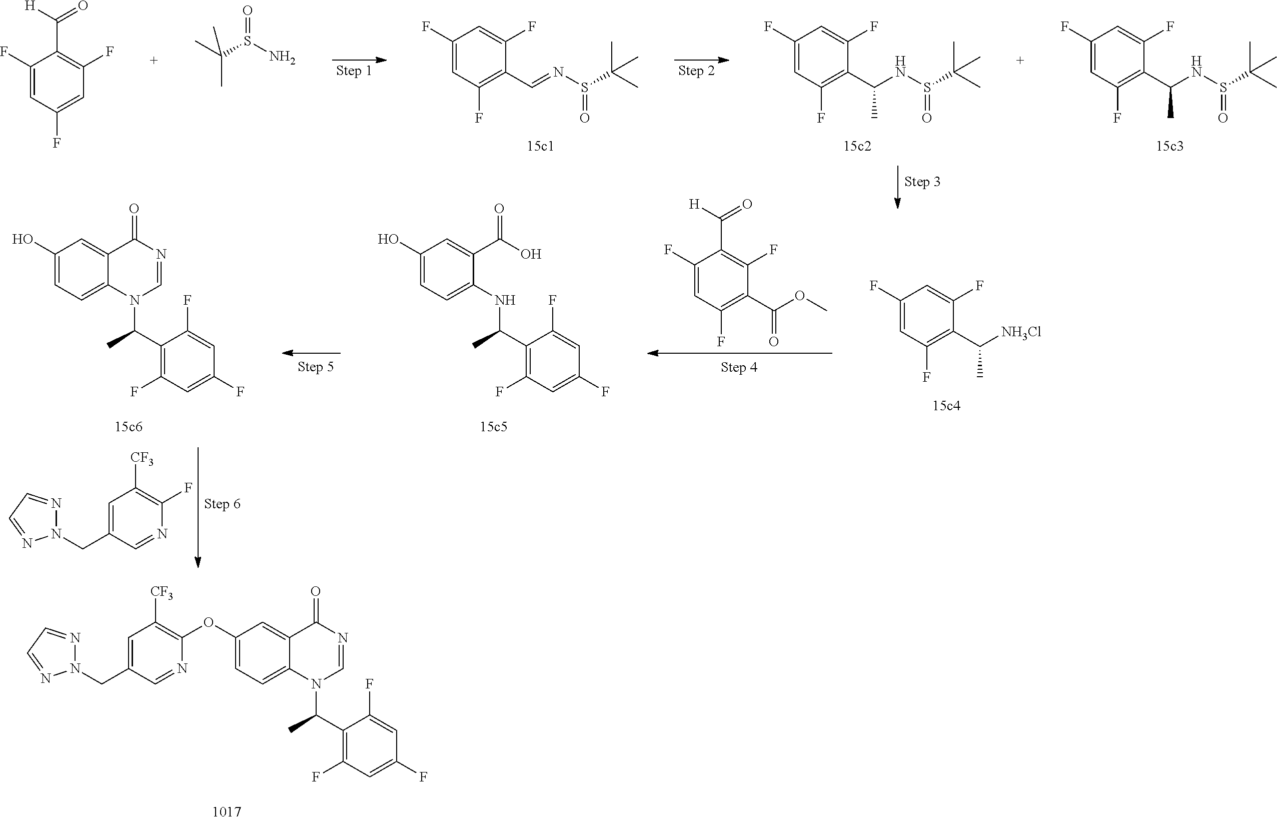

Reference for steps 1 to 3: Cogan, D. A.; Liu, G.; Ellman, J. Tetrahedron. 1999, 55, 8883, herein incorporated by reference.

### Step 1:

(S)-(−)-2-Methyl-2-propanesulfinamide (1.52 g, 12.5 mmol) is added to the 2,4,6-trifluorobenzaldehyde (2.0 g, 12.4 mmol) in DCE (30 mL) under a N₂ atmosphere. Ti(OEt)₄ (4.8 mL, 20 mmol) is then added in one portion. The mixture is warmed to 80° C. and stirred 2.5 h after which the TLC indicates completion of the reaction. Water (10 mL) is added to the vigorously stirred cold solution (ice-bath) and celite (20 g) is added to the suspension. The slurry is vigorously stirred at RT for 30 min. The mixture is filtered and the celite cake washed with DCM. The filtrate is concentrated, taken-up in EtOAc, washed with brine, dried with MgSO₄, filtered and concentrated to afford 15c1.

### Step 2:

To the imine 15c1 (3.28 g, 12.5 mmol) in DCM (50 mL) at −20° C. is slowly added 3.0 M MeMgBr in Et₂O (10 mL, 30 mmol). The mixture is stirred 1 h at −20° C. then 2 h at RT. A sat. aq. solution of NH₄Cl is added and the two phases are separated. The aqueous phase is reextracted with DCM (2×) and the combined organic phases are dried with MgSO₄, filtered and concentrated. Purification by combiflash separates diasteriomers 15c2 and 15c3. Absolute stereochemistry was assigned based on precedent described in the aforementioned reference

### Step 3:

To 15c2 (1.93 g, 6.91 mmol) in MeOH (10 mL) is added 4N HCl in dioxane. The solution is stirred 1 h at RT then concentrated to afford crude hydrochloride salt 15c4.

### Step 4:

To the aldehyde 15a3 (419 mg, 2.3 mmol) in DMSO (4 mL) is added the amine 15c4 (490 mg, 2.3 mmol) and K₂CO₃ (1.2 g, 9 mmol). The solution is stirred 4 h at 90° C. then 24 h at 120° C. 2.5N NaOH (9.7 mL, 24 mmol) is added and the solution is stirred 1 h at 50° C. Water is added and the resulting mixture is neutralized with 3N HCl. The resulting residue is separated and then taken up in MeOH (10 mL). The mixture is chilled to 0° C. then concentrated H₂SO₄ (160 μL, 2.56 mmol) and H₂O₂ 30% (290 μL, 2.56 mmol) are added. This solution is stirred 1 h at RT before being diluted with water and extracted with DCM (2×). The combined organic phases are dried with MgSO₄, filtered and concentrated to afford phenol 15c5.

### Step 5:

The carboxamide intermediate is prepared from acid 15c5 using the protocol described in example 5B step 2. Crude carboxamide is subjected to the protocol described in example 1A step 3, to obtain the phenol 15c6.

### Step 6:

To phenol 15c6 (75 mg, 0.23 mmol) in DMF (2 mL) are added Cs₂CO₃ (56 mg, 0.56 mmol) and fluoropyridine 15b1 (86 mg, 0.35 mmol). The reaction is heated to 120° C. and stirred for 30 min in a microwave. The mixture is diluted with AcOH (1 mL) and DMSO (2 mL) and the mixture is injected onto a semi-prep. HPLC to isolate 1017.

### Example 16A

### (Synthetic Method H): Preparation of Compound 1019

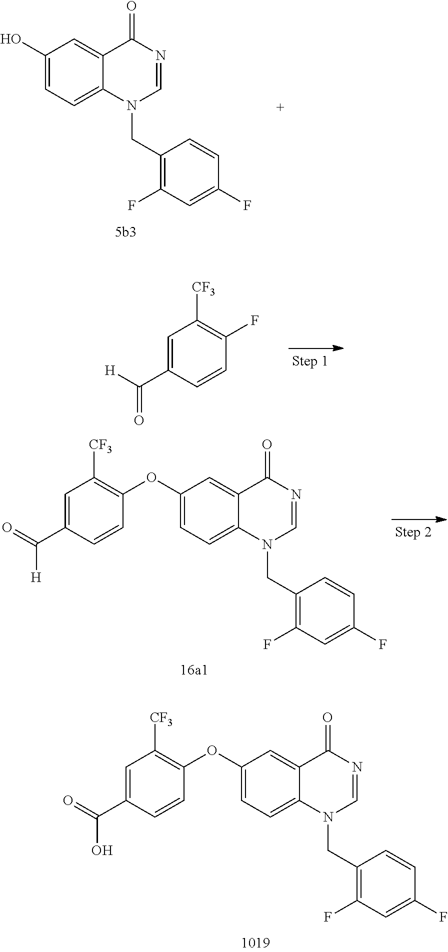

### Step 1:

Arylether 16a1 is prepared from phenol 5b3 and 4-fluoro-3-trifluoromethylbenzaldehyde using the protocol described in example 5C step 1.

### Step 2:

To a mixture of aldehyde 16a1 (40 mg, 0.09 mmol) in formic acid (95%, 0.5 mL) is added UHP (20 mg, 0.21 mmol). The mixture is stirred 1 h at RT before being concentrated. The residue is taken up in DMSO and AcOH then injected onto a prep. HPLC to isolate compound 1019.

### Example 17A

### Preparation of Compound 1020

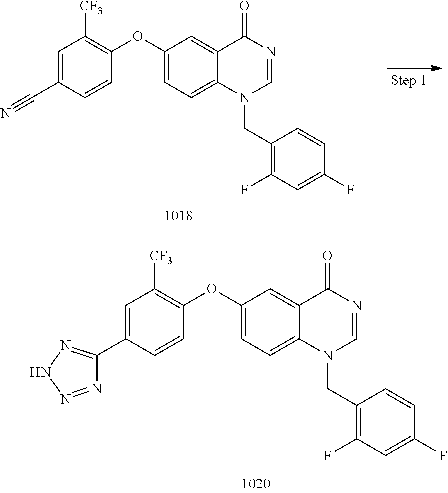

### Step 1:

To nitrile 1018 (60 mg, 0.21 mmol) in DMF (4 mL) is added NH₄Cl (32 mg, 0.6 mmol) and NaN₃ (39 mg, 0.6 mmol). This reaction mixture is heated 3 h at 90° C. in a sealed tube. The mixture is diluted in AcOH then injected onto a prep. HPLC to isolate compound 1020.

### Example 18A

### Preparation of Compound 1021

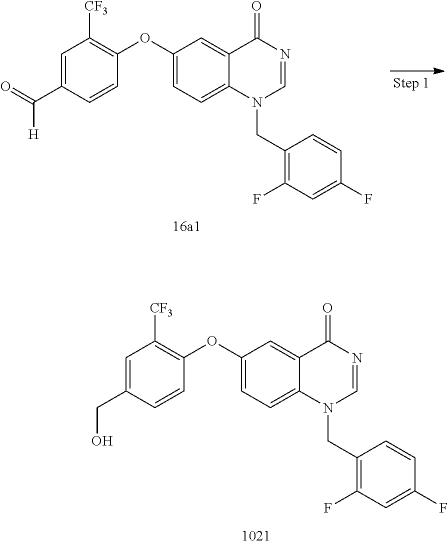

### Step 1:

To aldehyde 16a1 (66 mg, 0.14 mmol) in THF (2 mL) is added NaBH₄ (2 mg, 0.05 mmol). This reaction mixture is stirred 2 h at RT. The mixture is diluted in AcOH then injected onto a prep. HPLC to isolate compound 1021.

### Example 19A

### (Synthetic Method I): Preparation of Compound 1022

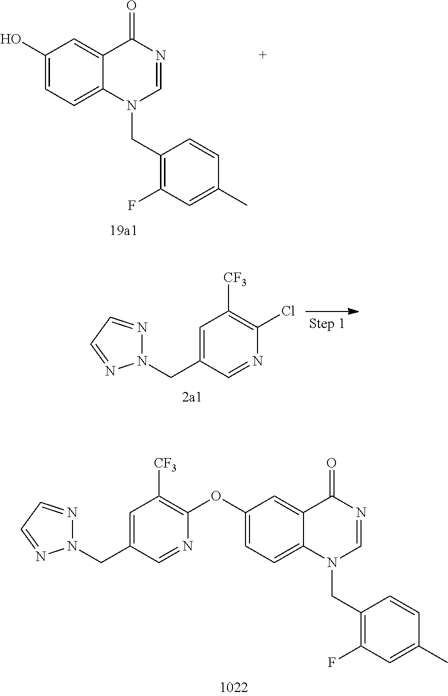

### Step 1:

Phenol 19a1 is prepared using the sequence described in example 5B.

To a mixture of phenol 19a1 (50 mg, 0.18 mmol) in DMF (1 mL) is added Cs₂CO₃ (86 mg, 0.26 mmol) and the chloropyridine 2a1 (55 mg, 0.21 mmol). The mixture is heated with stirring at 90° C. for 4 h. The mixture is then injected directly onto a prep. HPLC instrument to isolate 1022. The compound is repurified by prep. TLC (95:5 DCM/MeOH).

### Example 20A

### (Synthetic Method J): Preparation of Compound 1023

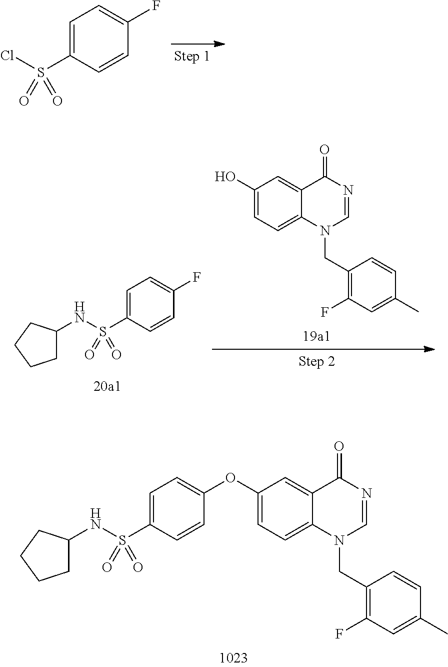

### Step 1:

To a mixture of 4-fluorophenylsulfonylchloride (500 mg, 2.57 mmol) in DCM (10 mL) is added NEt₃ (0.40 mL, 2.87 mmol) and cyclopentylamine (0.26 mL, 2.64 mmol). The mixture is stirred overnight at RT. The mixture is then diluted with DCM and washed with 1N HCl and brine. The organic phase is dried with MgSO₄, filtered and concentrated. Crude sulfonamide 20a1 is used without further purification.

### Step 2:

To a mixture of phenol 19a1 (100 mg, 0.25 mmol) in DMSO (2 mL) is added K₂CO₃ (111 mg, 0.80 mmol) and the fluoroarene 20a1 (120 mg, 0.49 mmol). The mixture is heated with stirring at 110° C. for 18 h. The mixture is filtered, diluted in DMSO/MeCN then injected directly onto a prep. HPLC instrument to isolate 1023.

### Example 21A

### (Synthetic Method K): Preparation of Compound 1025

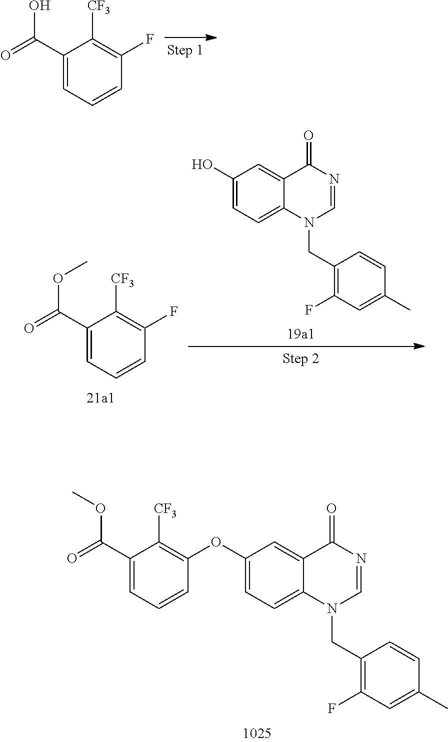

### Step 1:

To a mixture of 3-fluoro-2-trifluoromethylbenzoic acid (1.00 g, 4.81 mmol) in EtOAc/MeOH (45 mL, 7:2) is added a freshly prepared solution of diazomethane in ether until the characteristic yellow colour persists. The mixture is concentrated to afford methylester 21a1 which is utilized without further purification.

### Step 2:

Phenol 19a1 (200 mg, 0.50 mmol) is combined with fluoroarene 21a1 (130 mg, 0.60 mmol) and Cs₂CO₃ (410 mg, 1.25 mmol) in DMSO (2 mL). The mixture is stirred in a microwave at 90° C. for 10 min. An additional 1.2 eq of 21a1 is added and the mixture is submitted to the same microwave conditions. This process is repeated one additional time before the reaction is quenched with AcOH. The mixture is diluted in water and extracted with DCM. The organic phase is dried with MgSO₄, filtered and concentrated. Crude 1025 is purified by flash chromatography (DCM to 5% MeOH/DCM). The resulting material is then re-purified by prep. HPLC to afford compound 1025.

### Example 22A

### (Synthetic Method L): Preparation of Compound 1027

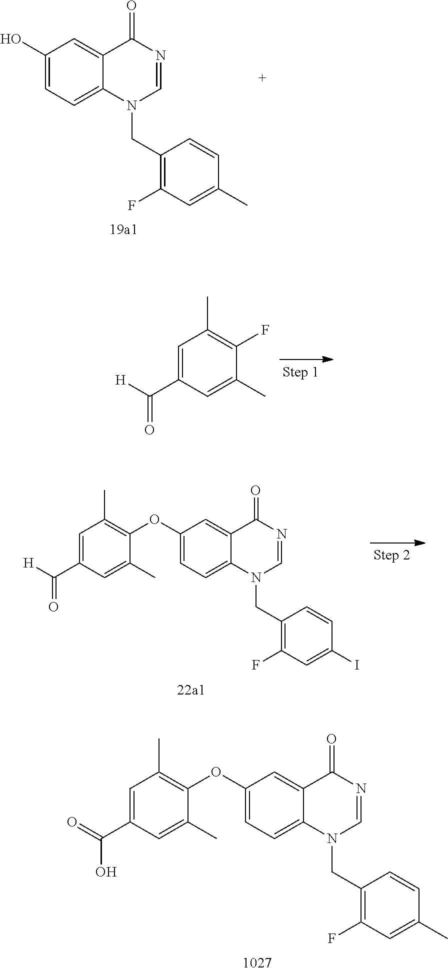

### Step 1:

Arylether 22a1 is prepared from phenol 19a1 and 2,6-dimethyl-4-fluorobenzaldehyde using the protocol described in example 15C step 6.

### Step 2:

To a mixture of aldehyde 22a1 (150 mg, 0.35 mmol) in dioxane (20 mL) is added NaH₂PO₄ (140 mg, 2.5 mmol) in water (6 mL) and sulfamic acid (110 mg, 1.1 mmol). The mixture is chilled to 0° C. before NaClO₂ (90 mg, 1.0 mmol) in water (6 mL) is added over a period of 10 min. The mixture is stirred for 15 min at 0° C. before being acidified to pH 2 with 1N HCl. The mixture is diluted with EtOAc then washed with water and brine. The organic phase is dried with MgSO₄, filtered and concentrated. Crude 1027 is purified by prep. HPLC to afford the final compound.

### Example 23A

### (Synthetic Method M): Preparation of Compound 1029

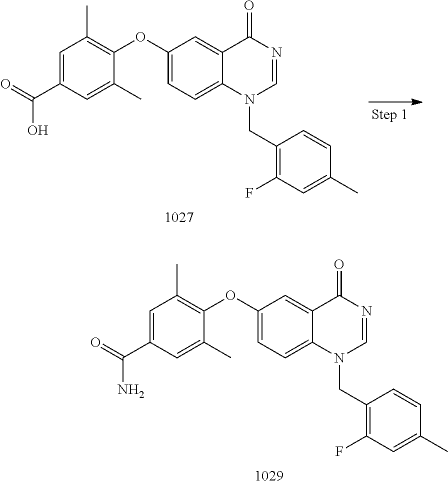

### Step 1:

Carboxamide 1029 is prepared from acid 1027 using the protocol described in example 5B step 2 with purification by prep HPLC.

### Example 24A

### Preparation of Intermediate 24A2

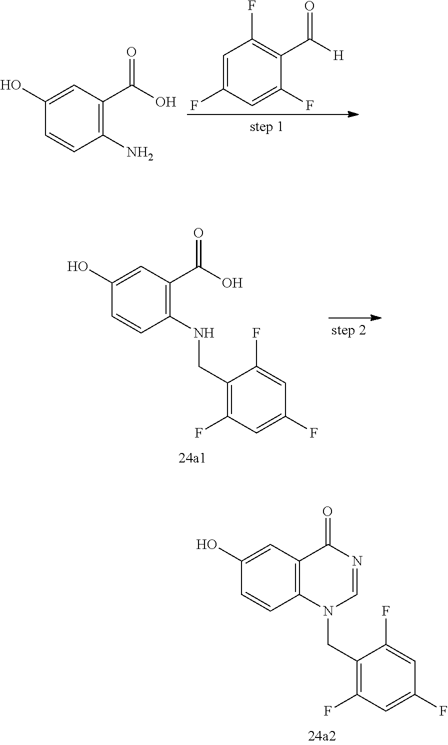

### Step 1

Intermediate 24a1 is prepared from 2-amino-5-hydroxybenzoic acid using the protocol described in example 5B step 1.

### Step 2

The anthranilic acid derivative 24a1 (1.0 g, 3.36 mmol, 1.0 eq) is dissolved in 2-methoxyethanol (7 mL) under Ar. To this mixture is added formamidine acetate (0.42 g, 4.0 mmol, 1.2 eq). The mixture is refluxed for 2 h (monitored by LC-MS). Additional formamidine acetate (0.28 g, 2.7 mmol, 0.8 eq) is added and the mixture is refluxed for another 2 h. The mixture is allowed to cool to RT before being filtered and washed with ethanol to afford quinazolinone 24a2. More product can be recovered from the filtrate by concentration and recrystallization from ethanol.

### Example 24B

### Preparation of Compound 2001

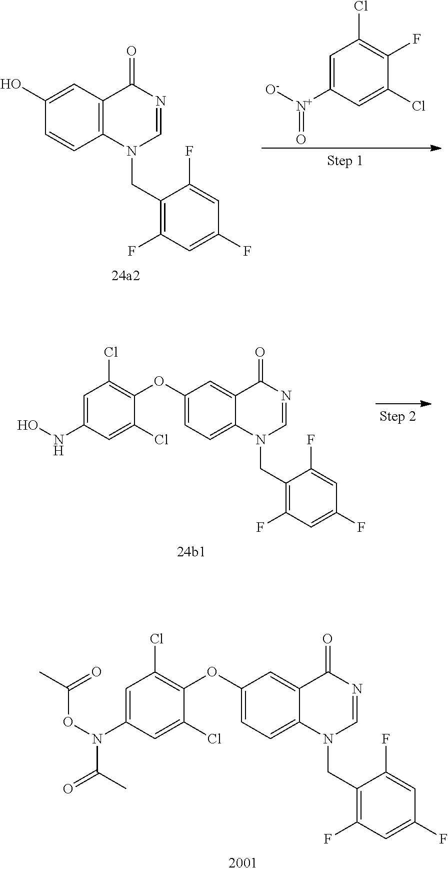

### Step 1:

Phenol 24a2 (100 mg, 0.33 mmol) is coupled to 3,5-dichloro-4-fluoronitrobenzene (84 mg, 0.40 mmol) using the protocol described in example 5C step 1. The mixture is diluted with EtOAc then washed with water and brine. The organic phase is dried with MgSO₄, filtered and concentrated. The residue is taken up in THF (8 mL). To the mixture is added 1N HCl (5 mL) and tin (110 mg, 0.90 mmol). The mixture is stirred 2 h at RT before being filtered through celite and concentrated. Crude 24b1 is utilized in the next step without further purification.

### Step 2:

To a mixture of hydroxylamide 24b1 (40 mg, 0.09 mmol) in DCM (2 mL) is added AcCI (8 μL, 0.10 mmol) and NEt₃ (28 μL, 0.20 mmol). The mixture is stirred for 1 h at RT before being concentrated. The residue is taken up in DMSO then injected onto a prep. HPLC to isolate 2001.

### Example 25A

### Preparation of Compound 2003

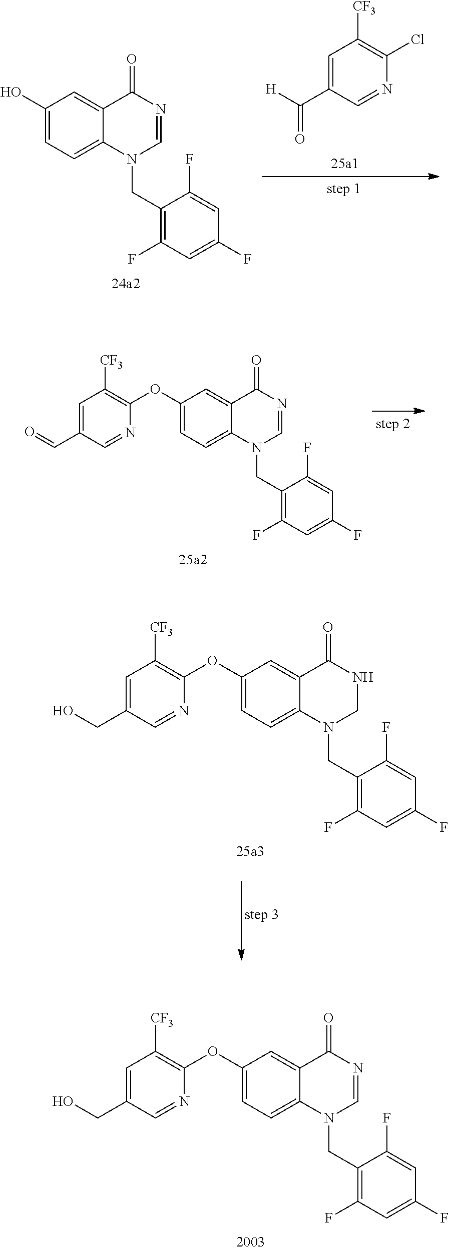

Aldehyde 25a1 is prepared using the protocol described in the following reference: WO 2008/019477, herein incorporated by reference.

### Step 1:

To a mixture of 24a2 (2.0 g, 6.5 mmol) and the aldehyde 25a1 (1.64 g, 7.8 mmol) in DMSO (30 mL) is added K₂CO₃ (2.25 g, 16.3 mmol). The mixture is stirred at 40° C. for 2 h. The reaction mixture is allowed to cool to RT and then poured into H₂O (500 mL) while stirring. The solid is collected by filtration, washed with H₂O and dried under high vacuum at <50° C. The solid is then triturated in Hex/EtOAc (4:1) overnight to give the desired product 25a2.

### Step 2:

To 25a2 (2.31 g, 4.8 mmol) in EtOH (50 mL) at 0° C. is added NaBH₄ (0.22 g, 2.8 mmol) in portions. The reaction mixture is stirred at 0° C. for 40 min before being quenched with H₂O, and extracted with EtOAc (3×). The combined EtOAc layer are washed with brine, dried with Na₂SO₄, filtered, and concentrated under vacuum to dryness to afford crude 25a3 which is utilized without further purification.

### Step 3:

The quinzolidinone 25a3 recovered in step 2 is taken up in MeOH (50 mL). To this mixture is added Na₂CO₃ (1.02 g, 9.6 mmol) and I₂ (1.71 g, 6.7 mmol). The reaction mixture is stirred at RT overnight. MeOH is removed under vacuum and the residue is diluted in EtOAc (100 mL) and washed with 20% Na₂S₂O₃. The organic phase is back extracted with EtOAc (2×). The combined EtOAc extracts are washed with brine, dried with Na₂SO₄, filtered, and concentrated under vacuum. The residue is triturated in EtOAc to afford compound 2003.

### Example 26A

### (Synthetic Method N): Preparation of Compound 2004

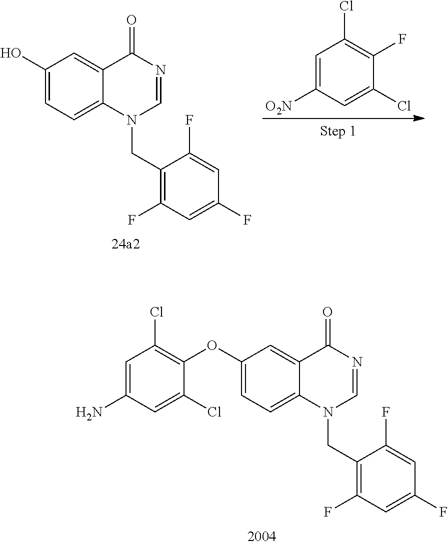

### Step 1:

Phenol 24a2 (100 mg, 0.33 mmol) is coupled to 3,5-dichloro-4-fluoronitrobenzene (84 mg, 0.40 mmol) using the protocol described in example 5C step 1. The mixture is diluted with EtOAc then washed with water and brine. The organic phase is dried with MgSO₄, filtered and concentrated. The residue is taken up in THF (8 mL). To the mixture is added 1N HCl (5 mL) and tin (110 mg, 0.90 mmol). The mixture is stirred 2 h at RT then heated to 60° C. and stirring is continued for 24 h. The mixture is concentrated and the residue is diluted with EtOAc then washed with sat. aq. NaHCO₃ and brine. The organic phase is dried with MgSO₄, filtered and concentrated. The residue is taken up in DMSO then injected onto a prep. HPLC to isolate 2004.

### Example 27A

### (Synthetic Method O): Preparation of Compound 2006

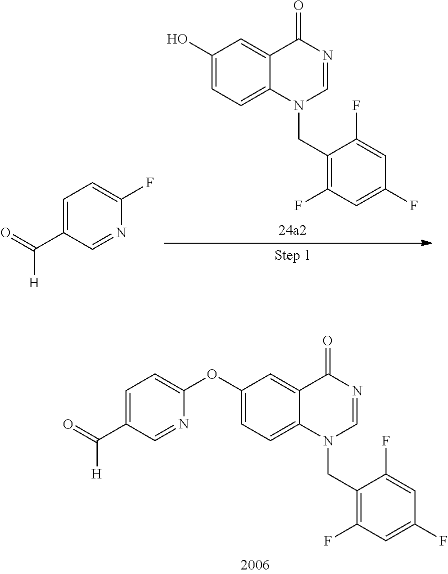

### Step 1:

Phenol 24a2 is coupled to 2-fluoro-5-pyridine carboxaldehyde using the protocol described in example 5C step 1. Purification by prep. HPLC affords 2006.

### Example 28A

### Preparation of Compound 2007

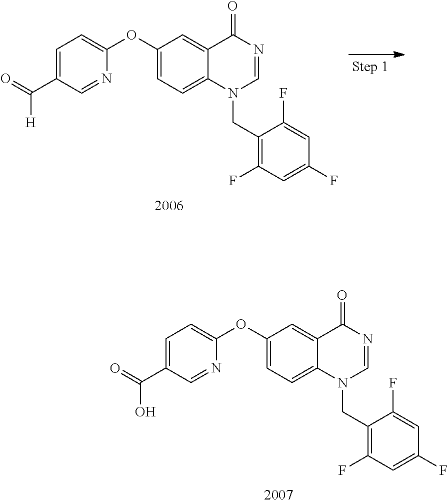

### Step 1:

To a mixture of aldehyde 2006 (70 mg, 0.17 mmol) in t-BuOH/2-Me-2-butene (5 mL, 3:2) at 0° C. is added 2 mL of an aq. solution of CaClO₂ (17 mg, 0.19 mmol) and NaH₂PO₄ monohydrate (130 mg, 0.94 mmol). The reaction mixture is stirred for 2 h at RT, partially concentrated, filtered and then purified by prep. HPLC to afford 2007.

### Example 29A

### Preparation of Intermediate 29A1

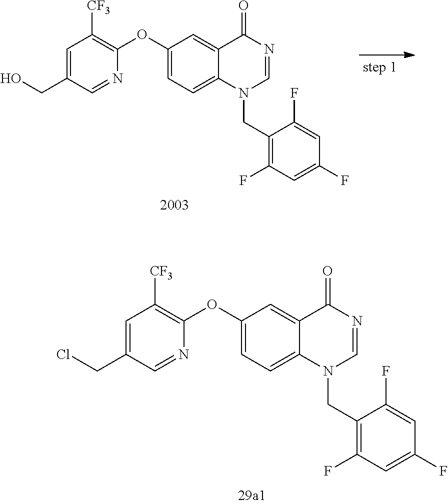

### Step 1:

To a mixture of alcohol 2003 (1.57 g, 3.1 mmol) in CHCl₃ (70 mL) is added SOCl₂ (0.71 mL, 9.7 mmol). The mixture is stirred at 40° C. for 2 h, then at RT overnight. The solvent is removed under vacuum and the residue is co-evaporated with CH₂Cl₂ (3×). The residue is triturated in Hex/EtOAc and filtered to afford benzyl chloride 29a1.

### Example 29B

### (Synthetic Method P): Preparation of Compounds 2008 & 2025

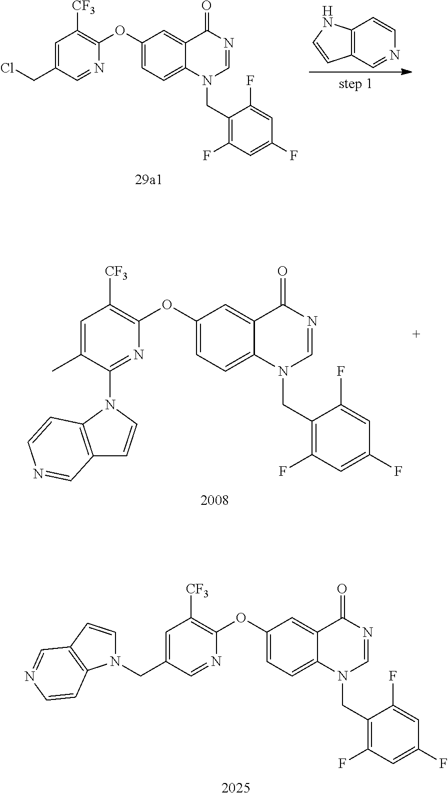

### Step 1:

To NaH (60% dispersion in mineral oil, 7.5 mg, 0.11 mmol) in DMF (2 mL) is added 4-azaindole (6 mg, 0.09 mmol). After stirring for 5 min, 29a1 (40 mg, 0.07 mmol) is added and the resulting solution is stirred 0.5 h at RT. The mixture is acidified with TFA before being injected directly onto a prep. HPLC to separate and isolate 2008 and 2025.

### Example 30A

### (Synthetic Method Q): Preparation of Compound 2009

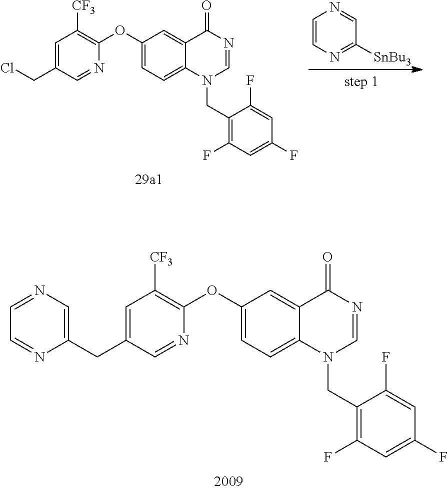

### Step 1:

To 29a1 (40 mg, 0.08 mmol) in DMF (2 mL) is added the stannane derivative (83 mg, 0.23 mmol) and Pd(PPh₃)₄ (11 mg, 0.01 mmol). The reaction is stirred 20 min at 120° C. in a microwave. The mixture is acidified with TFA before being injected directly onto a prep. HPLC to separate and isolate 2009.

### Example 31A

### Preparation of Compound 2011

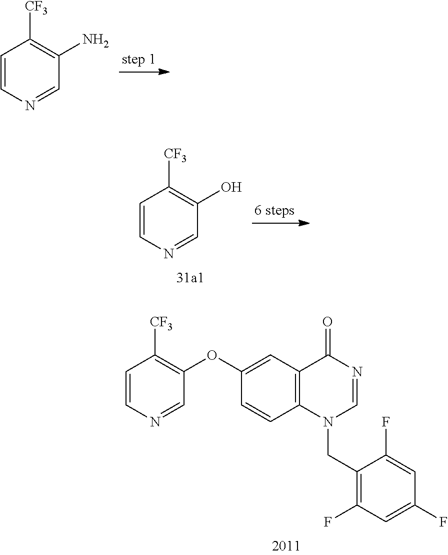

### Step 1:

A solution of 4-(trifluoromethyl)pyridin-3-amine (880 mg, 5.40 mmol) in 50% H₂SO₄ (12.25 mL) is cooled to −5° C. and a solution of NaNO₂ (447 mg, 6.48 mmol) in water (4.4 mL) is added slowly. The mixture is left to return to RT and stirring is continued for 30 min. The reaction mixture is heated to 100-110° C. for 2 h. The reaction medium is added dropwise to a sat. aq. NaHCO₃ solution (keep pH>7), and the mixture is extracted with Et₂O (2×) and EtOAc (3×). The organic phases are combined, washed with brine, dried over Na₂SO₄, filtered and concentrated under reduced pressure to provide hydroxypyridine 31a1.

Hydroxylpyridine 31a1 is used to synthesize 2011 using the same sequence described in example 12B (synthetic method F).

### Example 32A

### (Synthetic Method R): Preparation of Compound 2012

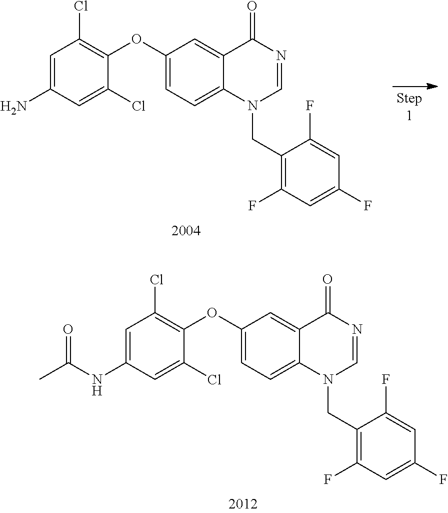

### Step 1:

Aniline 2004 is acylated to afford 2012 using the protocol described in example 24B step 2.

### Example 33A

### (Synthetic Method S): Preparation of Compound 2015

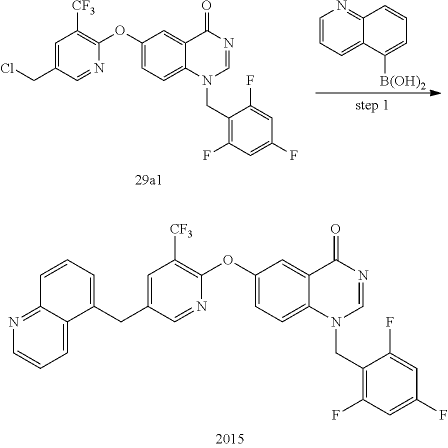

### Step 1:

To benzyl chloride 29a1 (41 mg, 0.07 mmol) in DMF (2 mL) is added the boronic acid (19 mg, 0.11 mmol) and K₃PO₄ (70 mg, 0.33 mmol). Ar is bubbled through the mixture for 10 min before Pd(OAc)₂ (4.5 mg, 0.02 mmol) and triphenylphosphine (9 mg, 0.04 mmol) are added. The reaction is stirred 20 min at 110° C. in a microwave. After the mixture cools to RT, the volatiles are removed under reduced pressure. The residue is taken up in DMSO (1 mL) and AcOH (0.5 mL) then injected onto a prep. HPLC to isolate 2015.

### Example 34A

### Preparation of Compound 2022

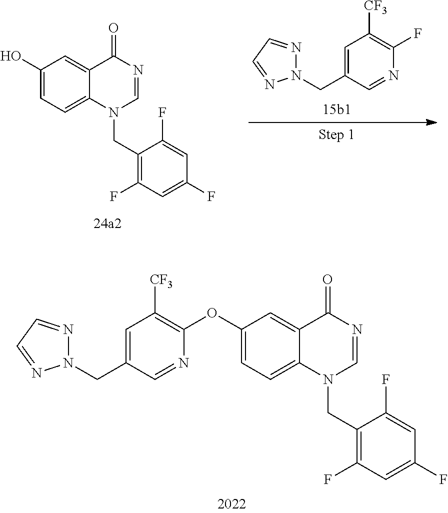

### Step 1:

Phenol 24a2 is coupled to fluoropyridine 15b1 using the protocol described in example 15C step 6. Purification by flash chromatography (6:1:1 EtOAc/acetone/Hex) affords 2022.

### Example 35A

### Preparation of Compound 2023

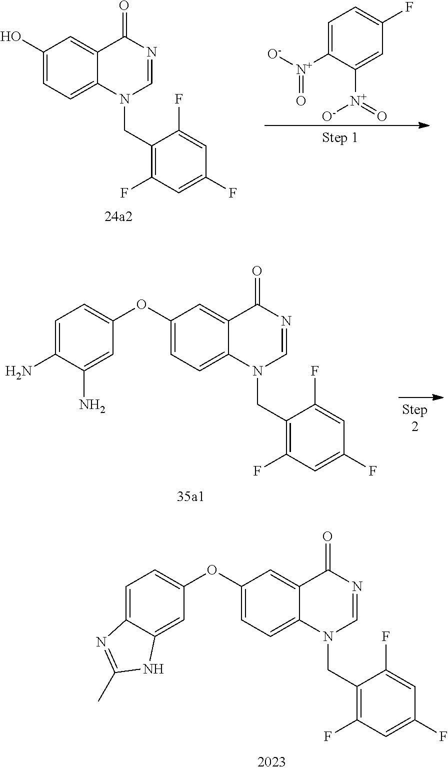

### Step 1:

i) Phenol 24a2 is coupled to 3,4-dinitrofluorobenzene using the conditions described in example 5C step 1. The reaction mixture is diluted in EtOAc and washed with water and brine. The organic phase is dried over MgSO₄, filtered and concentrated under vacuum to afford crude arylether which is utilized directly in the next step.

ii) To the residue in EtOH (8 mL) is added a sat. aq. solution of NH₄Cl (3 mL) and iron powder (55 mg, 1.0 mmol). This reaction mixture is stirred 20 h at reflux then diluted with EtOAc and washed (2×) with brine. The organic phase is dried over MgSO₄, filtered and concentrated under vacuum to afford 35a1 which is utilized without further purification.

### Step 2:

Reference: Beaulieu P. L.; Hache, B.; Van Moos, E. Synthesis 2003, 1683, herein incorporated by reference.

To the dianiline 35a1 (70 mg, 0.17 mmol) in DMF (2 mL) is added water (0.1 mL) then acetaldehyde (8 mg, 0.17 mmol) and Oxone® (74 mg, 0.12 mmol). The reaction is stirred 20 h at RT then diluted with AcOH (4 mL) and purified by semi-prep. HPLC to afford 2023.

### Example 36A

### (Synthetic Method T): Preparation of Compound 2026

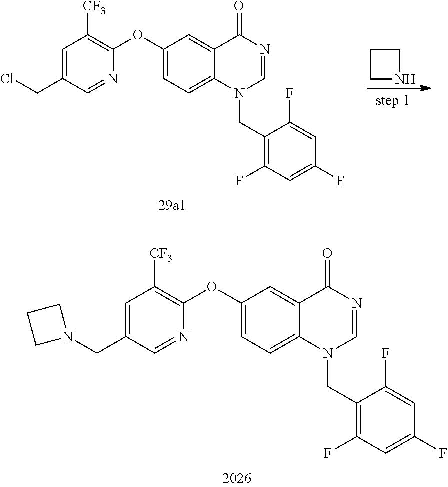

### Step 1:

To 29a1 (60 mg, 0.12 mmol) in DMF (2 mL) is added azetidine (10 mg, 0.18 mmol) and Et₃N (67 μL, 0.48 mmol). The solution is stirred 2.5 h at RT. The mixture is acidified with TFA before being injected onto a prep. HPLC to isolate 2026.

### Example 37A

### Preparation of Compound 2029

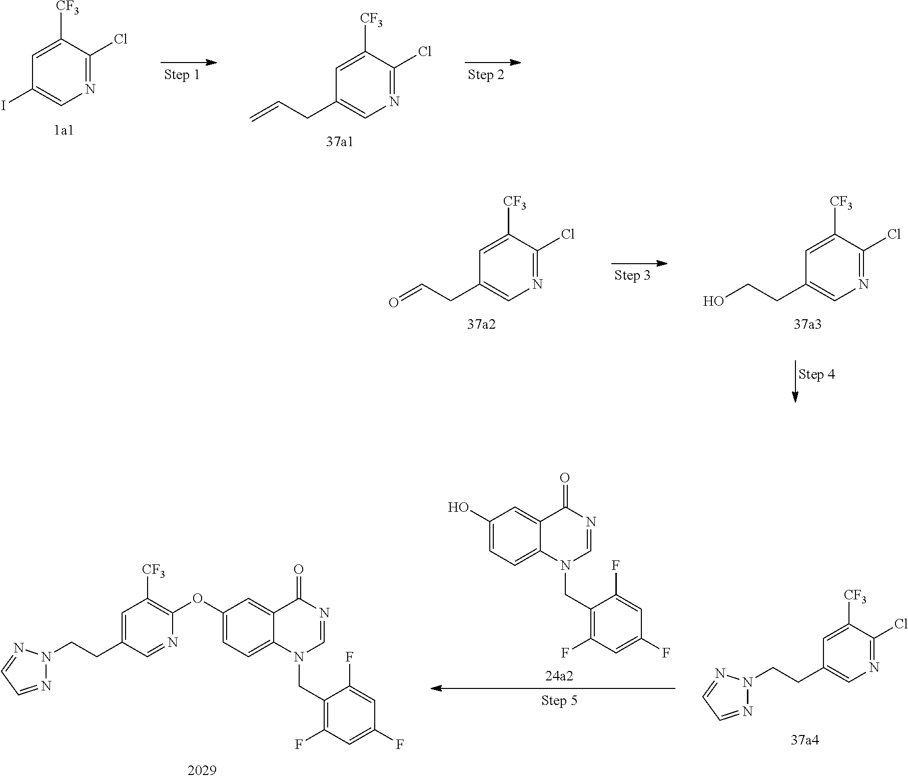

### Step 1:

To a solution of iodide 1a1 (300 mg, 0.98 mmol) in THF (3 mL) is added i-PrMgCl (0.54 mL 2.0 M soln in THF) at −40° C. The reaction mixture is stirred for 30 min and allyl bromide (0.13 mL, 1.5 mmol) is then added. This mixture is stirred at −40° C. for 15 min and then stirring is continued at RT for 30 min. The mixture is quenched with water and extracted with EtOAc (3×). The organic layers are combined, washed with brine, dried over anhydrous Na₂SO₄, filtered under vacuum and concentrated. Crude alkene 37a1 is employed without further purification in the subsequent step.

### Step 2:

Alkene 37a1 is transformed to aldehyde 37a2 using the procedure described in Step 3, Example 15A.

### Step 3:

Aldehyde 37a2 is reduced to alcohol 37a3 using the procedure described in Step 2, Example 25A.

### Step 4:

PPh₃ (30.5 g, 116 mmol) and 1,2,3-triazole (7.3 g, 106 mmol) are added to a mixture of alcohol 37a3 (23.0 g, 106 mmol) in anhydrous THF (200 mL). The solution is chilled to 0° C. and DEAD (23.5 g, 116 mmol) is added dropwise. The reaction mixture is stirred at 0° C. for 45 min then warmed to room temperature and stirred for 1 hr. Water (100 mL) is added and the mixture is extracted with EtOAc (3×200 mL). The organic layers are combined, washed with brine, dried over anhydrous Na₂SO₄, filtered and concentrated.

The crude material is dried, co-evaporated with toluene several times and dried under high vacuum until constant weight. The residue is taken up in hexanes cooled to 0° C. for 48 hrs. The desired product 37a4 is recovered by filtration.

### Step 5:

Phenol 24a2 is coupled to chloropyridine 37a4 using the protocol described in step 6 example 15C, to provide compound 2029.

### Example 38A

### (Synthetic Method U): Preparation of Compound 2030

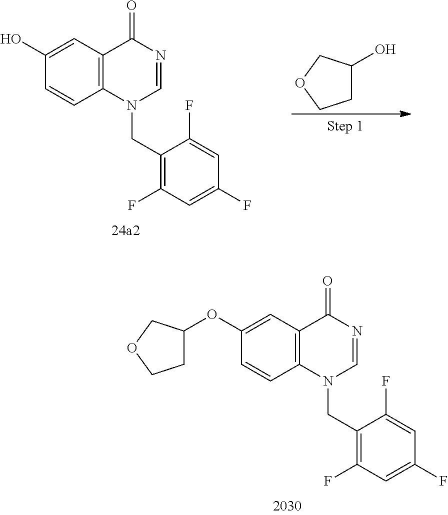

### Step 1:

To a mixture of 3-hydroxytetrahydrofuran (30 μL, 0.49 mmol) in THF (2 mL) at RT is added phenol 24a2 (50 mg, 0.16 mmol) and PPh₃ (70 mg, 0.26 mmol). DIAD (38 μL, 0.26 mmol) is added dropwise and the reaction mixture is then stirred overnight at RT. The mixture is filtered and concentrated. The residue is taken up in DMSO then injected onto a prep HPLC to isolate compound 2030.

### Example 39A

### (Synthetic Method V): Preparation of Compound 2032

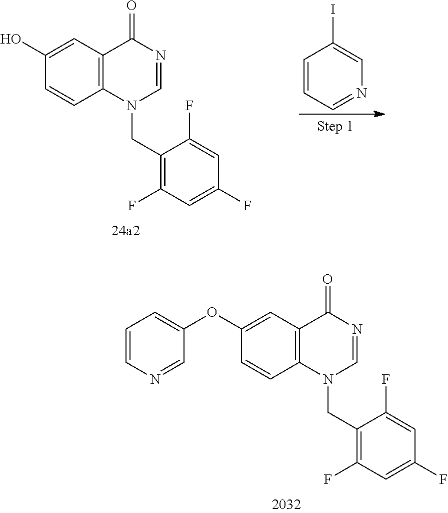

Reference: Buck, E.; Song, Z. J.; Tschaen, D.; Dormer, P. G.; Volante, R. P.; Reider, P. J. Org. Lett. 2002, 4, 1623, herein incorporated by reference.

### Step 1:

To a mixture of phenol 24a2 (60 mg, 0.20 mmol) in NMP (2 mL) at RT is added 3-iodopyridine (50 mg, 0.24 mmol), Cs₂CO₃ (195 mg, 0.60 mmol), 2,2,6,6-tetramethylheptane-3,5-dione (12 μL, 0.06 mmol) and CuCl (I) (6 mg, 0.06 mmol). This mixture is purged with Ar and heated 30 min at 160° C. in a microwave. The resulting mixture is filtered then directly purified by semi-prep. HPLC to afford, after lyophilisation, the desired product 2032.

### Example 40A

### Preparation of Compound 2033

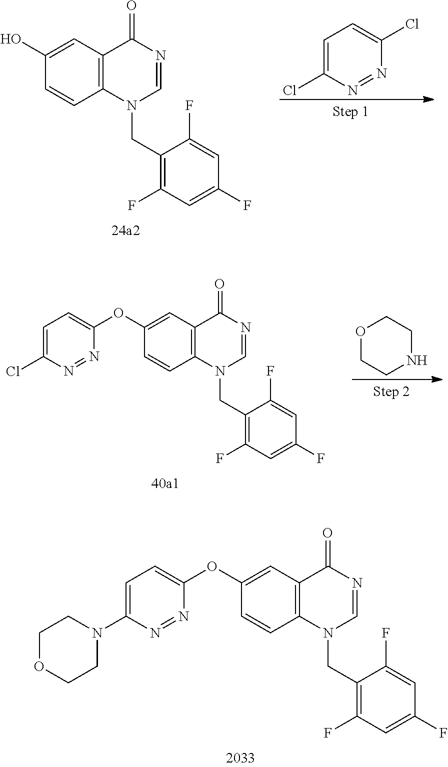

### Step 1:

Phenol 24a2 is coupled to 3,6-dichloropyridazine using the protocol described in example 5C, step 1. The product is used in the subsequent step without purification.

### Step 2:

Chloropyridazine 40a1 (60 mg, 0.14 mmol) is combined with morpholine (1 mL) and heated in a microwave at 140° C. for 20 min with stirring. The mixture is concentrated and the residue is taken up in DMSO and injected onto a prep HPLC to isolate compound 2033.

### Example 41A

### Preparation of Compounds 2038 & 2039

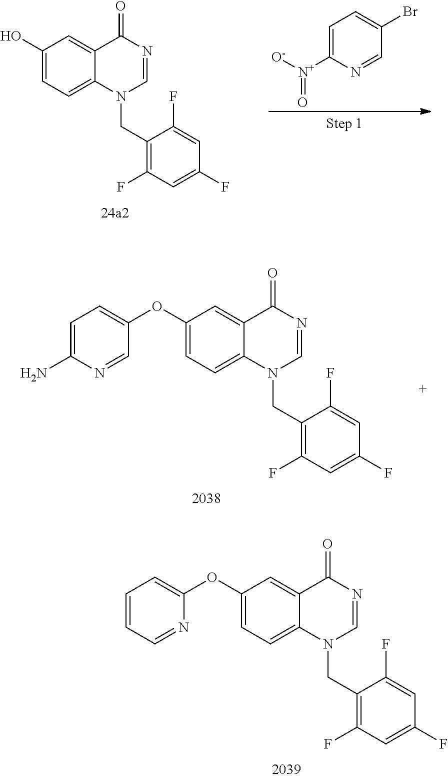

### Step 1:

i) To a solution of phenol 24a2 (240 mg, 0.57 mmol) in DMSO (3 mL) at RT is added the 5-bromo-2-nitropyridine (120 mg, 0.60 mmol) and K₂CO₃ (300 mg, 2.17 mmol). The mixture is heated to 100° C. and stirred 5 h. The mixture is then diluted with water and extracted (2×) with EtOAc, dried over MgSO₄, filtered and concentrated under vacuum.

ii) The resulting residue is dissolved in EtOH (5 mL) and Pd/C (10% w/w, 20 mg) is added. This mixture is stirred at RT for 2 h under 1 atm of H₂. The solution is filtered on celite and concentrated under vacuum. The residue is dissolved in EtOAc and extracted with 1N HCl (2×).

The organic and aqueous phases are separated and manipulated as follows:

The aqueous phase is basified with NaOH 10N and extracted (2×) with EtOAc, dried over MgSO₄, filtered and concentrated under vacuum. The residue is taken up in MeOH then Na₂CO₃ (200 mg, 3 mmol) and I₂ (145 mg, 0.57 mmol) are added and the mixture is stirred 1 h at RT. The mixture is filtered on Millex™, diluted with AcOH and injected onto a prep. HPLC to obtain aminopyridine derivative 2038. The organic phase is dried over MgSO₄, filtered and concentrated under vacuum. The residue is taken up in MeOH then Na₂CO₃ (200 mg, 3 mmol) and I₂ (145 mg, 0.57 mmol) are added and the mixture is stirred 1 h at RT. The mixture is then filtered on Millex™, diluted with AcOH then injected onto a prep. HPLC to obtain pyridine derivative 2039.

### Example 42A

### (Synthetic Method W): Preparation of Compound 2042

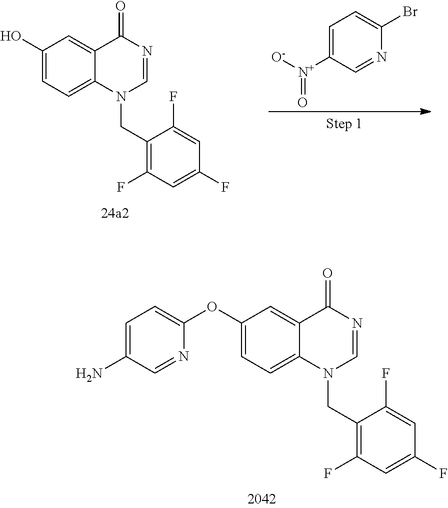

### Step 1:

i) To a solution of phenol 24a2 (60 mg, 0.20 mmol) in DMSO (2 mL) at RT is added the 2-bromo-5-nitropyridine (32 mg, 0.20 mmol) and K₂CO₃ (70 mg, 0.51 mmol). The mixture is heated to 60° C. and stirred for 1 h. The mixture is then diluted with EtOAc and washed with water and brine. The organic phase is dried over MgSO₄, filtered and concentrated.

ii) The resulting residue obtained is dissolved in EtOH (8 mL) and iron powder (39 mg, 0.70 mmol) and NH₄Cl (64 mg, 1.2 mmol) are added. This mixture is stirred at RT for 20 h. Additional iron (2 eq) and 1N HCl (3 mL) are added and stirring is continued for another 6 h. The mixture is then diluted with EtOAc and washed with sat. aq. NaHCO₃ and brine. The organic phase is dried over MgSO₄, filtered and concentrated. The residue is taken up in AcOH then injected onto a prep. HPLC to obtain the amino pyridine derivative 2042.

### Example 43A

### (Synthetic Method X): Preparation of Compound 2045

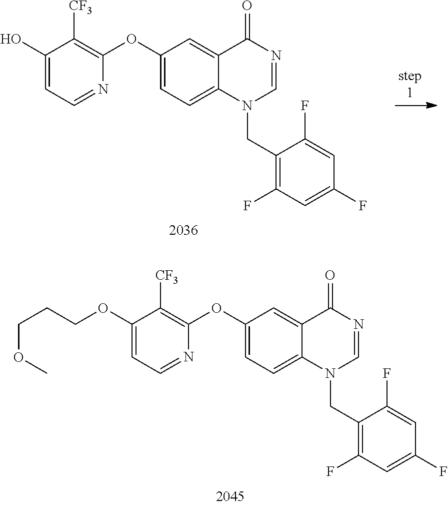

Reference: Yoakim, C.; Guse, I.; O'Meara, J.; Thavonekham, B. Synlett, 2003, 473, herein incorporated by reference.

### Step 1:

To a mixture of 3-methoxypropan-1-ol (21 μL, 0.21 mmol), compound 2036 (50 mg, 0.11 mmol) and 4-diphenylphosphinobenzoic acid 2-trimethylsilylethyl ester (0.5M in THF, 430 μL, 0.21 mmol) in THF (2 mL) is added DIAD (46 μL, 0.23 mmol). The reaction mixture is stirred 3 h at RT. To the mixture is added TBAF (1.0 M THF, 420 μL, 0.42 mmol). The mixture is stirred for an additional hour before being diluted with EtOAc and washed with water, 1N NaOH and brine. The organic phase is dried over MgSO₄, filtered and concentrated. The residue is taken up in DMSO then injected onto a prep HPLC to isolate compound 2045.

### Example 44A

### Preparation of Compound 2047

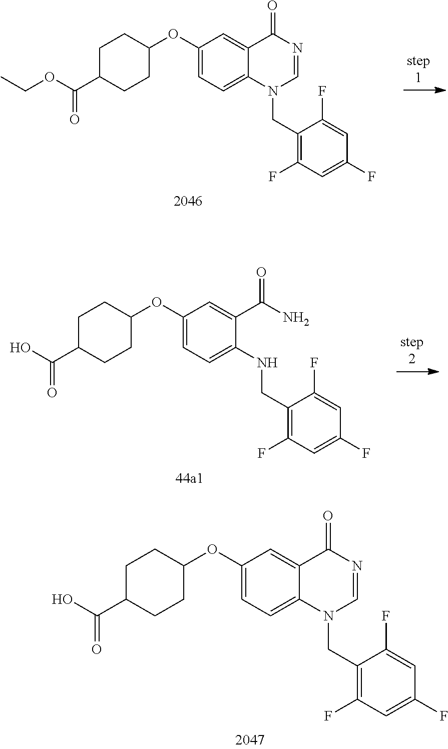

### Step 1:

To a mixture of ethylester 2046 (75 mg, 0.16 mmol) in THF (2 mL) and water (0.4 mL) is added LiOH (39 mg, 1.6 mmol). The reaction mixture is stirred overnight at RT. The mixture is concentrated and the residue is taken up in DMSO/AcOH then injected onto a prep HPLC to isolate acid 44a1.

### Step 2:

Carboxamide 44a1 is converted to quinazolinone 2047 as described in step 3 of example 1.

### Example 45A

### Preparation of Compounds 2049 & 2054

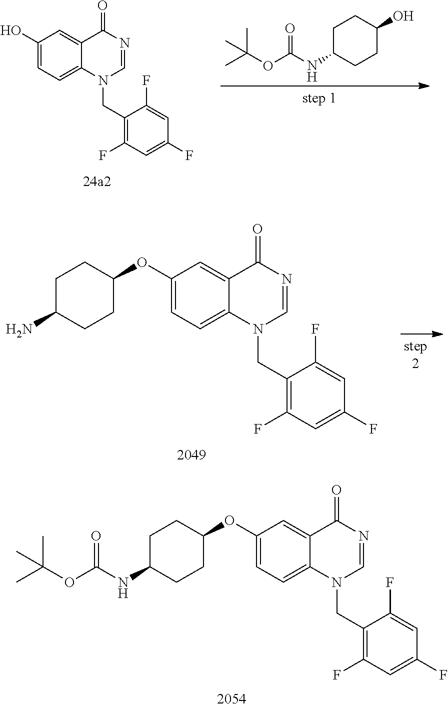

### Step 1:

i) trans-N-Boc-4-aminocyclohexanol is coupled to phenol 24a2 using the protocol described in example 38A (synthetic method U). Partial purification is accomplished by combiflash (3% MeOH in DCM).

ii) To a mixture of the Boc-protected intermediate (57 mg, 0.11 mmol) in DCM (4 mL) is added TFA (1 mL). The mixture is stirred at RT for 0.5 h. The mixture is concentrated and the residue is taken up in DMSO then injected onto a prep HPLC to isolate 2049.

### Step 2:

To a mixture of amine 2049 (4 mg, 0.01 mmol) in DCM (0.3 mL) is added Boc₂O (2 mg, 0.01 mmol) and 1N NaOH (10 μL, 0.01 mmol). The mixture is stirred at RT for 1 h. The mixture is concentrated and the residue is taken up in DMSO then injected onto a prep HPLC to isolate 2054.

### Example 46A

### Preparation of Compound 2050

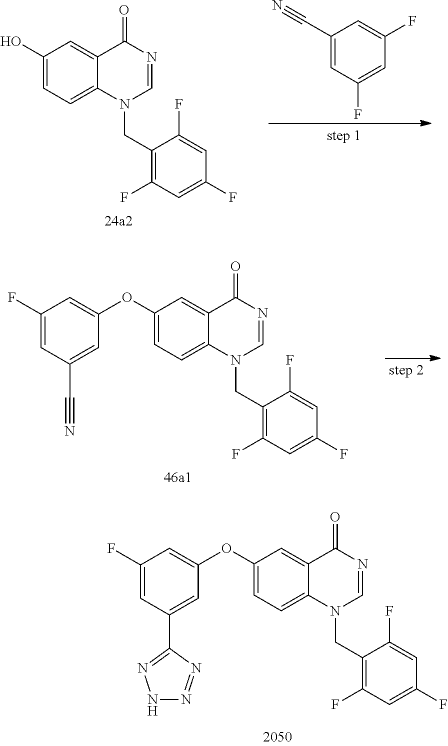

### Step 1:

3,5-difluorobenzonitrile is coupled to phenol 24a2 using the protocol described in example 5C (synthetic method B).

### Step 2:

To a mixture of nitrile 46a1 (25 mg, 0.06 mmol) in dioxane (0.5 mL) in a sealed tube vessel is added azidotributyltin (60 mg, 0.18 mmol). The mixture is heated to 100° C. and stirred for 20 h. The mixture is diluted in hexanes and the solid is collected. The solid residue is taken up in DMSO/AcOH then injected onto a prep HPLC to isolate 2050.

### Example 47A

### Preparation of Compound 2051

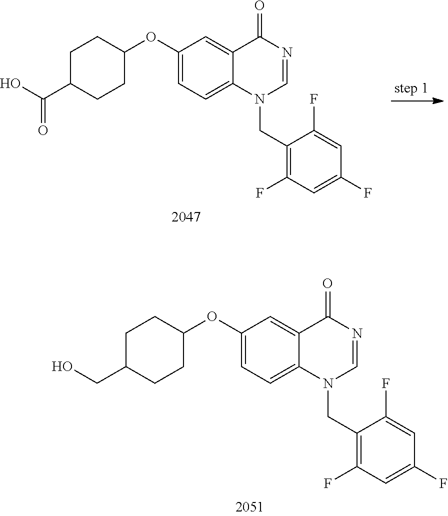

### Step 1:

To a mixture of acid 2047 (56 mg, 0.10 mmol) in THF (1 mL) is added a borane—THF complex (1.0 M in THF, 0.51 mL, 0.51 mmol). The mixture is stirred for 1 h at RT. The reaction is quenched with 1N HCl and stirred 0.5 h. The mixture is diluted with sat. aq. NaHCO₃ then extracted with EtOAc. The organic phase is dried over MgSO₄, filtered and concentrated. The residue is taken up in MeOH then Na₂CO₃ (110 mg, 1.02 mmol) and I₂ (78 mg, 0.31 mmol) are added. The mixture is stirred for 1 h at RT before being quenched by diluting the mixture with sat. aq. Na₂S₂O₃. The aqueous mixture is extracted with EtOAc. The organic phase is dried over MgSO₄, filtered and concentrated. The crude product is purified by combiflash (6% MeOH in DCM) then purified again by prep HPLC to afford compound 2051.

### Example 48A

### Preparation of Compounds 2052 & 2053

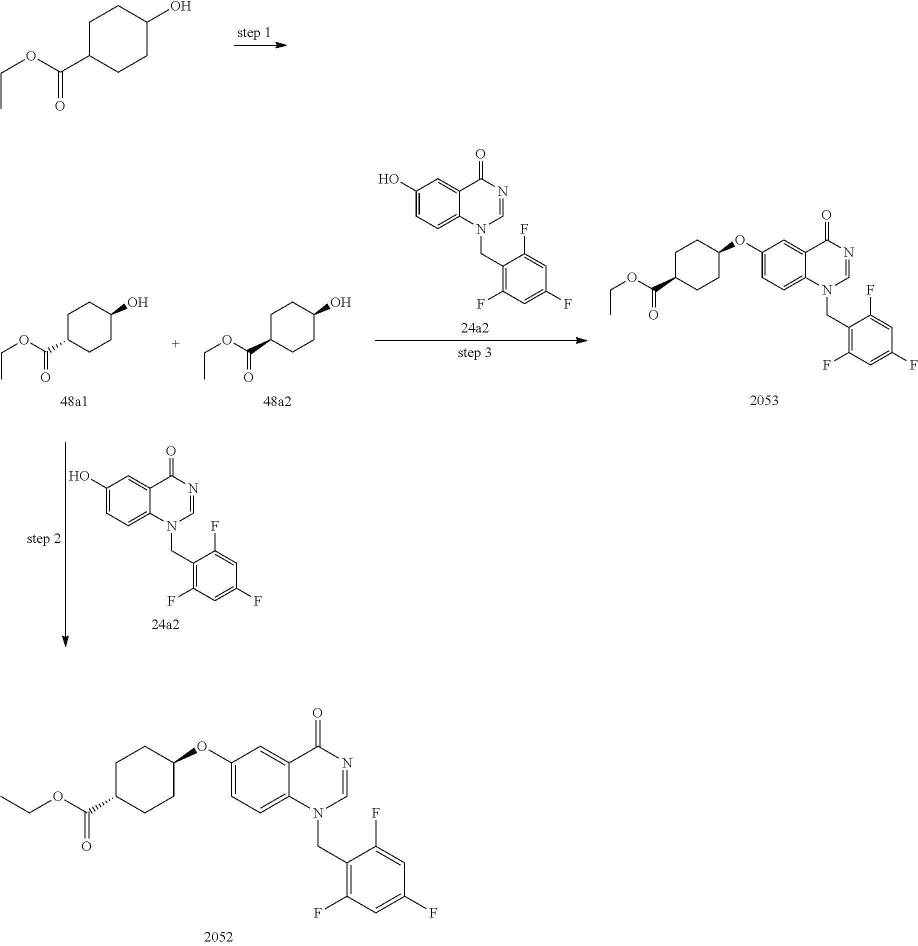

### Step 1:

Ethyl-4-hydroxycyclohexane carboxylate is separated by combiflash (20-50% EtOAc in Hex) to afford the trans- and cis-isomers 48a1 and 48a2.

### Steps 2 & 3:

The respective alcohols 48a1 and 48a2 are coupled to phenol 24a2 using the protocol described in example 38A (synthetic method U) to afford compounds 2052 and 2053.

### Example 49A

### (Synthetic Method Y): Preparation of Compound 2057

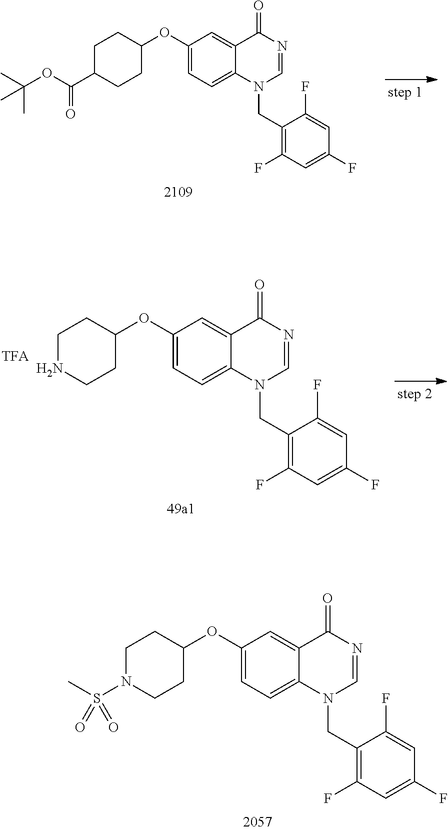

### Step 1:

The Boc group of 2109 is deprotected using the protocol described in step 1ii) of example 45. The crude TFA salt 49a1 is not purified.

### Step 2:

To a mixture of acid 49a1 (45 mg, 0.12 mmol) in DMSO (1 mL) is added NEt₃ (80 μL, 0.58 mmol) and MsCl (10 μL, 0.17 mmol). The mixture is stirred for 0.5 h at RT. The mixture is acidified with AcOH then injected onto a prep HPLC to isolate compound 2057.

### Example 50A

### Preparation of Compound 2060

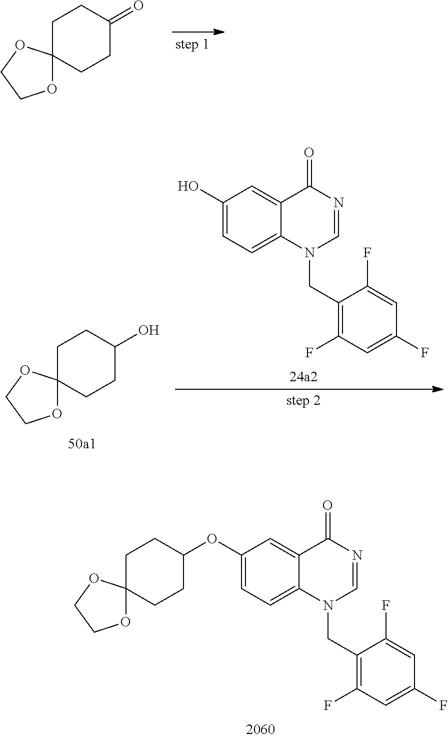

### Step 1:

1,4-cyclohexanedione monoethyleneketal is reduced to alcohol 50a1 using the protocol described in step 2 of example 25A.

### Step 2:

Alcohol 50a1 is coupled to phenol 24a2 using the protocol described in example 38A (synthetic method U) to afford compoud 2060.

### Example 51A

### Preparation of Compound 2061

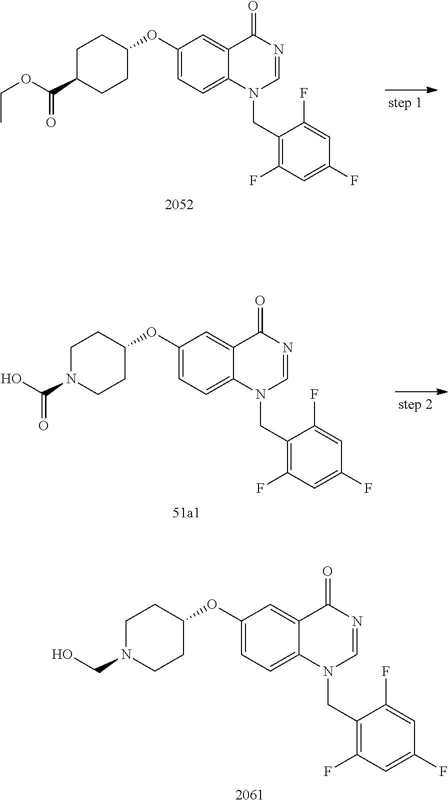

### Step 1:

To ester 2052 (160 mg, 0.35 mmol) in DMSO (2 mL) is added 10N NaOH (38 μL, 0.38 mmol). The mixture is stirred for 0.5 h at RT. Another equivalent of NaOH is added and the mixture is stirred for 20 min. The mixture is acidified with AcOH then injected onto a prep HPLC to isolate acid 51a1.

### Step 2:

Acid 51a1 is reduced to compound 2061 using the protocol described in step 1 of example 47A.

### Example 52A

### Preparation of Compound 2062

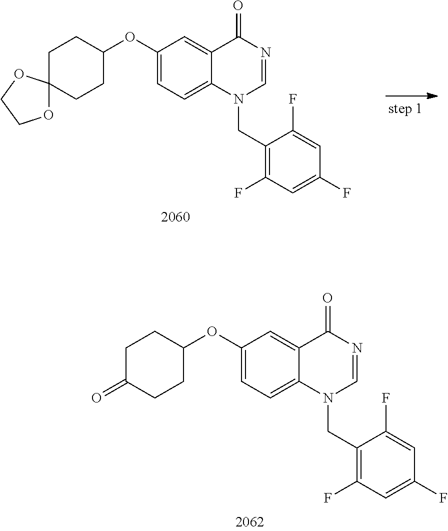

### Step 1:

To a mixture of ketal 2060 (30 mg, 0.05 mmol) in DCM (0.9 mL) and water (0.1 mL) is added TFA (0.1 mL). The mixture is stirred at RT for 2 h. The mixture is concentrated and the residue is taken up in DMSO then injected onto a prep. HPLC to isolate 2062.

### Example 53A

### Preparation of Compound 2063

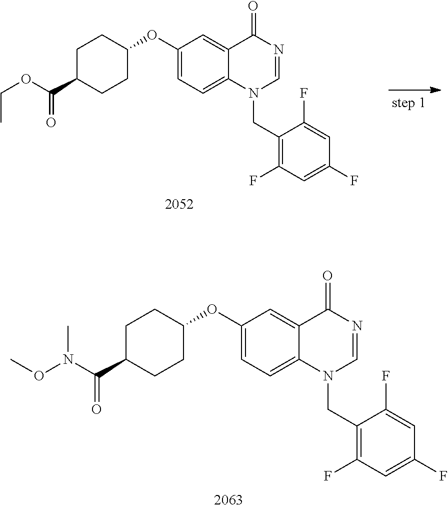

Reference: Williams, J., M.; Jobson, R. B.; Yasuda, N. Marchesini, G.; Dolling, U.-H. Tetrahedron Lett. 1995, 36, 5461, herein incorporated by reference.

### Step 1:

A stirring mixture of ester 2052 (48 mg, 0.10 mmol) and N,O-dimethylhydroxylamine.HCl (16 mg, 0.16 mmol) in THF (1 mL) is cooled to −15° C. i-PrMgCl (2.0 M in Et₂O, 156 μL, 0.31 mmol) is added and the mixture is stirred for 0.5 h. The mixture is diluted in sat. aq. NH₄Cl and extracted EtOAc (3×). The combined organic extracts are dried over MgSO₄, filtered and concentrated. The residue is taken up in DMSO then injected onto a prep. HPLC to isolate 2063.

### Example 54A

### Preparation of Compound 2065

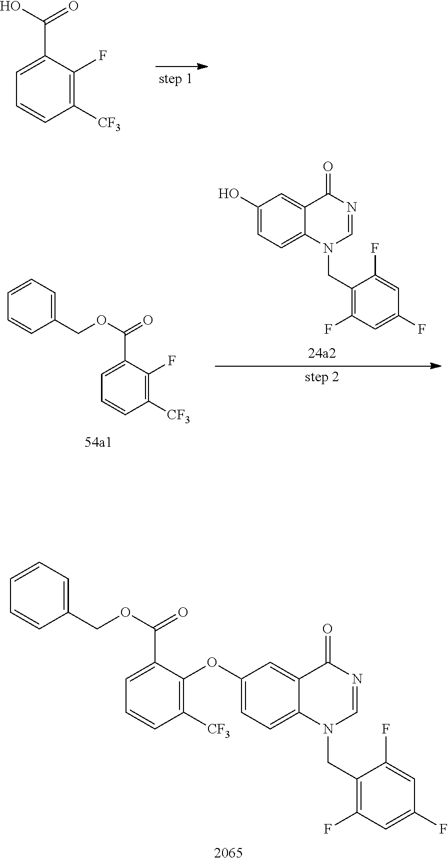

### Step 1:

To a mixture of 2-fluoro-3-trifluoromethylbenzoic acid (1.00 g, 4.8 mmol) in MeCN (10 mL) is added BnBr (0.63 mL, 5.3 mmol) and DBU (0.86 mL, 5.8 mmol). The mixture is stirred at RT overnight. The mixture is concentrated and the residue is diluted in EtOAc. The organic phase is washed with 1N HCl, water, 1N NaOH and brine then dried over MgSO₄, filtered and concentrated. The crude product is purified by flash chromatography to afford benzyl ester 54a1.

### Step 2:

Fluoroarene 54a1 is coupled to phenol 24a2 using the protocol described in example 13A (synthetic method G) to afford compound 2065.

### Example 55A

### Preparation of Compound 2066

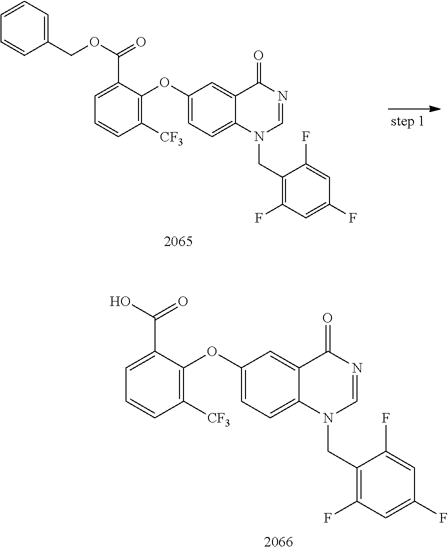

### Step 1:

To a mixture of ester 2065 (60 mg, 0.09 mmol) in DMSO (1 mL) is added 2.5N NaOH (0.2 mL, 0.50 mmol). The mixture is stirred for 2 h at RT. The mixture is acidified with AcOH then injected onto a prep HPLC to isolate acid 2066.

### Example 56A

### (Synthetic Method Z): Preparation of Compound 2067

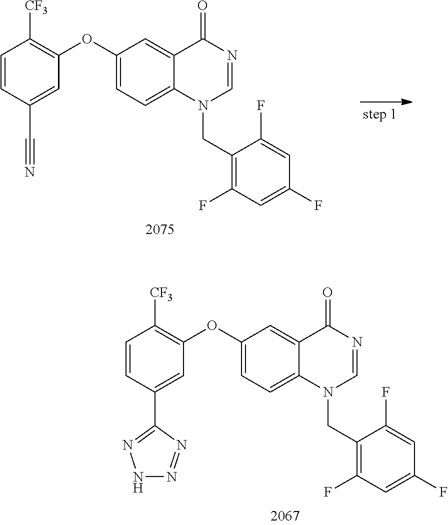

### Step 1:

Nitrile 2075 is converted to tetrazole derivative 2067 using the protocol described in step 2 example 46A.

### Example 57A

### Preparation of Compound 2068

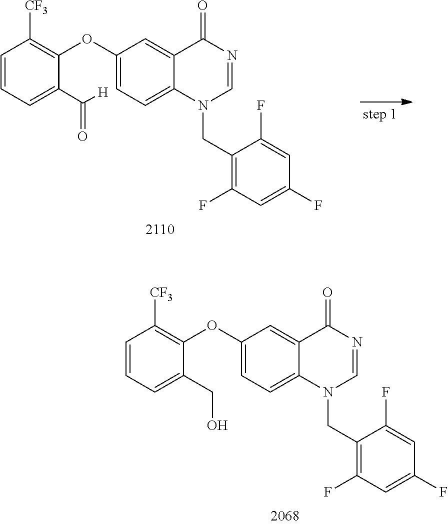

### Step 1:

Aldehyde 2110 is reduced to alcohol derivative 2068 using the protocol described in steps 2 & 3 of example 25A.

### Example 58A

### (Synthetic Method AA): Preparation of Compound 2070

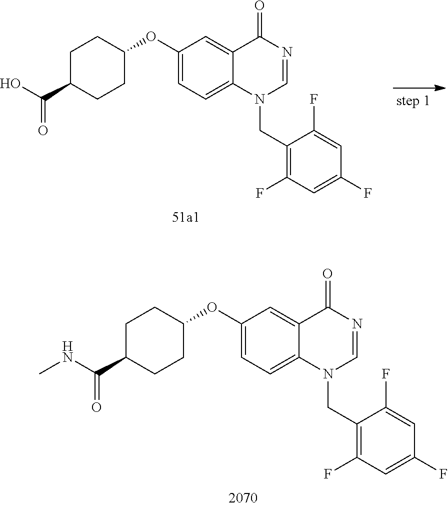

### Step 1:

To a mixture of acid 51a1 (30 mg, 0.07 mmol) and methylamine.HCl (8 mg, 0.11 mmol) in DMF (0.6 mL) is added NEt₃ (60 μL, 0.42 mmol) and HATU (29 mg, 0.08 mmol). The mixture is stirred for 3 h at RT. The mixture is acidified with AcOH then injected onto a prep HPLC to isolate amide 2070.

### Example 59A

### Preparation of Compound 2072

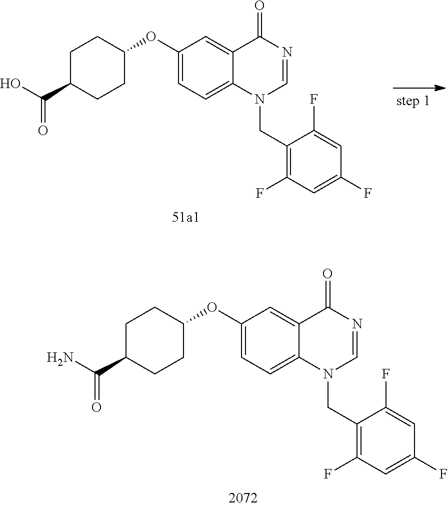

### Step 1:

To a mixture of acid 51a1 (23 mg, 0.05 mmol) in THF (1 mL), cooled to −15° C., are added N-methylmorpholine (8 μL, 0.07 mmol) and i-butylchloroformate (8 μL, 0.06 mmol). The mixture is stirred for 0.5 h. Saturated aq. NH₄OH (˜10 μL) is added and the mixture is allowed to warm to RT and stirring is continued for 3 h. The mixture is concentrated and the residue is taken up in AcOH then injected onto a prep HPLC to isolate carboxamide 2072.

### Example 60A

### Preparation of Compound 2076

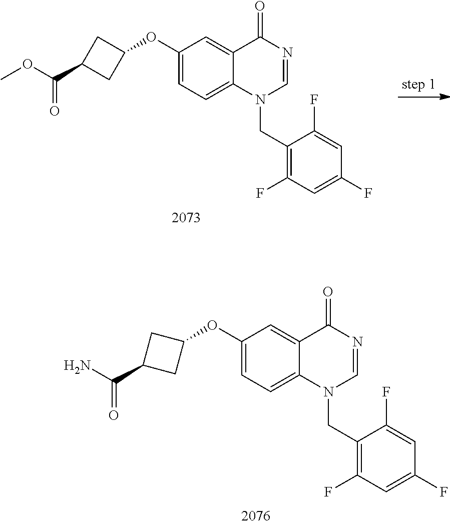

### Step 1:

To a mixture of methylester 2073 (54 mg, 0.13 mmol) in MeOH (1.3 mL) is added LiOH (31 mg, 1.3 mmol). The reaction mixture is stirred for 2 h at RT. The mixture is concentrated and the residue is taken up in DMSO/AcOH then injected onto a prep HPLC to isolate acid 2076.

### Example 61A

### Preparation of Compound 2077

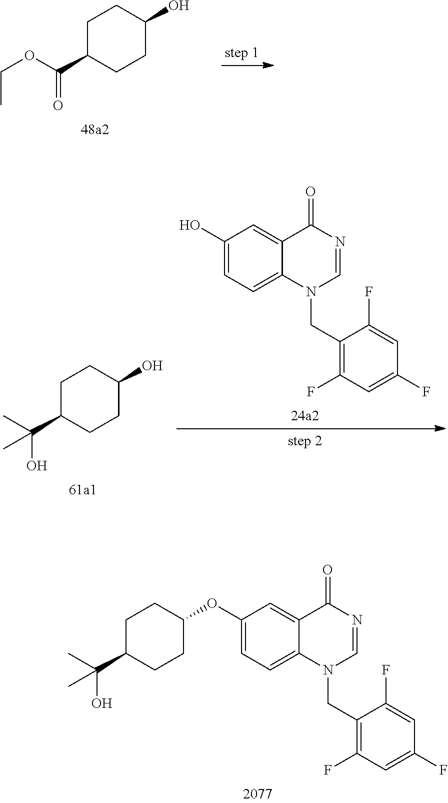

### Step 1:

To a mixture of ethylester 48a2 (100 mg, 0.58 mmol) in THF (6 mL), chilled to 0° C., is added MeMgBr (3.0 M in ether, 0.77 mL, 2.3 mmol). The reaction mixture is warmed to RT and stirred for 2 h. The mixture is diluted in 0.1N HCl then extracted with EtOAc (3×). The organic phase is dried over MgSO₄, filtered and concentrated. The crude product is filtered through a pad of silica gel (eluent: EtOAc) to afford diol 61a1.

### Step 2:

Diol 61a1 is coupled to phenol 24a2 using the protocol described in example 38A (synthetic method U) to afford compound 2077. The product is successively purified by prep HPLC and combiflash.

### Example 62A

### Preparation of Compounds 4022 & 2078

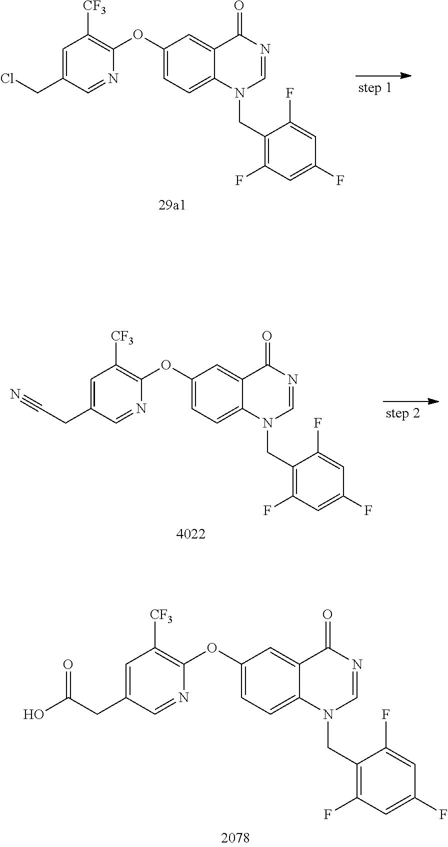

### Step 1:

To a mixture of benzylchloride 29a1 (320 mg, 0.63 mmol) in DCM (20 mL) is added Et₄NCN (170 mg, 1.1 mmol). The reaction mixture is stirred for 5 h at RT. The mixture is diluted in DCM and washed with 0.5N HCl, sat. aq. NaHCO₃, water and brine. The organic phase is dried over MgSO₄, filtered and concentrated. The crude nitrile 4022 is advanced to the next step without further purification.

### Step 2:

A mixture of nitrile 4022 (300 mg, 0.61 mmol) in AcOH (4 mL) and conc. H₂SO₄ (1 mL) is heated to 100° C. and stirred for 2 days. The mixture is then poured into water and neutralized by the careful addition of 1N NaOH until a precipitate is formed. The solid 2078 is collected by filtration and dried.

### Example 63A

### (Synthetic Method AB): Preparation of Compound 2079

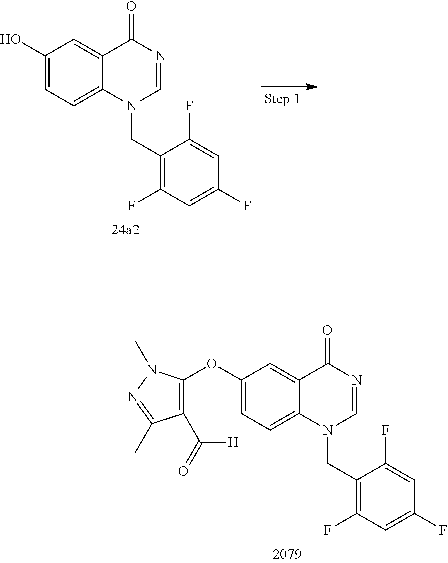

### Step 1:

Phenol 24a2 (250 mg, 0.50 mmol) is combined with 5-chloro-1,3-dimethyl-1H-pyrazole-4-carboxaldehyde (190 mg, 1.2 mmol) and Cs₂CO₃ (650 mg, 2.0 mmol) in DMSO (4 mL). The mixture is stirred in a microwave at 110° C. for 10 min. After cooling to RT, AcOH is added to the mixture which is filtered, then injected onto a HPLC to isolate compound 2079.

### Example 64A

### Preparation of Compound 2080

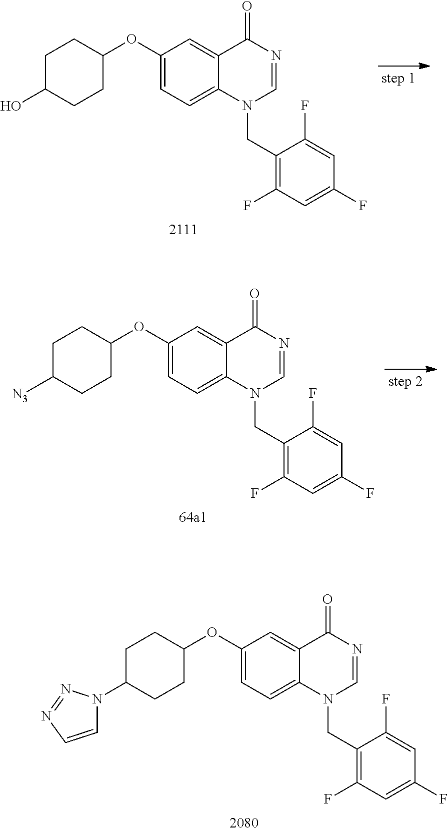

### Step 1:

To a mixture of alcohol 2111 (110 mg, 0.26 mmol) in THF (3 mL) are added PPh₃ (200 mg, 0.78 mmol), DPPA (170 μL, 0.78 mmol) and DIAD (160 mg, 0.78 mmol). The mixture is stirred for 0.5 h at RT. The mixture is then diluted in sat. aq. NH₄Cl then extracted with EtOAc (3×). The combined organic extracts are dried over MgSO₄, filtered and concentrated. Crude azide 64a1 is purified by combiflash (2-5% MeOH/DCM).

### Step 2:

To a mixture of azide 64a1 (25 mg, 0.06 mmol) in DCM (2 mL) is added trimethylsilylacetylene (160 μL, 1.16 mmol). The mixture is heated in a microwave at 130° C. for 30 min with stirring. This heating cycle is repeated 4 additional times. The mixture is concentrated and the residue is taken up in AcOH then injected onto a prep HPLC to isolate triazole derivative 2080.

### Example 65A

### (Synthetic Method AD): Preparation of Compound 2102

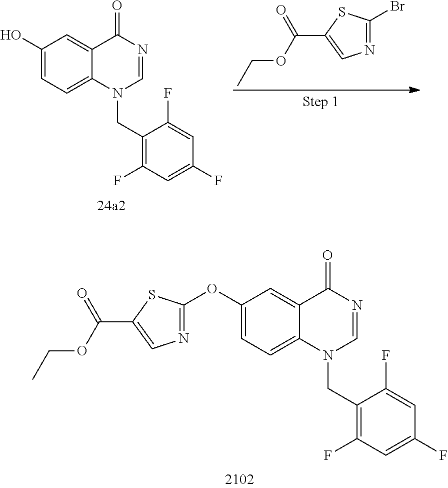

### Step 1:

To a mixture of phenol 24a2 (1.5 g, 3.6 mmol) in DMF (100 mL) are added ethyl-2-bromothiazole-5-carboxylate (1.0 g, 4.3 mmol) and K₂CO₃ (0.99 g, 11 mmol). The mixture is heated to 90° C. and stirred for 18 h under an Ar atmosphere. The mixture is diluted in water then extracted with EtOAc (2×). The combined organic extracts are dried over MgSO₄, filtered and concentrated. A sample of the crude product is purified by prep HPLC to isolate 2102.

### Example 65B

### (Synthetic Method AE): Preparation of Compound 2086

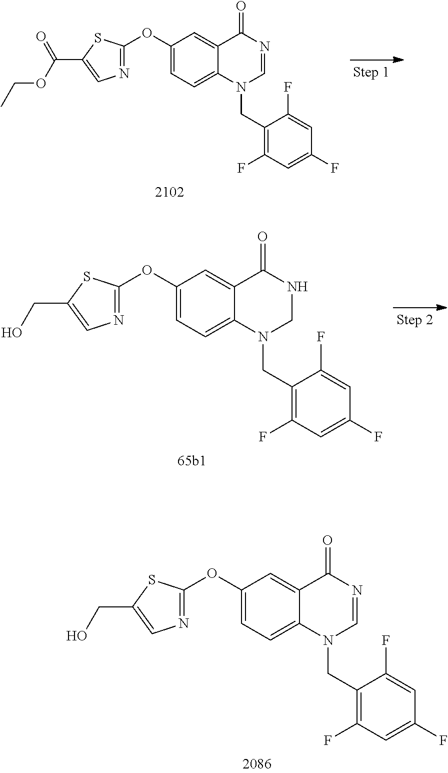

### Step 1:

To a crude mixture (not purified by prep HPLC) of ester 2102 (200 mg, 0.43 mmol) in THF (8 mL) chilled to 0° C. is added DiBAI-H (1.0 M in THF, 3.25 mL, 3.25 mmol). The mixture is warmed to RT and stirred for 18 h before being quenched by the addition of a sat. aq. solution of Rochelle's salt. The mixture is then extracted with EtOAc and the organic extract is concentrated. The crude product is purified by combiflash (0 to 10% MeOH/DCM) to afford alcohol 65b1.

### Step 2:

Alcohol 65b1 is converted to compound 2086 using the protocol described in step 3 of example 25A.

### Example 66A

### (Synthetic Method AF): Preparation of Compound 2088

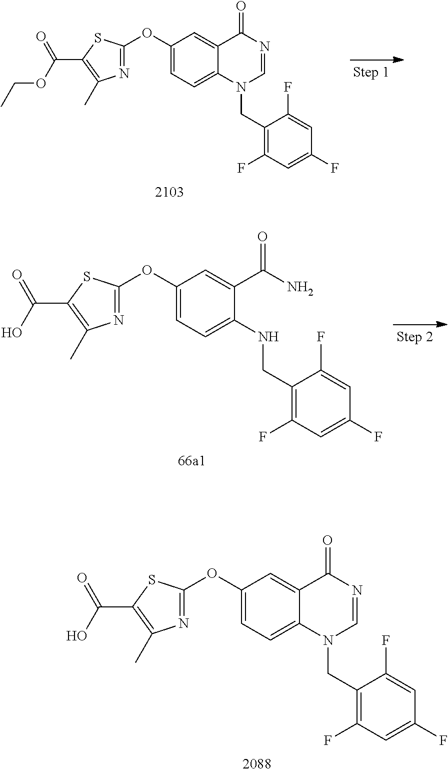

### Step 1:

To a mixture of ester 2103 (100 mg, 0.21 mmol) in DMSO (0.5 mL) chilled to 0° C. is added 1N NaOH (1.1 mL, 1.1 mmol). The mixture is allowed to warm to RT and is stirred for 3 h. The pH of the mixture is adjusted to −3-4 and the resulting precipitate is recovered by filtration and washed with 1N HCl. The crude acid 66a1 is dried under vacuum then advanced to the next step without further purification.

### Step 2:

Carboxamide 66a1 is converted to quinazolinone 2088 as described in step 3 of example 1A.

### Example 67A

### Preparation of Compound 2091

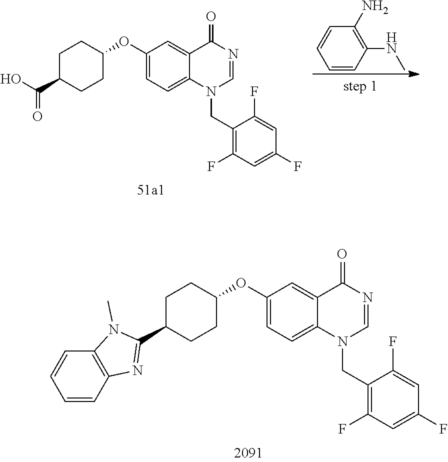

### Step 1:

To a mixture of acid 51a1 (40 mg, 0.09 mmol) and N-methyl-1,2-phenylenediamine (34 mg, 0.29 mmol) in DMF (0.6 mL) are added NEt₃ (38 μL, 0.27 mmol) and TBTU (35 mg, 0.11 mmol). The mixture is stirred for 1.5 h at RT. The mixture is diluted in EtOAc and washed with water and brine. The organic phase is dried over MgSO₄, filtered and concentrated. The residue is taken up in AcOH and the mixture is heated to 70° C. in an oil bath and stirred for 1 h. The mixture is filtered then injected onto a prep HPLC to isolate benzimidazole derivative 2091.

### Example 68A

### Preparation of Compound 2097

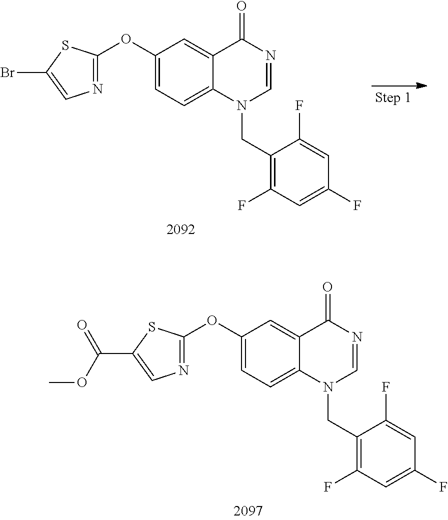

### Step 1:

To a mixture of bromide 2092 (25 mg, 0.05 mmol) in DMSO (0.7 mL) and MeOH (0.35 mL) is added NEt₃ (40 μL, 0.28 mmol) and Pd(dppf)Cl₂ (4.4 mg, 0.01 mmol). CO is bubbled through the mixture for 5 min before the mixture is heated to 85° C. and stirred for 36 h under 1 atm of CO (balloon). The mixture is diluted in MeCN, filtered then injected onto a prep HPLC to isolate methylester 2097.

### Example 69A

### Preparation of Compound 2100

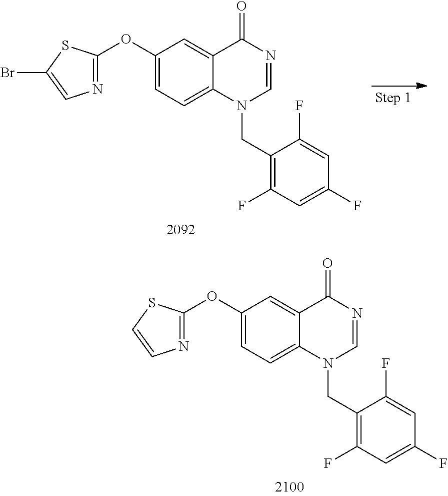

### Step 1:

To a mixture of bromide 2092 (11 mg, 0.02 mmol) in THF (0.5 mL) cooled to −78° C. is added i-PrMgCl.LiCl (1.3 M in THF, 20 μL, 0.03 mmol). The mixture is stirred for 2 h at −78° C. Saturated aq. NH₄Cl (1 mL) is added and the mixture is allowed to warm to RT. The mixture is diluted in MeCN, filtered then injected onto a prep HPLC to isolate methylester 2100.

### Example 70A

### Preparation of Intermediate 70a4

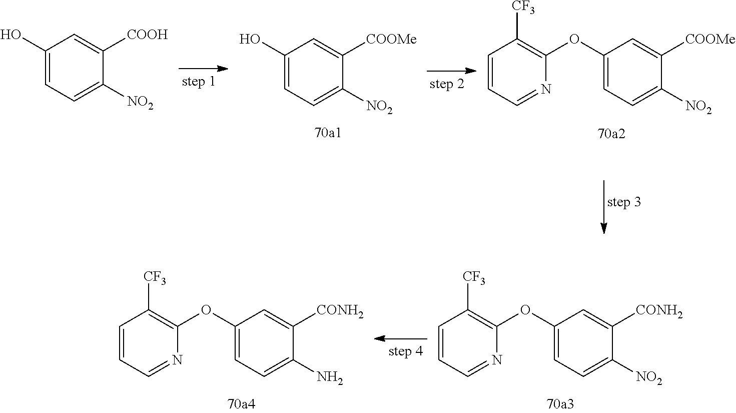

### Step 1:

BF₃-Et₂O (110 mL, 870 mmol) is added to 5-hydroxy-2-nitrobenzoic acid (15 g, 81.3 mmol) in MeOH (250 mL) at RT. Et₂O is distilled off until the temperature reaches 70° C. and the reaction mixture is heated to reflux overnight. BF₃-Et₂O (50 mL) is added to complete the reaction with an additional 24 h at reflux. MeOH is removed under vacuum and the residue is diluted in DCM (300 mL), washed with water, brine, dried over Na₂SO₄ and concentrated under vacuum to afford methylester 70a1.

### Step 2:

To a mixture of phenol 70a1 (10 g, 50.8 mmol) in DMSO (100 mL) are added at RT 2-fluoro-3-trifluoromethylpyridine (8.9 mL, 76 mmol) and K₂CO₃ (24.5 g, 180 mmol). The reaction is stirred overnight at 100° C. then cooled to RT. Water (300 mL) is added and the compound is extracted with EtOAc (3×). The combined organic layers are washed with water, brine, dried over Na₂SO₄ and concentrated under vacuum. Purification by column chromatography (20% EtOAc/Hex) affords compound 70a2.

### Step 3:

A mixture of 70a2 (4.0 g, 12.0 mmol) in NH₃/MeOH (50 mL) is stirred 24 h at 100° C. in a sealed tube. The mixture is concentrated and crude product is purified by flash chromatography (2% MeOH/DCM) to afford carboxamide 70a3.

### Step 4:

To a stirred mixture of nitroarene 70a3 (5.0 g, 15 mmol) in MeOH (150 mL), is added Raney-Ni (1.0 g). The reaction is stirred 24 h under H₂ pressure (15-20 psi) at RT. The reaction mixture is filtered through celite and the filtrate is concentrated under vacuum to afford 70a4.

### Example 70B

### (Synthetic Method AG): Preparation of Compound 3001

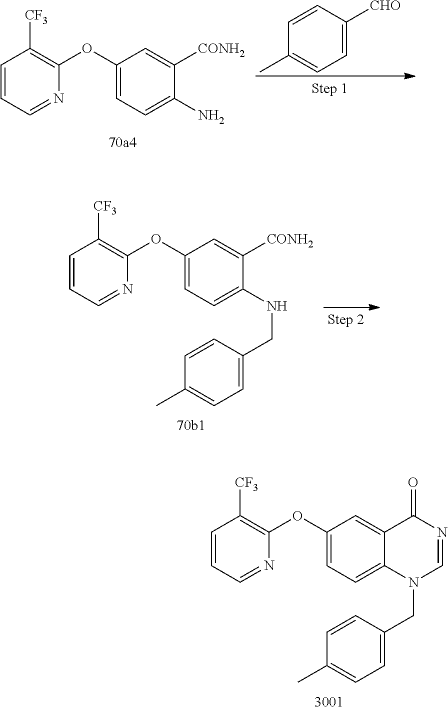

### Step 1:

To a stirred mixture of aniline 70a4 (0.50 g, 1.7 mmol) in THF/MeOH (17 mL, 1:1) at 0° C. are added 4-methylbenzaldehyde (0.81 g, 6.7 mmol), NaBH₃CN (0.42 g, 6.7 mmol) and a catalytic amount of AcOH. The reaction mixture is stirred at RT for 48 h. The solvent is removed under vacuum and the residue is taken up in DCM, washed with water, brine, dried over Na₂SO₄ and concentrated under vacuum. The crude product is purified by flash chromatography (20% AcOEt/petroleum ether) to afford intermediate 70b1.

### Step 2:

A mixture of carboxamide 70b1 (100 mg, 0.25 mmol) in (EtO)₃CH (15 mL) is heated to 120° C. and stirred for 5 h. The mixture is allowed to cool to RT whereupon a precipitate is formed. The solid is collected by filtration and dried to afford compound 3001.

### Example 71A

### (Synthetic Method AH): Preparation of Compound 3007

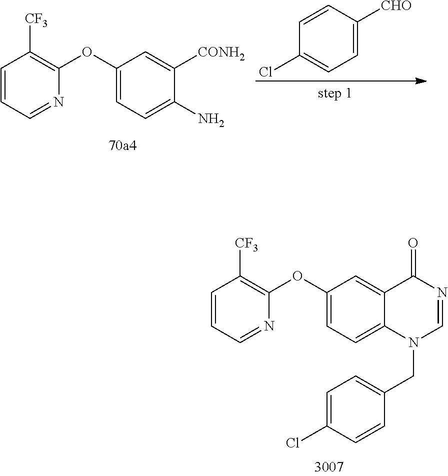

### Step 1:

Reductive amination methodology described in the following publication: Abdel-Magid, A. F.; Carson, K. G.; Harris, B. D.; Maryanoff, C. A.; Shah, R. D. J. Org. Chem. 1996, 61, 3849, herein incorporated by reference.

To a stirred mixture of aniline 70a4 (25 mg, 0.08 mmol) in DCE (1 mL) are added 4-chlorobenzaldehyde (12 mg, 0.08 mmol), Na(AcO)₃BH (25 mg, 0.12 mmol) and AcOH (5 μL). The mixture is agitated on an orbital shaker at RT overnight. Another portion of aldehyde, Na(AcO)₃BH and AcOH are added and the mixture is agitated for an additional 24 hr. Triethylorthoformate (0.5 mL) is added and the mixture is warmed to 80° C. and agitated overnight. The reaction mixture is filtered then injected onto a prep HPLC to isolate compound 3007.

### Example 72A

### (Synthetic Method AI, Step 2): Preparation of Compound 3023

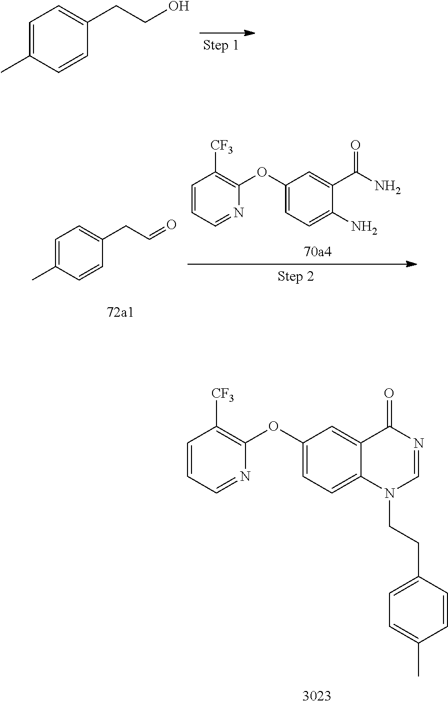

### Step 1:

To a mixture of 2-(4-methylphenyl)ethanol (100 mg, 0.73 mmol) in DCM (4 mL) is added Dess-Martin periodinane (340 mg, 0.80 mmol). The mixture is stirred for 2 h at RT. The resulting mixture is diluted with EtOAc and washed with sat. aq. NaHCO₃, 10% aq. citric acid and brine. The organic phase is dried over MgSO₄ and filtered and concentrated. Crude aldehyde 72a1 is utilized in the next step without further purification.

### Step 2:

To the aniline 70a4 (60 mg, 0.20 mmol) in 1:1 DCM/THF (3 mL) is added aldehyde 72a1 (27 mg, 0.20 mmol), Na(OAc)₃BH (170 mg, 0.80 mmol) and AcOH (10 μL). The reaction is stirred for 2 h at RT. The resulting mixture is diluted with EtOAc and washed with sat. aq. NaHCO₃ and brine then dried over MgSO₄, filtered and concentrated under vacuum. To the residue is added (MeO)₃CH (2 mL) and TFA (50 μL) and the reaction mixture is stirred for 2 h at RT. The resulting solution is concentrated and purified by semi-prep. HPLC to afford 3023.

### Example 73A

### (Synthetic Method AJ): Preparation of Compound 3025

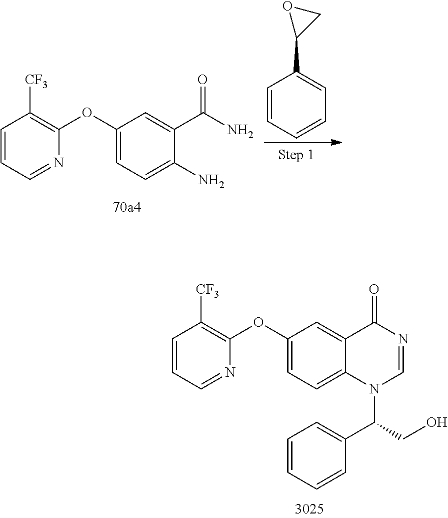

### Step 1:

i) To the aniline 70a4 (110 mg, 0.37 mmol) in cyclohexane/MeCN (1:1, 2 mL) is added S-styrene oxide (49 mg, 0.4 mmol) and BiCl₃ (20 mg, 0.06 mmol). This mixture is stirred during 20 h in a sealed tube at 60° C. The resulting solution is filtered on Millex™ and concentrated under vacuum.

ii) The residue obtained is dissolved in (MeO)₃CH (2 mL), TFA is added (20 μL) and the reaction is stirred for 1 h at RT. The resulting solution is concentrated, dissolved in DMSO/MeOH (1:1, 3 mL) and purified by semi-prep. HPLC to afford 3025.

### Example 74A

### Preparation of Compound 3027

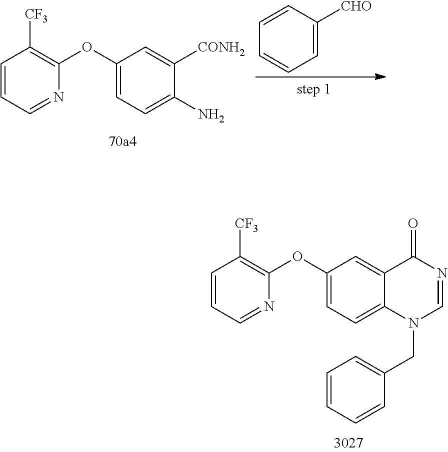

### Step 1:

To a stirred mixture of aniline 70a4 (40 mg, 0.13 mmol) in MeOH (2 mL) are added benzaldehyde (15 μL, 0.15 mmol), NaBH₃CN (13 mg, 0.20 mmol) and AcOH (20 μL). The reaction mixture is stirred at RT for 20 h. Water (45 μL) is added to the mixture before the solvent is removed under vacuum. The residue is taken up in (MeO)₃CH (2 mL) and TFA is added (20 μL). This mixture is stirred for 5 h at RT. The resulting solution is concentrated, dissolved in DMSO/AcOH (1:1, 2 mL) and purified by semi-prep. HPLC to afford 3027.

### Example 75A

### (Synthetic Method AK): Preparation of Compound 3028

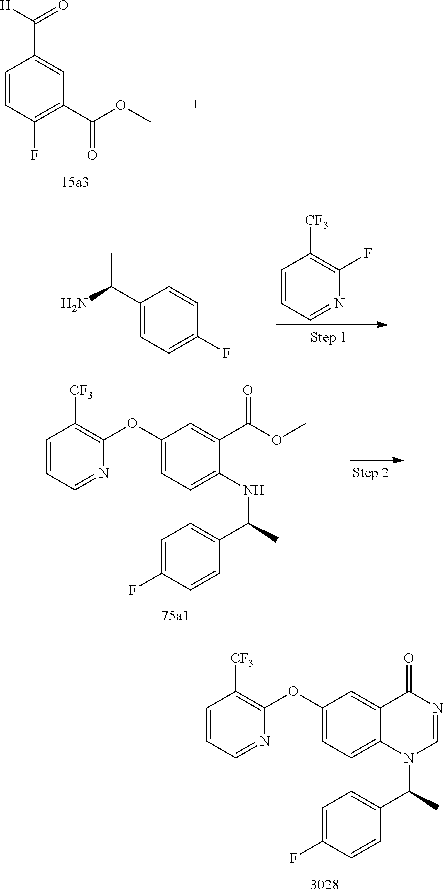

### Step 1:

i) To (S)-1-(4-fluorophenyl)ethylamine (170 mg, 1.2 mmol) is added K₂CO₃ (50 mg, 0.36 mmol) and a solution of fluoroarene 15a3 (55 mg, 0.30 mmol) in DMSO (0.5 mL). The reaction is agitated on an orbital shaker at 70° C. overnight. Water (1 mL) and HCl 12M (0.7 mL) are added and this reaction mixture is stirred for 2 h at 70° C. The resulting solution is diluted with EtOAc (4 mL), washed with brine (2×) and concentrated under vacuum.

ii) To the resulting residue is added MeOH (3 mL) and the reaction mixture is cooled to 2° C. and H₂O₂ 30% (45 μL, 0.40 mmol) and H₂SO₄ 16% (25 μL, 0.04 mmol) are added. The reaction is stirred for 6 h at 2° C. To the resulting solution is added brine (2 mL) and the desired product is extracted (2×) with EtOAc (4 mL). The combined organic phases are washed (2×) with brine, dried over MgSO₄, filtered and concentrated under vacuum.

iii) To the residue is added K₂CO₃ (150 mg, 1.08 mmol), DMSO (0.5 mL) and 2-fluoro-3-trifluoromethylpyridine (60 mg, 0.36 mmol). This mixture is stirred at 85° C. overnight. The resulting solution is filtered, washed with DMSO (1 mL) and AcOH (1 mL). The filtrate is diluted with AcOH (1 mL) and purified by semi-prep. HPLC to afford the product 75a1.

### Step 2:

i) To the ester 75a1 (75 mg, 0.028 mmol) in DMSO (1 mL) is added 1N NaOH (130 μL, 0.13 mmol) and this mixture is stirred 20 h at RT. THF (2 mL) and MgSO₄ are added and the solution is filtered on Millex™

ii) (NH₄)HCO₃ (10 mg, 0.13 mmol) and HATU (20 mg, 0.053 mmol) are added to the filtrate and the resulting mixture is stirred at RT for 1 h. The resulting solution is concentrated under nitrogen flow and (MeO)₃CH (2 mL) and TFA (50 μL, 0.65 mmol) are added. The reaction is stirred for 1 h at RT. The mixture is directly purified by semi-prep. HPLC to isolate compound 3028.

### Example 76A

### Preparation of Compound 3034

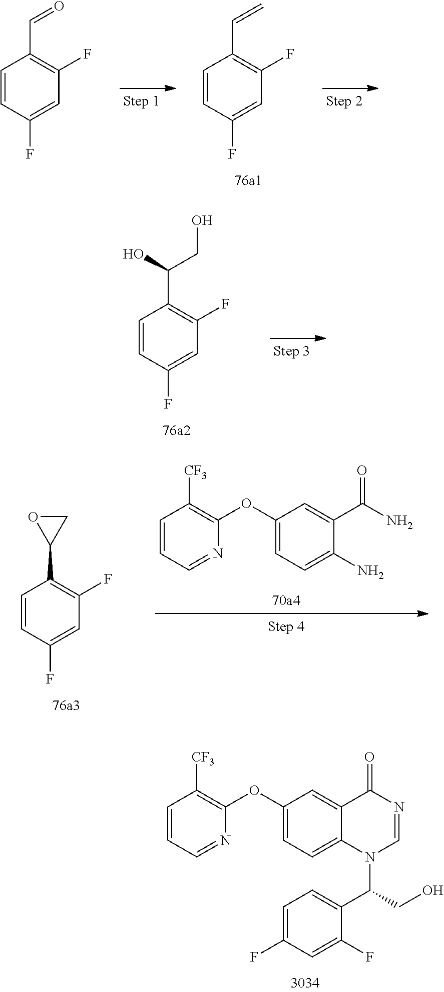

### Step 1:

To a mixture of MeP(Ph)₃Br (16.1 g, 45 mmol) in THF (130 mL) chilled to 0° C. is added KHMDS (0.91 M in THF, 49.5 mL, 45 mmol) and the mixture is allowed to warm to RT and is stirred for 15 min. The mixture is re-chilled to 0° C. and 2,4-difluorobenzaldehyde (3.0 mL, 27.4 mmol) is added as a solution in THF (10 mL). The mixture is allowed to warm to RT once again and is stirred for 30 min. The reaction is quenched with water and the resulting mixture is diluted with EtOAc and washed with water and brine. The organic phase is dried over MgSO₄, filtered and concentrated under vacuum. The crude product is filtered through a pad of silica gel (eluent: 10% EtOAc in Hex) to afford alkene 76a1.

### Step 2:

To a mixture of alkene 76a1 (322 mg, 2.3 mmol) in t-BuOH/H₂O (1:1, 12 mL) is added K₂OsO₄. 2H₂O (25 mg, 0.07 mmol), (DHQ)₂PHAL (53 mg, 0.07 mmol) and NMO (60% in water, 0.60 mL, 3.5 mmol). The mixture is stirred for 21 h at RT. The reaction mixture is diluted in toluene (10 mL) and aq. Na₂SO₃ (492 mg, 3.9 mmol in 5 mL of H₂O). The mixture is stirred for 2 h before 0.3M H₂SO₄ and sat. aq. Na₂SO₄ are added. The organic layer is separated then washed with sat. aq. Na₂SO₄. The organic phase is dried over MgSO₄, filtered and concentrated under vacuum. The crude diol 76a2 is used in the next step without further purification.

### Step 3:

To a mixture of tricyclohexylphosphine (467 mg, 1.7 mmol) in THF (6 mL) chilled to 5° C. is added DIAD (310 μL, 1.6 mmol). The mixture is allowed to warm to 15° C. and stirred for 10 min. To this is slowly added diol 76a2 (200 mg, 1.2 mmol) in THF (3 mL). The mixture is stirred for 2 h at RT and then the reaction mixture is concentrated. The residue is subjected to flash chromatography (1:9 EtOAc/Hex) to isolate epoxide 76a3.

### Step 4:

Epoxide 76a3 and aniline 70a4 are used to synthesize compound 3034 as described in example 73A (synthetic method AJ).

### Example 77A

### (Synthetic Method AL): Preparation of Compound 3035

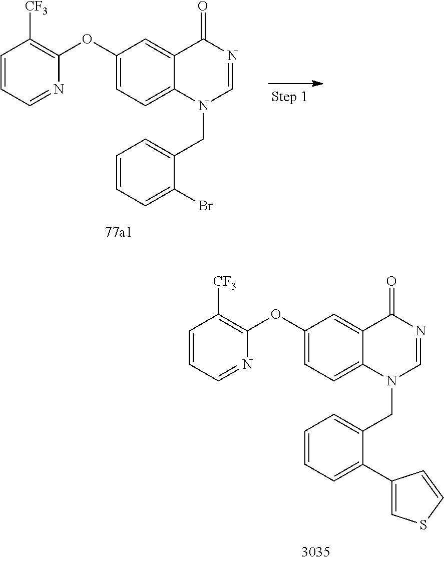

Compound 77a1 is synthesized as described in synthetic method AH.

### Step 1:

To the bromoarene 77a1 (70 mg, 0.15 mmol) in DMF (1.5 mL) and water (0.5 mL) are added K₂CO₃ (61 mg, 0.44 mmol), the 3-thiopheneboronic acid (28 mg, 0.22 mmol) and Pd(PPh₃)₄ (17 mg, 0.01 mmol). The reaction mixture is purged with Ar, sonicated for 5 min followed by stirring overnight at 80° C. The resulting mixture is acidified with AcOH and purified by semi-prep. HPLC to afford compound 3035.

### Example 78A

### (Synthetic Method AM): Preparation of Compound 3036

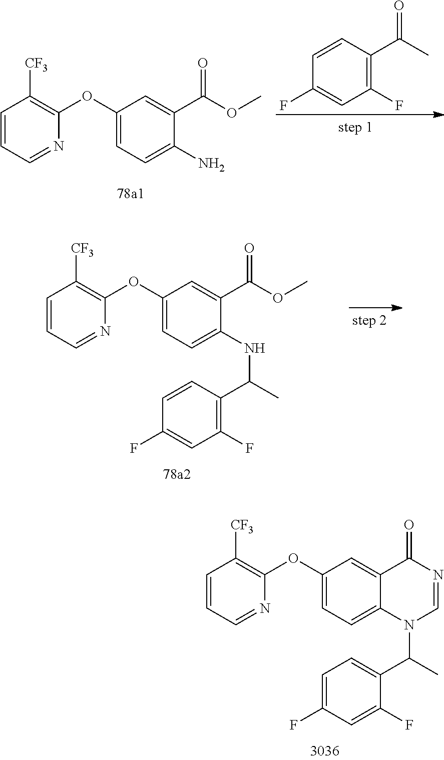

Intermediate 78a1 is prepared as described in PCT Int. Appl. WO 2008/019477, herein incorporated by reference.

### Step 1:

Reference: WO 2006/064286, herein incorporated by reference.

To a stirred mixture of aniline 78a1 (100 mg, 0.33 mmol) in THF (2 mL) are added 2,4-difluoroacetophenone (60 μL, 0.49 mmol) and Bu₂SnCl₂ (5 mg, 0.02 mmol). The reaction mixture is stirred at RT for 5 min before phenylsilane (45 μL, 0.36 mmol) is added. The mixture is warmed to 80° C. and is stirred for 3 h. Additional acetophenone, Bu₂SnCl₂ and phenylsilane are added iteratively until the reaction is complete. The mixture is concentrated and the crude product is purified by combiflash to afford intermediate 78a2.

### Step 2:

Intermediate 78a2 is converted to compound 3036 as described in example 6A steps 3 and 4.

### Example 79A

### (Synthetic Method AN): Preparation of Compound 3037

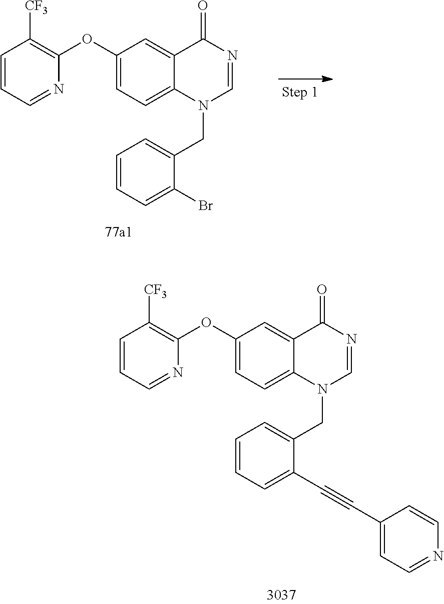

### Step 1:

To arylbromide 77a1 (70 mg, 0.15 mmol) in THF (1.5 mL) are added Et₃N (51 μL, 0.37 mmol), 4-ethynylpyridine (17 mg, 0.16 mmol), CuI (3 mg, 0.015 mmol) and Pd(PPh₃)₄ (17 mg, 0.015 mmol). The reaction mixture is purged with Ar, then heated at 65° C. in a sealed tube. The resulting mixture is acidified with TFA and injected onto a semi-prep. HPLC to isolate compound 3037.

### Example 80A

### Preparation of Compound 3041

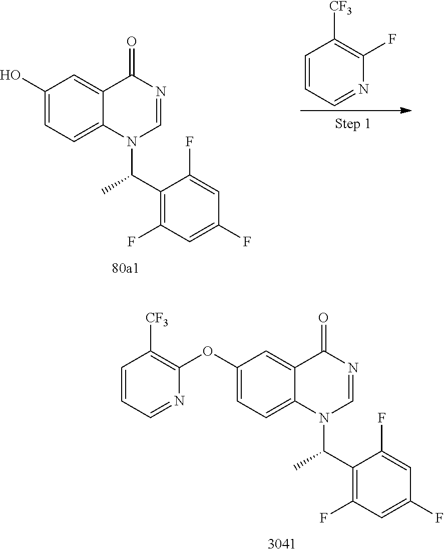

Intermediate 80a1 is synthesized using intermediate 15c3 as described in example 15C.

### Step 1:

Phenol 80a1 is coupled to 2-fluoro-3-trifluoromethylpyridine as described in example 27A (synthetic method O) to generate compound 3041.

### Example 81A

### Preparation of Compound 3043

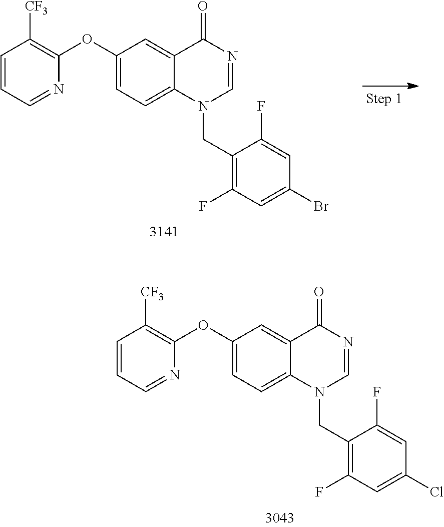

Reference: Arvela, R. K.; Leadbeater, N. E. Synlett 2003, 8, 1145, herein incorporated by reference.

### Step 1:

To a mixture of arylbromide 3141 (40 mg, 0.06 mmol) in DMF (0.4 mL) is added NiCl₂.6H₂O (18 mg, 0.08 mmol). The mixture is heated in a microwave at 170° C. for 5 min. The resulting solution is acidified with AcOH and injected onto a semi-prep. HPLC to isolate compound 3043.

### Example 82A

### (Synthetic Method AO): Preparation of Compound 3044

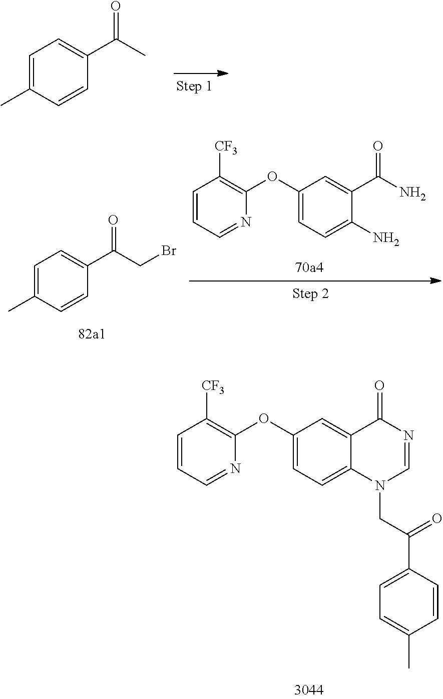

### Step 1:

To a solution of 4-methylacetophenone (930 mg, 6.9 mmol) in dioxane (30 mL) is slowly added over 60 min a solution of Br₂ (0.31 mL, 6.1 mmol) in dioxane (20 mL). The reaction mixture is stirred for 30 min and then quenched with a sat. aq. solution of NaHCO₃ and concentrated under vacuum. The resulting mixture is extracted with Et₂O (2×). The combined organic extracts are washed with water and brine then dried over MgSO₄, filtered and concentrated under vacuum to afford the α-bromoketone 82a1.

### Step 2:

To the aniline 70a4 (98 mg, 0.33 mmol) in DMF (2 mL) are added K₂CO₃ (110 mg, 0.99 mmol) and α-bromoketone 82a1 (120 mg, 0.58 mmol). The reaction mixture is stirred for 12 min at 100° C. in microwave. Another portion of α-bromoketone 82a1 is added and the reaction mixture is again stirred for 12 min at 100° C. in a microwave. The resulting solution is diluted in water, extracted with EtOAc (2×), passed through a IST® phase separator cartridge and concentrated under vacuum. The crude residue is diluted in CH(OMe)₃ (4 mL), TFA (0.1 mL, 1.3 mmol) is added and the reaction is stirred at RT overnight. The mixture is concentrated then the residue is taken up in DMSO and injected onto a semi-prep. HPLC to isolate compound 3044.

### Example 83A

### Preparation of Compound 3047

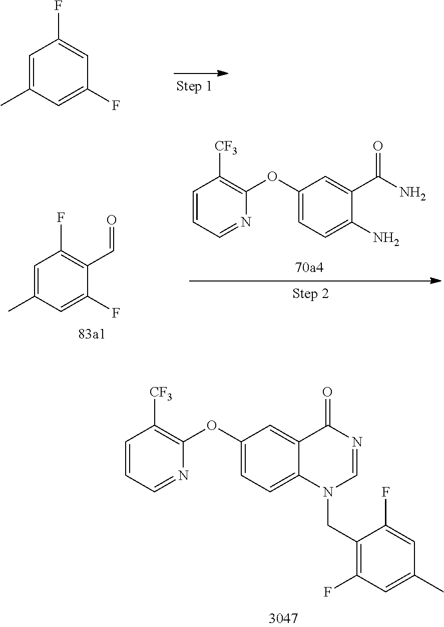

### Step 1:

To a mixture of 3,5-difluorotoluene (100 mg, 0.78 mmol) in THF (4 mL) cooled to −78° C. is slowly added n-BuLi (1.6 M in hexanes, 0.50 mL, 0.80 mmol). After stirring 20 min at −78° C., DMF (20 μL) is added and the mixture is allowed to warm to RT. The mixture is partitioned between EtOAc and water. The organic phase is separated and washed with brine. The organic is dried over MgSO₄, filtered and concentrated under vacuum to afford aldehyde 83a1 which is utilized in the next step without further purification.

### Step 2:

Intermediate 83a1 is coupled to aniline 70a4 and converted to compound 3047 as described in example 71A (synthetic method AH).

### Example 84A

### Preparation of Intermediate 84a4

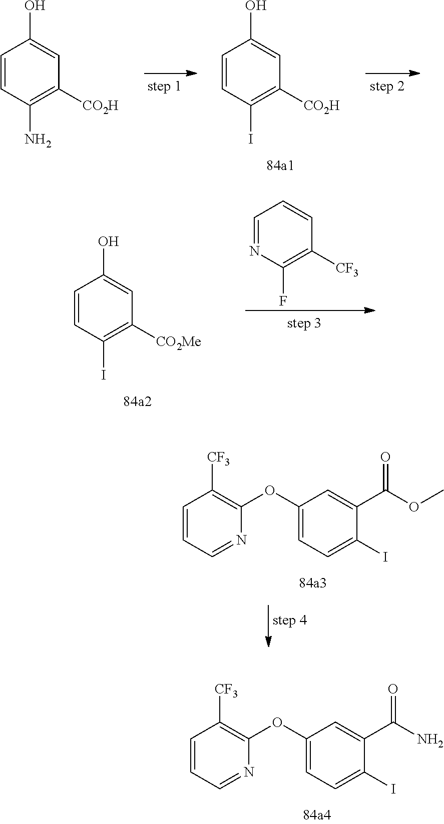

### Step 1:

To a mixture of 2-amino-5-hydroxybenzoic acid (25.0 g, 163 mmol) in water (500 mL) and H₂SO₄ 16M (33 mL) at 0° C. is added, over 30 min, a solution of NaNO₂ (20.9 g, 304 mmol) in 100 mL of water. The temperature is maintained at 0-5° C. during the addition. After diazotization, a solution of KI (67.6 g, 406 mmol) in 100 mL water is added and the resulting reaction mixture is heated to 80-90° C. for 1 h. The reaction mixture is cooled to 0° C. and the solid is collected by filtration and the filtrate is extracted with Et₂O. The combined organic layers are concentrated and combined with the filter solid. Treatment with charcoal in hot water and co-evaporation with MeOH (6×) provides iodide 84a1.

### Step 2:

To 84a1 (25.3 g, 96.0 mmol) in MeOH (1 L) is added H₂SO₄ 16M (12.0 mL, 115 mmol). The resulting reaction mixture is heated to reflux for 18 h then cooled down to RT. Most of the solvent is removed and the residue is partitioned between EtOAc and water. The combined organic layer are washed with brine, dried over Na₂SO₄, filtered and decolorized with active charcoal. The charcoal is filtered and the filtrate is concentrated and purified by ISCO flash chromatography (Hex:EtOAc 2:1) to obtain ester 84a2.

### Step 3:

To 84a2 (12.0 g, 43.2 mmol) in DMSO (200 mL) are added 2-fluoro-3-trifluoromethylpyridine (7.83 g, 47.5 mmol) and K₂CO₃ (14.9 g, 108 mmol). The reaction mixture is heated for 1.5 h at 80-82° C. The mixture is allowed to cool to RT and is poured into water and extracted with EtOAc. The organic layer is washed with brine, dried over Na₂SO₄ and concentrated. The residue is purified by ISCO flash chromatography (Hex:EtOAc 5:1 to 2:1, gradient) to afford 84a3.

### Step 4:

Ester 84a3 is converted to carboxamide 84a4 as described in example 12B steps 4 and 5.

### Example 84B

### (Synthetic Method AP): Preparation of Compound 3048

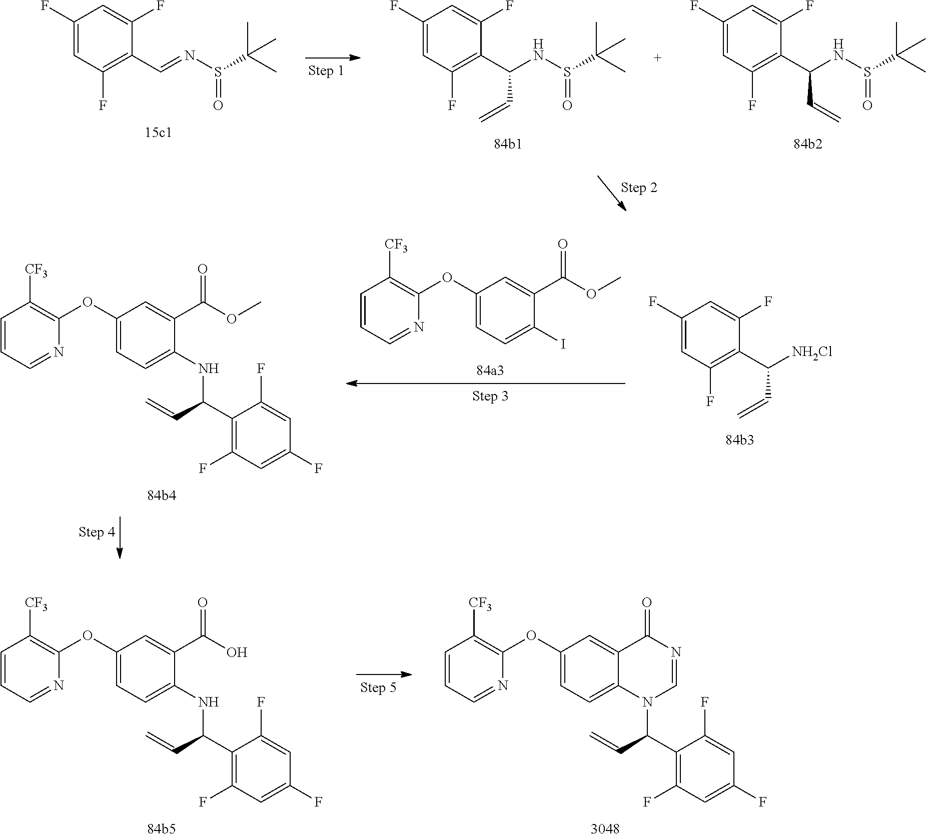

### Step 1:

To a solution of sulfinimine 15c1 (1.00 g, 3.8 mmol) in DCM (15 mL) is slowly added vinylmagnesium bromide (1.0 M in THF, 9.1 mL, 9.1 mmol) at −78° C. The reaction mixture is slowly warmed up to −10° C., stirred for 1 h and warmed to RT with continuous stirring for another hour. The reaction is quenched with a sat. aq. NH₄Cl solution and extracted with DCM (2×). The organic layers are combined, washed with brine, dried over anhydrous Na₂SO₄, filtered under vacuum and concentrated. Purification by flash chromatography using an AcOEt/Hex gradient isolates diastereoisomers 84b1 and 84b2. Absolute stereochemistry was assigned based on precedent described in the reference

### Step 2:

Sulfinamide 84b1 is converted to amine hydrochloride salt 84b3 as described in example 15C step 3.

### Step 3:

A solution of the iodide 84a3 (450 mg, 1.1 mmol), amine 84b3 (260 mg, 1.2 mmol) and Cs₂CO₃ (870 mg, 2.7 mmol) in toluene (5 mL) is degassed in a sonic bath for 5 min then Pd(OAc)₂ (36 mg, 0.05 mmol) and XANPHOS (49 mg, 0.09 mmol) are added. The mixture is degassed for 2 min. The reaction mixture is stirred for 1.5 h at 60° C., concentrated to dryness and purified by flash chromatography (10-90% AcOEt/Hex) to afford 84b4.

### Step 4:

To a mixture of 84b4 (260 mg, 0.54 mmol) in MeOH/THF (1:1, 4 mL) is added 5N aq. NaOH (0.54 mL, 2.70 mmol). The reaction is stirred for 4 h at RT, quenched with 1N HCl and extracted with AcOEt (3×). The organic layers are combined, washed with brine, dried over anhydrous Na₂SO₄, filtered under vacuum and concentrated to afford acid 84b5.

### Step 5:

Intermediate 84b5 is converted to compound 3048 as described in example 5B steps 2 and 3.

### Example 85A

### Preparation of Compound 3053

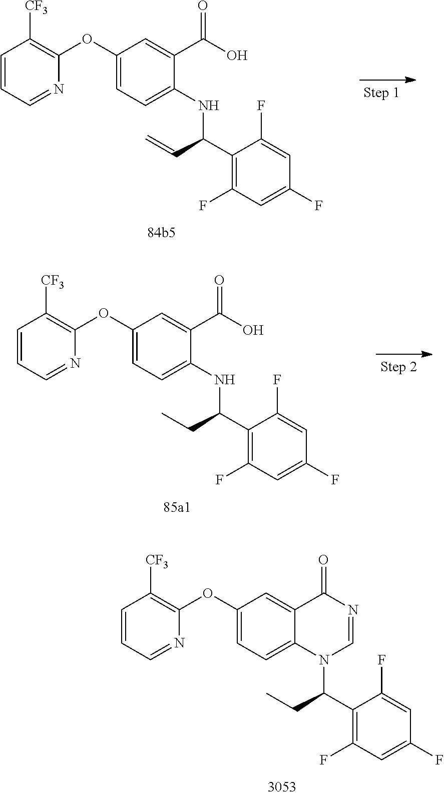

### Step 1:

To a mixture of 84b5 (30 mg, 0.06 mmol) in MeOH (3 mL) is added N₂H₄.H₂O (0.3 mL, 6.2 mmol). The reaction is warmed to 60° C. and stirred for 5 h. The mixture is concentrated and the residue is subjected to combiflash to isolate intermediate 85a1.

### Step 2:

Intermediate 85a1 is converted to compound 3053 as described in example 5B steps 2 and 3.

### Example 86A

### Preparation of Compounds 3055 & 3056

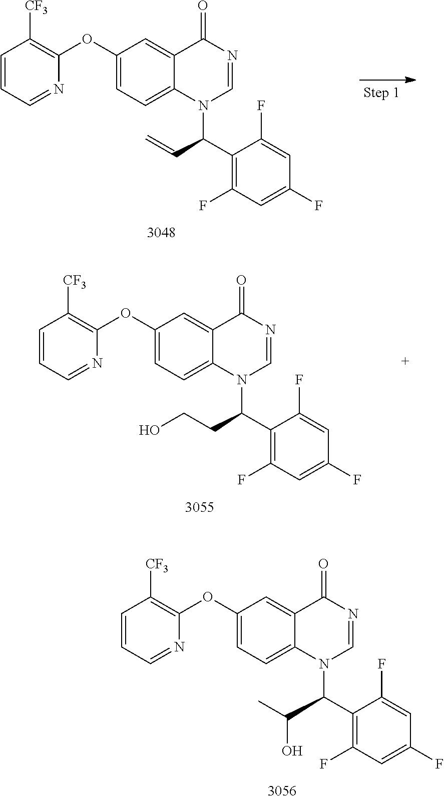

### Step 1:

i) To a mixture of 3048 (30 mg, 0.06 mmol) in THF (1 mL) at 0° C. is slowly added BH₃ (1M in THF, 126 μL, 0.13 mmol). The reaction mixture is stirred for 1 h at RT. To complete the reaction, 9-BBN (0.5 M in THF, 600 μL, 0.30 mmol) is added and the mixture is stirred for 2 h at 60° C. The solution is quenched at 0° C. with the addition of aq. 5N NaOH (0.60 mL, 0.63 mmol) and H₂O₂30% (142 μL, 1.27 mmol). This mixture is stirred for 1 h at RT and then extracted with AcOEt (3×). The organic layers are combined, washed with brine, dried over anhydrous Na₂SO₄, filtered under vacuum and concentrated.

ii) The residue is then combined with I₂ (16 mg, 0.06 mmol) and Na₂CO₃ (20 mg, 0.19 mmol) in DCM (1 mL) and stirred for 2 h at RT. The resulting solution is quenched with a sat. aq. Na₂S₂O₃, extracted with DCM (3×) and concentrated. The residue obtained is dissolved in MeOH (6 mL) and injected on a semi-prep. HPLC to separate alcohol compounds 3055 and 3056.

### Example 87A

### Preparation of Compound 3057

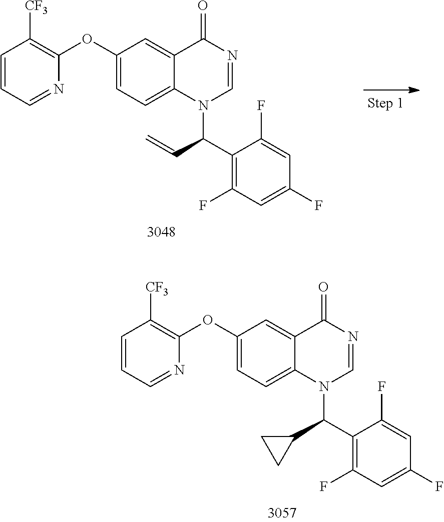

Reference: Suda, M. Synthesis 1981, 714, herein incorporated by reference.

### Step 1:

To a mixture of 3048 (20 mg, 0.04 mmol) in DCM (0.5 mL) at 0° C. is added diazomethane (0.7 M/Et₂₀, 1 mL). Pd(OAc)₂ (2 mg, 0.01 mmol) is added in one portion and the mixture is stirred at RT for 1 h. The mixture is concentrated partially under a nitrogen flow. The crude material is purified by prep-TLC (eluted with 100% AcOEt) to afford 3057.

### Example 88A

### (Synthetic Method AQ): Preparation of Compound 3058

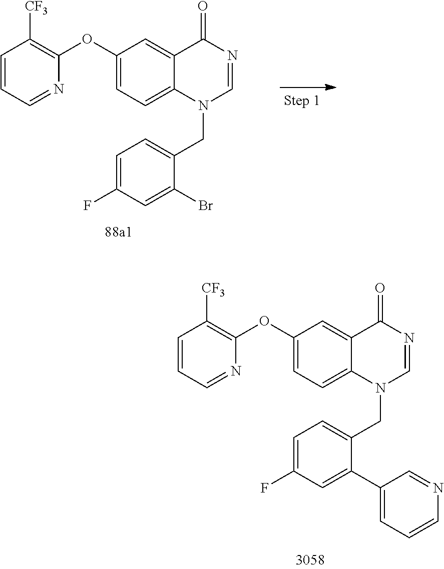

Compound 88a1 is synthesized as described in example 5B.

### Step 1:

To the bromoarene 88a1 (50 mg, 0.10 mmol) in DMF (2 mL) and water (0.2 mL) are added K₂CO₃ (56 mg, 0.41 mmol), the 3-pyridineboronic acid (25 mg, 0.20 mmol) and Pd(PPh₃)₄ (12 mg, 0.01 mmol). The reaction mixture is purged with Ar for 5 min before being heated to 125° C. for 20 min with stirring in a microwave. The resulting mixture is acidified with TFA, filtered then injected on a semi-prep. HPLC to isolate compound 3058.

### Example 89A

### Preparation of Compound 3064

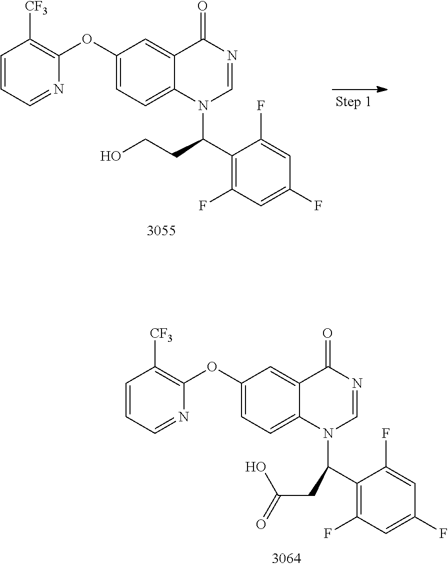

### Step 1:

To a mixture of 3055 (22 mg, 0.02 mmol) in MeCN/H₂O (0.3/0.2 mL) is added NaH₂PO₄ (0.67 M in water, 200 μL, 0.13 mmol) and TEMPO (1.2 mg, 0.01 mmol) followed by NaClO₂ (2.0 M in water, 50 μL, 0.10 mmol) and NaClO (50 μL of dilute Javel™; 30 μL in 0.5 mL of water). The reaction mixture is warmed to 45° C. and stirred for 2 h. Additional TEMPO (2 mg) is added and stirring is continued for an additional 2 h at 45° C. The mixture is acidified with AcOH, filtered and injected onto a prep HPLC to isolate 3064.

### Example 90A

### Preparation of Compound 3067

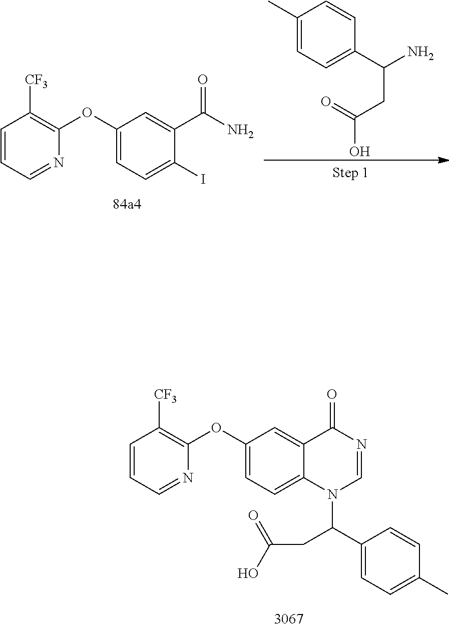

Reference: Ma, D; Xia, C. Org. Lett. 2001, 3, 2583, herein incorporated by reference.

### Step 1:

A mixture of iodide 84a4 (1.00 g, 2.5 mmol), 3-amino-3-(4-methylphenyl)butanoic acid (0.53 g, 2.9 mmol), K₂CO₃ (0.85 g, 6.1 mmol) and CuI (23 mg, 0.12 mmol) in DMF (10 mL) and water (0.2 mL) is heated to 160° C. with stirring for 25 min in a microwave. The reaction is diluted in 1N HCl and extracted with EtOAc (3×). The organic layers are combined, washed with brine, dried over anhydrous Na₂SO₄, filtered under vacuum and concentrated. Purification by flash chromatography (9:1 DCM/MeOH) and trituration (Et₂O) affords the coupled aniline.

ii) To a mixture of the aniline-carboxamide in (MeO)₃CH (10 mL) is added TFA (0.3 mL). The mixture is stirred 30 min at RT before it is concentrated to afford compound 3067.

### Example 91A

### Preparation of Compound 3080

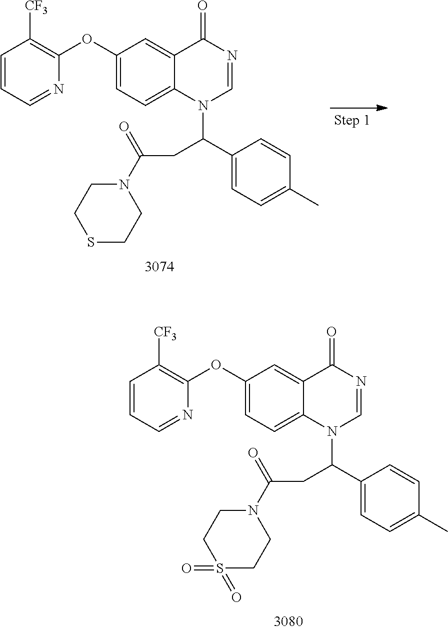

### Step 1:

To a mixture of thioether 3074 (23 mg, 0.04 mmol) in acetone (0.4 mL) and water (0.15 mL) is added Oxone® (100 mg, 0.17 mmol). This mixture is stirred for 1 h at RT. The mixture is diluted in MeCN, filtered then injected on a prep HPLC to isolate compound 3080.

### Example 92A

### (Synthetic Method AR): Preparation of Intermediate 92a2

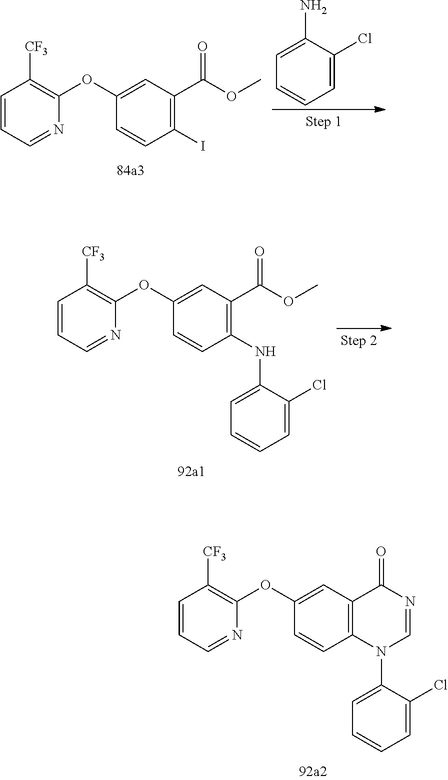

### Step 1:

Reference: Sanz, R.; Fernández, Y.; Castroviejo, M. P.; Pérez, A.; Fañanás, F. J. J. Org. Chem. 2006, 71, 6291-6294, herein incorporated by reference. Pd(OAc)₂/Xanthphos is found to be superior to Pd₂(dba)₃/BINAP for this coupling.

Aryliodide 84a3 (157 mg, 0.37 mmol) is combined with 2-chloroaniline (40 μL, 0.41 mmol) and Cs₂CO₃ (180 mg, 0.56 mmol) in toluene (2 mL) and the mixture is degassed (Ar bubbling). Pd(OAc)₂ (13 mg, 0.02 mmol) and Xanthphos (17 mg, 0.03 mmol) are added and the mixture is heated to 110° C. and stirred overnight. The mixture is concentrated and the residue is loaded directly onto a CombiFlash (hex/EtOAc, 5% to 100%) to isolate diarylaniline 92a1.

### Step 2:

Intermediate 92a1 is converted to 92a2 using the protocols described in steps 4-6 in example 12B.

### Example 92B

### (Synthetic Method AS): Preparation of Compound 3084

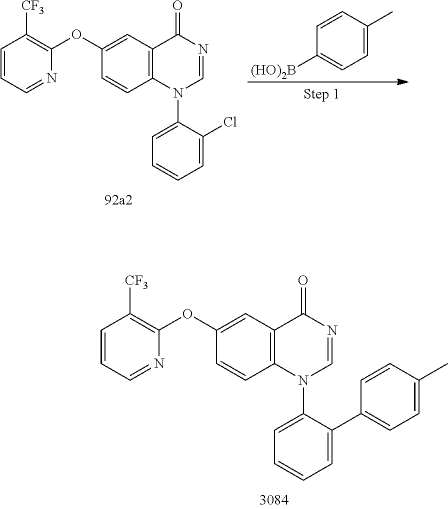

### Step 1:

Arylchloride 92a2 (50 mg, 0.12 mmol) is combined with 4-methylphenylboronic acid (25 mg, 0.18 mmol) and aq. Na₂CO₃ (2.0 M, 0.18 mL) in DMF (1 mL). Ar is bubbled through the mixture for 10 min before (Bu₃P)₂Pd (6 mg, 0.01 mmol) is added. The mixture is then heated to 150° C. in a microwave for 15 min with stirring. After cooling to RT, the reaction mixture is filtered then injected onto a prep. HPLC to isolate 3084.

### Example 93A

### (Synthetic Method AT): Preparation of Compound 3090

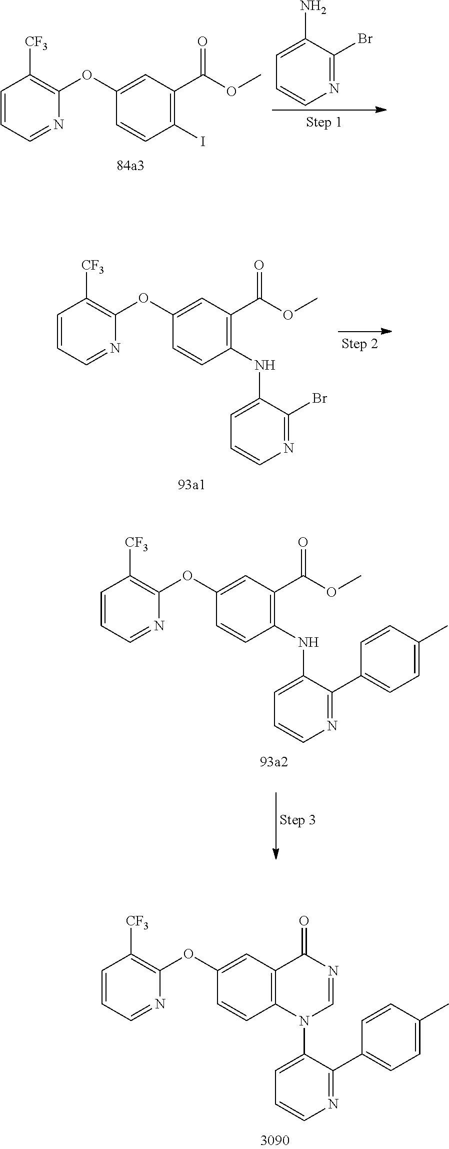

### Step 1:

Intermediate 84a3 is coupled to 3-amino-2-bromopyridine using the protocols described in example 92A step 1.

### Step 2:

Intermediate 93a1 is coupled to 4-methylphenylboronic acid using the protocol described in example 88A (synthetic protocol AQ).

### Step 3:

Intermediate 93a2 is converted to 3090 using the protocols described in example 12B steps 4-6.

### Example 94A

### Preparation of Compound 3093

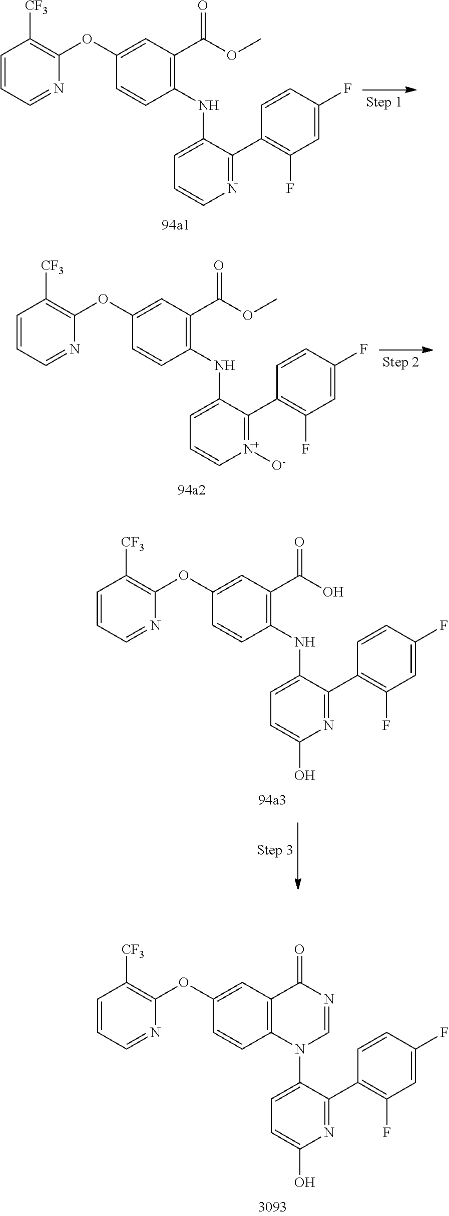

Intermediate 94a1 is synthesized as described in example 93A.

### Step 1:

To a mixture of pyridine 94a1 (640 mg, 1.3 mmol) in DCM (15 mL) is added mCPBA (550 mg, 2.6 mmol). The mixture is stirred for 1 h at RT. The reaction mixture is diluted in sat. aq. Na₂CO₃ then extracted with DCM (3×). The organic phase is passed through an IST® phase separator cartidge to afford N-oxide 94a2.

### Step 2:

Reference: Kanekiyo, N.; Kuwada, T.; Choshi, T.; Nobuhiro, J.; Hibino, S. J. Org. Chem. 2001, 66, 8793, herein incorporated by reference.

A mixture of N-oxide 94a2 (660 mg, 1.3 mmol) in Ac₂O (5 mL) is heated to 100° C. and stirred for 1 h. The reaction mixture is concentrated and dried in vacuo. The residue is diluted in THF/MeOH/water (5:2.5:2.5 mL) and 10N NaOH (2.5 mL, 25 mmol) is added. The mixture is stirred overnight at RT. The mixture is diluted in sat. aq. NH₄Cl then extracted with DCM. The aqueous phase is concentrated. The residue is taken up in MeOH and filtered to remove solids. The organic filtrate is concentrated then diluted in toluene and re-concentrated (2×) to afford crude pyridine/acid 94a3 which is utilized in the next step without further purification.

### Step 3:

Intermediate 94a3 is converted to 3093 using the protocols described in example 12B steps 5-6.

### Example 95A

### Preparation of Compound 3094

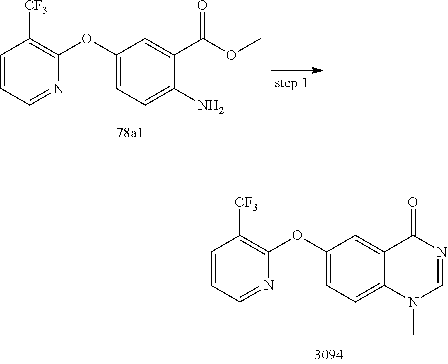

### Step 1:

i) To a stirred mixture of aniline 78a1 (300 mg, 0.96 mmol) in EtOH (4 mL) and AcOH (50 μL) are added NaCNBH₃ (25 mg, 0.40 mmol) and formaldehyde (35% in water, 29 μL, 0.34 mmol), The mixture is stirred at RT for 1 h. Another portion of NaCNBH₃ and formaldehyde are added and stirring is continued for an additional hour. The mixture is concentrated and then DMSO (3 mL) and 2.5 N NaOH (1 mL) are added. The mixture is stirred at RT for 1 h before being acidified with AcOH then partitioned between water and EtOAc. The organic phase is separated, washed with brine, dried over MgSO₄, filtered and concentrated.

ii) The crude acid intermediate is converted to 3094 using the protocols described in example 12B steps 5-6.

### Example 96A

### Preparation of Compound 3095

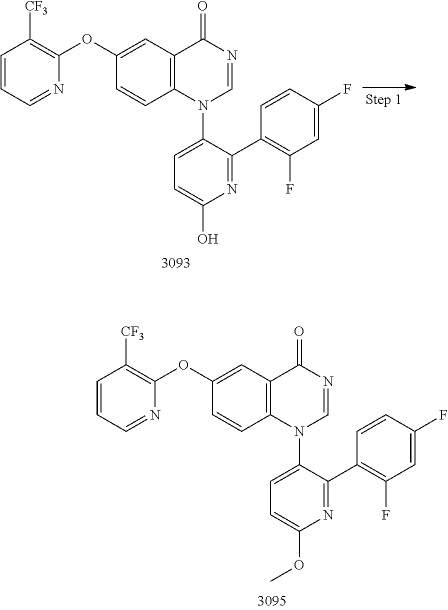

### Step 1:

To a stirred mixture of pyridone 3093 (25 mg, 0.05 mmol) in DCM/MeOH (1/0.1 mL) is added TMS-diazomethane (40 μL, 0.08 mmol). The mixture is stirred overnight at RT. The mixture is diluted in water then extracted with DCM. The organic phase is passed through a IST® phase separator cartridge then concentrated. The crude product is purified by prep TLC (10% MeOH in EtOAc) to afford methylether 3095.

### Example 97A

### Preparation of Compound 3096

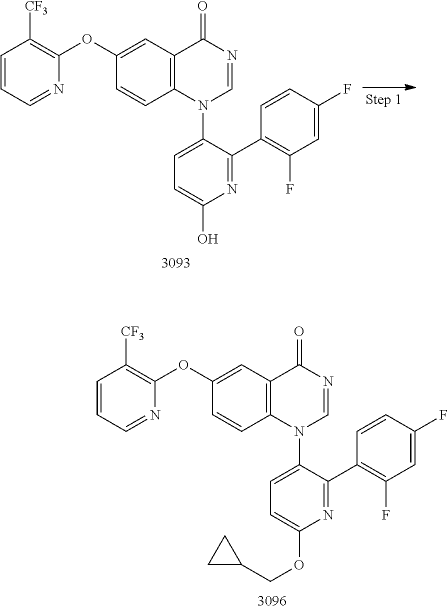

### Step 1:

To a stirred mixture of pyridone 3093 (25 mg, 0.05 mmol) in DMF (1 mL) are added K₂CO₃ (10 mg, 0.07 mmol) and cyclopropylmethyl bromide (13 μL, 0.13 mmol). The mixture is stirred overnight at RT. The mixture is diluted in water then extracted with DCM. The organic phase is passed through an IST phase separator cartridge then concentrated. The crude product is purified by prep HPLC to afford ether 3096.

### Example 98A

### (Synthetic Method AU): Preparation of Compound 3097

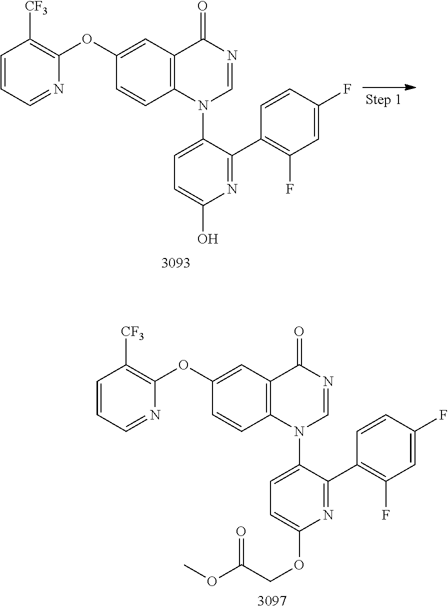

### Step 1:

To a stirred mixture of pyridone 3093 (34 mg, 0.07 mmol) in MeCN (1 mL) are added Cs₂CO₃ (32 mg, 0.10 mmol) and methyl-2-bromoacetate (7 μL, 0.07 mmol). The mixture is stirred for 1 h at RT. The mixture is diluted in water then extracted with DCM. The organic phase is passed through an IST phase separator cartridge then concentrated. The crude product is purified by prep HPLC to afford ether 3097.

### Example 99A

### Preparation of Compound 3100

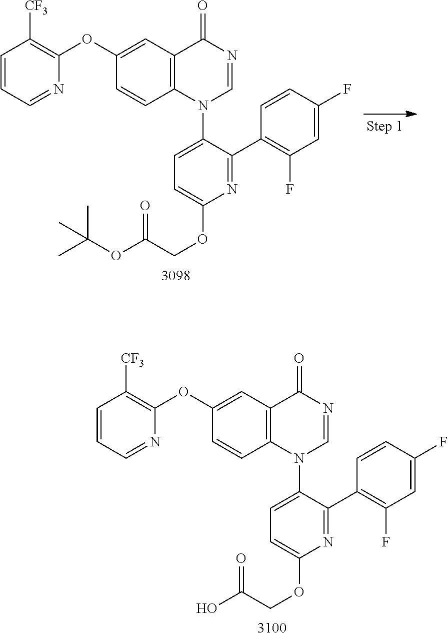

### Step 1:

To a stirred mixture of t-Bu ester 3098 (48 mg, 0.08 mmol) in DCM (1 mL) is added TFA (0.5 mL). The mixture is stirred for 2 h at RT. The mixture is diluted in water then extracted with DCM. The organic phase is passed through an IST® phase separator cartridge then concentrated. The crude product is purified by combiflash (0 to 10% MeOH in DCM) to afford acid 3100.

### Example 100A

### (Synthetic Method AV): Preparation of Compound 3101

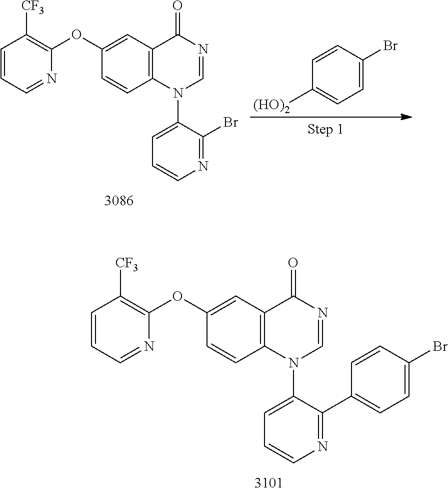

### Step 1:

Bromopyridine 3086 (11 mg, 0.02 mmol) is combined with 4-bromophenylboronic acid (7 mg, 0.03 mmol) and aq. Na₂CO₃ (2.0 M, 40 μL) in DMF (0.5 mL). Ar is bubbled through the mixture for 10 min before (Bu₃P)₂Pd (1.3 mg, 0.002 mmol) is added. The mixture is then heated to 65° C. and stirred for 16 h. After cooling to RT, the reaction mixture is filtered then injected onto a prep. HPLC to isolate 3101.

### Example 101A

### (Synthetic Method AW): Preparation of Compound 3110

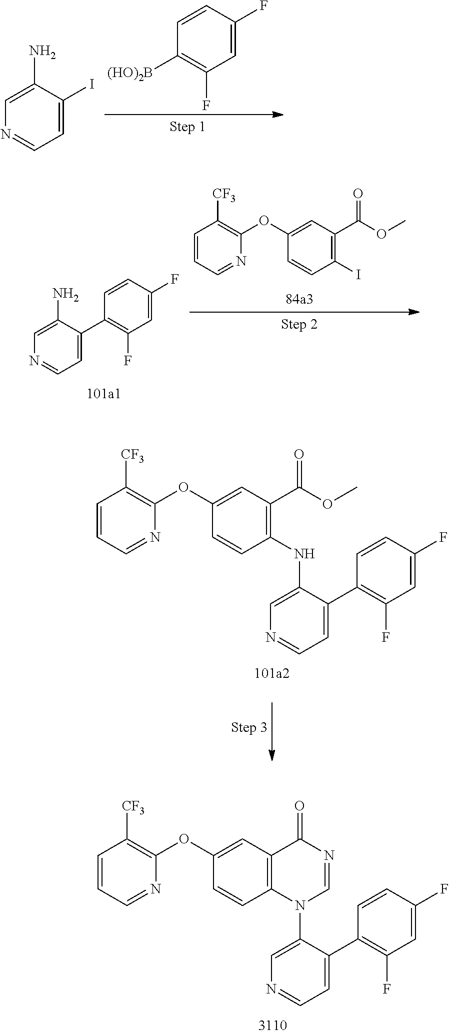

### Step 1:

3-Amino-4-iodopyridine is coupled to 2,4-difluorophenylboronic acid to form biaryl 101a1 using the protocol described in example 88A (synthetic method AQ).

### Step 2:

Aminopyridine 101a1 is coupled to iodoarene 84a3 using the protocol described in example 92A (synthetic method AR).

### Step 3:

Intermediate 101a2 is converted to 3110 using the protocols described in example 12B steps 4-6.

### Example 102A

### Preparation of Compound 3114

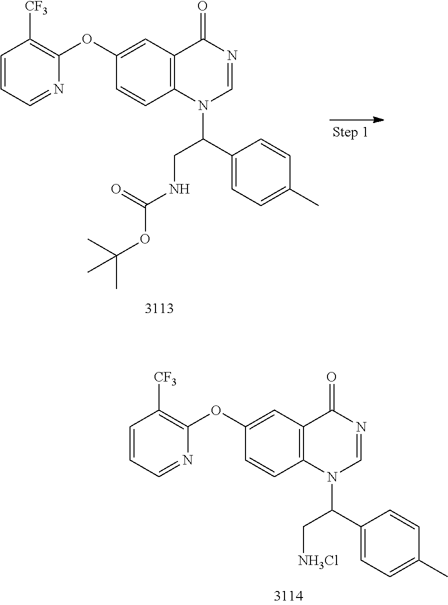

### Step 1:

To a stirred mixture of Boc-protected amine 3113 (330 mg, 0.61 mmol) in DCM (5 mL) is added 4N HCl in dioxane (1.0 mL, 4.0 mmol). The mixture is stirred overnight at RT. The mixture is concentrated then diluted in MeCN and re-concentrated (3×). The crude product is triturated in MeCN to afford hydrochloride salt 3114.

### Example 103A

### (Synthetic Method AX): Preparation of Compound 3116

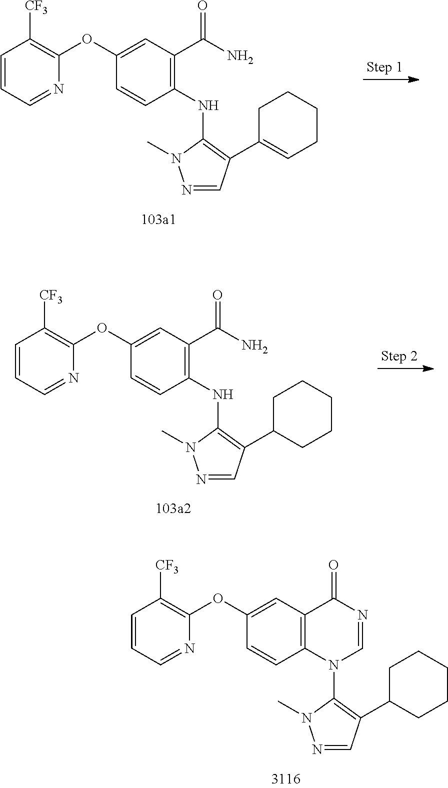

Intermediate 103a1 is prepared using synthetic method AT.

### Step 1:

A mixture of cyclohexene 103a1 (65 mg, 0.14 mmol) in EtOH (2 mL) is purged with Ar (3×) before 10% Pd/C (25 mg) is added. The flask is charged with 1 atm of H₂ and stirred overnight at RT. The reaction mixture is filtered then concentrated. The residue is diluted in (MeO)₃CH (3 mL) and treated with TFA (0.1 mL). The mixture is stirred for 1 day at RT. The mixture is concentrated and residue is taken up in DMSO, filtered and injected onto a prep. HPLC to isolate 103a2.

### Step 2:

Carboxamide 103a2 is converted to compound 3116 using the protocol described in example 1A, step 3.

### Example 104A

### Preparation of Compounds 3121 & 3123

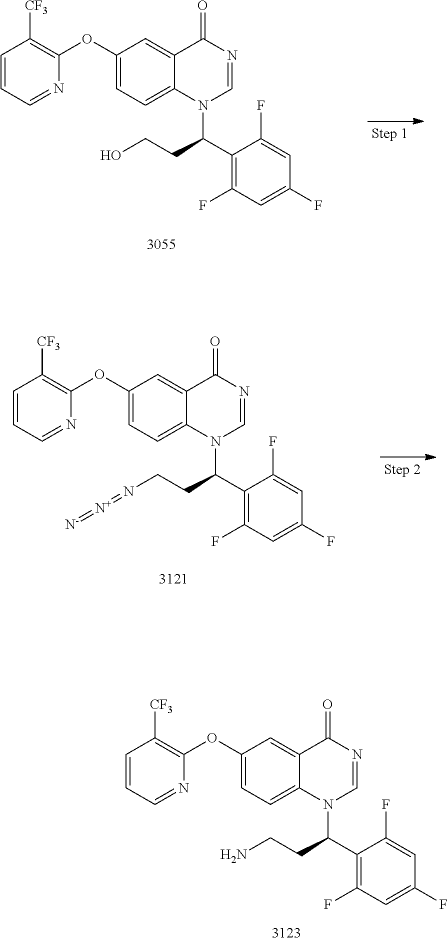

### Step 1:

To a solution of alcohol 3055 (200 mg, 0.40 mmol) in THF (2 mL) is added DBU (79 μL, 0.52 mmol) and DPPA (100 μL, 0.48 mmol) and NaN₃ (140 mg, 2.2 mmol). The mixture is stirred for 16 h at RT. The mixture is diluted in 1N HCl then extracted with EtOAc (3×). The organic layers are combined, washed with brine, dried over anhydrous Na₂SO₄, filtered under vacuum and concentrated. Purification by flash chromatography using (1:99 to 10:90) MeOH/DCM affords azide 3121.

### Step 2:

A mixture of 3121 (260 mg, 0.50 mmol) and 5% Pd/C (25 mg) in MeOH (5 mL) is stirred at RT under a H₂ atm (balloon) for 4 h. The mixture is filtered then concentrated. Purification by prep. HPLC affords 3123.

### Example 105A

### Preparation of Compound 3122

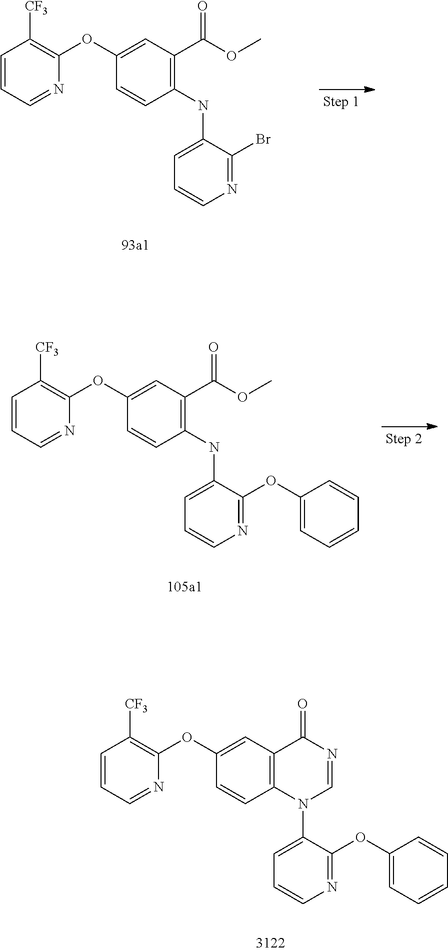

### Step 1:

Reference: Ma, D.; Cai, Q. Org. Lett. 2003, 5, 3799, herein incorporated by reference. In a microwave tube, bromopyridine 93a1 (300 mg, 0.64 mmol), phenol (105 mg, 1.12 mmol), Cs₂CO₃ (418 mg, 1.12 mmol) and N,N-dimethylglycine are combined in dioxane (1.7 mL). Ar is bubbled through the mixture for 10 min. CuI (17 mg, 0.09 mmol) is then added and mixture is heated to 150° C. in a microwave and stirred for 40 min. After cooling to RT, the mixture is diluted in EtOAc and washed with water and brine. The organic phase is dried over MgSO₄, filtered and concentrated under reduced pressure. Combiflash purification affords 105a1.

### Step 2:

Intermediate 105a1 is converted to 3122 using the protocols described in example 12B steps 4-6.

### Example 106A

### Preparation of Compound 3124

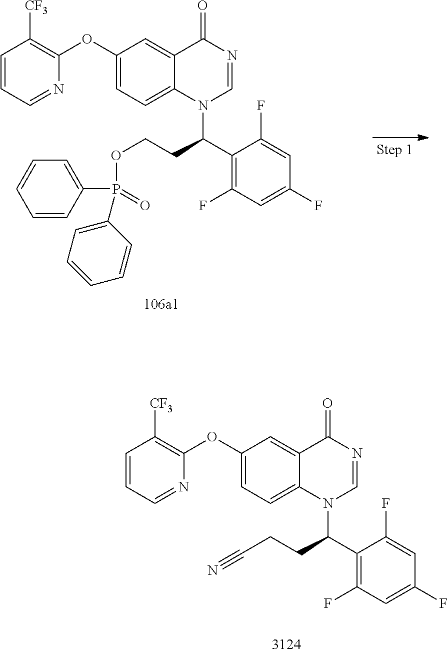

### Step 1:

To a mixture of the phosphonate 106a1 (15 mg, 0.02 mmol), isolated as a byproduct of example 104A step 1, in DMF (1 mL) is added KCN (27 mg, 0.41 mmol). The mixture is stirred for 1 h at RT then is warmed to 50° C. and stirred for 1 h. The mixture is warmed once again to 60° C. and stirred for 16 h. The mixture is diluted in water and extracted with EtOAc (3×). The organic layers are combined, washed with brine, dried over anhydrous Na₂SO₄, filtered and concentrated. Purification by prep-TLC using (5:95) MeOH/DCM affords nitrile 3124.

### Example 107A

### (Synthetic Method AY): Preparation of Compounds 3129 & 3130

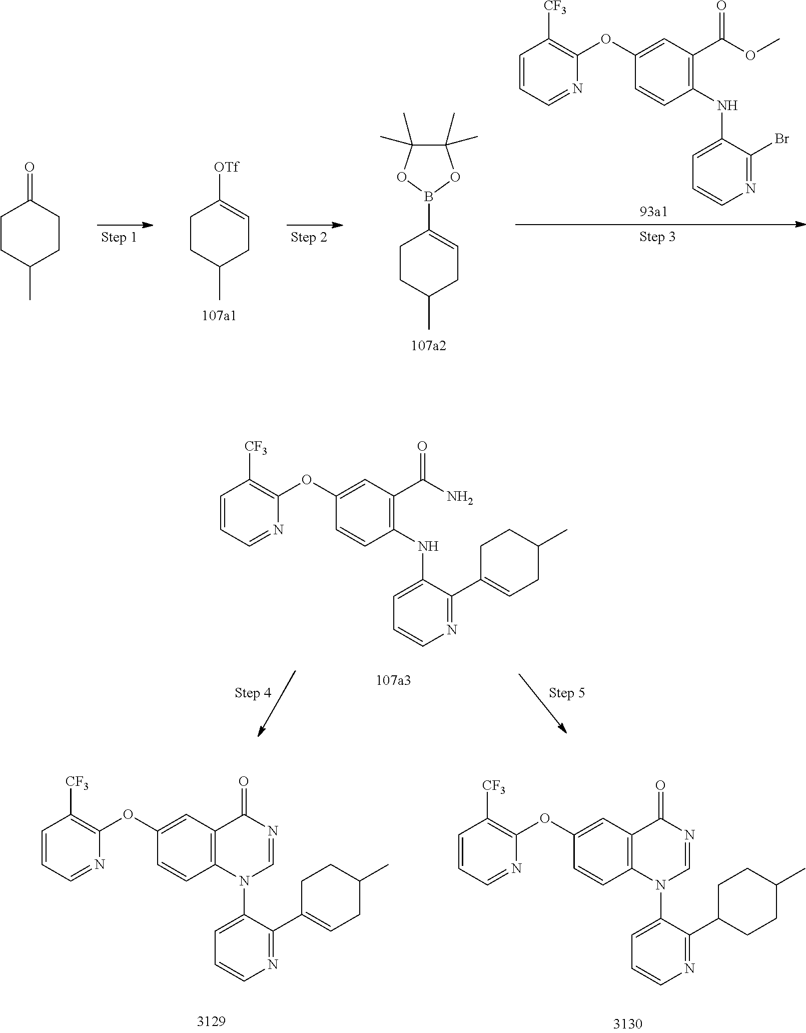

Reference for steps 1 & 2: Eastwood, P. R. Tet. Lett. 2000, 41, 3705, herein incorporated by reference.

### Step 1:

To a mixture of 4-methylcyclohexanone (0.5 mL, 4.1 mmol) in THF (10 mL) cooled to −78° C. is added LiHMDS (1.0 M in THF, 4.5 mL, 4.5 mmol). The mixture is allowed to warm to 0° C. over 45 min. This mixture is cooled to −78° C. once again and PhN(Tf)₂ is added as a mixture in THF (1 mL). The mixture is allowed to warm to RT and is stirred for 2 h. The mixture is diluted in sat. aq. NH₄Cl then extracted with Et₂O. The organic phase is dried with MgSO₄, filtered and concentrated. The crude product is purified by combiflash (5 to 50% Et₂O in Hex) to afford vinyltriflate 107a1.

### Step 2:

A mixture of vinyltriflate 107a1 (6500 mg, 2.7 mmol), bis(pinacolato)diboron (770 mg, 3.1 mmol), KOAc (780 mg, 8.0 mmol), PdCl₂(dppf) (97 mg, 0.13 mmol) and dppf (74 mg, 0.13 mmol) in dioxane (2 mL) is purged with Ar. The vessel is sealed then heated to 80° C. and stirred overnight. The mixture is concentrated and the residue is directly loaded onto a combiflash (eluting with 0 to 100% Et₂O in Hex) to isolate vinylboronate 107a2.

### Step 3:

i) Bromopyridine 93a1 is coupled to vinylboronate 107a2 using the protocol described in example 77A (synthetic method AL).

ii) The coupled product is converted to carboxamide 107a3 using the protocols described in example 12B steps 4-5.

### Step 4:

Carboxamide 107a3 is converted to compound 3129 using the protocols described in example 12B steps 6.

### Step 5:

Carboxamide 107a3 is converted to compound 3130 using the protocol described in example 103A (synthetic method AX).

### Example 108A

### (Synthetic Method AZ): Preparation of Compound 3132

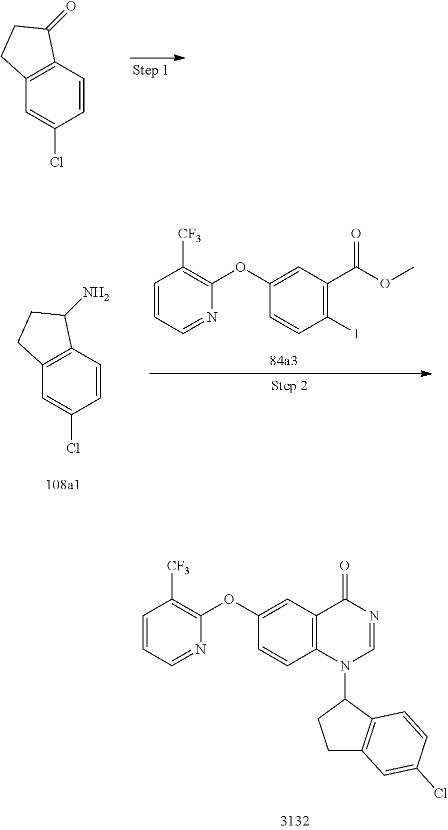

### Step 1:

A mixture of 5-chloroindanone (0.25 g, 1.5 mmol) and NH₄OAc (1.2 g, 15 mmol) in i-PrOH (15 mL) is stirred at RT for 1 h before NaBH₃CN (0.33 g, 5.3 mmol) is added. The mixture is heated to reflux and stirred for 3 h. The mixture is quenched by the addition of 5N NaOH (5 mL). The aqueous mixture is extracted with EtOAc (3×). The combined organic extracts are washed with brine, dried over MgSO₄, filtered and concentrated. The crude product is purified by flash chromatography (9:1 DCM/MeOH) to afford amine 108a1.

### Step 2:

Amine 108a1 is coupled to iodoarene 84a3 then converted to 3132 using the protocol described in example 92A (synthetic protocol AR).

### Example 109A

### Preparation of Compound 3134

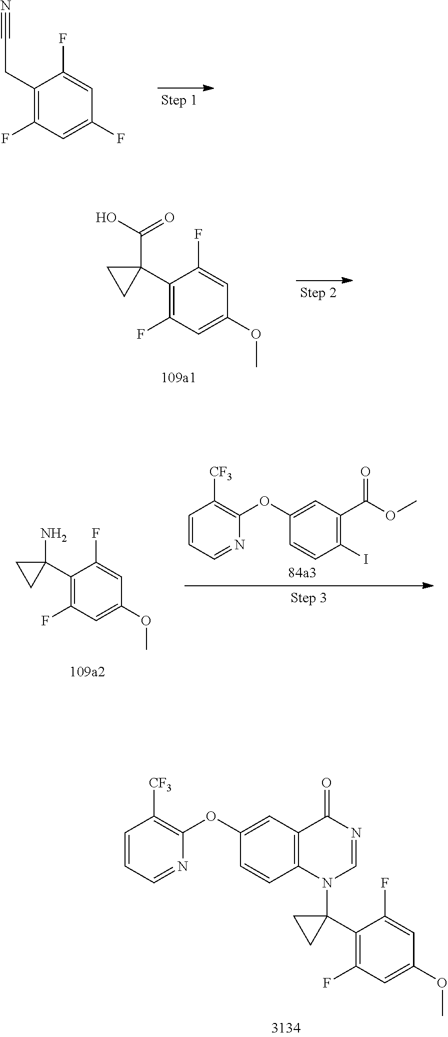

### Step 1:

To a stirring mixture of 2,4,6-trifluorobenzylcyanide (0.50 g, 2.9 mmol), TBAB (9 mg, 0.03 mmol) and KOH (60% in water, 0.98 mL, 10.5 mmol) is added 1,2-dibromoethane (1.0 mL) dropwise. The mixture is stirred at RT for 16 h. The mixture is diluted with water then extracted with Et₂O (3×). The combined organic extracts are washed with brine, dried over Na₂SO₄, filtered and concentrated. The residue is taken up in MeOH (1 mL) and 10N NaOH is added (4.0 mL). The mixture is heated to 100° C. and stirred for 2 h. The mixture is diluted with water then extracted with Et₂O. The aqueous phase is acidified with conc. aq. HCl (pH ˜1). The aqueous phase is extracted with EtOAc (3×). The combined organic extracts are washed with brine, dried over Na₂SO₄, filtered and concentrated. The crude product is purified by flash chromatography (19:1 DCM/MeOH) to afford acid 109a1.

### Step 2:

A mixture of acid 109a1 (400 mg, 1.9 mmol), DPPA (600 mg, 2.8 mmol) and NEt₃ (0.38 mL, 2.8 mmol) in DCM (5 mL) is stirred at RT for 2 h. The mixture is concentrated and the residue is subjected to flash chromatography (19:1 DCM/MeOH) to isolate the acylazide intermediate. The acylazide is taken up in dioxane (1 mL) and heated to 100° C. and stirred for 1 h. BnOH (1.0 mL) is added and stirring is continued at 100° C. for 2 h. The mixture is concentrated and the residue is subjected to flash chromatography (7:3 EtOAc/Hex) to isolate the Cbz-protected benzylamine intermediate. To a mixture of Cbz-benzylamine in EtOH is added Pd/C (5%, 50 mg). The vessel is charged with H₂ and the mixture is stirred for 4 h under 1 atm of H₂. The mixture is filtered then subjected to prep TLC plate to isolate amine 109a2.

### Step 3:

Amine 109a2 is coupled to iodoarene 84a3 then converted to 3134 using the protocol described in example 92A (synthetic protocol AR).

### Example 110A

### Preparation of Compound 3135

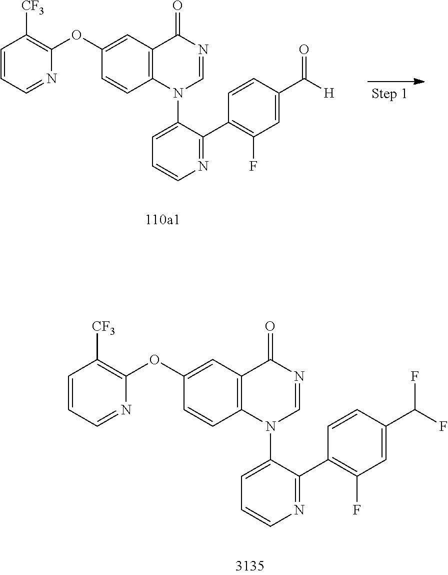

Pyridylbromide 3086 is coupled with 2-fluoro-4-formylphenylboronic acid to form 110a1 using the protocol described in example 77A (synthetic method AL).

### Step 1:

To a mixture of aldehyde 110a1 (87 mg, 0.17 mmol) in DCM (0.5 mL) is added Deoxofluor™ (54 μL, 0.29 mmol) dropwise. The mixture is stirred at RT for 3 days. The mixture is diluted in sat. aq. NaHCO₃ then extracted with DCM. The organic extract is dried over MgSO₄, filtered and concentrated. The crude product is purified by prep HPLC to afford compound 3135.

### Example 111A

### Preparation of Compound 3139

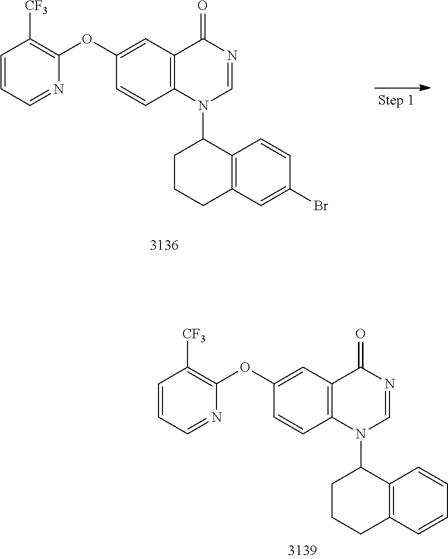

### Step 1:

To a mixture of arylbromide 3136 (50 mg, 0.10 mmol) in EtOAc/MeOH (1:1, 2 mL) is added 5% Pd/C (20 mg). The flask is charged with 1 atm of H₂ and stirred overnight at RT. The reaction mixture is filtered then concentrated. The residue is diluted in AcOH (3 mL) and injected onto a prep. HPLC to isolate compound 3139.

### Example 112A

### (Synthetic Method BA): Preparation of Compound 4008

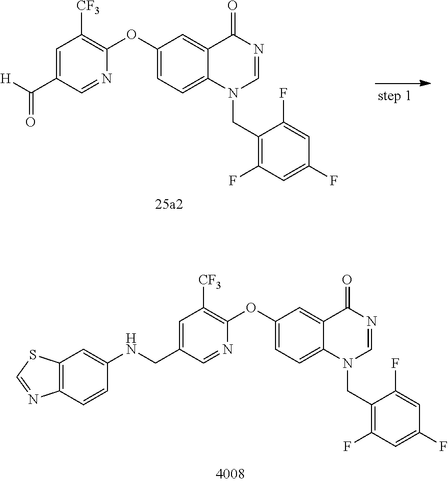

### Step 1:

To a mixture of aldehyde 25a2 (70 mg, 0.15 mmol) in DMF (1 mL) is added 6-aminobenzothiazole (45 mg, 0.30 mmol) and HCl (4N in dioxane, 76 μL, 0.31 mmol). After stirring for 30 min at RT, NaCNBH₃ (3 mg, 0.05 mmol) is added and the resulting mixture is stirred for 30 min at RT. The mixture is then diluted with AcOH and injected onto a prep. HPLC to isolate 4008.

### Example 113A

### Preparation of Compound 4016

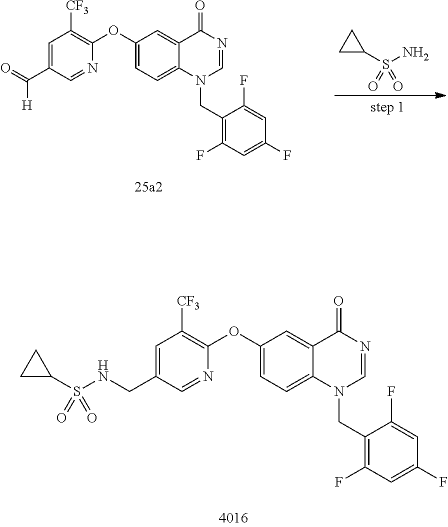

### Step 1:

A mixture of aldehyde 25a2 (60 mg, 0.13 mmol), cyclopropylsulfonamide (16 mg, 0.13 mmol) and molecular sieves (4 Å) in DCE (2 mL) is heated to 150° C. and stirred for 50 min. The mixture is allowed to cool to RT and NaBH₄ (8 mg, 0.20 mmol) is added portionwise. This mixture is stirred for 1 h at RT. The mixture is concentrated and the residue is taken up in MeOH (0.5 mL) and I₂ (50 mg) is added. This mixture is stirred for 1 h at RT. The reaction is quenched by the addition of solid Na₂S₂O₃. The mixture is diluted in DMSO/AcOH, filtered and injected onto a prep HPLC to isolate compound 4016.

### Example 114A

### Preparation of Compound 4018

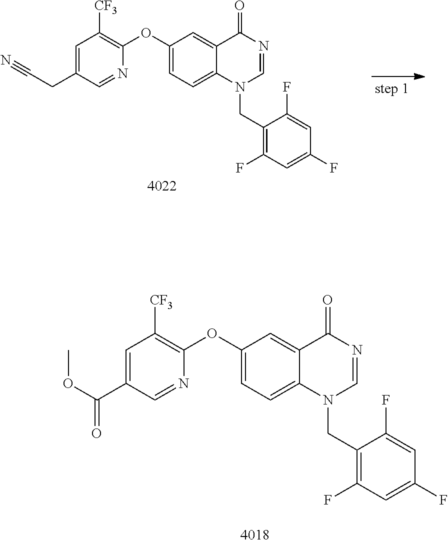

### Step 1:

A mixture of cyanide 4022 (50 mg, 0.10 mmol), Na₂CO₃ (54 mg, 0.51 mmol) and I₂ (44 mg, 0.17 mmol) in MeOH (5 mL) is stirred for 16 h at RT. The reaction is quenched by the addition of sat. aqueous Na₂S₂O₃ then extracted with EtOAc. The organic extract is dried over MgSO₄, filtered and concentrated. The crude product is taken up in DMSO/AcOH and injected onto a prep HPLC to afford compound 4018.

### Example 115A

### (Synthetic Method BB): Preparation of Compound 4020

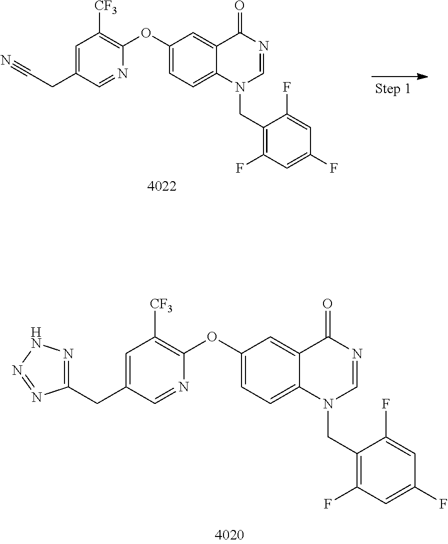

### Step 1:

To nitrile 4022 (100 mg, 0.20 mmol) in DMF (3 mL) is added NH₄Cl (26 mg, 0.49 mmol) and NaN₃ (79 mg, 1.2 mmol) in a sealed tube. This reaction mixture is degassed with Ar before being heated to 120° C. for 6 h. The mixture is diluted in EtOAc and washed with water and brine. The organic phase is dried over MgSO₄, filtered and concentrated. The crude product is taken up in DMSO/AcOH and injected onto a prep HPLC to afford compound 4020.

### Example 116A

### (Synthetic Method BC): Preparation of Compound 4021

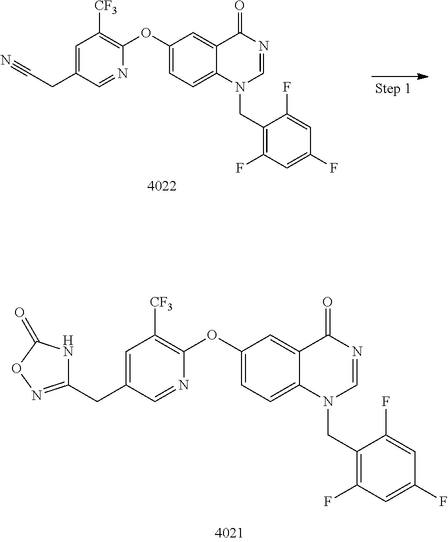

### Step 1:

To a mixture of nitrile 4022 (100 mg, 0.20 mmol) in MeOH (3 mL) is added NH₂OH.HCl (16 mg, 0.22 mmol) and NaHCO₃ (20 mg, 0.24 mmol). The mixture is heated to 70° C. and stirred for 5 h. The mixture is diluted in EtOAc and washed with water and brine. The organic phase is dried over MgSO₄, filtered and concentrated. The crude product is combined with CU (66 mg, 0.41 mmol) in dioxane (5 mL). The mixture is heated to 100° C. and stirred for 1 h. The mixture is diluted in EtOAc and washed with water and brine. The organic phase is dried over MgSO₄, filtered and concentrated. The residue is taken up in DMSO/AcOH and injected onto a prep HPLC to afford compound 4021.

### Example 117A

### Preparation of Compound 4053 & 4034

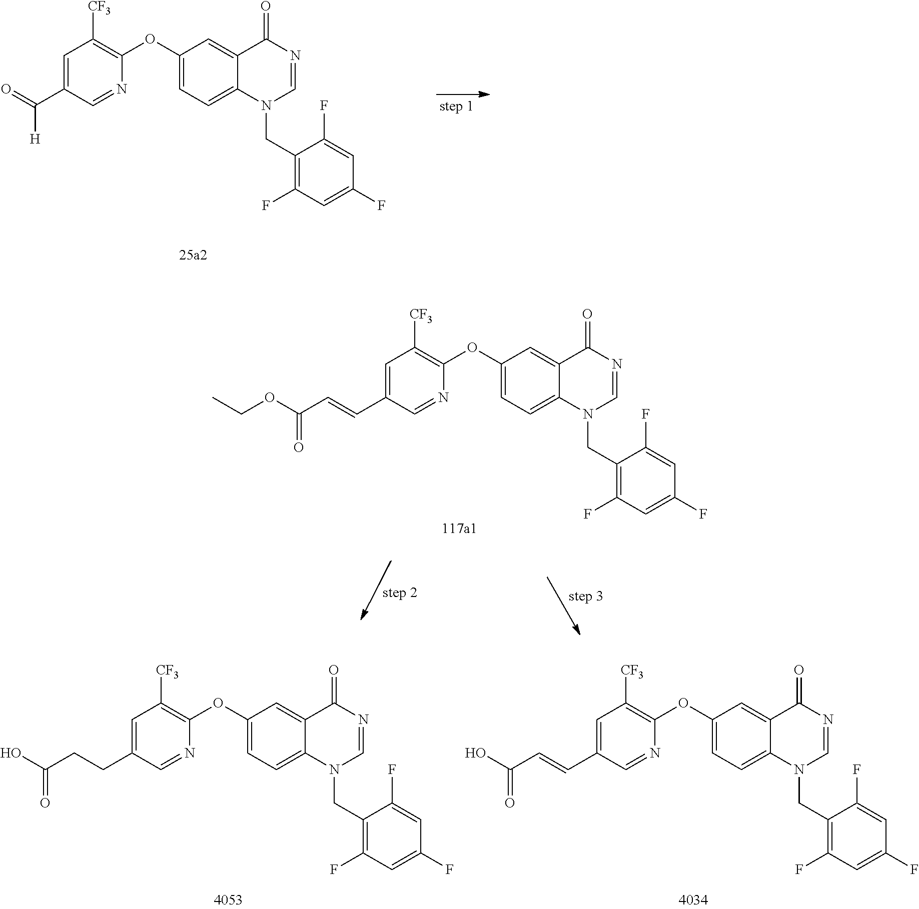

### Step 1:

A mixture of aldehyde 25a2 (300 mg, 0.63 mmol) and (carbethoxymethylene)triphenylphosphorane (220 mg, 0.63 mmol) in THF (10 mL) is heated to 80° C. and stirred for 3 h. The mixture is allowed to cool to RT, concentrated and the residue loaded onto a combiflash (0 to 5% MeOH in DCM) to isolate unsaturated ester 117a1.

### Step 2:

i) A mixture of unsaturated ester 117a1 (110 mg, 0.20 mmol) in EtOH (10 mL) is purged with Ar (3×) before 10% Pd/C (30 mg) is added. The flask is charged with 1 atm of H₂ and stirred for 5 h at RT. The reaction mixture is filtered then concentrated.

ii) The residue is taken up in DMSO and treated with 1N NaOH (1 mL) and stirred for 0.5 h at RT. The mixture is acidified with AcOH before being diluted in EtOAc and washed with water and brine. The organic phase is dried over MgSO₄, filtered and concentrated.

iii) The residue is taken up in MeOH and treated with Na₂CO₃ (42 mg, 0.40 mmol) and I₂ (61 mg, 0.24 mmol). The mixture is stirred for 2 h at RT. The reaction is quenched by the addition of sat. aq. Na₂S₂O₃. The mixture diluted in EtOAc and washed with water and brine. The organic phase is dried over MgSO₄, filtered and concentrated. The residue is diluted in DMSO and injected onto a prep HPLC to isolate compound 4053.

### Step 3:

Ester 117a1 is saponified to acid 4034 using the protocol described in section ii) of step 2 followed by purification by prep HPLC.

### Example 118A

### (Synthetic Method BD): Preparation of Compound 4033

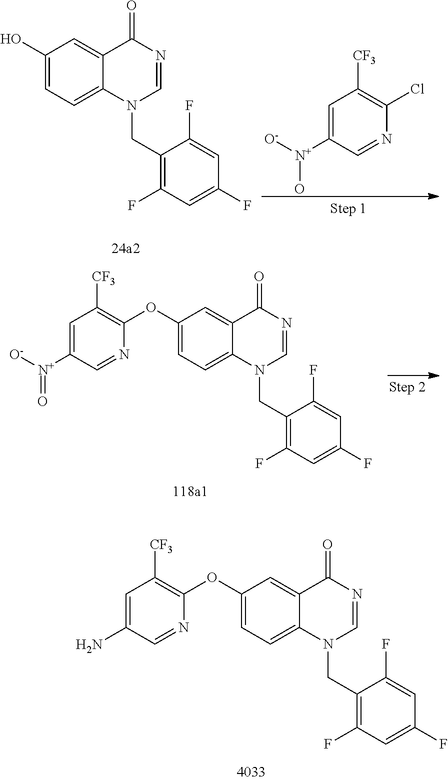

### Step 1:

Phenol 24a2 is coupled to 2-chloro-3-trifluoromethyl-5-nitropyridine using the protocol described in example 5C (synthetic method B) to form intermediate 118a1.

### Step 2:

To a mixture of nitroarene 118a1 (1.1 g, 2.1 mmol) in EtOH (30 mL) and 1N HCl (5 mL) zinc (powdered, 0.69 g, 11 mmol) is added. The mixture is stirred for 3 h at 100° C. The mixture is filtered and the filtrate is concentrated. The residue is subjected to combiflash (0 to 5% MeOH in DCM) to isolate compound 4033.

### Example 118B

### (Synthetic Method BE): Preparation of Compounds 4030 & 4031

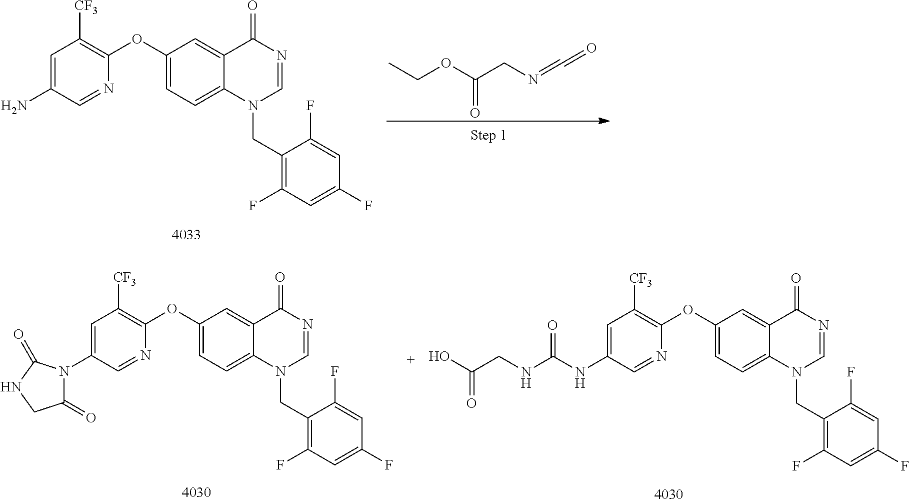

### Step 1:

To a mixture of aminopyridine 4033 (120 mg, 0.26 mmol) in dioxane (30 mL) is added and ethylisocyanatoacetate (40 mg, 0.31 mmol). The mixture is stirred for 1 h at 60° C. The mixture is concentrated and taken up in DMSO (3.5 mL) and 1N NaOH (0.5 mL, 0.5 mmol). The mixture is stirred for 10 min at 50° C. before being quenched by the addition of AcOH. The mixture is filtered then injected onto a prep HPLC to isolate compounds 4030 and 4031.

### Example 119A

### (Synthetic Method BF): Preparation of Compounds 4035 & 4036

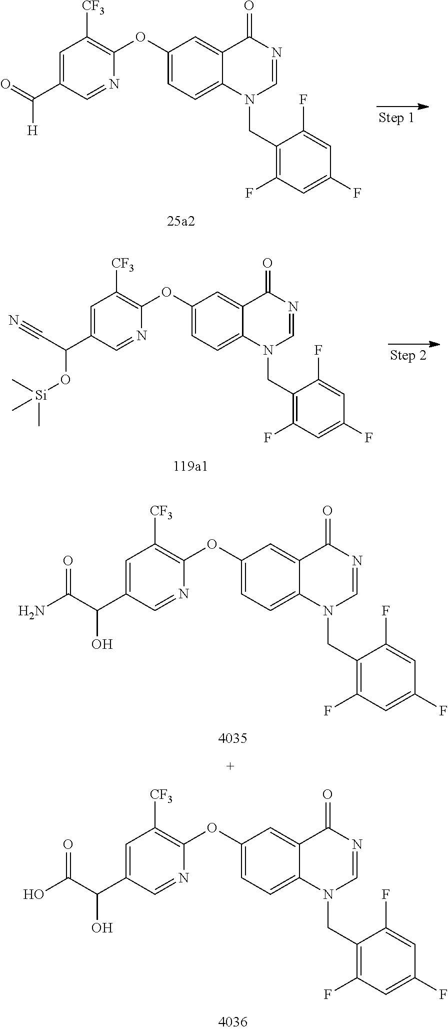

### Step 1:

To a chilled (0° C.) mixture of aldehyde 25a2 (300 mg, 0.63 mmol) in DCM (10 mL) is added TMSCN (0.25 mL, 1.9 mmol) and ZnI₂ (100 mg, 0.31 mmol). The mixture is stirred for 2 h at RT before being portioned between water and DCM. The aqueous phase is separated and extracted with DCM. The combined organic extracts are dried over MgSO₄, filtered and concentrated to afford intermediate 119a1 which is utilized without further purification.

### Step 2:

A mixture of nitrile 119a1 (100 mg, 0.17 mmol) in AcOH (30 mL) and conc H₂SO₄ (0.5 mL) is stirred for 1 h at 100° C. The mixture is diluted in AcOH, filtered then injected onto prep. HPLC to isolate compounds 4035 and 4036.

### Example 120A

### (Synthetic Method BG): Preparation of Compound 5004

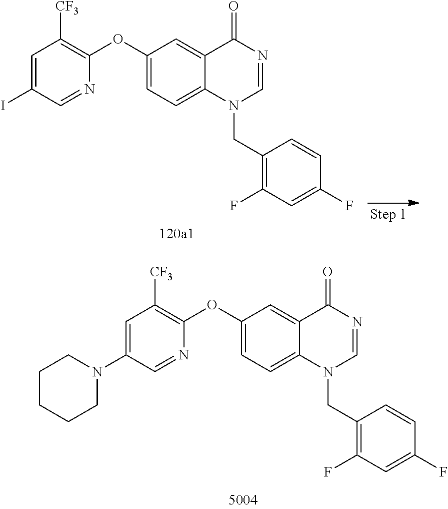

Intermediate 120a1 is synthesized from pyridylchloride 1a1 and phenol 5b3 using synthetic method I.

### Step 1:

To a mixture of 120a1 (56 mg, 0.10 mmol) in DMSO (2.5 mL) is added Pd(OAc)₂ (2 mg, 0.01 mmol), Cs₂CO₃ (180 mg, 0.56 mmol), rac-BINAP (8 mg, 0.01 mmol) followed by piperidine (44 μL, 0.50 mmol). The mixture is degassed with Ar then stirred overnight at 115° C. The mixture is diluted with DMSO and acidified with TFA then injected onto a semi-prep. HPLC to isolate compound 5004.

### Example 121A

### Preparation of Compound 5014

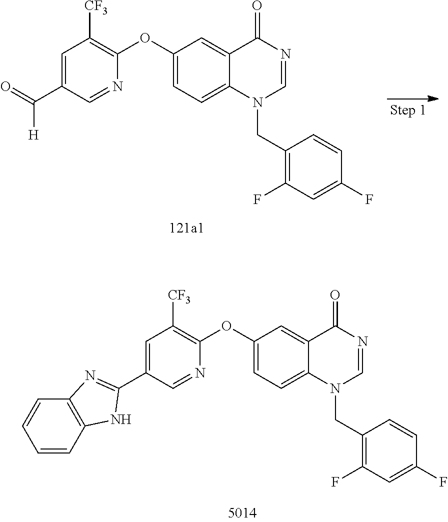

Intermediate 121a1 is synthesized from pyridylchloride 3a1 and phenol 5b3 using the protocol described in example 19A (synthetic method I).

### Step 1:

To a mixture of aldehyde 121a1 (80 mg, 0.17 mmol) in DMF (1 mL) and water (40 μL) is added 1,2-diaminobenzene (25 mg, 0.23 mmol) and Oxone® (70 mg, 0.11 mmol). The mixture is stirred for 2 h at RT. The mixture is diluted with water and the pH is adjusted to −6. The resulting precipitate is collected by filtration. The crude product is taken up in DMSO/MeCN (1:2) then purified by prep. HPLC to afford compound 5014.

### Example 122A

### (Synthetic Method BH): Preparation of Compound 5016

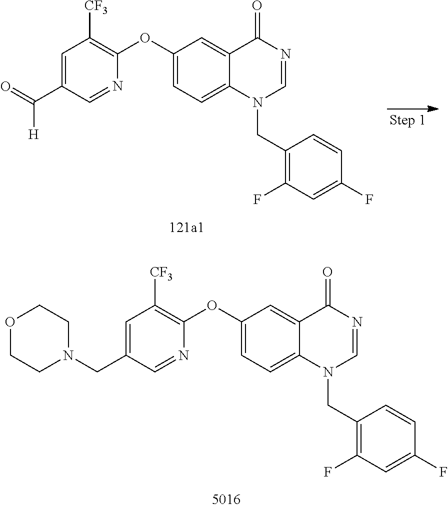

### Step 1:

To a mixture of aldehyde 121a1 (40 mg, 0.09 mmol) in EtOH (0.5 mL) is added AcOH (46 μL) and morpholine (14 μL, 0.16 mmol). After stirring for 30 min at 50° C., NaCNBH₃ (6 mg, 0.10 mmol) is added and the resulting mixture is stirred for 30 min at RT. The mixture is then diluted with water, filtered and injected onto a prep. HPLC to isolate compound 5016.

### Example 123A

### Preparation of Compound 5035

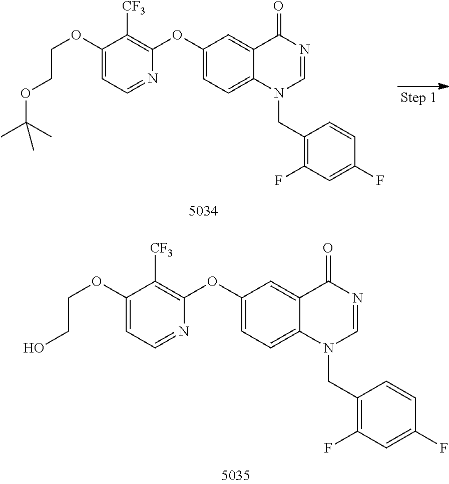

### Step 1:

To a mixture of t-Bu-ether 5034 (44 mg, 0.08 mmol) in DCM (2 mL) is added TFA (2 mL). The resulting mixture is stirred for 2 h at RT. The mixture is concentrated then diluted with DMSO, filtered and injected onto a prep. HPLC to isolate compound 5035.

### Example 124A

### Preparation of Compound 5036

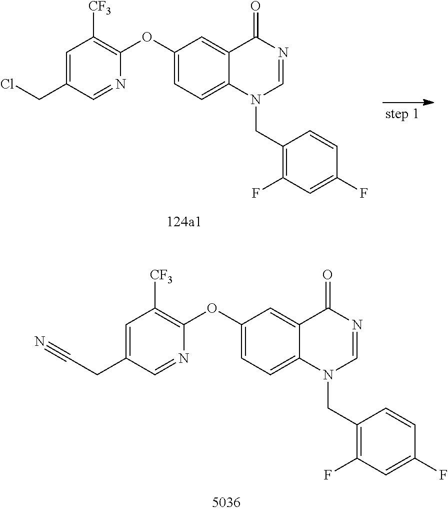

Intermediate 124a1 is prepared from aldehyde 121a1 using the protocols described in steps 2 of example 25A and step 1 of example 29A.

### Step 1:

To a mixture of 124a1 (35 mg, 0.06 mmol) in DMSO (2 mL) is added NaCN (6 mg, 0.13 mmol). The resulting mixture is stirred for 30 min at RT. The mixture is then acidified with TFA, filtered and injected onto a prep. HPLC to isolate compound 5036.

### Example 125A

### (Synthetic Method BI): Preparation of Compound 6003

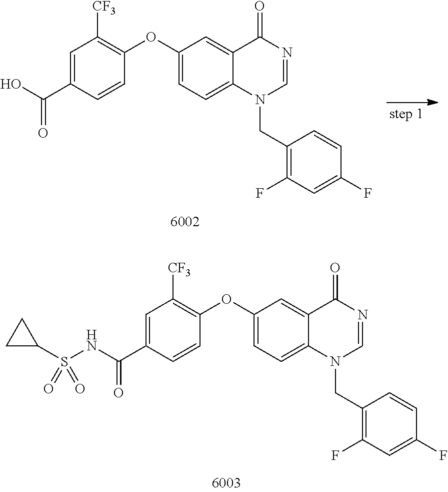

### Step 1:

To a mixture of acid 6002 (100 mg, 0.20 mmol) in THF (10 mL) is added CU (130 mg, 0.80 mmol). The resulting mixture is stirred for 30 min at reflux. The mixture is allowed to cool to RT and cyclopropanesulfonamide (97 mg, 0.80 mmol) is added followed by DBU (66 μL, 0.44 mmol). The resulting mixture is stirred for 3.5 h at RT. The mixture is acidified with AcOH, filtered and injected onto a prep. HPLC to isolate compound 6003.

### Example 126A

### (Synthetic Method BJ): Preparation of Compounds 6034, 6035 & 6037

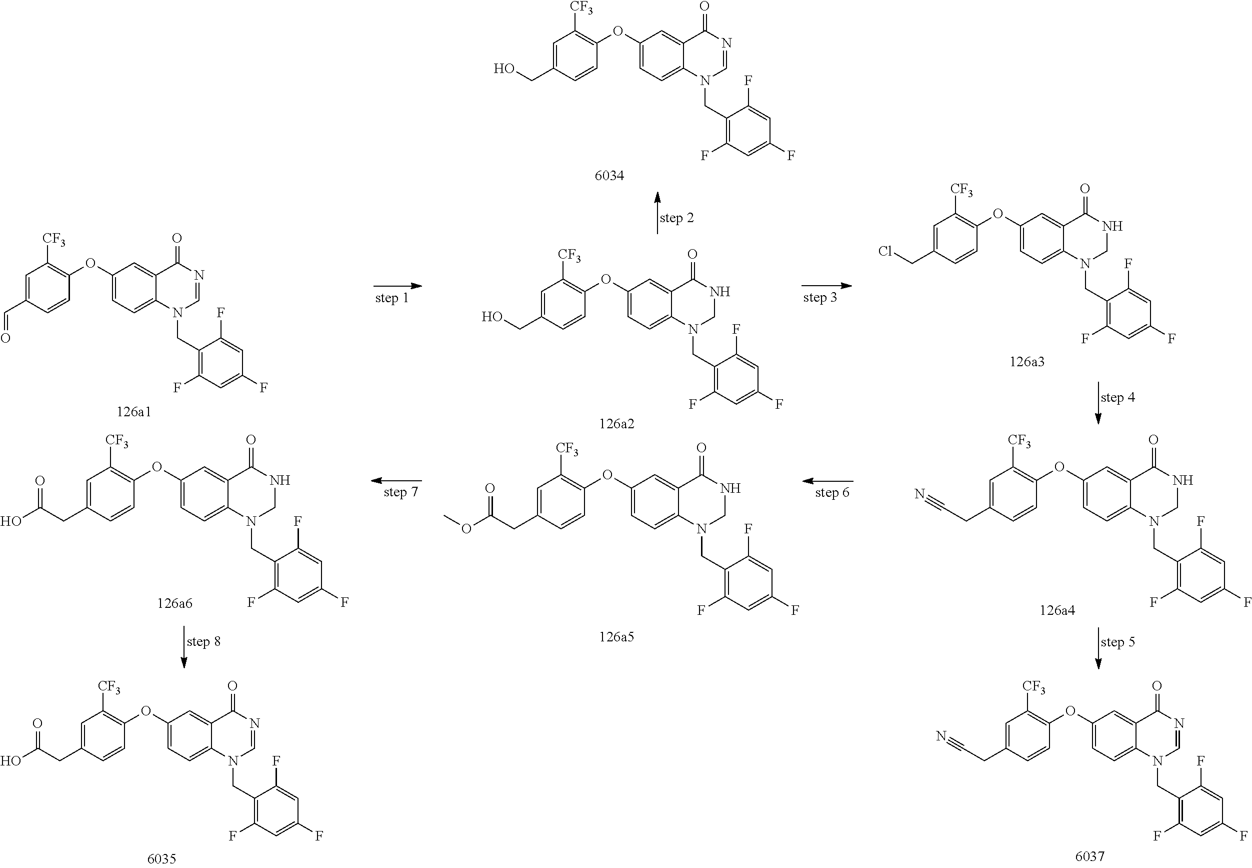

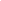

Aldehyde 126a1 is prepared from 4-fluoro-3-trifluoromethylbenzaldehyde and phenol

24a2 using the protocol described in example 13A (synthetic method G).

### Step 1:

Aldehyde 126a1 is reduced to alcohol 126a2 using the protocol described in step 2 of example 25A.

### Step 2:

Alcohol 126a2 is converted to compound 6034 using the protocol described in step 3 of example 25A.

### Step 3:

Alcohol 126a2 is converted to benzylchloride 126a3, using the protocol described in example 29A.

### Step 4:

Benzylchloride 126a3 is converted to benzylcyanide 126a4 using the protocol described in step 1 of example 62A.

### Step 5:

Benzylcyanide 126a4 is converted to compound 6037 using the protocol described in step 3 of example 25A.

### Step 6:

To a mixture of benzylcyanide 126a4 (1.65 g, 3.4 mmol) in MeOH (20 mL) is added a sat. solution of HCl in MeOH (80 mL). The mixture is stirred for 3 h at RT before it is purged with bubbling N₂. The mixture is concentrated and the residue is taken up in MeCN. To the mixture is added 1N aq. HCl (3 mL). The mixture is stirred for 1 h at RT.

The mixture is diluted in the EtOAc and washed with water and brine then dried over

MgSO₄, filtered and concentrated. The crude product is purified by combiflash (40 to 100% EtOAc in Hex) to afford ester 126a5.

### Step 7:

Methylester 126a5 is saponified to acid 126a6 using the protocol described in step 4 of example 12B.

### Step 8:

Acid 126a6 is converted to compound 6035 using the protocol described in step 3 of example 25A.

### Example 127A

### Preparation of Compound 6033

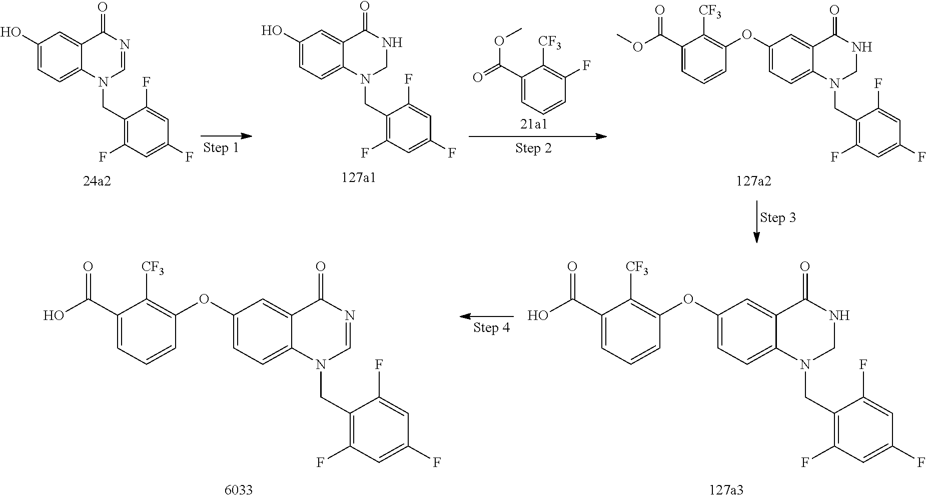

### Step 1:

Quinazolinone 24a2 is reduced to quinazolidinone 127a1 using the protocol described in step 2 of example 25A.

### Step 2:

Phenol 127a1 is coupled to F-arene 21a1 to form diarylether 127a2 using the protocol described in example 27A (synthetic method O).

### Step 3:

Methylester 127a2 is saponified to acid 127a3 using the protocol described in step 3 i) of example 6A.

### Step 4:

Quinazolidinone acid 127a3 is oxidized to quinazolinone 6033 using the protocol described in step 3 of example 25A.

### Example 128A

### (Synthetic Method BK): Preparation of Compound 7003

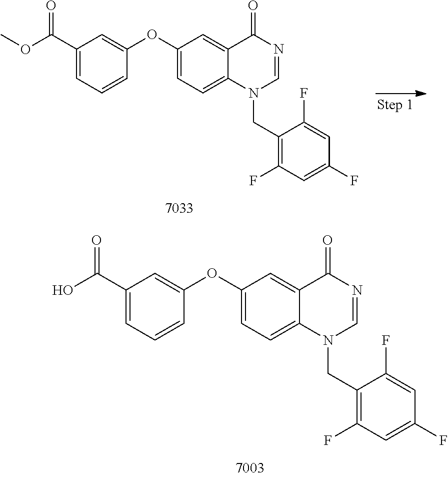

### Step 1:

i) Methylester 7033 is saponified to the acid using the protocol described in step 3 i) of example 6A.

ii) The resulting carboxamide is converted to quinazolinone 7003 using the protocol described in step 3 of example 1A.

### Example 129A

### (Synthetic Method BL): Preparation of Compound 7005

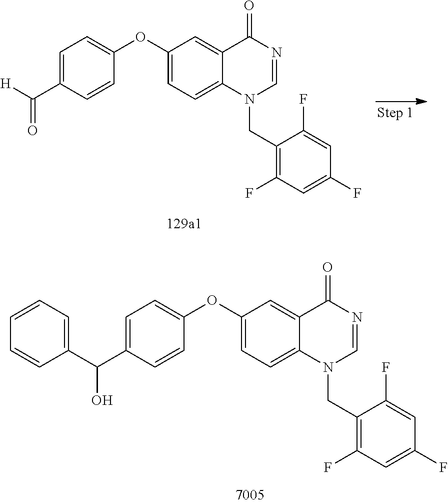

Aldehyde 129a1 is prepared from 4-fluorobenzaldehyde and phenol 24a2 using the protocol described in example 13A (synthetic method G).

### Step 1:

To 129a1 (62 mg, 0.15 mmol) in THF (4 mL) at 0° C. is slowly added PhMgBr (1.0 M in THF, 150 μL, 0.15 mmol). Additional PhMgBr is gradually added until the reaction is complete. The mixture is quenched with AcOH. The mixture is concentrated, diluted in AcOH, filtered then injected onto a semi-prep. HPLC to isolate compound 7005.

### Example 130A

### Preparation of Compound 7006

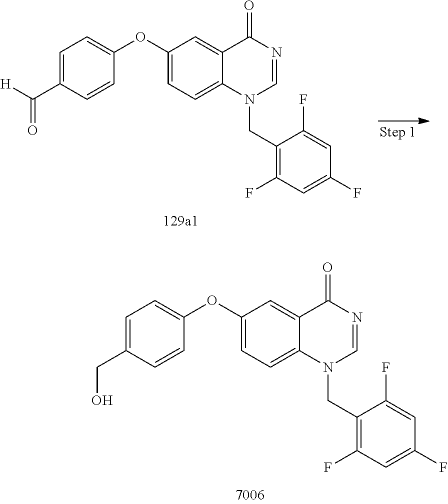

### Step 1:

Aldehyde 129a1 is reduced to alcohol 7006 using the protocol described in steps 2 and 3 of example 25A.

### Example 131A

### Preparation of Compounds 7010 & 7011

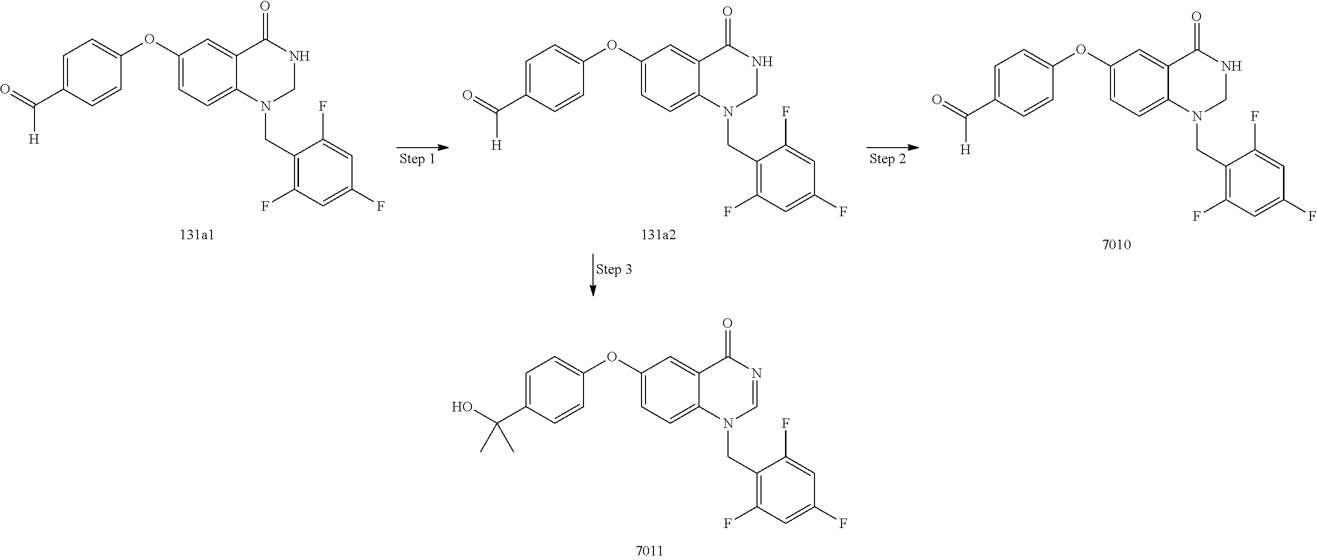

Aldehyde 131a1 is prepared from 4-fluorobenzaldehyde and phenol 127a1 using the protocol described in example 13A (synthetic method G).

### Step 1:

i) MeMgBr is added to aldehyde 131a1 as described in in example 129A (synthetic method BL).

ii) To the intermediate in DCM (10 mL) is added Dess-Martin periodinane (300 mg, 0.7 mmol). The mixture is stirred for 2 h at RT before it is partitioned between sat. aq. NaHCO₃ and EtOAc. The organic phase is separated and washed with brine, dried over MgSO₄, filtered and concentrated. The crude product is purified by combiflash (40%-90% EtOAc/Hex) to afford methylketone 131a2.

### Step 2:

Quinazolidinone 131a2 is oxidized to quinazolinone 7010 using the protocol described in step 3 of example 25A.

### Step 3:

Quinazolidinone 131a2 is converted to 7011 using the protocol described in example 129A (synthetic method BL) followed by the protocol described in step 3 of example 25A.

### Example 132A

### Preparation of Compound 7017

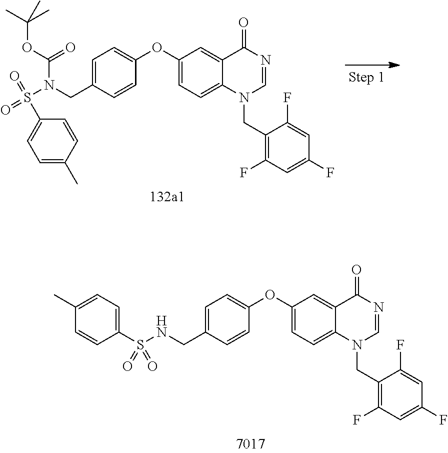

Intermediate 132a1 is prepared from N-Boc-toluenesulfonamide and alcohol 7006 using the protocol described in example 38A (synthetic method U).

### Step 1:

N-Boc group of 132a1 is removed to afford 7017 using the protocol described in step 1 of example 123A.

### Example 133A

### Preparation of Compound 7025

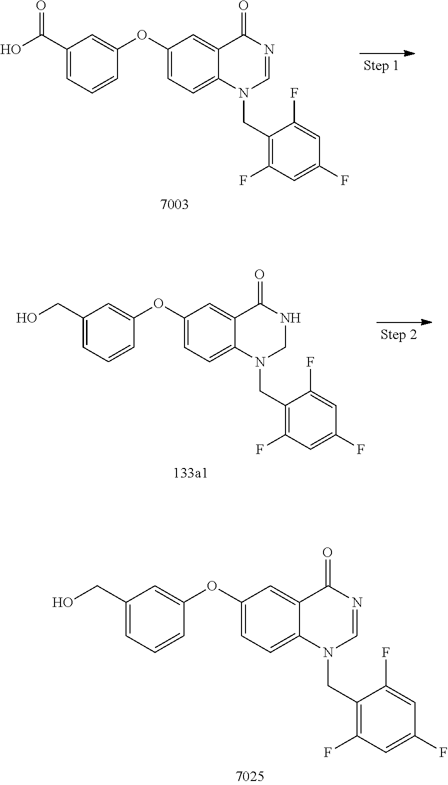

### Step 1:

To carboxylic acid 7003 (130 mg, 0.31 mmol) in THF (1.5 mL) is added DIPEA (65 μL, 0.37 mmol) and the BOP reagent (150 mg, 0.34 mmol). The mixture is stirred for 5 min before chilling to 0° C. and then NaBH₄ (24 mg, 0.62 mmol) is added. The mixture is allowed to warm to RT and is stirred for 1 h. An additional 2 eq. of NaBH₄ is added and the mixture is stirred overnight. The mixture is diluted in EtOAc and washed with 5% aq. HCl, sat. aq. NaHCO₃ and brine. The organic phase is dried over MgSO₄, filtered and concentrated to afford crude alcohol 133a1 that is utilized in the next step without further purification.

### Step 2:

Quinazolidinone 133a1 is oxidized to quinazolinone 7025 using the protocol described in step 3 of example 25A.

### Example 134A

### Preparation of Compound 7028

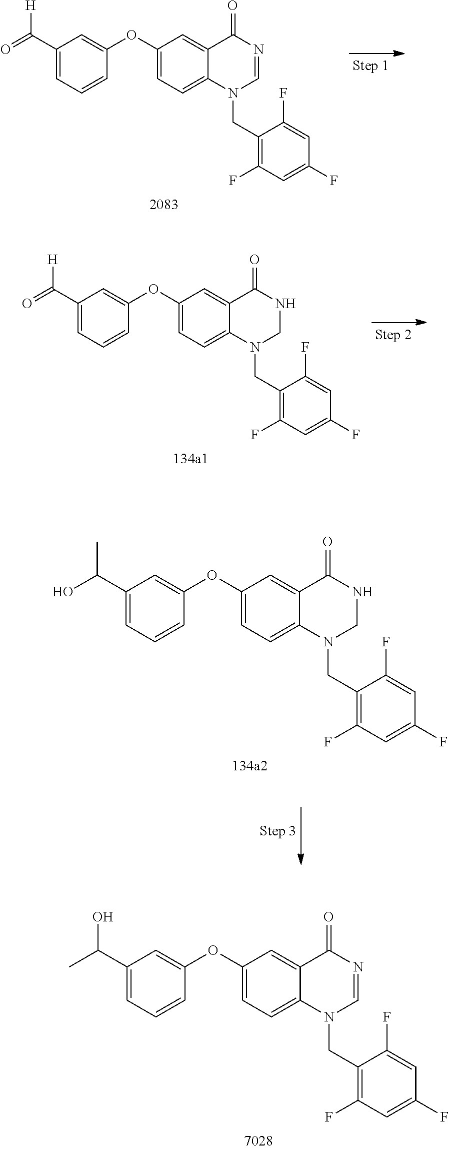

### Step 1:

A mixture of aldehyde 2083 (150 mg, 0.35 mmol) in MeOH (10 mL) is passed through an H-cube hydrogenation reactor three times at 40° C. and 60 bar with a 1 mL/min flow rate. The fractions were concentrated to afford quinazolidinone 134a1 that is utilized in the next step without further purification.

### Step 2:

MeMgBr is added to quinazolidinone 134a1 to form alcohol 134a2 using the protocol described in example 129A (synthetic method BL).

### Step 3:

Quinazolidinone 134a2 is oxidized to quinazolinone 7028 using the protocol described in step 3 of example 25A.

### Example 135A

### Preparation of Compound 7029

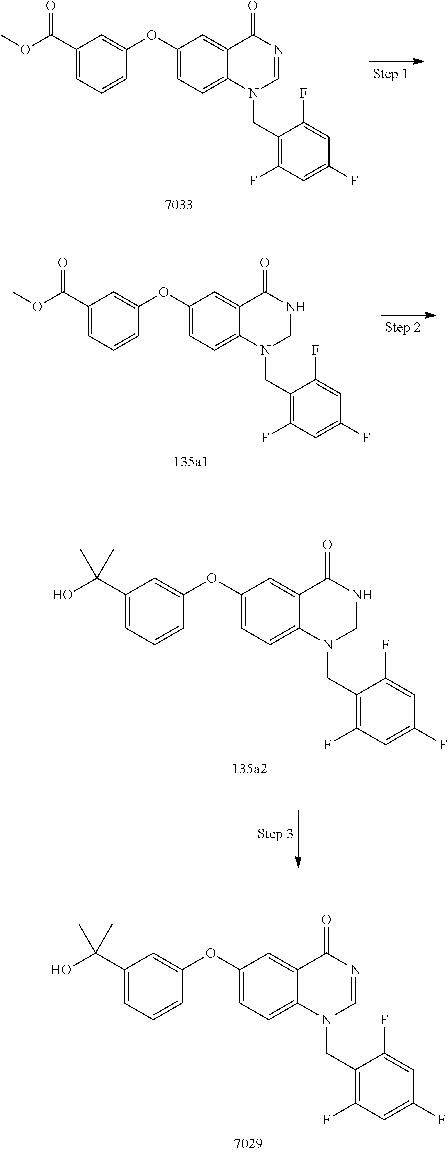

### Step 1:

To a mixture of ester 7033 (138 mg, 0.31 mmol) in THF (3 mL) cooled to −78° C. is dropwise added DiBAI-H (1.0 M in THF, 1.1 mL, 1.1 mmol). The mixture is stirred for 1 h at −78° C. before the mixture is allowed to warm to 0° C. and stirred for an additional hour. Another 1.75 eq. of DiBAI-His added and the mixture is stirred at 0° C., gradually warming to RT overnight. The mixture is chilled to 0° C. once again and 3.5 eq of DiBAI-His added. The mixture is diluted in DCM and washed with a sat. aq. mixture of Rochelle's salt. The aqueous phase is back-extracted with DCM (2×). The combined organic phases are washed with brine, dried over MgSO₄, filtered and concentrated. The crude product is purified by combiflash (0 to 5% MeOH in DCM) to afford quinazolidinone 135a1.

### Step 2:

MeMgBr is added to quinazolidinone 135a1 form alcohol 135a2 using the protocol described in example 129A (synthetic method BL).

### Step 3:

Quinazolidinone 135a2 is oxidized to quinazolinone 7029 using the protocol described in step 3 of example 25A.

### Example 136A

### Preparation of Compound 7063

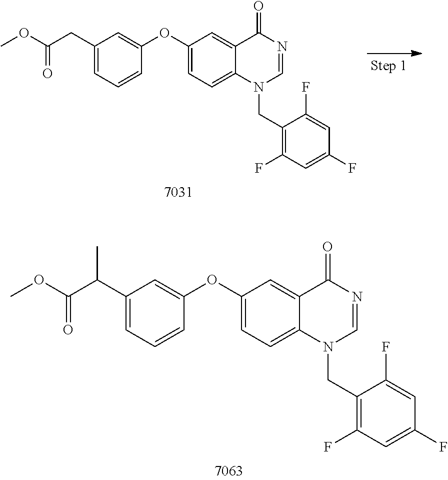

### Step 1:

To a mixture of ester 7031 (250 mg, 0.55 mmol) in THF (10 mL) cooled to −78° C. is added NaHMDS (1.0 M in THF, 0.61 mL, 0.61 mmol). The mixture is stirred for 0.5 h at −78° C. before MeI (1.0 M in THF, 0.55 mL, 0.55 mmol) is added. The mixture is stirred for 1 h at −78° C. before being allowed to warm to RT. The mixture is concentrated and the residue is taken up in CH₃CN/water, filtered then injected onto a prep HPLC to isolate compound 7063.

### Example 137A

### Preparation of Compound 7064

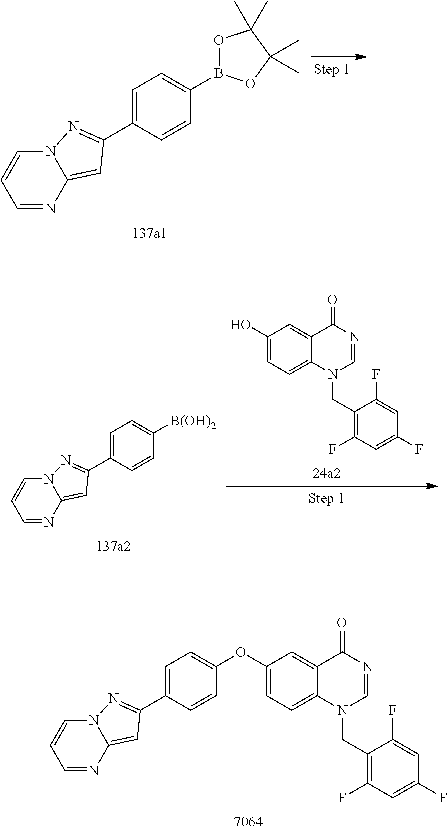

Boronic acid 137a1 is prepared according to the procedure described in WO 2009/000818, herein incorporated by reference.

### Step 1:

Reference: Gouts, S. J.; Adams, J.; Krolikowski, D.; Snow, R. J. Tetrahedron Lett. 1994, 35, 5109, herein incorporated by reference.

To a mixture of boronic ester 137a1 (150 mg, 0.47 mmol) in acetone (20 mL) and water (10 mL) are added NaIO₄ (300 mg, 1.4 mmol) and NH₄OAc (110 mg, 1.4 mmol). The mixture is stirred at RT for 16 h before 2N NaOH (30 mL) is added. The mixture is stirred for 1 h then the reaction is diluted in 1N HCl (pH ˜1). The organic phase is extracted with DCM (3×). The combined organic phases are washed with brine, dried over Na₂SO₄, filtered and concentrated to afford boronic acid 137a2.

### Step 2:

Phenol 24a2 is coupled to boronic acid 137a2 to form compound 7064 using the protocol described in example 144A (synthetic method AC).

### Example 138A

### Preparation of Compound 8001

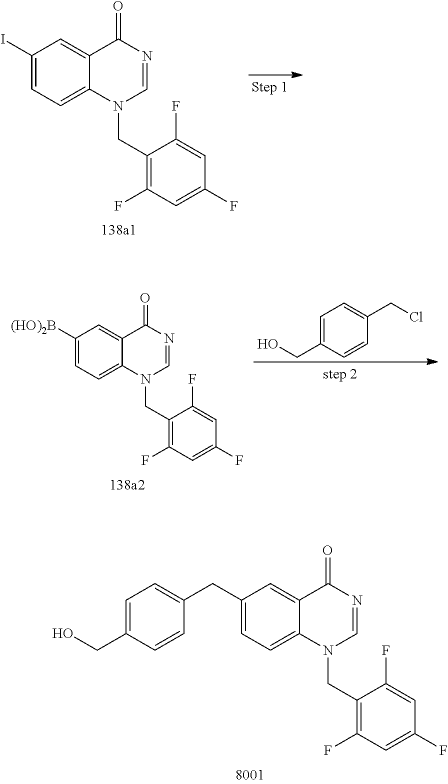

Aryl iodide 138a1 is prepared from 5-iodo-2-aminobenzoic acid as described in example 5B.

### Step 1:

To mixture of iodide 138a1 (600 mg, 1.4 mmol) in DMF (6 mL), degassed with Ar, at RT, are added bispinacolatoborane (370 mg, 1.5 mmol), KOAc (380 mg, 4.0 mmol) and Pd(dppf)Cl₂-DCM complex (120 mg, 0.15 mmol). The reaction is stirred at 95° C. for 1 h. The crude reaction mixture is diluted with water and the product is extracted with EtOAc (3×). The combined organic phases are washed with water, brine, dried over MgSO₄, filtered and concentrated. The crude mixture is further purified by combiflash (3% MeOH/AcOEt) to afford boronic acid 138a2.

### Step 2:

4-(chloromethyl)benzylalcohol is coupled to boronic acid 138a2 to form compound 8001 using the protocol described in example 33A (synthetic method S).

### Example 139A

### (Synthetic Method BM): Preparation of Compound 8002

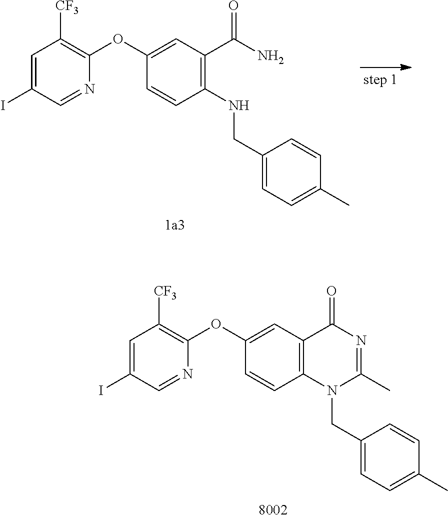

### Step 1:

To a mixture of 1a3 (11 mg, 0.02 mmol) in trimethylorthoacetate (0.2 mL) is added TFA (25 μL). The mixture is stirred for 2 h at RT. The mixture is filtered then injected onto a prep HPLC to isolate compound 8002.

### Example 140A

### (Synthetic Method BN): Preparation of Compound 8004

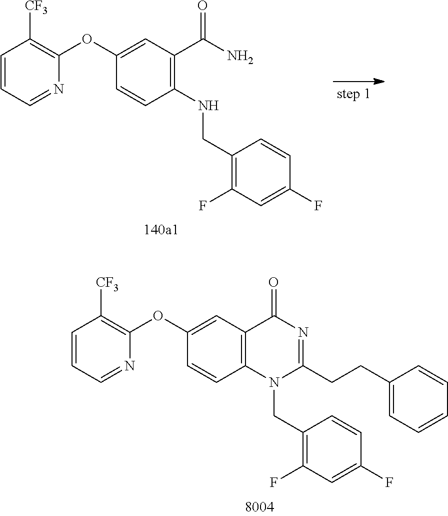

Carboxamide 140a1 is prepared from aniline 70a4 and 2,4-difluorobenzaldehyde using the protocol described in step 1 of example 5B.

### Step 1:

To a mixture of 140a1 (30 mg, 0.07 mmol) in hydrocinnamoyl chloride (0.5 mL) is added TFA (25 μL). The mixture is warmed to 50° C. and stirred for 16 h. The mixture is diluted in AcOH, filtered then injected onto a prep HPLC to isolate compound 8004.

### Example 141A

### Preparation of Compound 8005

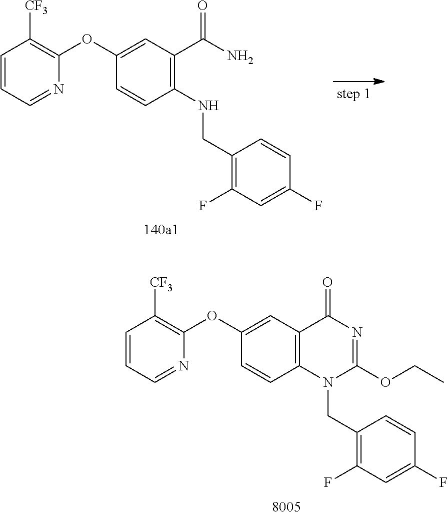

### Step 1:

To a mixture of 140a1 (30 mg, 0.07 mmol) in tetraethylorthocarbonate (0.5 mL) is added TFA (25 μL). The mixture is heated to 120° C. and stirred for 1 h. The mixture is concentrated then diluted in DMSO, filtered then injected onto a prep HPLC to isolate compound 8005.

### Example 142A

### Preparation of Compound 8007

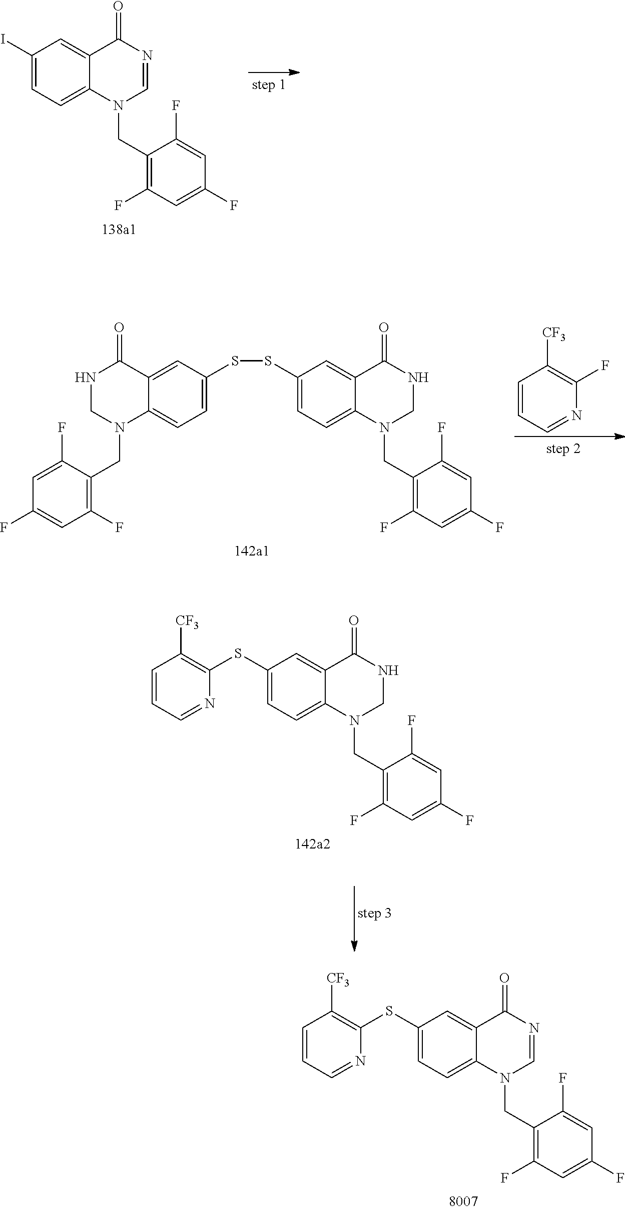

### Step 1:

Reference: Takagi, K. Chem. Lett. 1985, 14, 1307, herein incorporated by reference.

To a mixture of iodide 138a1 (500 mg, 1.2 mmol) in DMF (2.5 mL), degassed with Ar, at RT, are added NiBr₂ (27 mg, 0.12 mmol), thiourea (140 mg, 1.8 mmol) and NaCNBH₃ (11 mg, 0.18 mmol). The reaction is stirred at 120° C. for 0.5 h in a microwave (2×). The crude reaction mixture is diluted in EtOAc/Et₂O. The organic phase is washed with 10% aq. citric acid, water and brine. The organic phase is dried over MgSO₄, filtered and concentrated to afford the crude disulfide 142a1 that is utilized in the next step without further purification.

### Step 2:

Reference (disulfide reduction): Vidya Sagar Reddy, G.; Venkat Rao, G.; lyengar, D. S. Synth. Comm. 2000, 30, 859, herein incorporated by reference.

To a mixture of disulfide 142a1 (100 mg, 0.16 mmol) in EtOH (6 mL) are added Indium powder (140 mg, 1.2 mmol) and NH₃Cl (64 mg, 1.2 mmol). The reaction is stirred for 1 h at reflux. The mixture is concentrated to dryness and the residue is taken up in DMF. To the mixture is added 2-fluoro-3-trifluoromethylpyridine (50 mg, 0.30 mmol) and Cs₂CO₃ (220 mg, 0.67 mmol). The mixture is stirred for 15 h at 85° C. The crude reaction mixture is diluted in EtOAc then washed with water and brine. The organic phase is dried over MgSO₄, filtered and concentrated to afford the crude thioether 142a2 that is utilized in the next step without further purification.

### Step 3:

Quinazolidinone 142a2 is oxidized to quinazolinone 8007 using the protocol described in step 3 of example 25A.

### Example 143A

### Preparation of Compound 8009

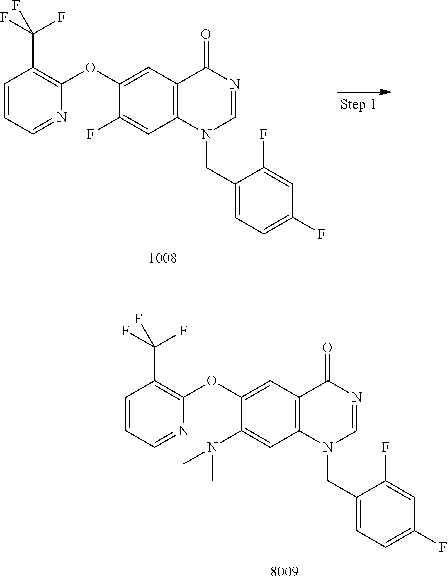

### Step 1:

To 1008 (35 mg, 0.08 mmol) in THF (1 mL) is added Me₂NH₂/THF (2 M, 40 μL, 0.08 mmol) and Et₃N (28 μL, 0.20 mmol). The reaction is stirred for 20 h at RT. The solution is then diluted with AcOH (2 mL) and purified by semi-prep. HPLC to afford 8009.

### Example 144A

### (Synthetic Method AC): Preparation of Compound 2083

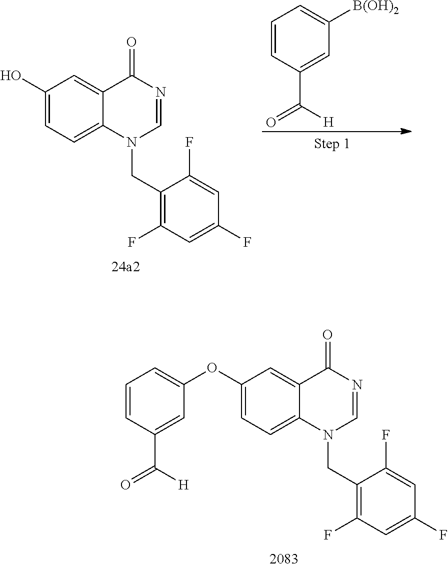

### Step 1:

Reference: Chan, D. M. T.; Monaco, K. L.; Wang, R.-P.; Winters, M. P. Tet. Lett. 1998, 39, 2933, herein incorporated by reference.

To a mixture of phenol 24a2 (500 mg, 1.2 mmol) in DCM (30 mL) are added 3-formylphenylboronic acid (357 mg, 2.4 mmol), NEt₃ (1.5 mL, 11 mmol), Cu(OAc)₂ (432 mg, 2.4 mmol) and activated 4 Å molecular sieves (350 mg). The mixture is stirred overnight at RT open to the air. The reaction mixture is then filtered through a pad of celite. The filtrate is concentrated and the crude product is purified by combiflash (0 to 5% MeOH/DCM) to afford compound 2083.

### Example 145A

### Preparation of Compound 2107

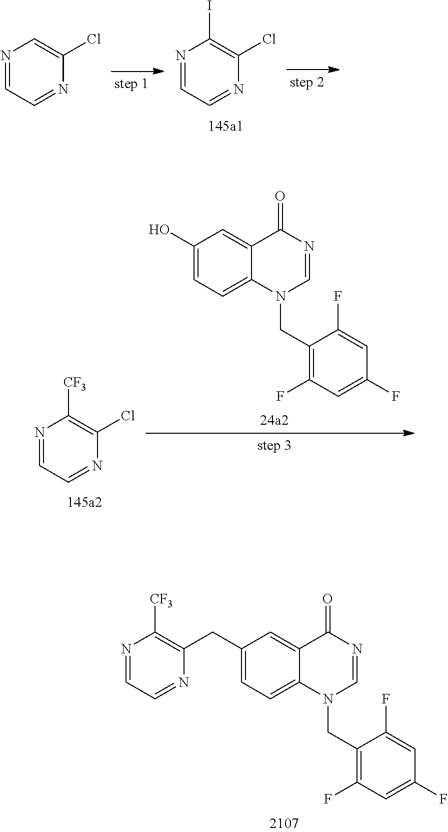

### Step 1:

A solution of n-BuLi (2.5 M in hexanes, 36 mL, 91 mmol) is diluted in anhydrous THF (200 mL) under Ar. The mixture is cooled to −70° C. then 2,2,6,6-tetramethylpiperidine (15 g, 110 mmol) is added dropwise. The solution is allowed to warm to 0° C. then is kept at that temperature and stirred for 30 min. The mixture is cooled to −70° C. once again and chloropyrazine (8.0 g, 70 mmol) is added dropwise over 30 min. This mixture is stirred for 30 min at −70° C. A mixture of I₂ (21 g, 84 mmol) in anhydrous dry THF (20 mL) is added dropwise to the reaction mixture. Stirring is continued at −70° C. for 2 h before the mixture is allowed to warm to ambient temperature. MeOH (5 mL) is added to quench the mixture. The mixture is evaporated to dryness and the residue is diluted in EtOAc (50 mL) then filtered through a pad of silica gel. The filtrate is evaporated to dryness and the residue is purified by combiflash (Hex/DCM, 2:1) to afford 2-chloro-3-iodopyrazine 145a1.

### Step 2:

Reference: Su, D.-B.; Duan, J.-X.; Chen, Q.-Y Tet. Lett. 1991, 32, 7689, herein incorporated by reference.

A mixture of dry KF (4.4 g, 76 mmol), CuI (11 g, 57 mmol), ClCF₂COOMe (11 g, 76 mmol), and 2-chloro-3-iodopyrazine 145a1 (9.2 g, 38 mmol) in anhydrous DMF (40 mL) is heated with stirring to 115° C. under Ar and kept at this temperature for 3 h. The mixture is allowed to cool to RT before the mixture is quenched with water (20 mL). The mixture is made acidic with HCl (˜pH=1). The mixture is extracted with ether (4×200 mL) then the combined organic extracts are washed with sat. aq. NaHCO₃ (200 mL) and sat. aq. NaS₂O₃ (2×50 mL). The organic layer is dried over anhydrous Na₂SO₄. The mixture is carefully concentrated then the residue is distilled under high vacuum (1 Torr, ˜70° C.) to afford 2-chloro-3-trifluoromethylpyrazine 145a2.

### Step 3:

Chloropyrazine 145a2 is coupled to phenol 24a2 using the protocol described in synthetic method Ito afford compound 2107.

### Example 146A

### (Synthetic Method BO): Preparation of Compound 6040

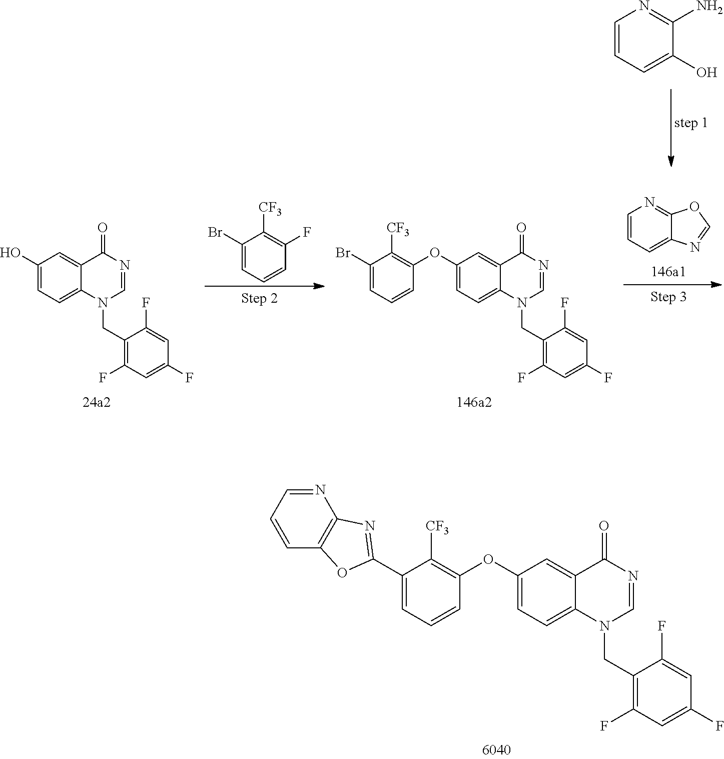

### Step 1:

To 2-amino-3-hydroxypyridine (3.0 g, 27 mmol) in trimethyl orthoformate (20 mL) is added TFA (0.5 mL). This reaction mixture is heated 5 h at 120° C. in a sealed tube. The reaction mixture is concentrated under vacuum to dryness and the crude material is purified by flash chromatography (100% Hex to 6:4 Hex/EtOAc) to afford intermediate 146a1.

### Step 2:

To compound 24a2 (600 mg, 1.4 mmol) in DMF (10 mL) is added 2-bromo-6-fluorobenzotrifluoride (693 mg, 2.9 mmol) and Cs₂CO₃ (1.4 g, 4.3 mmol). The reaction mixture is heated at 40° C. for 3 days. The reaction mixture is then cooled to ambient temperature, the solution is decanted and the solid is washed with EtOAc (3×). The combined organic phases are washed with 1N HCl, dried over MgSO₄, filtered and concentrated. The crude product is purified by combiflash (0 to 10% MeOH in DCM) to afford bromide 146a2.

### Step 3:

Reference: McClure, M. S.; Glover, B.; McSorley, E.; Millar, A.; Osterhout, M. H.; Roschangar, F. Org. Lett. 2001, 3, 1677 and Pivsa-Art, S.; Satoh, T.; Kawamura, Y.; Miura, M.; Nomura M. Bull. Chem. Soc. Jpn. 1998, 71, 467, herein incorporated by reference.

To bromide 146a2 (114 mg, 0.22 mmol) in DMF (4 mL) is added the intermediate 146a1 (88 mg, 0.73 mmol), KOAc (42 mg, 0.43 mmol), TBAB (69 mg, 0.22 mmol), CuI (82 mg, 0.43 mmol) and bis(tri-t-butylphosphine)palladium(0) (11 mg, 0.02 mmol). Ar is bubbled through the mixture for 10 min, the vial is then capped and the reaction is stirred 10 min at 170° C. in a microwave. After the mixture cools to RT, the mixture is diluted with MeOH, filtered and directly injected onto a prep. HPLC to isolate 6040.

### Example 147A

### Preparation of Compound 4054

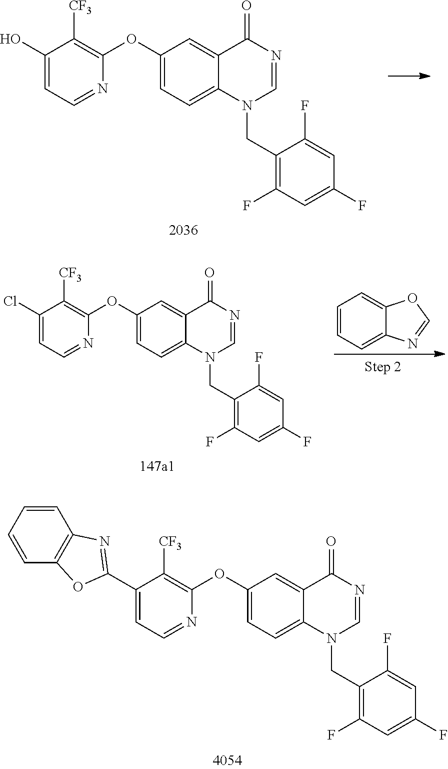

### Step 1:

Compound 2036 (500 mg, 1.1 mmol) in POCl₃ (6 mL) is stirred for 16 h at RT in a seal vial. The reaction mixture is concentrated under vacuum to dryness and the crude material is purified by flash chromatography (100% DCM to 10:1 DCM/MeOH) and then triturated in EtOAc to afford intermediate 147a1.

### Step 2:

Intermediate 147a1 is converted to compound 4054 as described in example 146A step 3.

### Example 148A

### (Synthetic Method BP): Preparation of Compound 6041

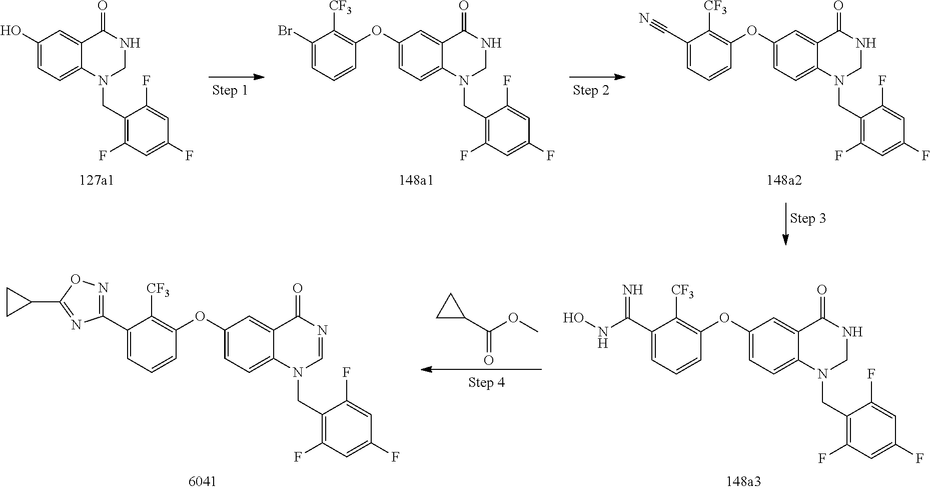

### Step 1:

To compound 127a1 (7.9 g, 26 mmol) in DMSO (46 mL) are added 2-bromo-6-fluorobenzotrifluoride (7.5 g, 31 mmol) and K₂CO₃ (7.1 g, 51 mmol). The reaction mixture is heated at 100° C. for 16 h. The reaction mixture is then cooled to ambient temperature, and water (300 mL) is added. The product is extracted with ethyl acetate (3×150 mL). The combined organic phases are washed with brine, dried over MgSO₄, filtered and concentrated. The crude product is purified by combiflash (0 to 0.25% MeOH in DCM) to afford bromide 148a1.

### Step 2:

To a solution of bromide 148a1 (5.02 g, 9.45 mmol) in DMAc (50 mL) is added Zn(CN)₂ (2.25 g, 19.2 mmol) and Ar is bubbled through the mixture for 20 min. Pd(PPh₃)₄ (0.90 g, 0.77 mmol) is added and Ar is bubbled through the mixture for 10 min. The reaction mixture is then heated to 110° C. for 3 h and then diluted with ethyl acetate (1 L), washed with brine, dried over Na₂SO₄ and filtered. The organic phase is concentrated and the crude product is purified by combiflash (1 to 10% EtOAc in DCM) to afford compound 148a2.

### Step 3:

To compound 148a2 (500 mg, 1.0 mmol) in MeOH (10 mL) is added hydroxylamine 50 wt % solution in water (220 μL, 3.3 mmol). The mixture is stirred for 16 h at 70° C. before being concentrated to afford crude intermediate 148a3. The residue is used as such without further purification.

### Step 4:

To the intermediate 148a3 (80 mg, 0.16 mmol) in NaOEt, 21 wt % solution in ethanol (0.71 mL, 1.6 mmol) is added methyl cyclopropanecarboxylate (39 mg, 0.39 mmol). The mixture is stirred for 1 h at 80° C. AcOH (1 mL) is added to the reaction mixture before being concentrated. The residue is taken up in MeOH (1 mL) and iodine (119 mg, 0.47 mmol) is added. The reaction mixture is stirred for 1 h at RT. The excess of I₂ is destroyed by the addition of an aq. solution of Na₂S₂O₃. The mixture is diluted with MeOH, filtered and directly injected onto a prep. HPLC to isolate 6041.

### Example 149A

### Preparation of Compound 8010

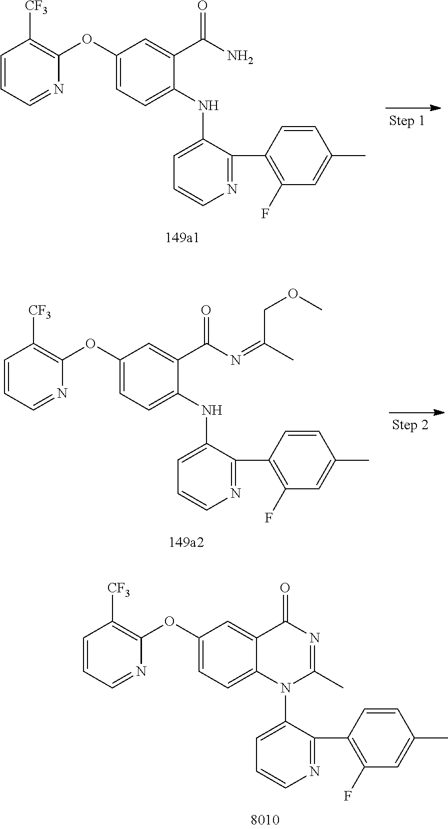

Intermediate 149a1 is synthesized as described in example 93A

### Step 1:

Reference: Sasada, T.; Kobayashi, F.; Sakai, N.; Konakahara, T. Org. Lett. 2009, 11, 2161, herein incorporated by reference.

To a suspension of compound 149a1 (190 mg, 0.39 mmol) in MeCN (500 μL) is added triethyl orthoacetate (1 mL) and ZnCl₂ (54 mg, 0.39 mmol). The mixture is stirred for 1 h at 90° C. in a seal tube before being concentrated. The crude product is purified by combiflash (100% Hex to 100% EtOAc) to afford intermediate 149a2.

### Step 2:

Compound 149a2 (120 mg, 0.22 mmol) in AcOH (2 mL) is stirred for 2 h at 90° C. before being concentrated. The crude product is dissolved in DMSO, filtered and directly injected onto a prep. HPLC to isolate 8010.

### Example 150A

### Preparation of Compound 8011

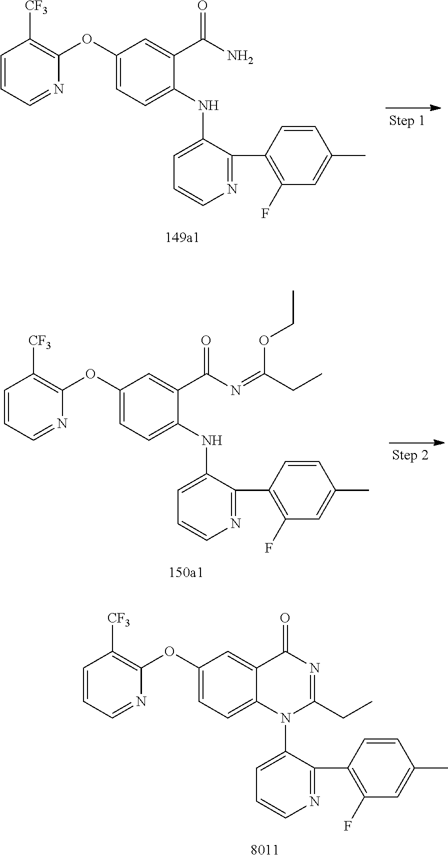

### Step 1:

Intermediate 149a1 is converted to intermediate 150a1 using triethyl orthopropionate as described in example 149A step 1.

### Step 2:

To a suspension of compound 150a1 (14 mg, 0.03 mmol) in 1,1,1,3,3,3-hexamethyldisilazane (800 μL) is added pyridine p-toluenesulfonate (6 mg, 0.03 mmol). The vial is then capped and the reaction is stirred 20 min at 215° C. in a microwave. After the mixture cools to RT, the mixture is diluted with EtOAc (15 mL), washed with an aq. sat. NaHCO₃. The organic phase is dried over MgSO₄, filtered and concentrated. The crude product is dissolved in DMSO, filtered and directly injected onto a prep. HPLC to isolate 8011.

### Example 151A

### Preparation of Compound 8012

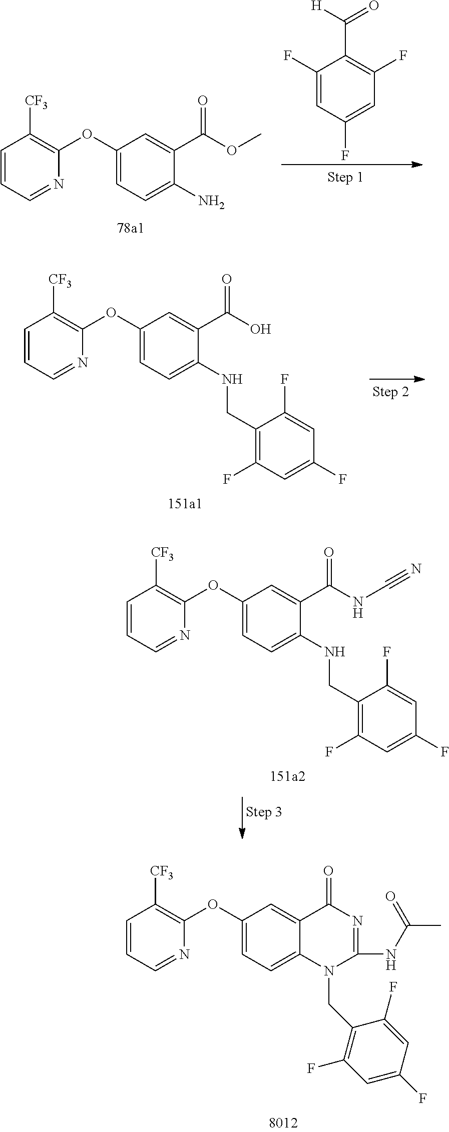

### Step 1:

The reductive amination and hydrolysis to form 151a1 are performed as described in example 12B steps 3 and 4.

### Step 2:

To a mixture of acid 151a1 (420 mg, 0.94 mmol) and cyanamide (42 mg, 9.2 mmol) in DMF (5 mL) is added NEt₃ (1 mL, 7.2 mmol) and HATU (526 mg, 1.4 mmol). The mixture is stirred for 1 h at RT and then diluted with EtOAc. The organic solution is washed with aq. sat. NH₄Cl and brine. The organic phase is dried over Na₂SO₄, filtered and concentrated. The crude product 151a2 is used as such without purification.

### Step 3:

Compound 151a2 (170 mg, 0.37 mmol) in AcOH (4 mL) is stirred for 2 h at 90° C. before being concentrated. The crude product is dissolved in DMSO, filtered and directly injected onto a prep. HPLC to isolate 8012.

### Example 152A

### Preparation of Compound 1031

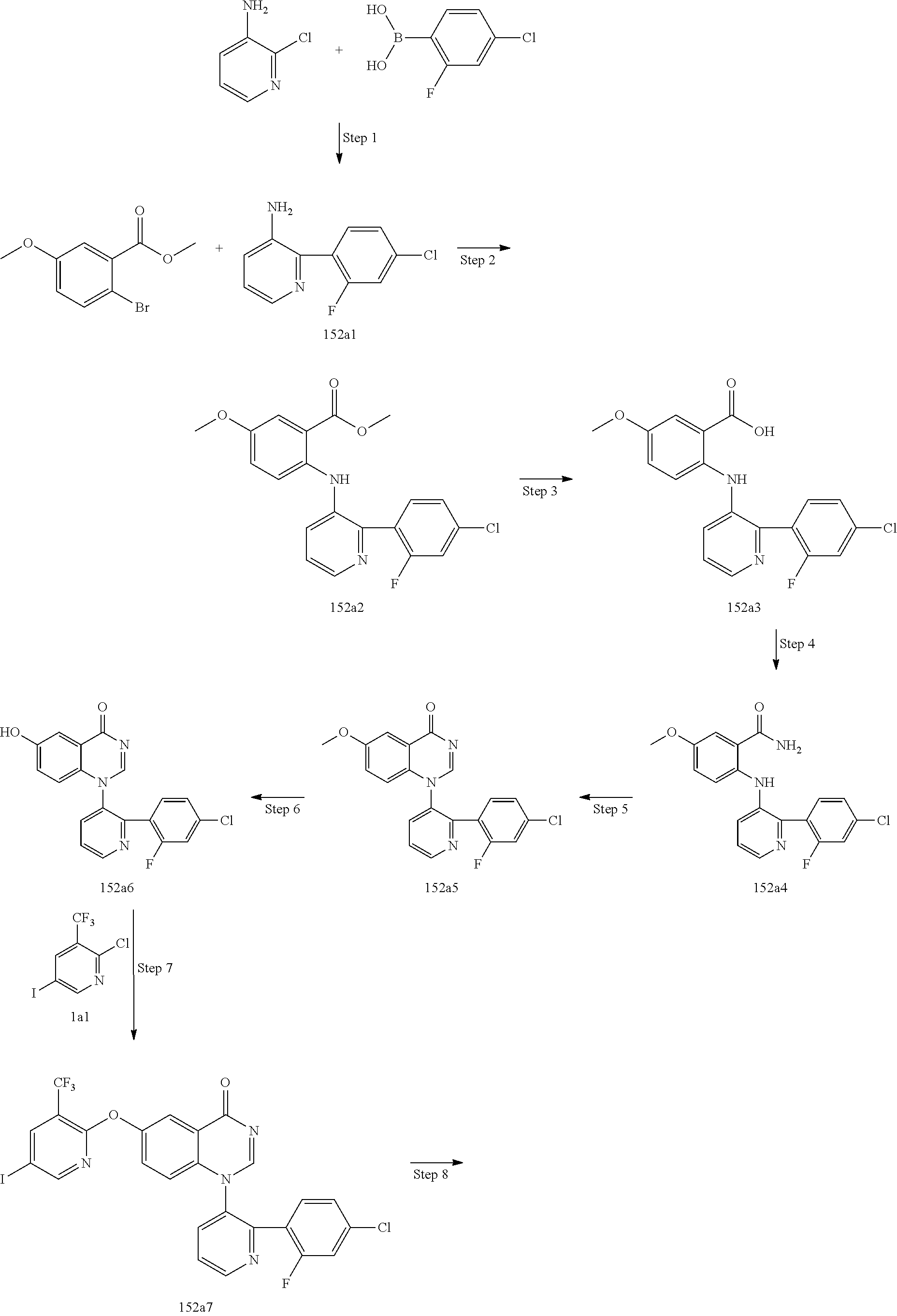

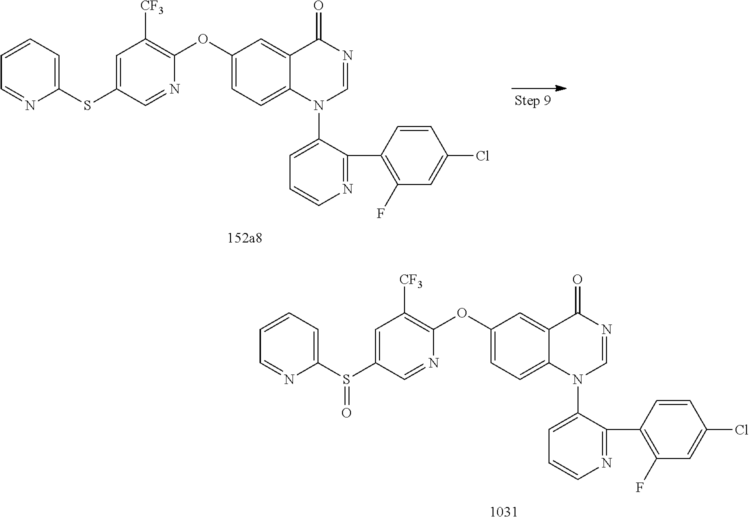

### Step 1:

Argon is bubbled through a mixture of 2-chloropyridin-3-amine (15 g, 120 mmol) and 4-chloro-2-fluorophenylboronic acid (20 g, 115 mmol) in 1,4-dioxan (400 mL) and water (100 mL). K₂CO₃ (32 g, 230 mmol) is added followed by (Ph₃P)₄Pd (6.7 g, 5.8 mmol) and this reaction mixture is stirred at 100° C. for 4 h at RT, overnight. The reaction mixture is diluted with water and the product is extracted with EtOAc (2×). The combined organic layers are dried over Na₂SO₄, filtered and concentrated. The crude product is purified by chromatography on silica (20 to 25% EtOAc in Hex) to afford intermediate 152a1.

### Step 2:

Reference: Sanz, R.; Fernández, Y.; Castroviejo, M. P.; Pérez, A.; Fañanás, F. J. J. Org. Chem. 2006, 71, 6291-6294, herein incorporated by reference.

A suspension of methyl 2-bromo-5-methoxybenzoate (24 g, 99 mmol), compound 152a1 (22 g, 99 mmol), BINAP (6.2 g, 9.9 mmol), Pd₂(dba)₃ (9.1 g, 9.9 mmol) and K₂CO₃ (68 g, 490 mmol) in degassed toluene (400 mL) is heated to 100° C. for 16 h. The reaction mixture is concentrated and the crude product is purified by chromatography on silica (15 to 35% EtOAc in Hex) to afford intermediate 152a2.

### Step 3:

To compound 152a2 (25 g, 65 mmol) in THF (320 mL) is added 2 N aq. NaOH (320 mL, 6500 mmol) and the solution is stirred for 16 h at 50° C. Diethyl ether is added and the organic phase is extracted with water (3×). The aqueous layers are combined and acidified to pH ˜5-6 with a 1M aqueous HCl solution. The formed solid is collected by filtration and dried under vacuum and over KOH to afford compound 152a3.

### Step 4:

To compound 152a3 (19.9 g, 53.4 mmol) in DMF (600 ml) is added HATU (26.4 g, 69.4 mmol) and Et₃N (29.7 mL, 214 mmol). Ammonium bicarbonate (13.9 g, 176 mmol) is then added followed by another portion of Et₃N (29.7 mL, 214 mmol). The solution is stirred at RT for 2 h. The mixture is diluted with water (1.5 L) and the formed solid is filtered off, washed with water and dried on a stream of air to yield compound 152a4.

### Step 5:

To compound 152a4 (15.5 g, 41.7 mmol) in trimethyl orthoformate (330 mL) is added TFA (37.0 mL, 500 mmol). The solution is stirred for 2 h at RT. The reaction mixture is concentrated and the crude product is purified by chromatography on silica (4 to 40% THF in EtOAc) to afford intermediate 152a5.

### Step 6:

To compound 152a5 (3.1 g, 8.1 mmol) in DCM (600 mL) is added boron tribromide (6.14 mL, 65.0 mmol). The mixture is stirred at RT for 10 days and is diluted with water. The DCM is removed under vacuum and the remaining water layer is neutralized to pH 5.5. The formed solid is collected by filtration and dried under vacuum. The product is recrystallized from MeCN/DCM to afford compound 152a6.

### Step 7:

To compound 152a6 (100 mg, 0.27 mmol) in DMF (2 mL) are added reagent 1a1 (100 mg, 0.33 mmol) and Cs₂CO₃ (115 mg, 0.35 mmol). The reaction mixture is heated at 70° C. for 16 h. The reaction mixture is then cooled to ambient temperature, and water is added. The product is extracted with EtOAc (3×). The combined organic phases are washed with brine, dried over MgSO₄, filtered and concentrated. The crude product 152a7 is used as such without purification.

### Step 8:

Reference: Carril, M.; SanMartin, R.; Dominguez, E.; Tellitu I. Chem. Eur. J. 2007, 13, 5100, herein incorporated by reference.

To crude compound 152a7 (170 mg, 0.27 mmol) in DMF (2 mL) are added 2-mercaptopyridine (33 mg, 0.30 mmol), CuI (26 mg, 0.14 mmol) and trans-1,2-diaminocyclohexane (10 μL, 0.08 mmol). The reaction mixture is heated at 150° C. for 1 h. The reaction mixture is then cooled to ambient temperature, and water is added. The product is extracted with EtOAc (3×). The combined organic phases are washed with brine, dried over MgSO₄, filtered and concentrated. The crude product 152a8 is used as such without further purification.

### Step 9:

To crude compound 152a8 (52 mg, 0.08 mmol) in acetone (0.8 mL) and water (0.3 mL) is added Oxone™ (56 mg, 0.09 mmol). The reaction mixture is stirred at RT for 16 h. The reaction mixture is then filtered and concentrated. The crude product is dissolved in DMSO, filtered and directly injected onto a prep. HPLC to isolate 1031.

### Example 153A

### Preparation of Compound 4029

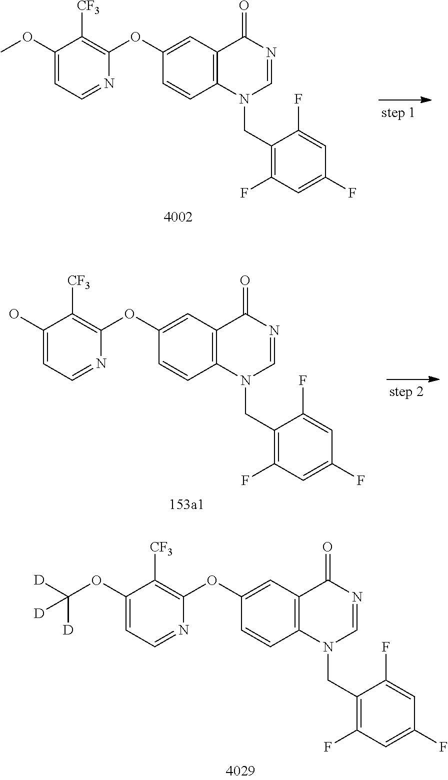

### Step 1:

Compound 4002 is converted to quinazolinone 153a1, in an analogous manner to synthetic method C.

### Step 2:

Compound 4029 is prepared from 153a1 in an analogous manner using commercially-available D₃COD as a reagent using synthetic method U.

### Example 154A

### Preparation of Compound 154a9

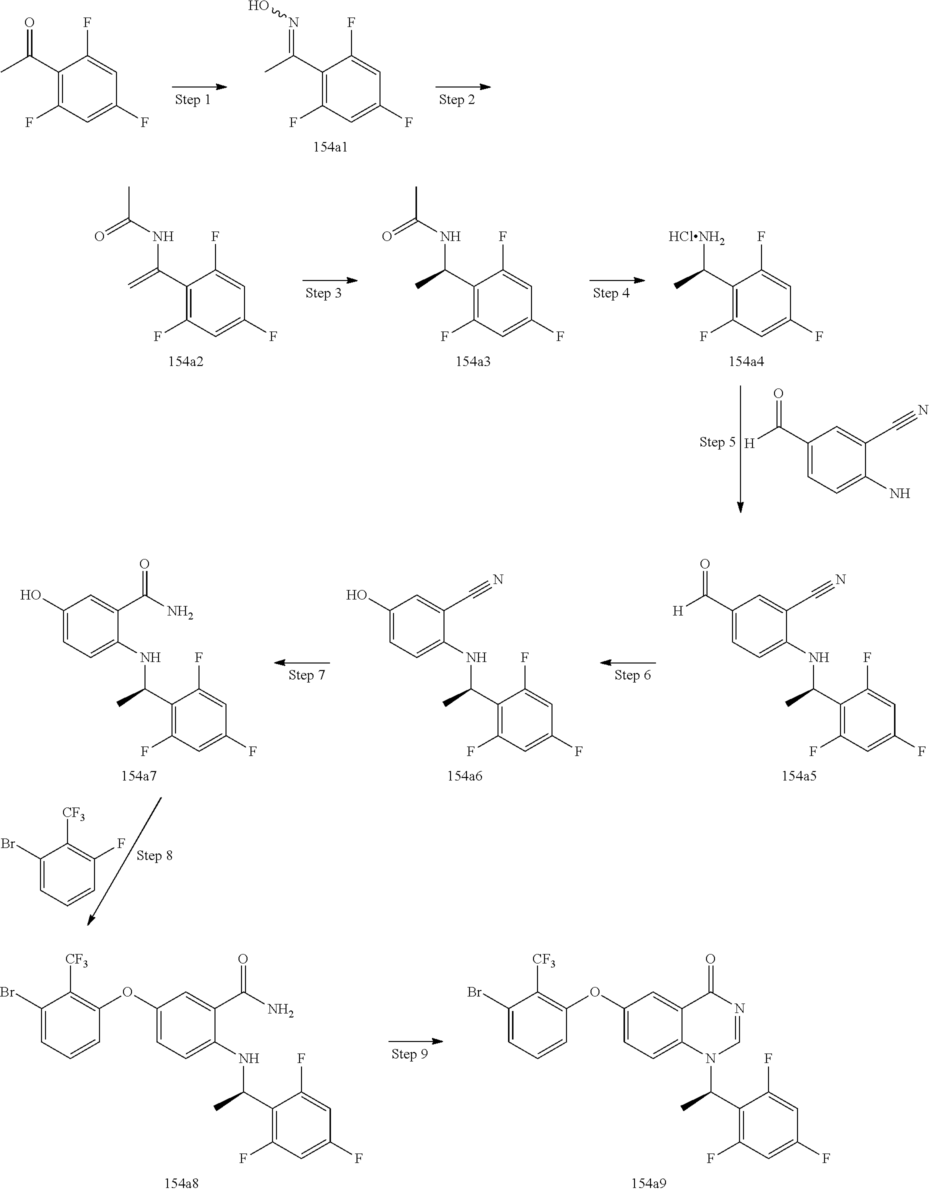

### Step 1:

2,4,6-Trifluoroacetophenone (100 g, 574 mmol) is dissolved in EtOH (1 L), followed by the addition of NaHCO₃ (135 g, 1.61 mol) and hydroxylamine hydrochloride (127 g, 1.49 mol). The reaction mixture is stirred at 90° C. for 16 h. The resulting mixture is cooled to RT, diluted in EtOAc (1.5 L) and washed with water (1 L). The organic phase is dried over anhydrous Na₂SO₄ and concentrated to dry to afford 154a1 which is used as is in the next step without further purification.

### Step 2:

In a 3-neck round bottom flask equipped with mechanical stirrer and condenser, 154a1 (105 g, 556 mmol) is dissolved in THF (1 L), glacial AcOH (60 mL, 1.11 mol) and Ac₂O (103 mL, 1.11 mol). The resulting mixture is purged with argon for 20 min before anhydrous Fe(OAc)₂ (193 g, 1.11 mol) is added. The reaction mixture is stirred overnight at 65° C. under argon then filtered. The filtrate is neutralized to approximately pH 8 using NaHCO₃, diluted by EtOAc (2 L) and washed with water (1 L). The filter cake is washed with THF (3 L) and the filtrate is combined with the EtOAc phase from the extraction. The combined organic phase is concentrated to dryness and the residue is washed through a pad of silica with EtOAc to afford enamide 154a2.

### Step 3:

Enamide 154a2 (102 g, 474 mmol) is suspended in HPLC grade MeOH (2 L) and the mixture is purged with argon for 20 min before RR-Me-BPE-Rh ((+)-1,2-Bis((2R,5R)-2,5-dimethylphospholano)ethane(1,5-cyclooctadiene) rhodium(I) tetrafluoroborate) (500 mg, 0.90 mmol, 0.2%) is added. The resulting mixture is stirred under 100 psi of hydrogen for 6 h and then filtered and concentrated to dryness to afford N-acyl amine 154a3 which requires no further purification.

### Step 4:

N-Acyl amine 154a3 (103 g, 474 mmol) is suspended in 4 M HCl (1.2 L) and the reaction mixture is heated at reflux for 16 h. The resulting mixture is concentrated in vacuo and the residue is re-dissolved in MeOH (20 mL) and precipitated with Et₂O to afford amine 154a4 as the hydrochloride salt.

### Step 5:

A mixture of 2-fluoro-5-formylbenzonitrile (41.1 g, 276 mmol), 154a4 (70 g, 331 mmol) and NEt₃ (96.1 mL, 689 mmol) in DMSO (410 mL)/water (50 mL) is stirred at 75° C. for 5 days. The cooled reaction mixture is diluted with EtOAc (2 L), washed with brine, dried over Na₂SO₄, filtered and concentrated under vacuum to give aldehyde 154a5 which is used without further purification in the next step.

### Step 6:

To an ice-cooled mixture of aldehyde 154a5 (170 g, 276 mmol) in MeOH (1.3 mL) is successively added 30% aqueous H₂O₂ (114 mL) and concentrated H₂SO₄ (46.4 mL). The reaction mixture is stirred at 0° C. for 7 h and then placed in a refrigerator for 14 h. The reaction mixture is diluted with water (1.2 L), filtered and the filtrate is treated with ice water. The resulting solid is dried under high vacuum to afford phenol 154a6 which is used as such in the next synthetic step.

### Step 7:

Concentrated H₂SO₄ (450 mL) is added carefully to nitrile 154a6 (69.7 g, 239 mmol). The reaction mixture is stirred at RT for 3 days and then poured over ice (—4 L). The pH is adjusted to approximately 2 using aqueous 10 N NaOH. The resulting solid is collected by filtration. The pH of the filtrate is then adjusted to approximately 3. The resulting solid is collected by filtration. The pH of the filtrate is then adjusted to approximately 4 and the resulting solid is collected by filtration. The filtrate is extracted with EtOAc (3×), and the combined organic extracts are washed with brine and dried over anhydrous Na₂SO₄. The solution is filtered and concentrated in vacuo. The combined solids are partitioned between a mixture of water (200 mL) and EtOAc (2 L). Saturated aqueous NaHCO₃ solution is added to the suspension until homogeneity. The organic layer is separated and washed with brine and then dried over anhydrous Na₂SO₄. The solution is filtered and concentrated in vacuo, and the resulting residue is treated with EtOAc and hexane until a solid is formed. Carboxamide 154a7 is recovered by filtration.

### Step 8:

To a mixture of carboxamide 154a7 (29.0 g; 93.4 mmol) in DMF (210 mL) is added Cs₂CO₃ (76.1 g; 233.6 mmol). 3-fluoro-2-(trifluoromethyl)bromobenzene (22.7 g; 93.4 mmol) is then added and the reaction mixture is stirred at RT for 60 h. The reaction mixture is diluted with EtOAc and water, washed with brine (3×) and dried over anhydrous Na₂SO₄. After filtering, the solvent is removed in vacuo. The residue is triturated with EtOAc and hexanes to afford diarylether 154a8.

### Step 9:

Diarylether 154a8 is converted to quinazolinone 154a9 using a protocol analogous to that described in example 1 step 3. Quinazolinone 154a9 is purified by flash chromatography (5 to 30% MeOH in CH₂Cl₂).

### Example 154B

### Preparation of Compound 154b1

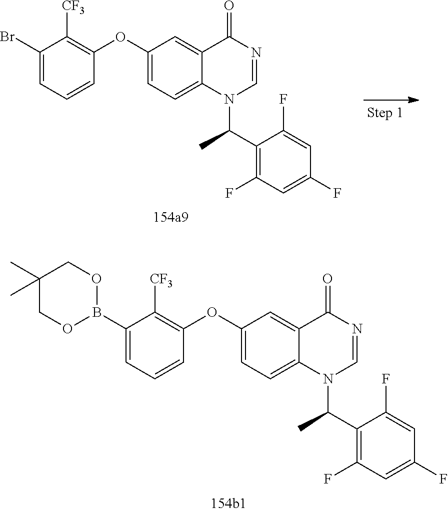

### Step 1:

Arylbromide 154a9 (5.00 g, 9.20 mmol) is combined with bis(neopentylglycolato) diboron (3.12 g, 13.8 mmol) and KOAc (3.06 g, 32.2 mmol) in DMF (35 mL). Argon is bubbled through the reaction mixture for 45 min before (dppf)PdCl₂ (0.67 g, 0.92 mmol) is added. Argon is then again bubbled through the reaction mixture for 25 min. The reaction mixture is warmed to 95° C. and stirred for 8 h. The reaction mixture is then diluted in EtOAc, washed with water and filtered through celite. The organic phase is separated, and then washed with water (2×) and brine (2×). The organic phase is dried over MgSO₄, filtered and concentrated. The residue is subjected to flash chromatography (0 to 10% MeOH in EtOAc) to afford boronic ester 154b1.

### Example 154C

### Preparation of Intermediate 154c1

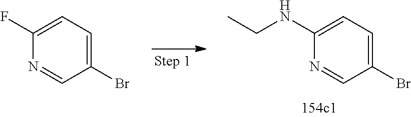

### Step 1:

To a mixture of 2-fluoro-5-bromopyridine (18.0 g, 176 mmol) in DMSO (180 mL) is added ethylamine hydrochloride (16.6 g, 205 mmol) and DIPEA (53.5 mL, 307 mmol). The mixture is heated to 100° C. and is stirred overnight. An additional equivalent of ethylamine hydrochloride and DIPEA are added and stirring at 100° C. is continued for 16 h. The mixture is diluted in DCM and the organic phase is washed with water and brine. The organic phase is dried over anhydrous Na₂SO₄, filtered and concentrated. The residue is triturated in Et₂O and hexanes to afford ethylaminopyridine 154c1.

### Example 154D

### (Synthetic Method BQ)

### Preparation of Compound 9001

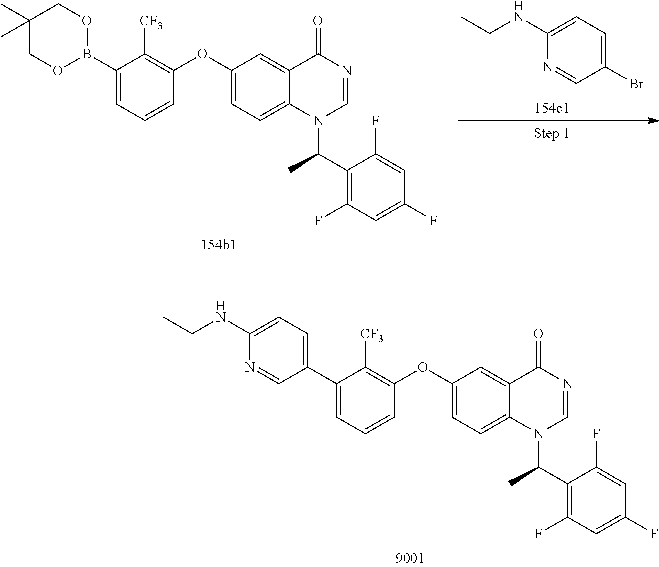

### Step 1:

Boronic ester 154b1 (3.00 g, 5.21 mmol) is combined with ethylaminopyridine 154c1 (1.57 g, 7.8 mmol) and Na₂CO₃ (1.66 g, 15.6 mmol) in 2-methyl-THF (60 mL) and water (6 mL). The mixture is sonicated under a flow of argon for 15 min before (Cy₃P)₂Pd (0.35 g, 0.52 mmol) is added. The mixture is warmed to 80° C. and stirred for 4 h. The mixture is diluted in EtOAc and washed with brine (2×). The organic phase is dried over anhydrous Na₂SO₄, filtered and concentrated. The residue is subjected to flash chromatography (0 to 8% MeOH in EtOAc) to afford compound 9001.

### Example 155A

### Preparation of Compound 155a1

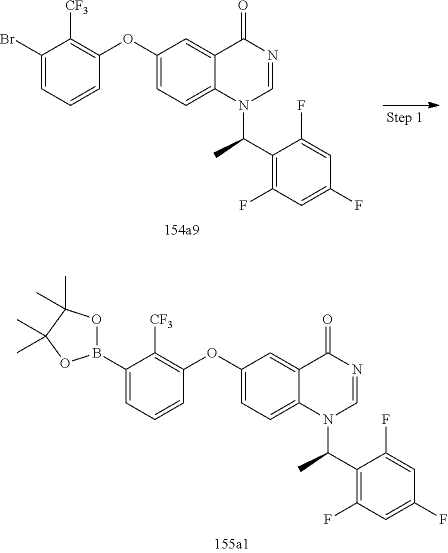

### Step 1:

Arylbromide 154a9 is coupled with bis(pinacolato)diboron analogously to the procedure described in example 154b step 1 to produce boronic ester 155a1.

### Example 155D

### (Synthetic Method BR)

### Preparation of Compound 9006

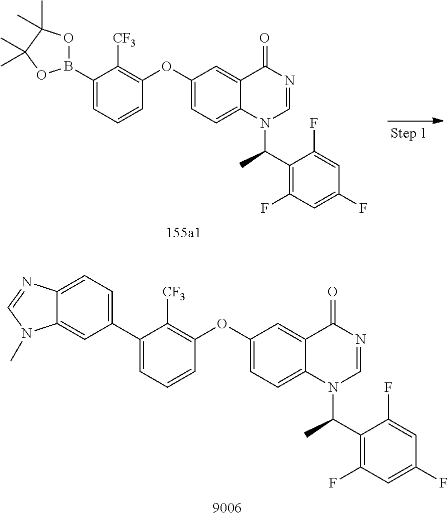

### Step 1:

Boronic ester 155a1 (60 mg, 0.10 mmol) is combined with 6-bromo-1-methyl-1H-benzo[D]imidazole (43 mg, 0.20 mmol) and 2.0 M aqueous Na₂CO₃ (150 μL, 0.30 mmol) in DMF (1 mL). The mixture is sonicated under a flow of argon for 15 min before (Ph₃P)₄Pd (25 mg, 0.02 mmol) is added. The mixture is warmed to 120° C. and stirred for 10 min. The mixture is diluted in MeOH and filtered. The solution is injected onto a preparative HPLC to isolate compound 9006.

### Example 156A

### Preparation of Compound 9011

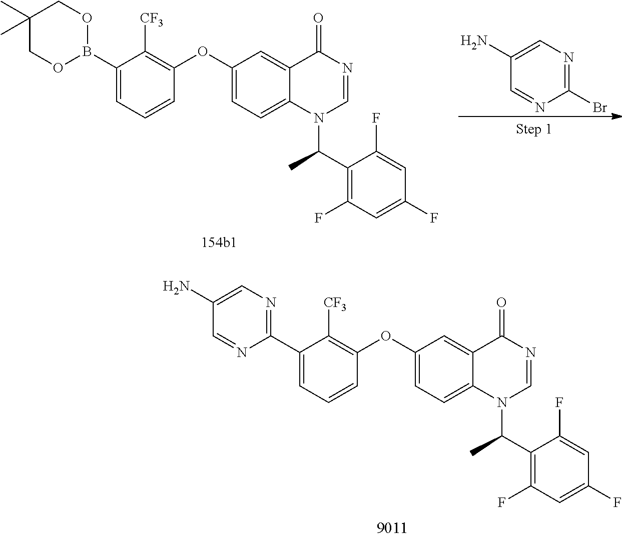

### Step 1:

Boronic ester 154b1 (1.20 g, 2.1 mmol) is combined with 5-amino-2-bromopyrimidine (0.91 g, 5.2 mmol) and Na₂CO₃ (0.66 g, 6.2 mmol) in 2-methyl-THF (24 mL) and water (2.4 mL). The reaction mixture is sonicated under a flow of argon for 15 min before (Cy₃P)₂Pd (0.069 g, 0.10 mmol) is added. The reaction mixture is warmed to 80° C. and stirred for 12 h. The reaction mixture is diluted in EtOAc and washed with brine (2×). The organic phase is dried over anhydrous Na₂SO₄, filtered and concentrated. The residue is subjected to automated flash chromatography (0 to 8% MeOH in EtOAc). The fractions containing the partially purified product are concentrated and the residue is purified again by flash chromatography (Combiflash, 0 to 8% MeOH in DCM) to isolate compound 9011.

### Example 157A

### Preparation of Compound 9025

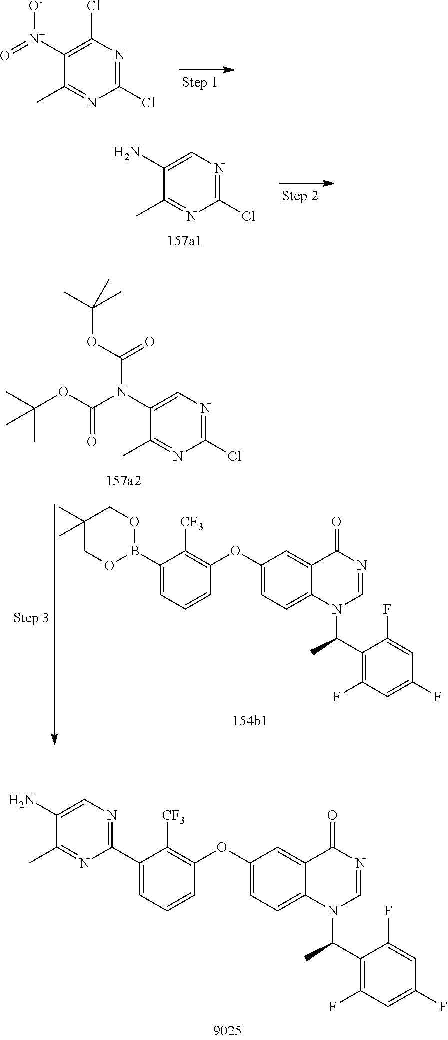

### Step 1:

A mixture of 2,4-dichloro-6-methyl-5-nitropyrimidine (15 g, 72 mmol), NaHCO₃ (7.5 g, 89 mmol) and 10% Pd/C (1.7 g) in EtOH (450 mL) in a 1 L hydrogenation bomb is degassed with argon for 15 min. The reactor is charged with H₂ (15 psi) and the reaction mixture is stirred at RT. Hydrogen is constantly added to the reaction mixture until the pressure remains at 15 psi. After stirring for 2 h, the reaction mixture is degassed with argon and filtered through a bed of celite, washing with EtOH. The combined washes are concentrated in vacuo and the residue is taken up in EtOH (500 mL). The reaction mixture is filtered and the filtrate is preabsorbed on silica gel. Purification by flash chromatography (Combiflash, 10% to 50% EtOAc in hexanes) affords 5-amino-2-chloro-4-methylpyrimidine 157a1.

### Step 2:

Boc₂O (37 g, 70 mmol) is added to a mixture of aminopyrimidine 157a1 (7.8 g, 54 mmol), DMAP (1 g, 8.2 mmol) and Et₃N (39 mL, 280 mmol). The reaction mixture is stirred at RT for 18 h and then the solvent is removed in vacuo. The residue is preabsorbed on silica gel (100 g). Purification by flash chromatography (Combiflash, eluting with 0% to 10% EtOAc in hexanes) affords bis-Boc protected aminopyrimidine 157a2.

### Step 3:

Boronic ester 154b1 (1.20 g, 2.1 mmol) is combined with 5-(bis-N-Boc-amino)-2-chloropyrimidine 157a2 (1.07 g, 3.1 mmol) and Na₂CO₃ (0.66 g, 6.2 mmol) in 2-methyl-THF (12 mL) and water (1.2 mL). The reaction mixture is sonicated under a flow of argon for 15 min before (Cy₃P)₂Pd (0.14 g, 0.21 mmol) is added. The reaction mixture is then warmed to 140° C. and is stirred for 1 h in a microwave. The reaction mixture is diluted in EtOAc and washed with brine (2×). The organic phase is dried over anhydrous Na₂SO₄, filtered and concentrated. The residue is subjected to flash chromatography (Combiflash, 60 to 100% acetone in hexanes) twice to afford compound 9025.

### Example 158A

### Inhibition of NS5B RNA Dependent RNA Polymerase Activity

Representative compounds of the invention are tested for inhibitory activity against the hepatitis C virus RNA dependent polymerase (NS5B), using the assay described in WO2009/018657, herein incorporated by reference.

### Example 159A

### Cell-Based Luciferase Reporter HCV RNA Replication Assay Cell Culture

Huh-7 cells with a stable subgenomic HCV replicon that encodes a modified luciferase reporter gene (expressed as a luciferase-FMDV2A-neomycin phosphotransferase gene fusion) were established as previously described (Lohman et al., 1999. Science 285: 110-113; Vroljik et al., 2003 J. Virol Methods 110:201-209), with the exception that replicon cells were selected with 0.25 mg/mL G418. The amount of luciferase expressed by selected cells directly correlates with the level of HCV replication. These cells, designated as MP-1 cells, are maintained in Dulbecco's Modified Earle Medium (DMEM) supplemented with 10% FBS and 0.25 mg/mL neomycin (standard medium). The cells are passaged by trypsinization and frozen in 90% FBS/10% DMSO. During the assay, DMEM medium supplemented with 10% FBS, containing 0.5% DMSO and lacking G418, was used (Assay Medium). The day of the assay, MP-1 cells are trypsinized and diluted to obtain 15 000 cells/70 μL in Assay Medium. 70 μL are distributed into each well of a black 96-well ViewPlate™ (Packard). The plate is then incubated at 37° C. until compound addition.

### Reagents and Materials:

### Cell Culture

| Product | Company | Catalog # | Storage |
|---|---|---|---|
| DMEM | Wisent Inc. | 10013CV | 4° C. |
| DMSO | Sigma | D-2650 | RT |
| Dulbecco's PBS | Gibco-BRL | 14190-136 | RT |
| Fetal Bovine Serum | HyClone | SV30014 | −20° C./4° C. |
| Geneticin (G418) | Gibco-BRL | 10131-027 | −20° C./4° C. |
| Trypsin-EDTA | Gibco-BRL | 25300-054 | −20° C./4° C. |
| ViewPlateTM-96, Black | Packard | 6005182 | RT |
| Backing tape, Black | Packard | 6005189 | RT |
| PVDF 0.22 μm Filter | Millipore | MAGVS2210 | RT |
| Unit | | | |
| Deep-Well Titer Plate | Beckman | 267007 | RT |
| Polypropylene | | | |

### Luciferase Assay

| Product | Company | Catalog # | Storage |
|---|---|---|---|
| Glo Lysis Buffer | Promega | E266A | 4° C. |
| Bright-Glo Luciferase Assay | Promega | E2620 | −20° C. |
| System | | | |

### Preparation of Test Compound:

The test compound in 100% DMSO is first diluted in Assay Medium, lacking G418 to a final DMSO concentration of 0.5%. The solution is sonicated for 15 min. Into column 3 of a Polypropylene Deep-Well Titer Plate, the appropriate volume is transferred into Assay Medium to obtain the starting concentration (2×) to be tested. In columns 4 to 11 add 400 μL of Assay Medium (containing 0.5% DMSO). Serial dilutions (⅓) are prepared by transferring 200 μL from column 3 to column 4, then from column 4 to column 5, serially through to column 11 (no compound is included in column 12).

### Addition of Test Compound to Cells:

A volume of 70 μL from each well of the compound dilution plate is transferred to a corresponding well of the Cell Plate. (Three columns will be used as the “No inhibition control”; nine [9] columns are used for the dose response). The cell culture plate is incubated at 37° C., 5% CO₂ for 28 hours.

### Luciferase Assay:

Following the incubation period, the medium is aspirated from the 96-well assay plate and a volume of 50 μL of 1× Glo Lysis Buffer (Promega) previously warmed to room temperature was added to each well. The plate was incubated at room temperature for 10 min with occasional shaking. A black tape was put at the bottom of the plate. 50 μL of Bright-Glo luciferase substrate (Promega) previously warmed to room temperature was added to each well followed by gentle mixing. The luminescence was determined on a Packard Topcount instrument using the Data Mode Luminescence (CPS) with a count delay of 1 min and a count time of 2 sec.

The luminescence determination (CPS) in each well of the culture plate was a measure of the amount of HCV RNA replication in the presence of various concentrations of inhibitor. The % inhibition was calculated with the following equation:

inhibition=100−[CPS(inhibitor)/CPS(control)×100]

A non-linear curve fit with the Hill model was applied to the inhibition-concentration data, and the 50% effective concentration (EC₅₀) was calculated by the use of SAS software (Statistical Software; SAS Institute, Inc. Cary, N.C.).

### Tables of Compounds

The following tables list compounds representative of the invention. All of the compounds listed in Tables 1 to 9 below are tested in the assay of Example 158A or the assay of Example 159A or both. Each compound has an IC₅₀ or EC₅₀ value of below 40 μM, or both an IC₅₀ and EC₅₀ value of below 40 μM. Representative IC₅₀ and EC₅₀ data for selected compounds of the invention is provided in Table 10.

Retention times (t_{R}) for each compound are measured using the standard analytical HPLC conditions described in the Examples. Retention times for each compound measured using the analytical HPLC conditions described in the Examples are identified by an asterix (*) in the tables. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions. The synthetic method used to generate each compound in Tables 1 to 9 is identified in the table. A person skilled in the art will recognize that obvious modifications to the synthetic methods, including the amount of time indicated to perform the various steps, may be required to generate each of the specific compounds listed in Tables 1 to 9.

TABLE 1

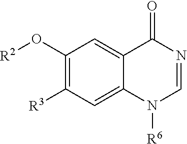

| | | | | | | Example/ |
|---|---|---|---|---|---|---|
| | | | | t_{R} | MS | Synthetic |
| Cpd | R² | R³ | R⁶ | (min) | (M + H)⁺ | Method |
| 1001 | 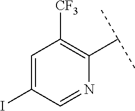 | H | 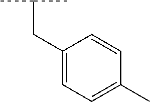 | 5.35 | 538.1 | Ex. 1A |
| 1002 | 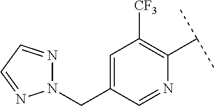 | H | 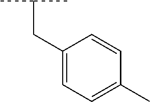 | 4.73 | 493.2 | Ex. 2A |
| 1003 | 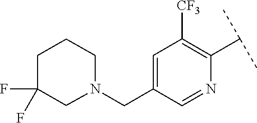 | H | 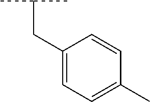 | 4.05 | 545.2 | Ex. 3A |
| 1004 | 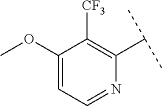 | H | 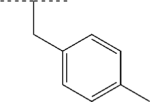 | 4.77 | 442.2 | Ex. 4B Met. A |
| 1005 | 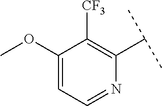 | H | 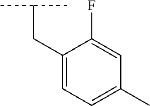 | 5.76 | 460.1 | Met. A |
| 1006 | 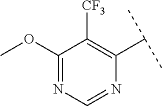 | H | 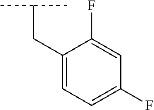 | 5.88 | 465.1 | Ex. 5C Met. B |
| 1007 | 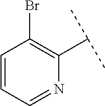 | H | 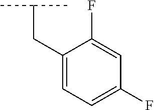 | 5.28 | 444.0/ 446.0 | Met. B |
| 1008 | 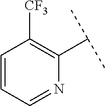 | F | 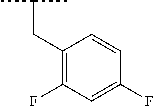 | 5.62 | 452.0 | Ex. 6A |
| 1009 | 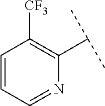 | —OCH₃ | 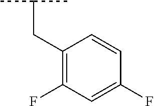 | 5.52 | 464.1 | Ex. 7A |
| 1010 | 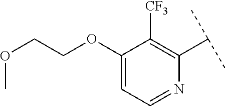 | H | 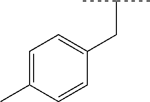 | 5.16 | 486.2 | Ex. 9A Met. D |
| 1011 | 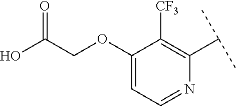 | H | 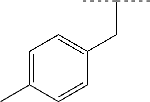 | 4.63 | 486.2 | Ex. 10A Met. E |
| 1012 | 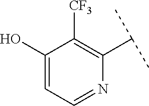 | H | 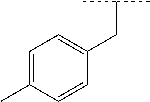 | 4.61 | 428.2 | Ex. 8A Met. C |
| 1013 | 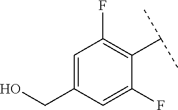 | H | 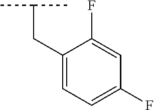 | 4.83 | 431.2 | Ex. 11A |
| 1014 | 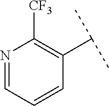 | H | 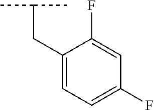 | 5.20 | 434.1 | Ex. 12B Met. F |
| 1015 | 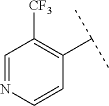 | H | 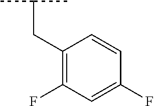 | 4.37 | 434.1 | Ex. 13A Met. G |
| 1016 | 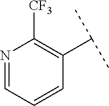 | H | 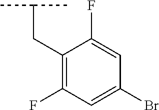 | 5.76 | 512.1 | Ex. 14A |
| 1017 | 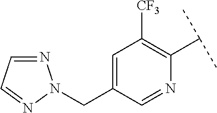 | H | 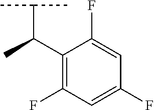 | 4.95 | 547.1 | Ex. 15C |
| 1018 | 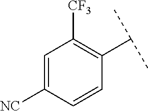 | H | 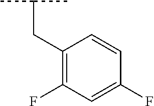 | 4.49 | 458.1 | Met. B |
| 1019 | 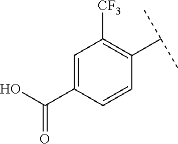 | H | 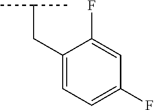 | 5.34 | 477.1 | Ex. 16A Met. H |
| 1020 | 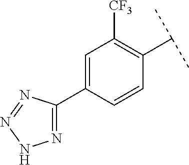 | H | 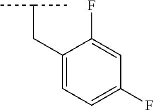 | 5.30 | 501.0 | Ex. 17A |
| 1021 | 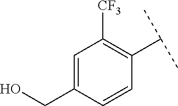 | H | 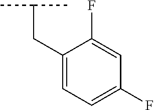 | 5.13 | 463.2 | Ex. 18A |
| 1022 | 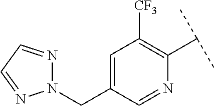 | H | 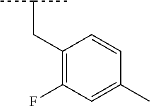 | 5.37 | 511.0 | Ex. 19A Met. I |
| 1023 | 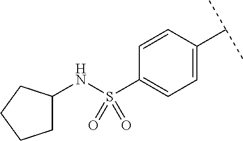 | H | 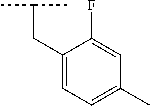 | 2.49 | 508.1 | Ex. 20A Met. J |
| 1024 | 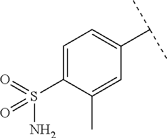 | H | 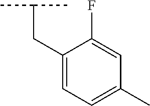 | 1.91 | 454.1 | Met. J |
| 1025 | 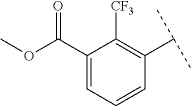 | H | 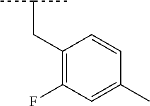 | 2.47 | 487.1 | Ex. 21A Met. K |
| 1026 | 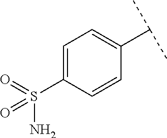 | H | 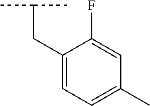 | 1.81 | 440.1 | Met. J |
| 1027 | 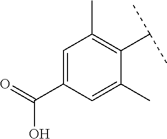 | H | 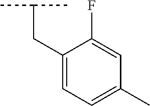 | 5.26 | 433.0 | Ex. 22A Met. L |
| 1028 | 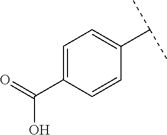 | H | 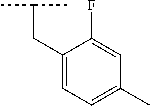 | 4.84 | 405.0 | Met. L |
| 1029 | 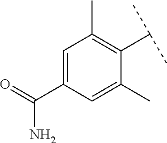 | H | 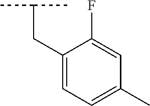 | 4.71 | 432.0 | Ex. 23A Met. M |
| 1030 | 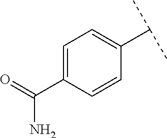 | H | 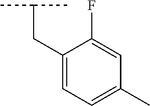 | 4.24 | 404.0 | Met. M |
| 1031 | 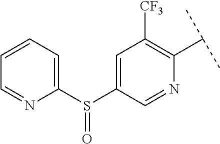 | H | 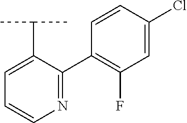 | 2.29 | 638.1 | Ex. 152A |

TABLE 2

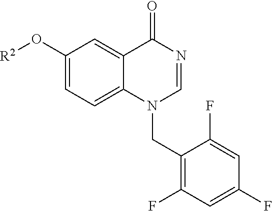

| | | | | Example/ |
|---|---|---|---|---|
| | | | MS | Synthetic |
| Cpd | R² | t_{R} (min) | (M + H)⁺ | Method |
| 2001 | 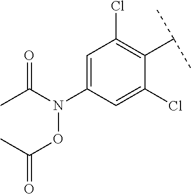 | 5.84 | 568.1 | Ex. 24B |
| 2002 | 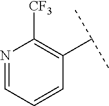 | 5.20 | 452.1 | Met. F |
| 2003 | 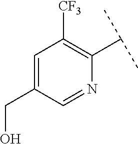 | 4.38 | 482.1 | Ex. 25A |
| 2004 | 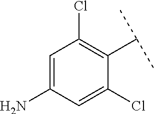 | 5.29 | 468.1 | Ex. 26A Met. N |
| 2005 | 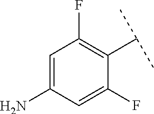 | 4.83 | 434.1 | Met. N |
| 2006 | 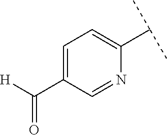 | 4.31 | 412.1 | Ex. 27A Met. O |
| 2007 | 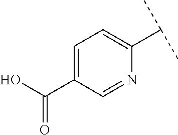 | 4.06 | 428.1 | Ex. 28A |
| 2008 | 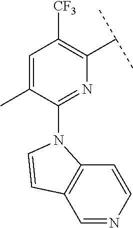 | 4.62 | 582.2 | Ex. 29B Met. P |
| 2009 | 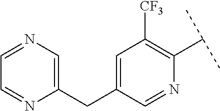 | 4.90 | 544.1 | Ex. 30A Met. Q |
| 2010 | 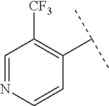 | 4.38 | 452.1 | Met. O |
| 2011 | 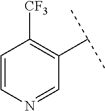 | 4.87 | 452.3 | Ex. 31A |
| 2012 | 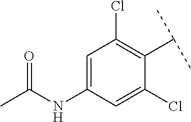 | 5.34 | 508.2 | Ex. 32A Met. R |
| 2013 | 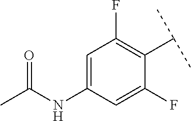 | 4.95 | 476.3 | Met. R |
| 2014 | 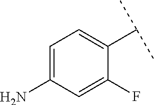 | 3.73 | 416.1 | Met. N |
| 2015 | 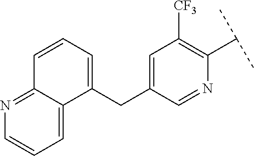 | 4.58 | 593.2 | Ex. 33A Met. S |
| 2016 | 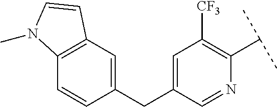 | 6.51 | 595.2 | Met. S |
| 2017 | 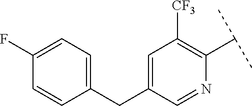 | 6.31 | 560.2 | Met. S |
| 2018 | 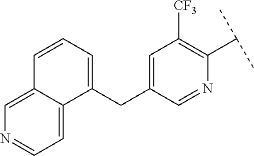 | 4.61 | 593.2 | Met. S |
| 2019 | 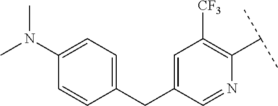 | 4.64 | 585.3 | Met. S |
| 2020 | 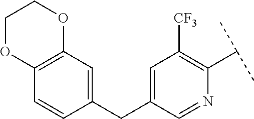 | 6.14 | 600.2 | Met. S |
| 2021 | 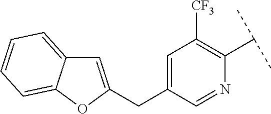 | 6.56 | 582.2 | Met. S |
| 2022 | 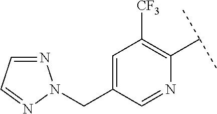 | 4.89 | 533.1 | Ex. 34A |
| 2023 | 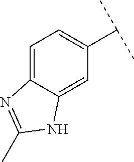 | 3.38 | 437.2 | Ex. 35A |
| 2024 | 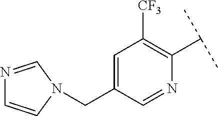 | 3.96 | 532.3 | Met. P |
| 2025 | 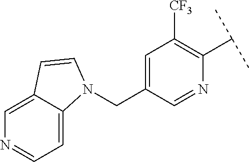 | 4.32 | 582.3 | Ex. 29B Met. P |
| 2026 | 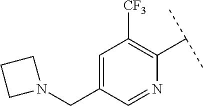 | 3.85 | 521.3 | Ex. 36A Met. T |
| 2027 | 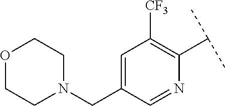 | 3.84 | 551.3 | Met. T |
| 2028 | 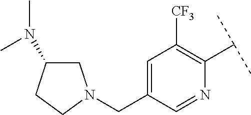 | 3.64 | 578.3 | Met. T |
| 2029 | 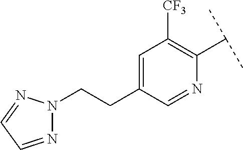 | 5.02 | 547.1 | Ex. 37A |
| 2030 | 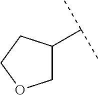 | 3.22 | 377.1 | Ex. 38A Met. U |
| 2031 | 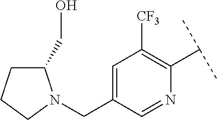 | 3.88 | 565.2 | Met. T |
| 2032 | 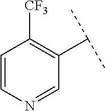 | 2.91 | 384.1 | Ex. 39A Met. V |
| 2033 | 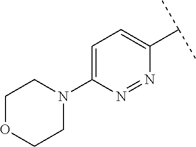 | 3.15 | 470.1 | Ex. 40A |
| 2034 | 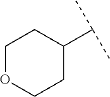 | 3.53 | 391.1 | Met. U |
| 2035 | 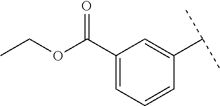 | 5.18 | 455.1 | Met. V |
| 2036 | 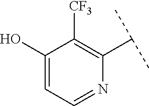 | 4.40 | 468.1 | Met. C |
| 2037 | 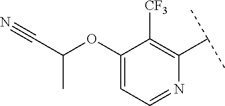 | 5.25 | 521.1 | Met. D |
| 2038 | 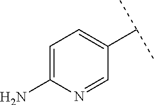 | 3.00 | 399.0 | Ex. 41A |
| 2039 | 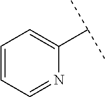 | 4.40 | 384.0 | Ex. 41A |
| 2040 | 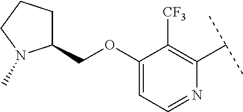 | 3.95 | 565.2 | Met. U |
| 2041 | 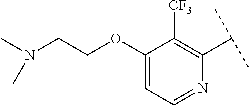 | 3.73 | 539.2 | Met. U |
| 2042 | 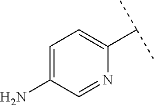 | 3.12 | 399.1 | Ex. 42A Met. W |
| 2043 | 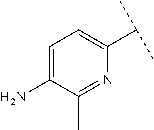 | 3.18 | 413.1 | Met. W |
| 2044 | 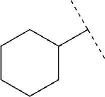 | 5.22 | 389.1 | Met. U |
| 2045 | 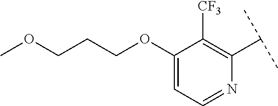 | 5.24 | 540.1 | Ex. 43A Met. X |
| 2046 | 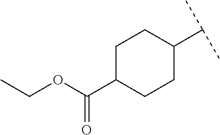 | 5.12 | 461.1 | Met. U |
| 2047 | 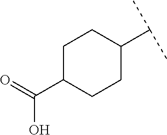 | 4.13 | 433.1 | Ex. 44A |
| 2048 | 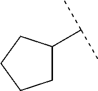 | 4.91 | 375.1 | Met. U |
| 2049 | 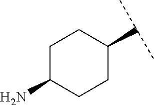 | 2.89 | 404.1 | Ex. 45A |
| 2050 | 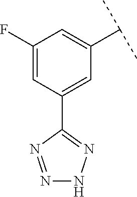 | 4.92 | 469.0 | Ex. 46A |
| 2051 | 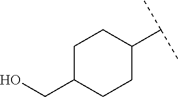 | 4.03 | 419.1 | Ex. 47A |
| 2052 | 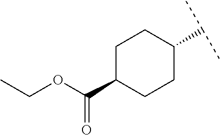 | 5.16 | 461.1 | Ex. 48A |
| 2053 | 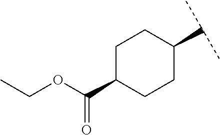 | 5.00 | 461.1 | Ex. 48A |
| 2054 | 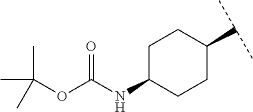 | 5.15 | 504.1 | Ex. 45A |
| 2055 | 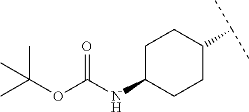 | 5.22 | 504.1 | Met. U |
| 2056 | 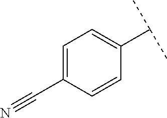 | 5.25 | 408.0 | Met. O |
| 2057 | 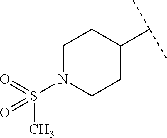 | 4.03 | 468.0 | Ex. 49A Met. Y |
| 2058 | 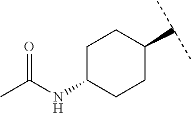 | 3.74 | 446.1 | Met. R |
| 2059 | 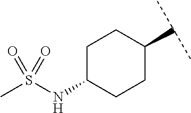 | 4.08 | 482.0 | Met. Y |
| 2060 | 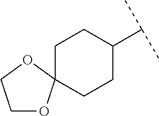 | 4.39 | 447.1 | Ex. 50A |
| 2061 | 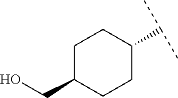 | 4.00 | 419.1 | Ex. 51A |
| 2062 | 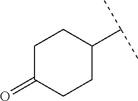 | 3.92 | 403.0 | Ex. 52A |
| 2063 | 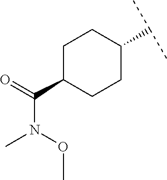 | 4.40 | 476.1 | Ex. 53A |
| 2064 | 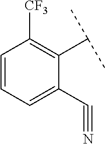 | 5.73 | 476.0 | Met. G |
| 2065 | 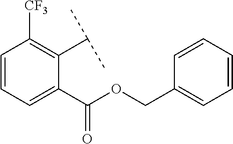 | 6.63 | 585.0 | Ex. 54A |
| 2066 | 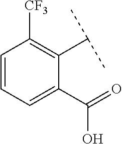 | 5.14 | 495.0 | Ex. 55A |
| 2067 | 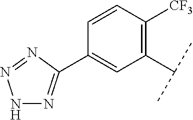 | 5.52 | 519.0 | Ex. 56A Met. Z |
| 2068 | 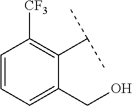 | 5.17 | 481.0 | Ex. 57A |
| 2069 | 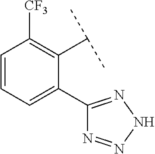 | 4.98 | 519.1 | Met. Z |
| 2070 | 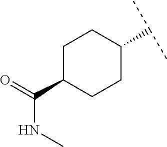 | 3.75 | 446.1 | Ex. 58A Met. AA |
| 2071 | 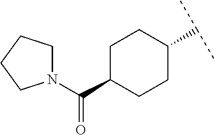 | 4.46 | 486.2 | Met. AA |
| 2072 | 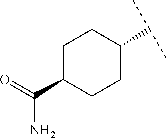 | 3.59 | 432.1 | Ex. 59A |
| 2073 | 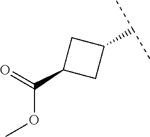 | 4.39 | 419.0 | Met. U |
| 2074 | 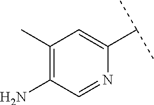 | 3.38 | 413.1 | Met. W |
| 2075 | 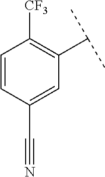 | 6.01 | 476.0 | Met. G |
| 2076 | 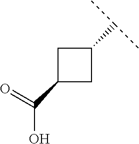 | 3.82 | 405.0 | Ex. 60A |
| 2077 | 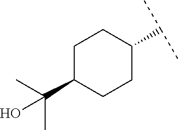 | 4.45 | 447.1 | Ex. 61A |
| 2078 | 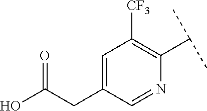 | 5.50 | 509.9 | Ex. 62A |
| 2079 | 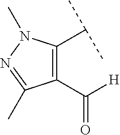 | 4.00 | 429.0 | Ex. 63A Met. AB |
| 2080 | 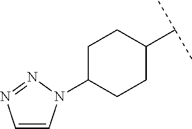 | 4.09 | 456.1 | Ex. 64A |
| 2081 | 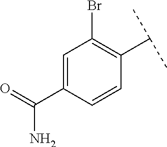 | 4.53 | 503.9/ 505.9 | Met. M |
| 2082 | 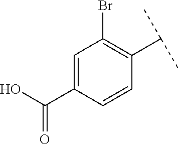 | 5.14 | 504.9/ 506.9 | Met. L |
| 2083 | 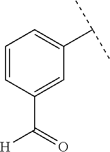 | 2.02 | 411.0 | Ex. 144A Met. AC |
| 2084 | 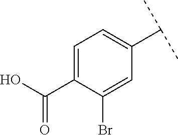 | 4.96 | 505.0/ 507.0 | Met. L |
| 2085 | 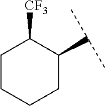 | 2.52 | 457.1 | Met. F |
| 2086 | 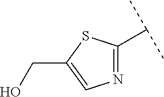 | 1.48 | 420.1 | Ex. 65B Met. AE |
| 2087 | 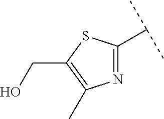 | 1.55 | 434.0 | Met. AD/ AE |
| 2088 | 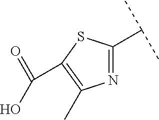 | 1.72 | 448.0 | Ex. 66A Met. AF |
| 2089 | 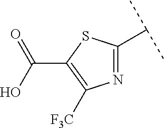 | 2.00 | 502.0 | Met. AF |
| 2090 | 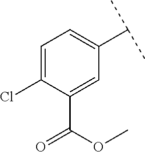 | 2.37 | 475.0 | Met. AC |
| 2091 | 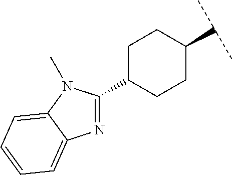 | 4.21 | 519.2 | Ex. 67A |
| 2092 | 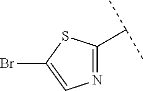 | 2.44 | 467.9/ 469.9 | Met. AD |
| 2093 | 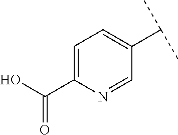 | 3.68 | 428.1 | Met. L |
| 2094 | 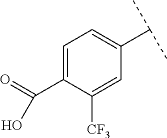 | 6.10 | 495.0 | Met. L |
| 2095 | 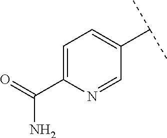 | 3.92 | 427.1 | Met. M |
| 2096 | 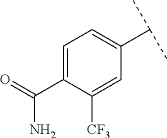 | 4.52 | 494.1 | Met. M |
| 2097 | 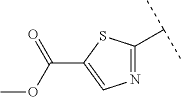 | 2.11 | 448.0 | Ex. 68A |
| 2098 | 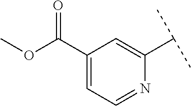 | 2.02 | 442.0 | Met. K |
| 2099 | 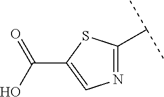 | 1.68 | 434.0 | Met. AF |
| 2100 | 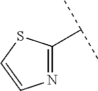 | 1.86 | 390.0 | Ex. 69 |
| 2101 | 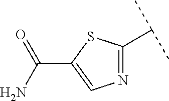 | 1.49 | 433.0 | Met. M |
| 2102 | 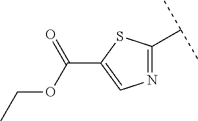 | 2.41 | 462.0 | Ex. 65A Met. AD |
| 2103 | 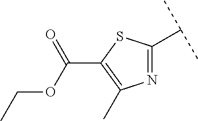 | 2.57 | 476.0 | Met. AD |
| 2104 | 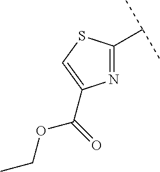 | 2.16 | 462.0 | Met. AD |
| 2105 | 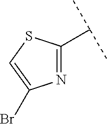 | 2.34 | 467.9/ 469.9 | Met. AD |
| 2106 | 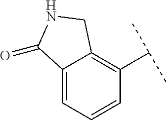 | 1.70 | 438.1 | Met. F |
| 2107 | 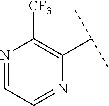 | 5.05 | 453.1 | Ex. 145A |
| 2108 | 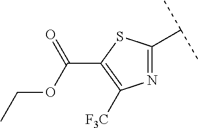 | 2.64 | 530.0 | Met. AD |
| 2109 | 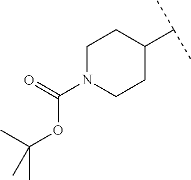 | 5.24 | 490.1 | Met. U |
| 2110 | 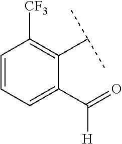 | 5.61 | 479.0 | Met. G |
| 2111 | 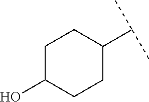 | 3.58 | 405.1 | Met. U |

TABLE 3

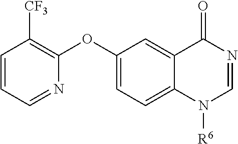

| | | | | Example/ |
|---|---|---|---|---|
| | | | MS | Synthetic |
| Cpd | R⁶ | t_{R} (min) | (M + H)⁺ | Method |
| 3001 | 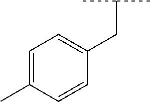 | 5.73 | 412.1 | Ex. 70B Met. AG |
| 3002 | 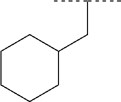 | 5.79 | 404.1 | Met. AG |
| 3003 | 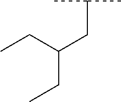 | 5.70 | 392.1 | Met. AG |
| 3004 | 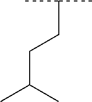 | 5.36 | 378.1 | Met. AG |
| 3005 | 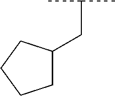 | 5.52 | 390.1 | Met. AG |
| 3006 | 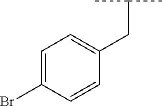 | 6.10 | 475.9/ 477.9 | Met. AG |
| 3007 | 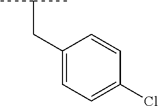 | 5.53 | 432.1 | Ex. 71A Met. AH |
| 3008 | 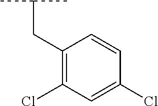 | 6.19 | 466.1 | Met. AH |
| 3009 | 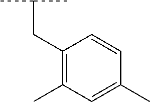 | 5.72 | 426.2 | Met. AH |
| 3010 | 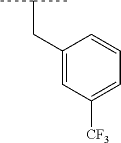 | 5.80 | 466.1 | Met. AH |
| 3011 | 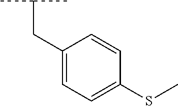 | 5.48 | 444.1 | Met. AH |
| 3012 | 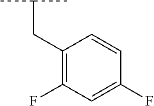 | 5.30 | 434.1 | Met. AH |
| 3013 | 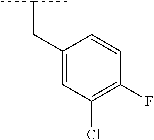 | 5.65 | 450.1 | Met. AH |
| 3014 | 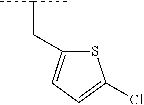 | 5.59 | 438.1 | Met. AH |
| 3015 | 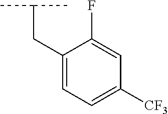 | 6.06 | 484.1 | Met. AH |
| 3016 | 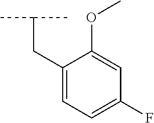 | 5.24 | 446.2 | Met. AH |
| 3017 | 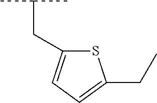 | 5.69 | 432.1 | Met. AH |
| 3018 | 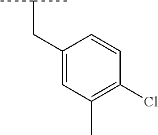 | 5.95 | 446.1 | Met. AH |
| 3019 | 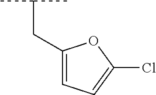 | 5.17 | 422.1 | Met. AH |
| 3020 | 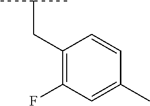 | 5.54 | 430.2 | Met. AH |
| 3021 | 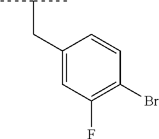 | 5.82 | 494.0/ 496.0 | Met. AH |
| 3022 | 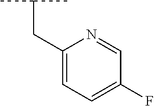 | 4.54 | 417.1 | Met. AH |
| 3023 | 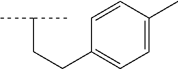 | 5.70 | 426.1 | Ex. 72 Met. AI |
| 3024 | 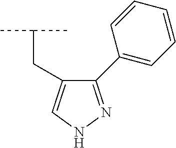 | 4.74 | 464.1 | Met. AI |
| 3025 | 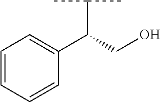 | 4.86 | 428.1 | Ex. 73A Met. AJ |
| 3026 | 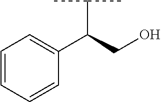 | 4.87 | 428.1 | Met. AJ |
| 3027 | 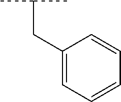 | 5.20 | 398.1 | Ex. 74A |
| 3028 | 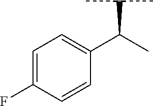 | 5.37 | 430.1 | Ex. 75A Met. AK |
| 3029 | 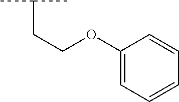 | 5.40 | 428.1 | Met. AK |
| 3030 | 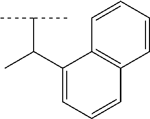 | 5.88 | 462.1 | Met. AK |
| 3031 | 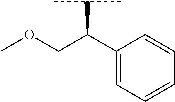 | 5.44 | 442.1 | Met. AK |
| 3031 | 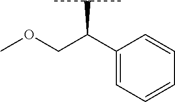 | 5.44 | 442.1 | Met. AK |
| 3032 | 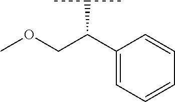 | 5.44 | 442.1 | Met. AK |
| 3033 | 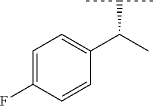 | 5.39 | 430.1 | Met. AK |
| 3034 | 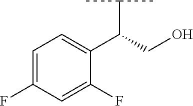 | 5.21 | 464.1 | Ex. 76A |
| 3035 | 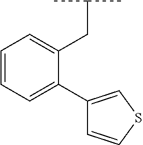 | 6.13 | 480.1 | Ex. 77A Met. AL |
| 3036 | 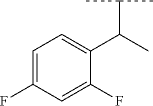 | 5.19 | 448.1 | Ex. 78A Met. AM |
| 3037 | 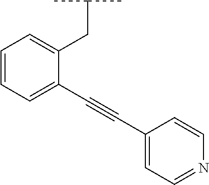 | 4.50 | 499.2 | Ex. 79A Met. AN |
| 3038 | 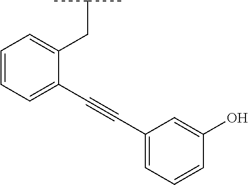 | 6.33 | 514.2 | Met. AN |
| 3039 | 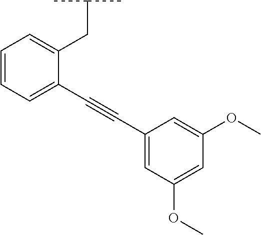 | 7.10 | 558.2 | Met. AN |
| 3040 | 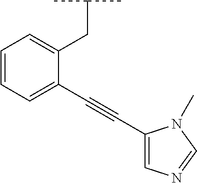 | 4.37 | 502.2 | Met. AN |
| 3041 | 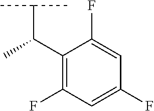 | 5.62 | 466.2 | Ex. 80A |
| 3042 | 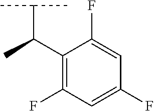 | 5.62 | 466.2 | Met. O |
| 3043 | 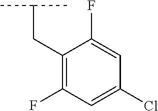 | 5.30 | 468.1 | Ex. 81A |
| 3044 | 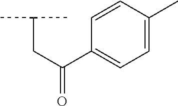 | 5.37 | 440.2 | Ex. 82A Met. AO |
| 3045 | 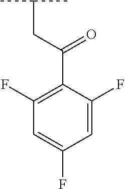 | 5.24 | 478.1 (M − H) only | Met. AO |
| 3046 | 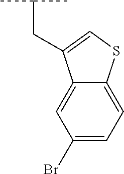 | 6.13 | 531.0/ 533.0 | Met. AM |
| 3047 | 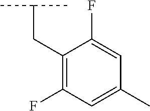 | 5.85 | 448.0 | Ex. 83A |
| 3048 | 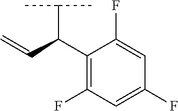 | 5.27 | 478.1 | Ex. 84B Met AP |
| 3049 | 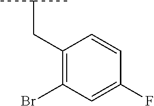 | 5.29 | 494.0/ 496.0 | Met. AG |
| 3050 | 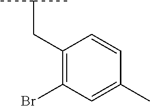 | 5.50 | 490.0/ 492.0 | Met. AG |
| 3051 | 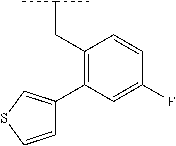 | 5.61 | 498.1 | Met. AL |
| 3052 | 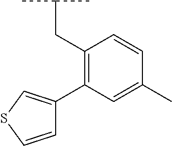 | 5.74 | 494.1 | Met. AL |
| 3053 | 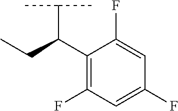 | 5.60 | 480.1 | Ex. 85A |
| 3054 | 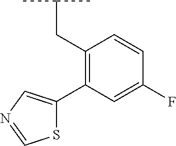 | 4.98 | 499.1 | Met. Q |
| 3055 | 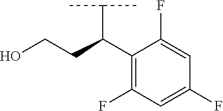 | 4.91 | 496.1 | Ex. 86A |
| 3056 | 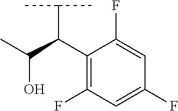 | 5.13 | 496.1 | Ex. 86A |
| 3057 | 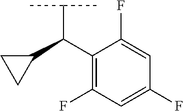 | 5.62 | 492.1 | Ex. 87A |
| 3058 | 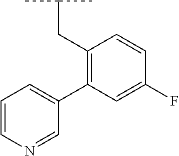 | 4.00 | 493.0 | Ex. 88A Met. AQ |
| 3059 | 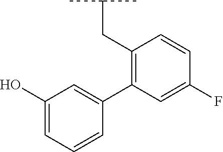 | 5.16 | 508.0 | Met. AQ |
| 3060 | 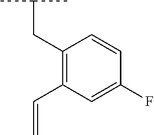 | 5.19 | 442.0 | Met. Q |
| 3061 | 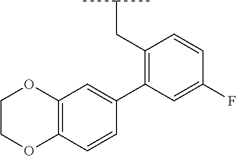 | 5.67 | 550.1 | Met. AQ |
| 3062 | 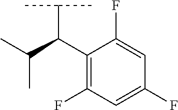 | 5.87 | 494.1 | Met. AP |
| 3063 | 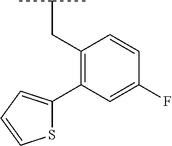 | 5.66 | 498.0 | Met. AQ |
| 3064 | 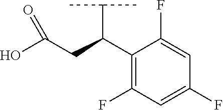 | 5.05 | 510.0 | Ex. 89A |
| 3065 | 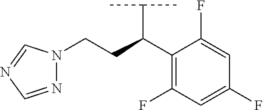 | 4.89 | 547.1 | Met. U |
| 3066 | 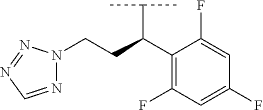 | 5.33 | 548.1 | Met. U |
| 3067 | 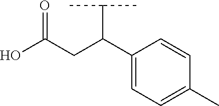 | 5.11 | 468.0 | Ex. 90A |
| 3068 | 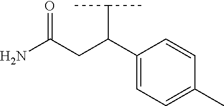 | 4.79 | 469.1 | Met. M |
| 3069 | 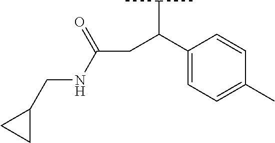 | 5.42 | 523.2 | Met. AA |
| 3070 | 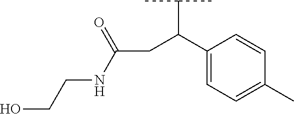 | 4.69 | 513.1 | Met. AA |
| 3071 | 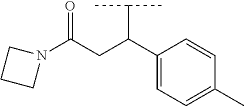 | 5.11 | 509.0 | Met. AA |
| 3072 | 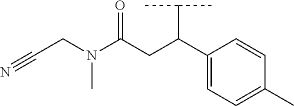 | 5.32 | 522.0 | Met. AA |
| 3073 | 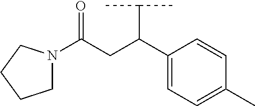 | 5.32 | 523.1 | Met. AA |
| 3074 | 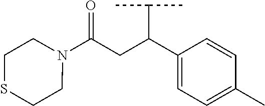 | 5.57 | 555.1 | Met. AA |
| 3075 | 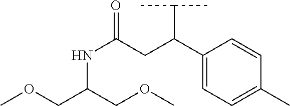 | 5.17 | 571.1 | Met. AA |
| 3076 | 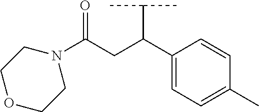 | 5.12 | 539.1 | Met. AA |
| 3077 | 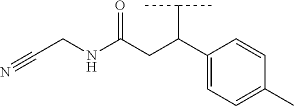 | 5.10 | 508.1 | Met. AA |
| 3078 | 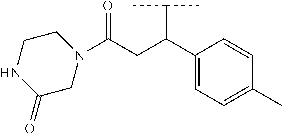 | 4.74 | 552.1 | Met. AA |
| 3079 | 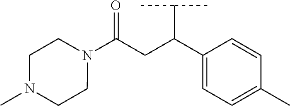 | 4.33 | 552.1 | Met. AA |
| 3080 | 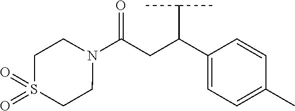 | 5.15 | 587.1 | Ex. 91A |
| 3081 | 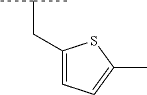 | 5.28 | 418.1 | Met. AH |
| 3082 | 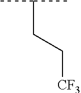 | 4.70 | 404.1 | Met. AH |
| 3083 | 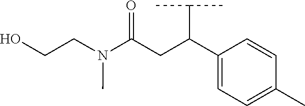 | 4.83 | 527.1 | Met. AA |
| 3084 | 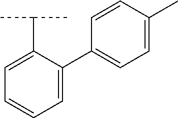 | 6.28 | 474.2 | Ex. 92B Met. AS |
| 3085 | 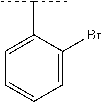 | 5.59 | 462.1/ 464.1 | Met. AR |
| 3086 | 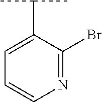 | 5.05 | 463.0/ 465.0 | Met. AR |
| 3087 | 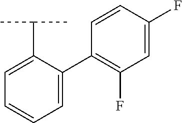 | 6.26 | 496.1 | Met. AS |
| 3088 | 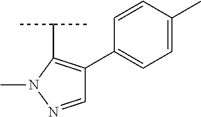 | 6.04 | 478.0 | Met. AR/AS |
| 3089 | 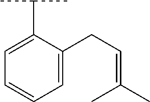 | 6.43 | 452.0 | Met. Q |
| 3090 | 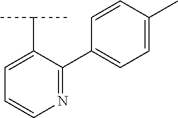 | 5.43 | 475.2 | Ex. 93A Met. AT |
| 3091 | 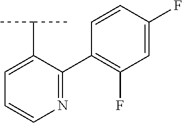 | 5.63 | 497.2 | Met. AT |
| 3092 | 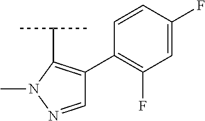 | 5.99 | 500.2 | Met. AT |
| 3093 | 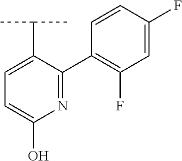 | 4.69 | 513.0 | Ex. 94A |
| 3094 | 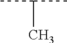 | 3.63 | 322.0 | Ex. 95A |
| 3095 | 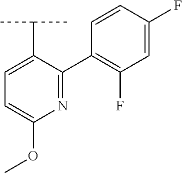 | 6.80 | 527.0 | Ex. 96A |
| 3096 | 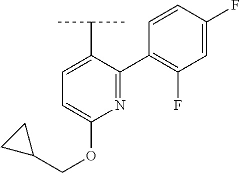 | 7.52 | 567.0 | Ex. 97A |
| 3097 | 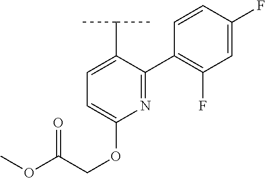 | 6.60 | 585.0 | Ex. 98A Met. AU |
| 3098 | 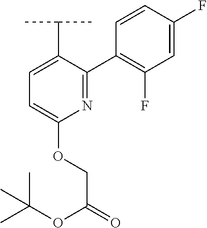 | 7.48 | 627.1 | Met. AU |
| 3099 | 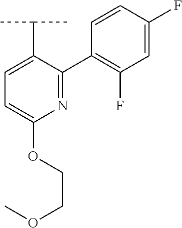 | 6.70 | 571.0 | Met. AU |
| 3100 | 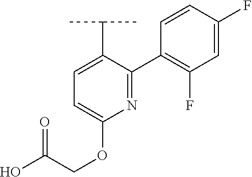 | 5.85 | 571.1 | Ex. 99A |
| 3101 | 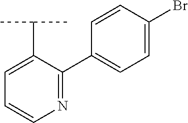 | 5.78 | 539.0/ 541.0 | Ex. 100A Met. AV |
| 3102 | 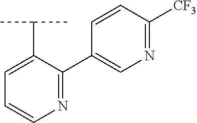 | 5.49 | 530 | Met. AV |
| 3103 | 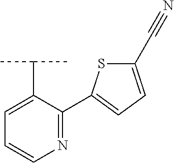 | 5.70 | 492.0 | Met. AV |
| 3104 | 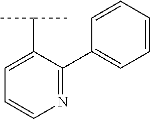 | 5.15 | 461.0 | Met. AV |
| 3105 | 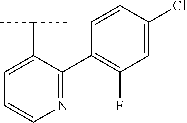 | 5.69 | 513.0 | Met. AV |
| 3106 | 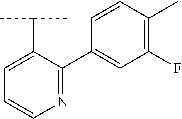 | 5.68 | 493.0 | Met. AV |
| 3107 | 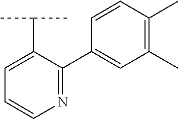 | 5.65 | 489.0 | Met. AV |
| 3108 | 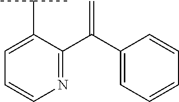 | 5.18 | 487.0 | Met. AV |
| 3109 | 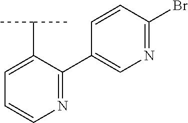 | 5.16 | 540.0/ 542.0 | Met. AV |
| 3110 | 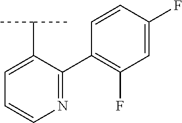 | 5.78 | 497.1 | Ex. 101A Met. AW |
| 3111 | 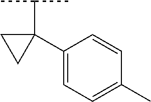 | 5.70 | 438.0 | Met. AR |
| 3112 | 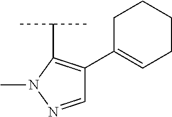 | 6.07 | 468.0 | Met. AT |
| 3113 | 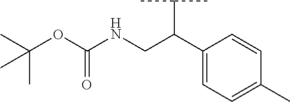 | 5.86 | 541.1 | Met. AR |
| 3114 | 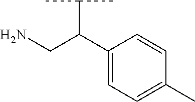 | 4.43 | 441.1 | Ex. 102A |
| 3115 | 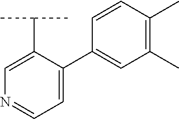 | 5.89 | 475.0 | Met. AW |
| 3116 | 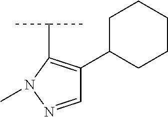 | 6.31 | 470.2 | Ex. 103A Met. AX |
| 3117 | 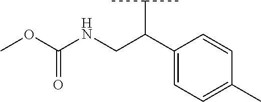 | 5.24 | 499.1 | Met. R |
| 3118 | 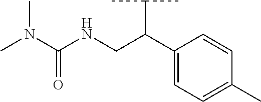 | 4.98 | 512.1 | Met. R |
| 3119 | 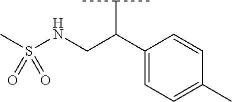 | 5.10 | 519.0 | Met. R |
| 3120 | 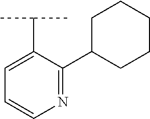 | 5.69 | 467.2 | Met. AX |
| 3121 | 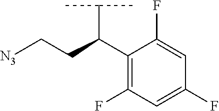 | 5.82 | 521.0 | Ex. 104A |
| 3122 | 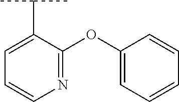 | 5.71 | 477.0 | Ex. 105A |
| 3123 | 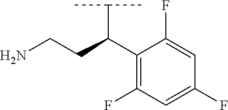 | 4.30 | 495.0 | Ex. 104A |
| 3124 | 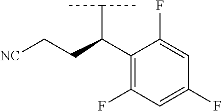 | 5.34 | 505.0 | Ex. 106A |
| 3125 | 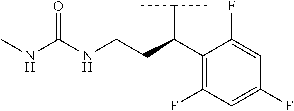 | 4.74 | 552.0 | Met. R |
| 3126 | 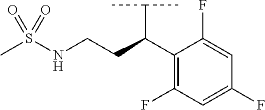 | 5.04 | 572.9 | Met. R |
| 3127 | 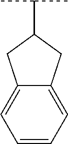 | 4.93 | 424.0 | Met. AR |
| 3128 | 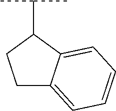 | 4.92 | 424.0 | Met. AR |
| 3129 | 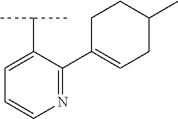 | 5.54 | 479.1 | Ex. 107A Met. AY |
| 3130 | 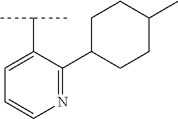 | 6.09 | 481.0 | Ex. 107A Met. AY |
| 3131 | 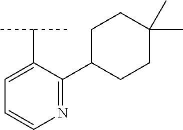 | 6.29 | 495.0 | AY |
| 3132 | 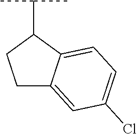 | 5.38 | 458.1 | Ex. 108A Met. AZ |
| 3133 | 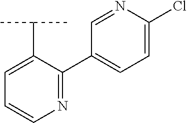 | 5.07 | 496.0 | Met. AV |
| 3134 | 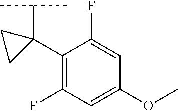 | 5.72 | 489.9 | Ex. 109A |
| 3135 | 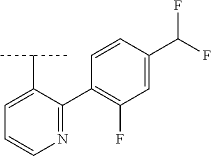 | 3.93 | 529.1 | Ex. 110A |
| 3136 | 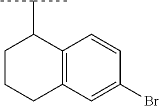 | 5.75 | 516.0/ 518.0 | Met. AZ |
| 3137 | 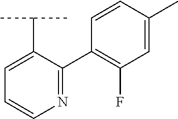 | 6.04 | 493.1 | Met. AL |
| 3138 | 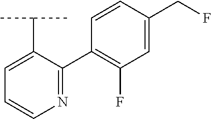 | 5.88 | 511.1 | Met. AL |
| 3139 | 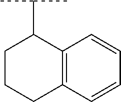 | 5.21 | 438.1 | Ex. 111A |
| 3140 | 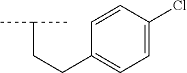 | 5.96 | 446.1 | Met. AH |
| 3141 | 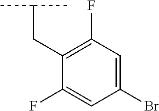 | 6.11 | 512.1/ 514.1 | Met. AH |

TABLE 4

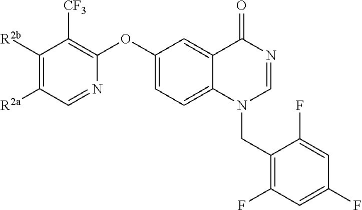

| | | | | | Example/ |
|---|---|---|---|---|---|
| | | | t_{R} | MS | Synthetic |
| Cpd | R^{2a} | R^{2b} | (min) | (M + H)⁺ | Method |
| 4001 | H | H | 5.61 | 452.1 | Met. AH |
| 4002 | H | —OCH₃ | 4.94 | 482.0 | Met. G |
| 4003 | H | 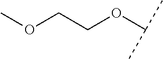 | 5.08 | 526.1 | Met. D |
| 4004 | H | 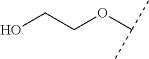 | 4.31 | 512.1 | Met. D |
| 4005 | H | 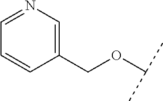 | 4.11 | 559.1 | Met. X |
| 4006 | H | 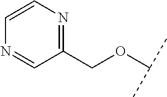 | 4.95 | 560.1 | Met. U |
| 4007 | 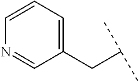 | H | 4.19 | 543.2 | Met. S |
| 4008 | 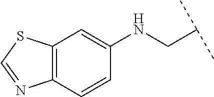 | H | 5.98 | 614.1 | Ex. 112A Met. BA |
| 4009 | 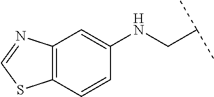 | H | 5.97 | 614.1 | Met. BA |
| 4010 | 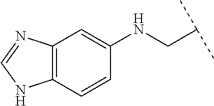 | H | 4.32 | 597.2 | Met. BA |
| 4011 | 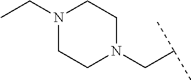 | H | 4.03 | 578.2 | Met. BA |
| 4012 | 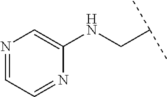 | H | 5.10 | 559.2 | Met. BA |
| 4013 | 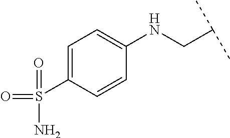 | H | 5.47 | 636.1 | Met. BA |
| 4014 | 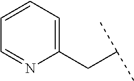 | H | 4.14 | 543.2 | Met. Q |
| 4015 | 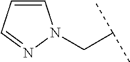 | H | 4.93 | 532.3 | Met. P |
| 4016 | 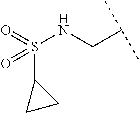 | H | 5.54 | 585.0 | Ex. 113A |
| 4017 | 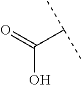 | H | 5.25 | 496.0 | Met. L |
| 4018 | 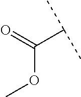 | H | 6.02 | 510.0 | Ex. 114A |
| 4019 | 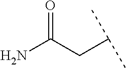 | H | 4.59 | 506.9 | Met. M |
| 4020 | 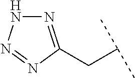 | H | 4.90 | 534.0 | Ex. 115A Met. BB |
| 4021 | 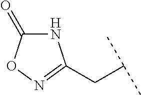 | H | 5.23 | 550.0 | Ex. 116A Met. BC |
| 4022 | 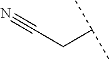 | H | 5.51 | 491.0 | Ex. 62A |
| 4023 | 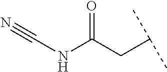 | H | 5.29 | 533.9 | Met. AA |
| 4024 | 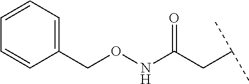 | H | 5.82 | 615.0 | Met. AA |
| 4025 | 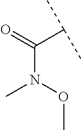 | H | 5.49 | 539.0 | Met. AA |
| 4026 | 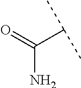 | H | 4.76 | 495.0 | Met. M |
| 4027 | 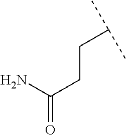 | H | 4.78 | 523.1 | Met. M |
| 4028 | 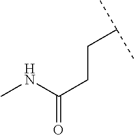 | H | 4.92 | 537.1 | Met. AA |
| 4029 | H | 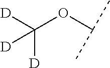 | 5.73 | 485.1 | Ex. 153A |
| 4030 | 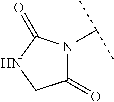 | H | 4.90 | 550.0 | Ex. 118B Met. BE |
| 4031 | 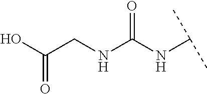 | H | 4.73 | 568.0 | Ex. 118B Met. BE |
| 4032 | 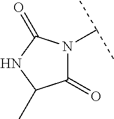 | H | 5.19 | 564.0 | Met. BE |
| 4033 | 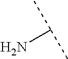 | H | 4.89 | 467.1 | Ex. 118A Met. BD |
| 4034 | 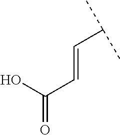 | H | 5.46 | 522.1 | Ex. 117A |
| 4035 | 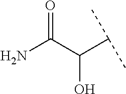 | H | 4.45 | 525.1 | Ex. 119A Met. BF |
| 4036 | 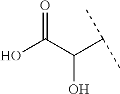 | H | 4.66 | 526.1 | Ex. 119A Met. BF |
| 4037 | 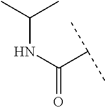 | H | 5.70 | 537.0 | Met. AA |
| 4038 | 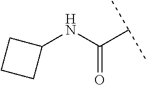 | H | 5.94 | 549.0 | Met. AA |
| 4039 | 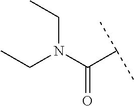 | H | 5.71 | 551.0 | Met. AA |
| 4040 | 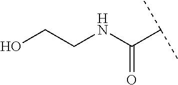 | H | 4.62 | 539.0 | Met. AA |
| 4041 | 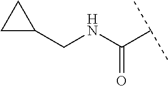 | H | 5.88 | 549.0 | Met. AA |
| 4042 | 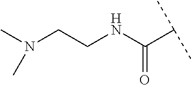 | H | 4.24 | 566.0 | Met. AA |
| 4043 | 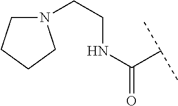 | H | 4.35 | 592.0 | Met. AA |
| 4044 | 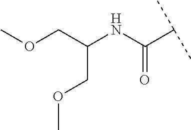 | H | 5.38 | 597.0 | Met. AA |
| 4045 | 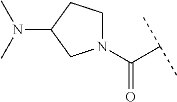 | H | 4.12 | 592.1 | Met. AA |
| 4046 | 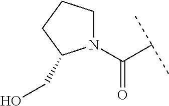 | H | 4.97 | 579.0 | Met. AA |
| 4047 | 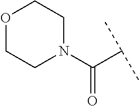 | H | 5.11 | 565.0 | Met. AA |
| 4048 | 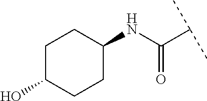 | H | 5.00 | 593.0 | Met. AA |
| 4049 | 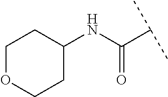 | H | 5.27 | 579.0 | Met. AA |
| 4050 | 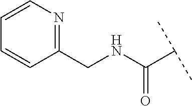 | H | 4.33 | 586.0 | Met. AA |
| 4051 | 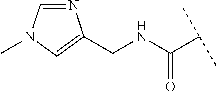 | H | 4.29 | 589.0 | Met. AA |
| 4052 | 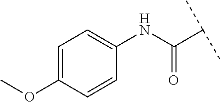 | H | 6.44 | 601.1 | Met. AA |
| 4053 | 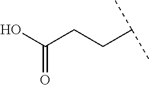 | H | 5.20 | 524.1 | Ex. 117A |
| 4054 | H | 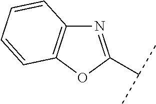 | 6.65 | 569.2 | Ex. 147A |

TABLE 5

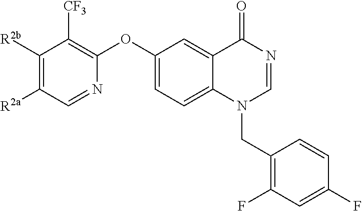

| | | | | | Example/ |
|---|---|---|---|---|---|
| | | | t_{R} | MS | Synthetic |
| Cpd | R^{2a} | R^{2b} | (min) | (M + H)⁺ | Method |
| 5001 | 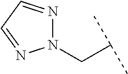 | H | 5.62 | 515.1 | Met. I |
| 5002 | 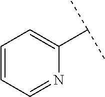 | H | 4.95 | 511.1 | Met. Q |
| 5003 | H | —OH | 4.50 | 450.1 | Met. C |
| 5004 | 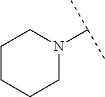 | H | 5.54 | 517.3 | Ex. 120A Met. BG |
| 5005 | H | 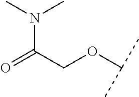 | 4.57 | 535.2 | Met. D |
| 5006 | H | 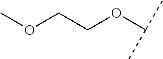 | 5.07 | 508.2 | Met. D |
| 5007 | H | 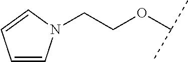 | 5.76 | 543.2 | Met. D |
| 5008 | 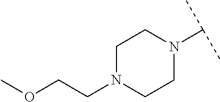 | H | 4.26 | 576.3 | Met. BG |
| 5009 | 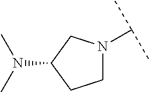 | H | 4.24 | 546.2 | Met. BG |
| 5010 | H | 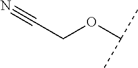 | 4.95 | 489.1 | Met. D |
| 5011 | H | 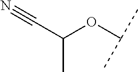 | 5.33 | 503.1 | Met. D |
| 5012 | 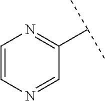 | H | 5.26 | 512.1 | Met. Q |
| 5013 | 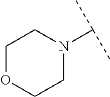 | H | 5.11 | 519.2 | Met. BG |
| 5014 | 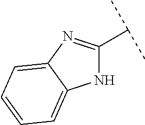 | H | 1.97* | 550.0 | Ex. 121A |
| 5015 | H | —OCH₃ | 5.60 | 464.1 | Met. I |
| 5016 | 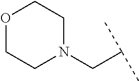 | H | 4.15 | 533.2 | Ex. 122A Met. BH |
| 5017 | 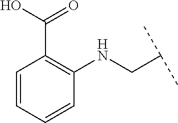 | H | 6.39 | 583.2 | Met. BH |
| 5018 | 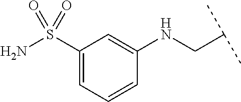 | H | 5.71 | 618.1 | Met. BH |
| 5019 | 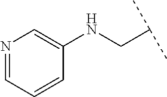 | H | 4.44 | 540.2 | Met. BH |
| 5020 | 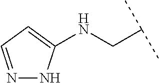 | H | 4.52 | 529.2 | Met. BH |
| 5021 | 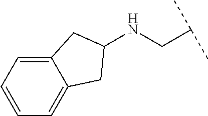 | H | 5.01 | 579.2 | Met. BH |
| 5022 | 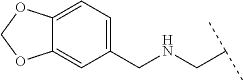 | H | 4.84 | 597.2 | Met. BH |
| 5023 | 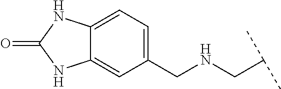 | H | 4.32 | 595.2 | Met. BH |
| 5024 | 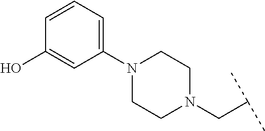 | H | 4.71 | 624.2 | Met. BH |
| 5025 | 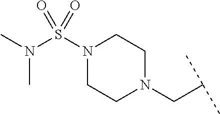 | H | 4.64 | 639.2 | Met. BH |
| 5026 | 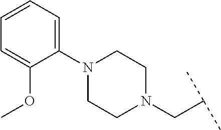 | H | 5.07 | 638.2 | Met. BH |
| 5027 | 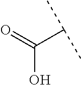 | H | 5.33 | 478.1 | Met. H |
| 5028 | 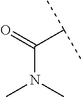 | H | 5.07 | 505.2 | Met. AA |
| 5029 | 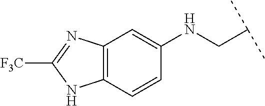 | H | 5.77 | 647.2 | Met. BA |
| 5030 | 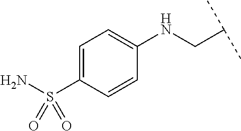 | H | 5.49 | 618.1 | Met. BH |
| 5031 | 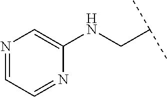 | H | 5.15 | 541.2 | Met. BH |
| 5032 | 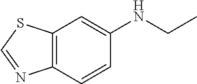 | H | 6.01 | 596.1 | Met. BA |
| 5033 | 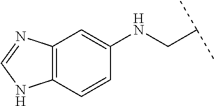 | H | 4.65 | 579.2 | Met. BA |
| 5034 | H | 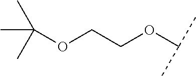 | 5.95 | 550.2 | Met. U |
| 5035 | H | 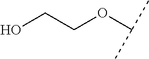 | 4.31 | 494.2 | Ex. 123A |
| 5036 | 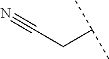 | H | 4.86 | 473.1 | Ex. 124A |
| 5037 | 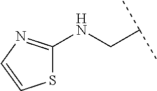 | H | 4.37 | 546.1 | Met. BH |

TABLE 6

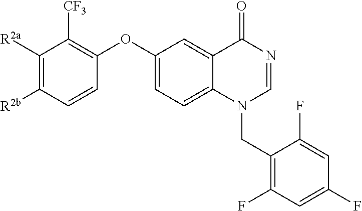

| | | | t_{R} | MS | Synthetic |
|---|---|---|---|---|---|
| Cpd | R^{2a} | R^{2b} | (min) | (M + H)⁺ | Method |
| 6001 | —NH₂ | H | 4.65 | 466.1 | N |
| 6002 | 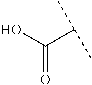 | H | 5.06 | 495.2 | Met. L |
| 6003 | 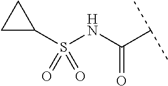 | H | 5.81 | 598.0 | Ex. 125A Met. BI |
| 6004 | 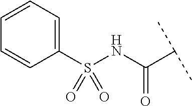 | H | 6.35 | 634.0 | Met. BI |
| 6005 | 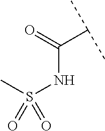 | H | 5.44 | 572.0 | Met. BI |
| 6006 | 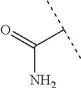 | H | 4.84 | 494.0 | Met. M |
| 6007 | H | H | 6.07 | 451.0 | Met. F |
| 6008 | 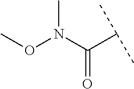 | H | 5.59 | 538.0 | Met. AA |
| 6009 | 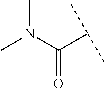 | H | 5.24 | 521.9 | Met. AA |
| 6010 | 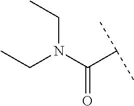 | H | 5.86 | 550.0 | Met. AA |
| 6011 | 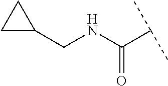 | H | 5.98 | 5.98 | Met. AA |
| 6012 | 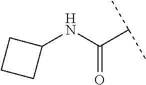 | H | 6.05 | 547.9 | Met. AA |
| 6013 | 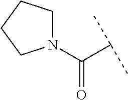 | H | 5.59 | 547.9 | Met. AA |
| 6014 | 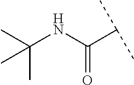 | H | 6.39 | 549.9 | Met. AA |
| 6015 | 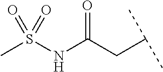 | H | 5.33 | 586.1 | Met. BI |
| 6016 | 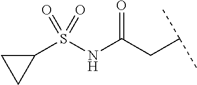 | H | 5.67 | 612.1 | Met. BI |
| 6017 | 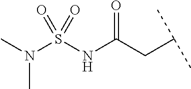 | H | 5.70 | 615.1 | Met. BI |
| 6018 | 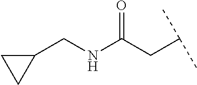 | H | 5.70 | 562.1 | Met. AA |
| 6019 | 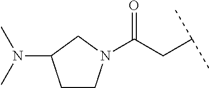 | H | 4.35 | 605.2 | Met. AA |
| 6020 | 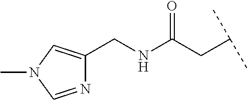 | H | 4.40 | 602.1 | Met. AA |
| 6021 | 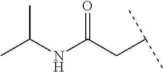 | H | 5.54 | 550.1 | Met. AA |
| 6022 | 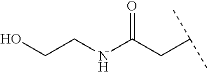 | H | 4.66 | 552.1 | Met. AA |
| 6023 | 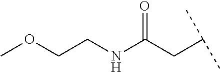 | H | 5.12 | 566.1 | Met. AA |
| 6024 | 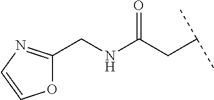 | H | 5.11 | 589.1 | Met. AA |
| 6025 | 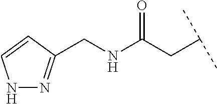 | H | 4.83 | 588.1 | Met. AA |
| 6026 | 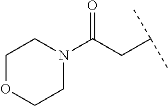 | H | 5.26 | 578.1 | Met. AA |
| 6027 | 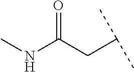 | H | 4.97 | 522.1 | Met. AA |
| 6028 | 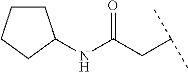 | H | 5.97 | 576.2 | Met. AA |
| 6029 | 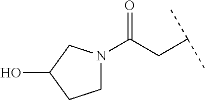 | H | 4.80 | 578.1 | Met. AA |
| 6030 | 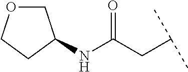 | H | 5.05 | 578.1 | Met. AA |
| 6031 | 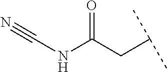 | H | 5.46 | 533.1 | Met. AA |
| 6032 | 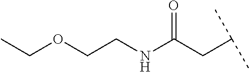 | H | 5.37 | 580.1 | Met. AA |
| 6033 | H | 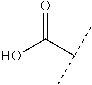 | 1.92* | 494.9 | Ex. 127A |
| 6034 | 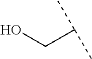 | H | 5.20 | 481.1 | Ex. 126A Met. BJ |
| 6035 | 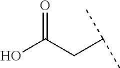 | H | 5.34 | 509.1 | Ex. 126A Met. BJ |
| 6036 | 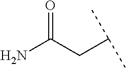 | H | 4.81 | 508.1 | Met. M |
| 6037 | 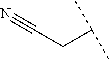 | H | 5.83 | 490.1 | Ex. 126A Met. BJ |
| 6038 | H | 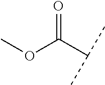 | 2.48* | 509.0 | Met. K |
| 6039 | 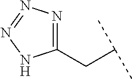 | H | 5.04 | 533.1 | Met. BB |
| 6040 | H | 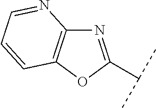 | 5.67 | 569.2 | Ex. 146A Met. BO |
| 6041 | H | 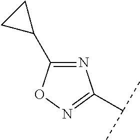 | 6.32 | 559.2 | Ex. 148A Met. BP |
| 6042 | H | 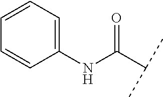 | 5.73 | 570.2 | Met. AA |
| 6043 | H | 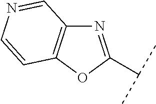 | 2.34 | 569.1 | Met. BO |
| 6044 | H | 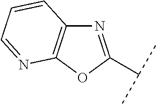 | 5.57 | 569.1 | Met. BO |
| 6045 | H | 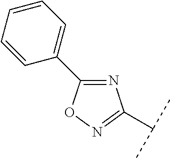 | 7.07 | 595.2 | Met. BP |
| 6046 | H | 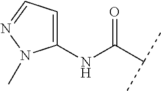 | 4.59 | 574.2 | Met. AA |
| 6047 | H | 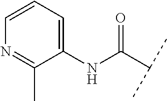 | 3.80 | 585.2 | Met. AA |
| 6048 | H | 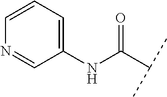 | 1.43 | 571.2 | Met. AA |

TABLE 7

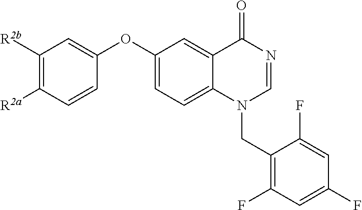

| | | | | | Example/ |
|---|---|---|---|---|---|
| | | | t_{R} | MS | Synthetic |
| Cpd | R^{2a} | R^{2b} | (min) | (M + H)⁺ | Method |
| 7001 | 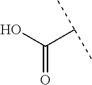 | H | 4.44 | 427.2 | Met. L |
| 7002 | H | H | 5.37 | 383.2 | Met. F |
| 7003 | H | 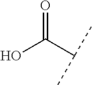 | 4.09 | 427.1 | Ex. 128A Met. BK |
| 7004 | 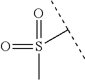 | H | 4.61 | 461.1 | Met. O |
| 7005 | 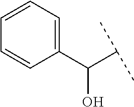 | H | 5.63 | 489.2 | Ex. 129A Met. BL |
| 7006 | 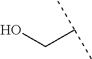 | H | 4.33 | 413.0 | Ex. 130A |
| 7007 | 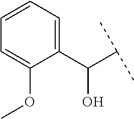 | H | 5.71 | 519.1 | Met. BL |
| 7008 | 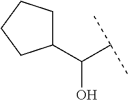 | H | 5.94 | 481.1 | Met. BL |
| 7009 | 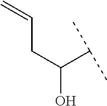 | H | 5.22 | 453.1 | Met. BL |
| 7010 | 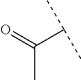 | H | 5.08 | 425.1 | Ex. 131A |
| 7011 | 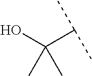 | H | 4.88 | 441.1 | Ex. 131A |
| 7012 | 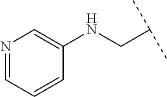 | H | 3.97 | 489.0 | Met. BA |
| 7013 | 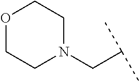 | H | 3.51 | 482.0 | Met. BA |
| 7014 | 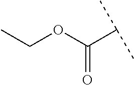 | H | 5.48 | 455.0 | Met. G |
| 7015 | OH | H | 4.47 | 399.0 | Met. H |
| 7016 | 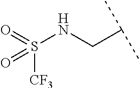 | H | 6.11 | 543.9 | Met. U |
| 7017 | 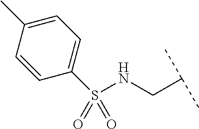 | H | 6.04 | 566.0 | Ex. 132A |
| 7018 | 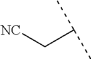 | H | 4.98 | 422.1 | Met. BJ |
| 7019 | 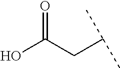 | H | 4.51 | 441.1 | Met. BJ |
| 7020 | 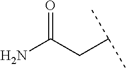 | H | 3.95 | 440.1 | Met. M |
| 7021 | 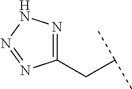 | H | 4.42 | 465.1 | Met. BB |
| 7022 | 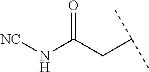 | H | 4.76 | 465.1 | Met. AA |
| 7023 | 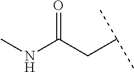 | H | 4.23 | 454.1 | Met. AA |
| 7024 | 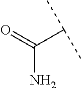 | H | 3.99 | 426.0 | Met. M |
| 7025 | H | 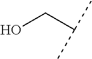 | 1.80* | 413.0 | Ex. 133A |
| 7026 | H | 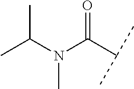 | 2.03* | 482.0 | Met. AA |
| 7027 | H | 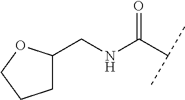 | 1.85* | 510.0 | Met. AA |
| 7028 | H | 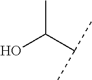 | 1.89* | 427.1 | Ex. 134A |
| 7029 | H | 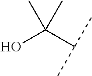 | 2.01* | 441.1 | Ex. 135A |
| 7030 | 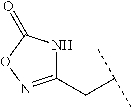 | H | 4.77 | 481.0 | Met. BC |
| 7031 | H | 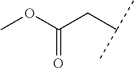 | 2.28* | 455.1 | Met. F |
| 7032 | H | 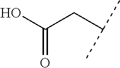 | 1.99* | 441.1 | Met. BK |
| 7033 | H | 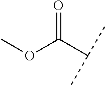 | 2.28* | 441.0 | Met. AC |
| 7034 | H | 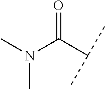 | 1.80* | 454.0 | Met. AA |
| 7035 | H | 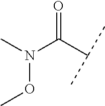 | 1.94* | 470.0 | Met. AA |
| 7036 | H | 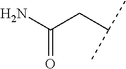 | 1.66* | 440.0 | Met. M |
| 7037 | H | 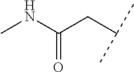 | 1.73* | 454.1 | Met. AA |
| 7038 | 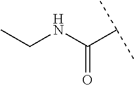 | H | 4.50 | 454.0 | Met. AA |
| 7039 | 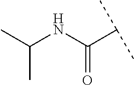 | H | 4.84 | 468.0 | Met. AA |
| 7040 | 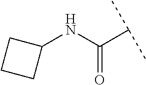 | H | 5.06 | 480.0 | Met. AA |
| 7041 | 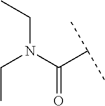 | H | 5.06 | 482.0 | Met. AA |
| 7042 | 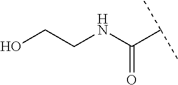 | H | 3.87 | 470.0 | Met. AA |
| 7043 | 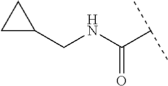 | H | 5.04 | 480.0 | Met. AA |
| 7044 | 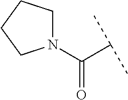 | H | 4.78 | 480.0 | Met. AA |
| 7045 | 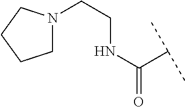 | H | 3.75 | 523.0 | Met. AA |
| 7046 | 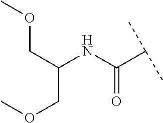 | H | 4.62 | 528.0 | Met. AA |
| 7047 | 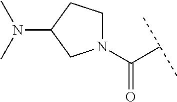 | H | 3.50 | 523.1 | Met. AA |
| 7048 | 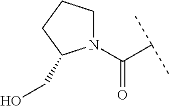 | H | 4.36 | 510.0 | Met. AA |
| 7049 | 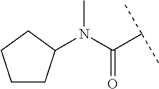 | H | 5.60 | 508.0 | Met. AA |
| 7050 | 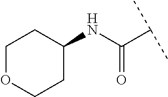 | H | 4.50 | 510.0 | Met. AA |
| 7051 | 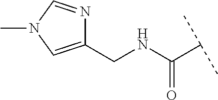 | H | 3.65 | 520.0 | Met. AA |
| 7052 | 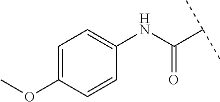 | H | 5.65 | 532.0 | Met. AA |
| 7053 | 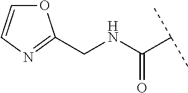 | H | 4.38 | 507.0 | Met. AA |
| 7054 | 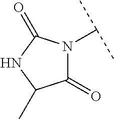 | H | 4.38 | 495.1 | Met. BE |
| 7055 | 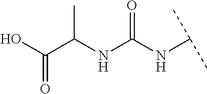 | H | 4.29 | 513.1 | Met. BE |
| 7056 | H | 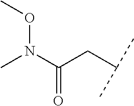 | 2.05* | 484.1 | Met. AA |
| 7057 | H | 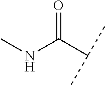 | 1.72* | 440.1 | Met. AA |
| 7058 | H | 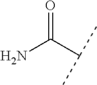 | 1.63* | 426.1 | Met. M |
| 7059 | H | 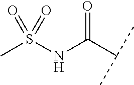 | 1.88* | 504.0 | Met. BI |
| 7060 | H | 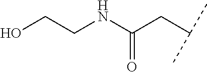 | 1.63* | 484.1 | Met. AA |
| 7061 | H | 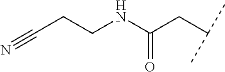 | 1.7* | 493.1 | Met. AA |
| 7062 | H | 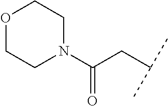 | 1.85* | 510.1 | Met. AA |
| 7063 | H | 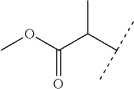 | 2.40* | 469.0 | Ex. 136 |
| 7064 | 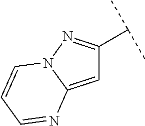 | H | 5.09 | 500.1 | Ex. 137 |

TABLE 8

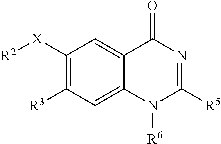

| | | | | | | | | Example/ |
|---|---|---|---|---|---|---|---|---|
| | | | | | | t_{R} | MS | Synthetic |
| Cpd | X | R² | R³ | R⁵ | R⁶ | (min) | (M + H)⁺ | Method |
| 8001 | CH₂ | 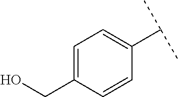 | H | H | 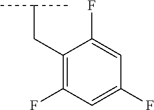 | 4.26 | 411.1 | Ex. 138A |
| 8002 | O | 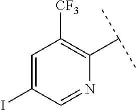 | H | —CH₃ | 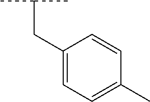 | 5.14 | 552.1 | Ex. 139A Met BM |
| 8003 | O | 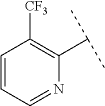 | H | —CH₃ | 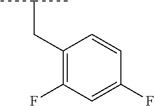 | 4.90 | 448.1 | Met. BM |
| 8004 | O | 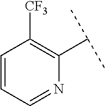 | H | 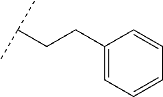 | 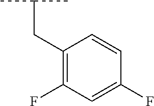 | 6.30 | 538.1 | Ex. 140A Met. BN |
| 8005 | O | 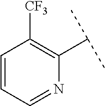 | H | 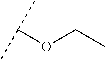 | 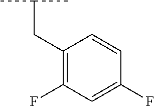 | 6.59 | 478.1 | Ex. 141A |
| 8006 | O | 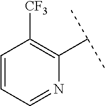 | F | 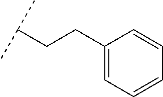 | 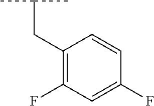 | 6.53 | 556.1 | Met. BN |
| 8007 | S | 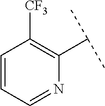 | H | H | 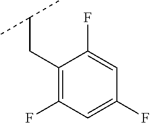 | 5.21 | 467.9 | Ex. 142A |
| 8008 | O | 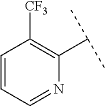 | H | —CH₃ | 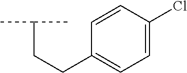 | 5.66 | 460.1 | Met. BM |
| 8009 | O | 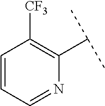 | —N(CH₃)₂ | H | 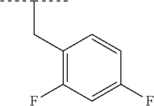 | 5.40 | 477.1 | Ex. 143A |
| 8010 | O | 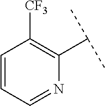 | H | —CH₃ | 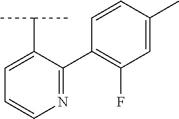 | 5.25 | 507.2 | Ex. 149A |
| 8011 | O | 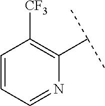 | H | 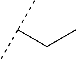 | 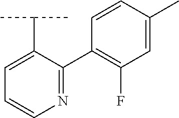 | 4.96 | 521.1 | Ex. 150A |
| 8012 | O | 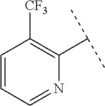 | H | 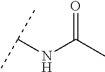 | 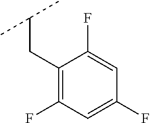 | 2.64 | 509.1 | Ex. 151A |

TABLE 9

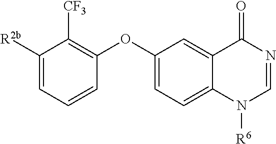

| | | | | | Example/ |
|---|---|---|---|---|---|
| | | | t_{R} | MS | Synthetic |
| Cpd | R^{2b} | R⁶ | (min) | (M + H)⁺ | Method |
| 9001 | 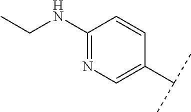 | 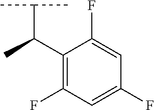 | 1.87* | 585.3 | Ex. 154D Met. BQ |
| 9002 | 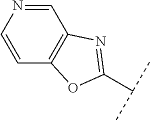 | 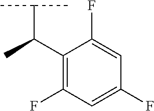 | 1.88* | 583.1 | BO |
| 9003 | 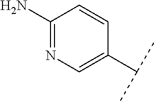 | 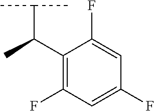 | 1.72* | 557.3 | AL |
| 9004 | 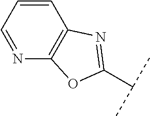 | 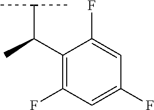 | 6.01 | 583.2 | BO |
| 9005 | 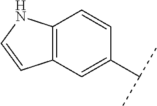 | 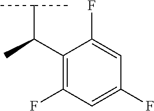 | 6.61 | 580.2 | AQ |
| 9006 | 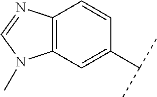 | 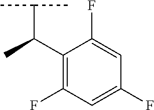 | 1.84* | 595.1 | Ex. 155 Met. BR |
| 9007 | 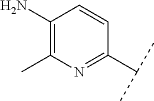 | 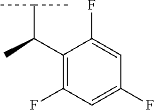 | 1.70* | 571.2 | BQ |
| 9008 | 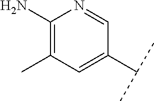 | 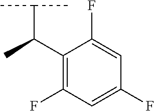 | 1.81* | 571.2 | BR |
| 9009 | 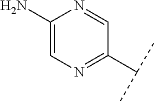 | 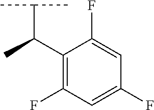 | 1.67* | 558.1 | BR |
| 9010 | 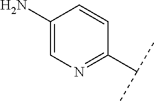 | 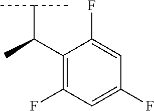 | 1.69* | 557.1 | BQ |
| 9011 | 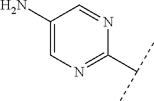 | 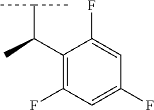 | 1.57* | 558.1 | Ex. 156A |
| 9012 | 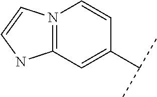 | 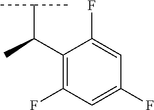 | 4.04 | 581.1 | BR |
| 9013 | 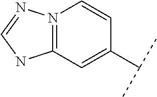 | 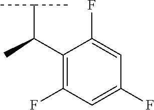 | 4.04 | 582.2 | BQ |
| 9014 | 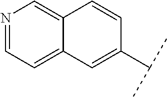 | 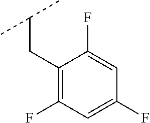 | 4.48 | 578.2 | AQ |
| 9015 | 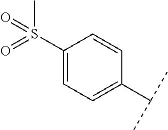 | 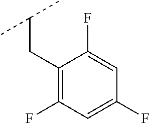 | 2.32* | 605.1 | AQ |
| 9016 | 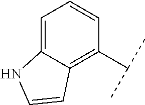 | 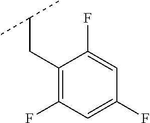 | 2.50* | 566.2 | AQ |
| 9017 | 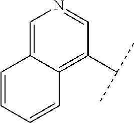 | 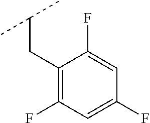 | 2.48* | 578.2 | AQ |
| 9018 | 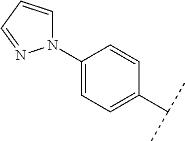 | 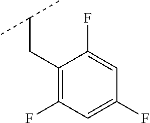 | 2.56* | 593.2 | AQ |
| 9019 | 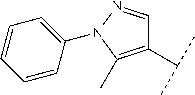 | 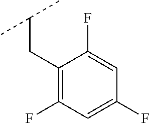 | 2.56* | 607.1 | AQ |
| 9020 | 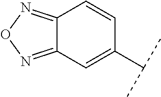 | 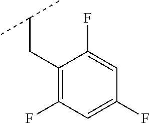 | 2.61 | 569.1 | AQ |
| 9021 | 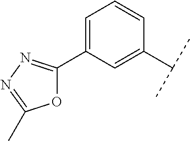 | 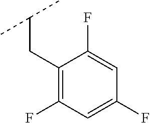 | 2.47* | 609.1 | AQ |
| 9022 | 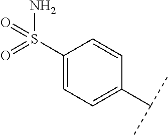 | 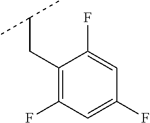 | 2.21* | 606.1 | AQ |
| 9023 | 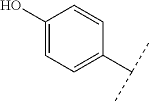 | 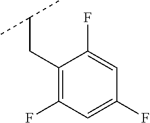 | 2.41* | 543.1 | AQ |
| 9024 | 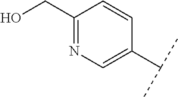 | 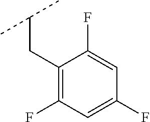 | 3.80 | 558.3 | BR |
| 9025 | 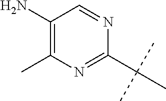 | 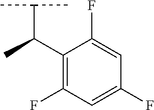 | 1.61* | 572.3 | Ex. 157A |
| 9026 | 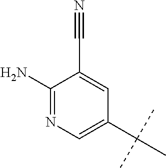 | 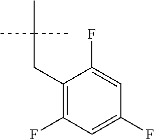 | 1.75* | 568.1 | BR |

TABLE 10

| | IC₅₀ (nM) | EC₅₀ (nM) |
|---|---|---|
| Compound | Ex 158A | Ex 159A |
| 1008 | 240 | 650 |
| 1030 | 610 | 8 500 |
| 2061 | 530 | 2 500 |
| 3001 | 450 | 2 500 |
| 3052 | 110 | 210 |
| 3090 | 130 | 82 |
| 4003 | 67 | 61 |
| 4020 | 78 | 5 400 |
| 5001 | 240 | 470 |
| 6036 | 18 | 60 |
| 6047 | 15 | 10 |
| 7034 | 4 900 | 5 700 |
| 8009 | 25 000 | >10 000 |
| 8011 | 34 500 | 4 800 |
| 9001 | 23 | |
| 9003 | 18 | 4 |
| 9011 | 7.8 | |
| 9016 | 25 | 2.2 |
| 9019 | 51 | 183 |
| 9020 | 107 | 199 |
| 9025 | 14 | |
| 9026 | 28 | |

Each of the references, including all patents, patent applications and publications, listed in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A compound of formula (I): 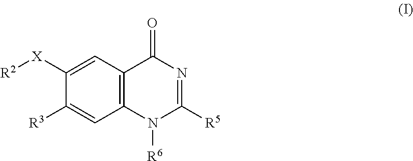
wherein:
X is selected from O, CH₂ and S;
R² is (C₃₋₆)cycloalkyl, aryl or Het, all of which being optionally substituted with 1 to 5 R²⁰ substituents, wherein R²⁰ in each case is independently selected from:
a) halo, cyano, oxo or nitro;
b) R⁷, —C(═O)—R⁷, —C(═O)OR⁷, —SR⁷, —SOR⁷, —SO₂R⁷, —(C₁₋₆)alkylene-R⁷, —(C₁₋₆)alkylene-C(═O)R⁷, —(C₁₋₆)alkylene-C(═O)OR⁷, —(C₁₋₆)alkylene-OR⁷, —(C₁₋₆)alkylene-SR⁷, —(C₁₋₆)alkylene-SOR⁷ or —(C₁₋₆)alkylene-SO₂R⁷;
wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, (C₃₋₇)spirocycloalkyl optionally containing 1 to 3 heteroatom selected from N, O and S, aryl and Het;
wherein the (C₁₋₆)alkyl, (C₂₋₆)alkenyl, (C₂₋₆)alkynyl, (C₁₋₆)haloalkyl and (C₃₋₇)cycloalkyl are optionally substituted with 1 to 5 substituents each independently selected from —OH, oxo, —(C₁₋₆)alkyl (optionally substituted with —O—(C₁₋₆)alkyl), halo, —(C₁₋₆)haloalkyl, (C₃₋₇)cycloalkyl, —O—(C₁₋₆)alkyl, cyano, COOH, —N(R⁸)R⁹, —C(═O)N(R⁸)R⁹, (C₃₋₇)spirocycloalkyl optionally containing 1 to 3 heteroatoms selected from N, O and S, aryl, —(C₁₋₆)alkyl-aryl, Het and —(C₁₋₆)alkyl-Het; and
wherein each of the aryl and Het is optionally substituted with 1 to 3 substituents each independently selected from:
i) halo, cyano, oxo, thioxo, imino, —OH, —COOH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, SO₂NH₂, —SO₂—NH(C₁₋₆)alkyl, —SO₂—N((C₁₋₆)alkyl)₂, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl or Het, wherein each of the aryl and Het is optionally substituted with halo, OH, (C₁₋₆)alkyl or —O(C₁₋₆)alkyl; and
c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, —(C₁₋₆)alkylene-SO₂—N(R⁸)R⁹ or —(C₁₋₆)alkylene-NR⁹—SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
R⁸ is in each instance independently selected from H, (C₁₋₆)alkyl, (C₃₋₇)cycloalkyl, —C(═O)R⁷ and —C(═O)OR⁷; and
R⁹ is in each instance independently selected from halo, cyano, R⁷, OR⁷, —(C₁₋₆)alkylene-R⁷, —SO₂R⁷, —C(═O)R⁷, —OC(═O)R⁷, —C(═O)OR⁷ and —C(═O)N(R⁸)R⁷; wherein R⁷ and R⁸ are as defined above;
or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
wherein the heterocycle is optionally substituted with 1 to 3 substituents each independently selected from (C₁₋₆)alkyl optionally substituted with OH, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R³ is selected from H, halo, (C₁₋₆)alkyl, (C₁₋₆)haloalkyl, —O—(C₁₋₆)alkyl, —S—(C₁₋₆)alkyl, cyano, —NH₂, —NH(C₁₋₆)alkyl and —N((C₁₋₆)alkyl)₂;
R⁵ is selected from H, (C₁₋₆)alkyl, (C₃₋₇)cycloalkyl, (C₃₋₇)cycloalkyl-(C₁₋₆)alkyl-, —O—(C₁₋₆)alkyl, —S—(C₁₋₆)alkyl, cyano, —NH₂, —NH(C₁₋₆)alkyl, —N((C₁₋₆)alkyl)₂, —NHC(═O)—(C₁₋₃)alkyl, aryl, aryl-(C₁₋₆)alkyl-, Het or Het —(C₁₋₆)alkyl-; wherein the (C₁₋₆)alkyl, aryl, aryl-(C₁₋₆)alkyl-, Het or Het —(C₁₋₆)alkyl- are optionally substituted with 1 to 4 substituents each independently selected from (C₁₋₆)alkyl, halo, —OH, —COOH, —O(C₁₋₆)alkyl, —C(═O)—(C₁₋₆)alkyl, —C(═O)—O—(C₁₋₆)alkyl, cyano, —NH₂, —NH(C₁₋₆)alkyl, and —N((C₁₋₆)alkyl)₂;
R⁶ is selected from (C₁₋₈)alkyl, (C₂₋₈)alkenyl, (C₂₋₈)alkynyl, (C₃₋₇)cycloalkyl, aryl and Het,
wherein said R⁶ can be optionally substituted with 1 to 6 R²¹ substituents,
wherein R²¹ in each case is independently selected from:
c) halo, NH₂, NO₂, cyano, azido or oxo;
d) R²¹⁰, OR²¹⁰, NR²¹⁰R²¹¹, SR²¹⁰, SOR²¹⁰, SO₂R²¹⁰, C(═O)R²¹⁰, C(═O)OR²¹⁰, C(═O)NR²¹⁰R²¹¹, NR²¹¹C(═O)R²¹², NR²¹¹C(═O)OR²¹², NR²¹¹C(═O)NR²¹¹R²¹², NR²¹¹SO₂R²¹⁰, NR²¹¹SO₂NR²¹⁰R²¹² and SO₂NR²¹⁰R²¹¹;
wherein R²¹⁰ is selected from H, (C₁₋₈)alkyl, (C₁₋₈)haloalkyl, (C₂₋₈)alkenyl, (C₂₋₈)alkynyl, (C₃₋₇)cycloalkyl, (C₈₋₇)cycloalkenyl, (C₃₋₇)spirocycloalkyl optionally containing 1 to 3 heteroatom selected from N, O and S, C(═O)R²¹¹, C(═O)OR²¹¹, aryl and Het, all of which can be optionally substituted with 1 to 6 substituents selected from OH, NH₂, cyano, oxo, NO₂, halo, R²¹², OR²¹¹, SR²¹¹, NR²¹¹R²¹², NR²¹¹C(═O)R²¹², NR²¹¹C(═O)OR²¹², NR²¹¹C(═O)NR²¹¹R²¹², NR²¹¹SO₂R²¹⁰, NR²¹¹SO₂NR²¹⁰R²¹², C(═O)R²¹¹, C(═O)OR²¹¹, C(═O)NR²¹¹R²¹²,
and wherein R²¹¹ is selected from H, (C₁₋₆)alkyl, and (C₃₋₇)cycloalkyl;
and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl and Het, all of which being optionally substituted with 1 to 6 substituents selected from OH, NH₂, cyano, oxo, NO₂, halo, (C₁₋₆)alkyl, (C₃₋₇)cycloalkyl, (C₁₋₆)haloalkyl, O—(C₁₋₆)alkyl, S—(C₁₋₆)alkyl, NH(C₁₋₆)alkyl, N((C₁₋₆)alkyl)₂, aryl and Het, wherein aryl and Het can be optionally substituted with 1 to 3 substituents selected from OH, halo, (C₁₋₃)alkyl and —O(C₁₋₃)alkyl;
or R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
or a salt thereof.

2. The compound according to claim 1, or a pharmaceutically acceptable salt thereof, wherein X is O.

3. The compound according to claim 1, or a pharmaceutically acceptable salt thereof, wherein R² is selected from the following formulas: 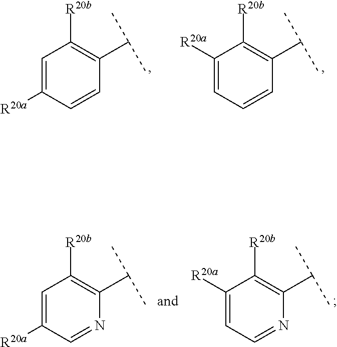
wherein R^{20b} is selected from H, halo, (C₁₋₆)alkyl, (C₁₋₆)haloalkyl, (C₃₋₇)cycloalkyl and —O—(C₁₋₆)haloalkyl; and R^{20a} is R²⁰ as defined in claim 1.

4. The compound according to claim 1, or a pharmaceutically acceptable salt thereof, wherein R²⁰ is selected from:
a) halo or cyano;
b) R⁷, —C(═O)—R⁷, —C(═O)OR⁷, —OR⁷ or —(C₁₋₆)alkylene-C(═O)OR⁷;
wherein R⁷ is in each instance independently selected from H, (C₁₋₄)alkyl, phenyl and Het;
wherein the (C₁₋₄)alkyl is optionally substituted with 1 to 3 substituents each independently selected from —OH, halo, (C₃₋₇)cycloalkyl, —O—(C₁₋₃)alkyl, cyano, COOH, —N(R⁸)R⁹, —C(═O)N(R⁸)R⁹, aryl and Het; and
wherein each of the phenyl and Het is optionally substituted with 1 to 3 substituents each independently selected from:
i) halo, cyano, oxo, —OH, —COOH, —O—(C₁₋₆)alkyl, SO₂NH₂, —SO₂—NH(C₁₋₃)alkyl, —SO₂—N((C₁₋₃)alkyl)₂, —NH₂, —NH(C₁₋₃)alkyl, —N((C₁₋₃)alkyl)₂;
ii) (C₁₋₄)alkyl optionally substituted with —OH or —O—(C₁)alkyl; and
iii) phenyl or Het, wherein each of the phenyl and Het is optionally substituted with 1 to 3 substituents selected from the group consisting of halo, OH or—O(C₁)alkyl;
wherein each Het is selected from: 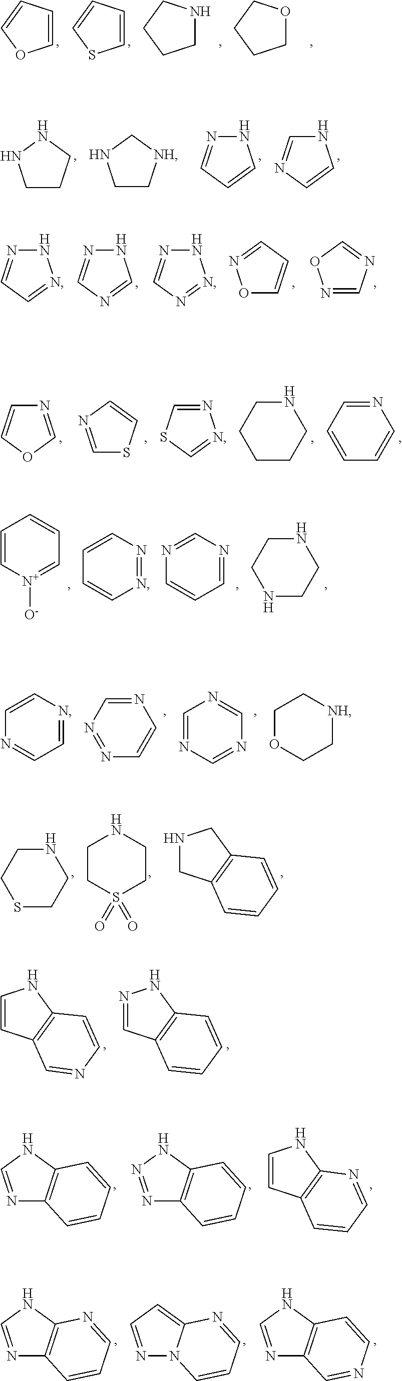 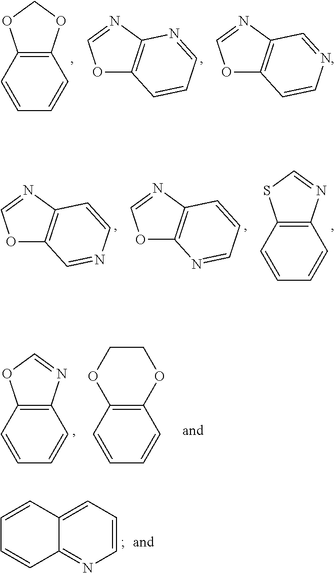
c) —N(R⁸)R⁹, —C(═O)—N(R⁸)R⁹, —SO₂—N(R⁸)R⁹, —(C₁₋₃)alkylene-N(R⁸)R⁹ or —(C₁₋₃)alkylene-C(═O)—N(R⁸)R⁹; wherein the (C₁₋₃)alkylene is optionally substituted with 1 or 2 substituents each independently selected from —OH and —O—(C₁₋₃)alkyl;
R⁸ is in each instance independently selected from H and (C₁₋₃)alkyl; and
R⁹ is in each instance independently selected from halo, cyano, R⁷, OR⁷, —SO₂R⁷, —C(═O)R⁷ and —C(═O)OR⁷; wherein R⁷ is as defined above;
or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
wherein the heterocycle is optionally substituted with 1 to 3 substituents each independently selected from (C₁₋₃)alkyl optionally substituted with —OH, —O(C₁₋₃)alkyl, —NH₂, —NH(C₁₋₃)alkyl and —N((C₁₋₃)alkyl)₂.

5. The compound according to claim 4, or a pharmaceutically acceptable salt thereof, wherein R²⁹ is selected from: H, F, Cl, Br, OH, CF₃, (C₁₋₃)alkyl, O—(C₁₋₃)alkyl, (C₁₋₃)alkyl-COOH, (C₁₋₃)alkyl-CONH₂, NH₂, NH(C₁₋₃)alkyl, N((C₁₋₃)alkyl)₂, phenyl or Het, wherein the phenyl and Het are optionally substituted with 1 to 3 substituents selected from the group consisting of halo, OH, (C₁₋₃)alkyl, —NH₂, —NH(C₁₋₃)alkyl, —N((C₁₋₃)alkyl)₂, O—(C₁₋₃)alkyl, phenyl or Het, wherein each Het is selected from:

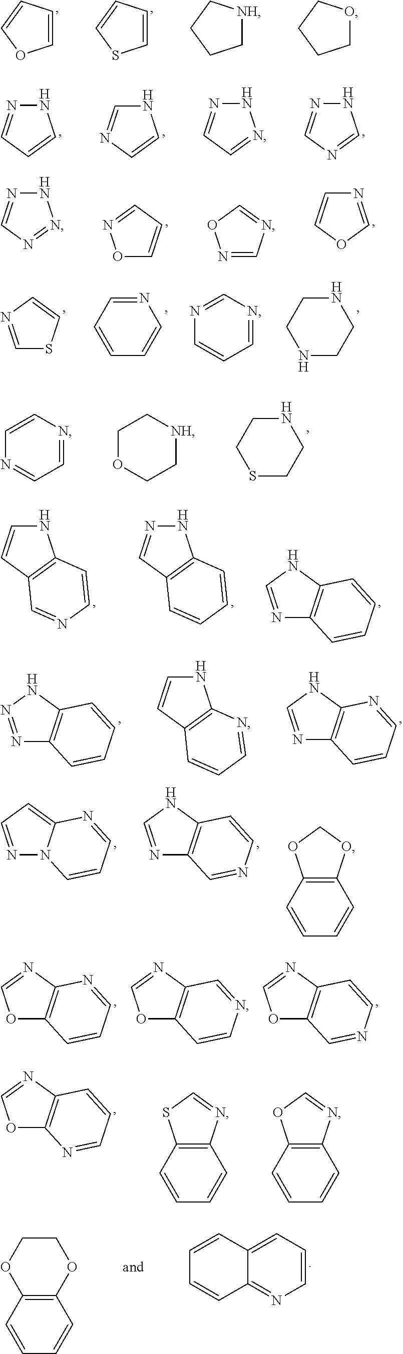

6. The compound according to claim 1, or a pharmaceutically acceptable salt thereof, wherein R³ is H.

7. The compound according to any claim 1, or a pharmaceutically acceptable salt thereof, wherein R⁵ is H.

8. The compound according to claim 1, or a pharmaceutically acceptable salt thereof, wherein R⁶ is selected from: 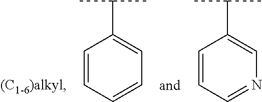
wherein R⁶ is optionally substituted with 1 to 3 R²¹ substituents
wherein R²¹ is selected from:
a) halo, NH₂, cyano, azido or oxo;
b) R²¹⁰, OR²¹⁰, NR²¹⁰R²¹¹, C(O)R²¹⁰, C(═O)OR²¹⁰, —C(═O)NR²¹⁰R²¹¹, NR²¹¹C(═O)R²¹², NR²¹¹C(═O)OR²¹², NR²¹¹C(═O)NR²¹¹R²¹² and NR²¹¹SO₂R²¹⁰;
wherein R²¹⁰ is selected from H, (C₁₋₆)alkyl, (C₂₋₆)alkenyl, (C₃₋₆)cycloalkyl, (C₆₋₇)cycloalkenyl, (C₃₋₇)spirocycloalkyl, aryl and Het, all of which can be optionally substituted with 1 to 6 substituents selected from OH, NH₂, cyano, oxo, halo, R²¹², OR²¹¹, SR²¹¹, NR²¹¹R²¹², C(═O)R²¹¹, C(═O)OR²¹¹ and C(═O)NR²¹¹R²¹²,
and wherein R²¹¹ is selected from H and (C₁₋₆)alkyl;
and wherein R²¹² is selected from H, (C₁₋₆)alkyl, (C₂₋₆)alkenyl, (C₂₋₆)alkynyl, (C₁₋₆)haloalkyl, —O—(C₁₋₆)alkyl, (C₃₋₇)cycloalkyl, (C₃₋₇)cycloalkenyl, aryl and Het, all of which being optionally substituted with 1 to 3 substituents selected from OH, halo, (C₁₋₆)alkyl, (C₃₋₇)cycloalkyl, O—(C₁₋₆)alkyl, S—(C₁₋₆)alkyl, NH(C₁₋₆)alkyl, N((C₁₋₆)alkyl)₂, aryl and Het, wherein aryl and Het can be optionally substituted with 1 to 3 substituents selected from OH, halo, (C₁₋₃)alkyl and —O(C₁₋₃)alkyl;
or R²¹⁰ and R²¹¹, or R²¹¹ and R²¹² together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂; wherein the heterocycle is optionally substituted with 1 to 3 substituents each independently selected from (C₁₋₆)alkyl, (C₁₋₆)haloalkyl, halo, oxo, OH, —O(C₁₋₆)alkyl and —NH₂.

9. The compound of formula (I) according to claim 8, or a pharmaceutically acceptable salt thereof, wherein R²¹ is selected from F, Cl, Br; OH, NH₂, (C₁₋₃)alkyl, (C₂₋₄)alkenyl, aryl or Het, wherein (C₁₋₃)alkyl, (C₂₋₄)alkenyl, aryl and Het are optionally substituted with halo, OH, (C₁₋₃)alkyl, (C₃₋₆)cycloalkyl, O—(C₁₋₃)alkyl, C(═O)N((C₁₋₃)alkyl)₂, NHC(═O)(C₁₋₃)alkyl, NHC(═O)NH(C₁₋₃)alkyl, phenyl or Het wherein Het is a 5 to 7 membered heterocycle having 1 to 2 N atoms and 0 to 2 heteroatoms each independently selected from O and S.

10. The compound of formula (I) according to claim 1, or a pharmaceutically acceptable salt thereof, having the formula: 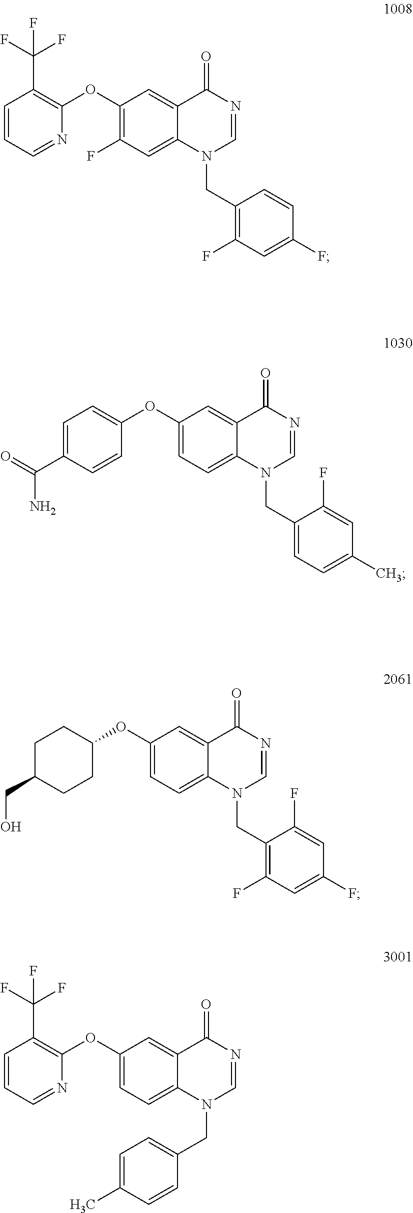 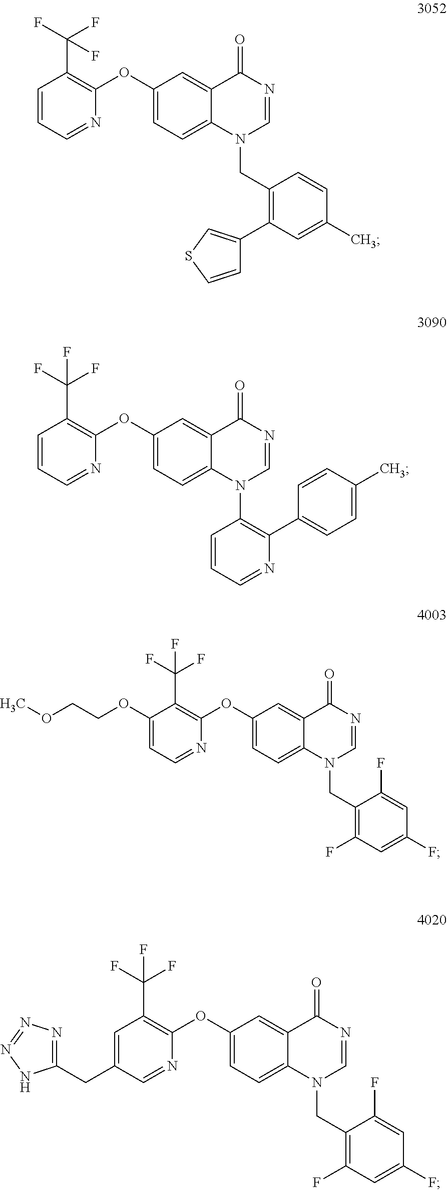 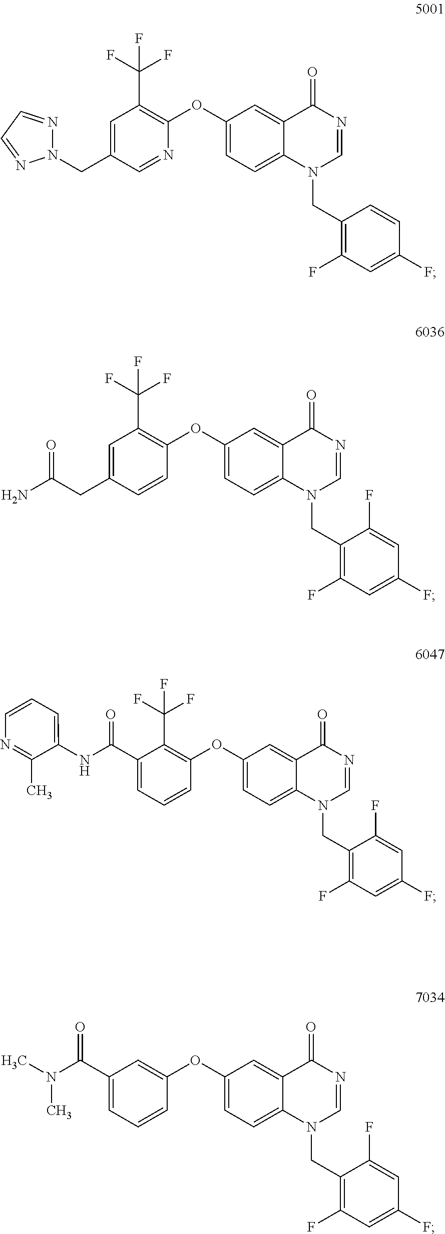 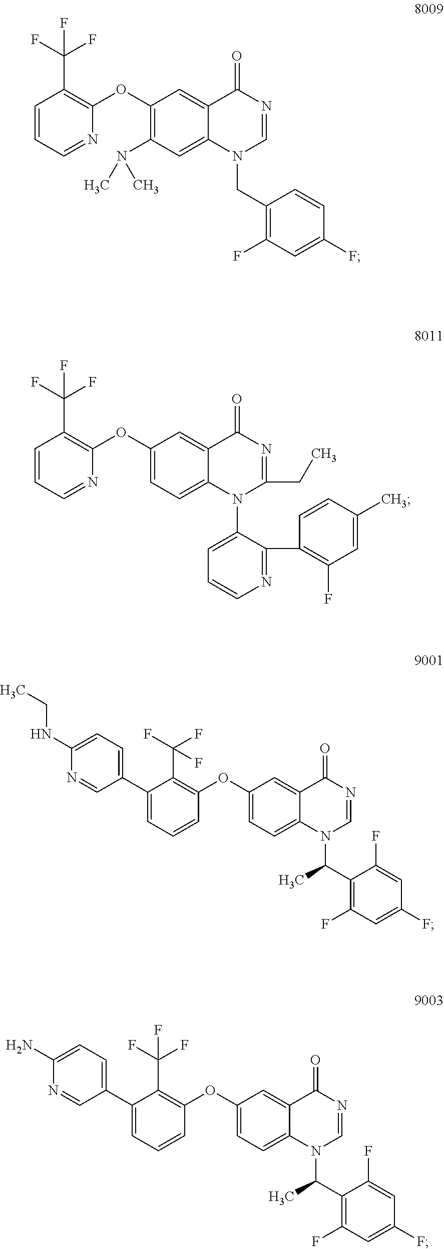 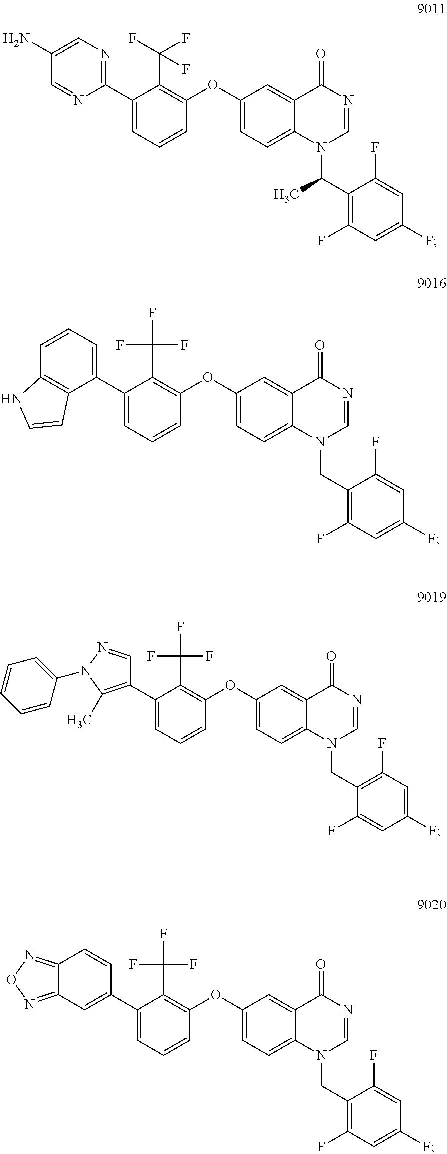 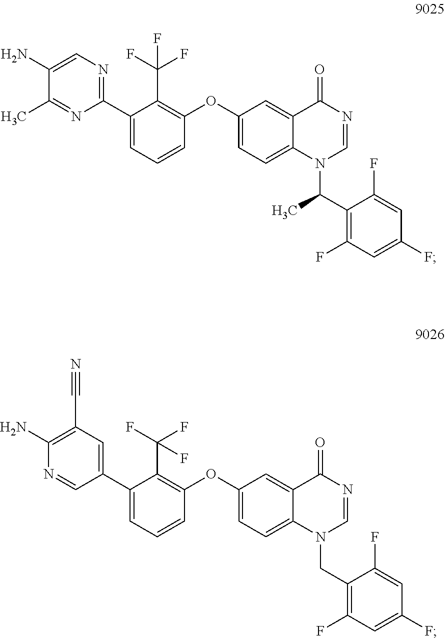
or a pharmaceutically acceptable salt thereof.

11. A compound represented by a formula selected from the group consisting of:

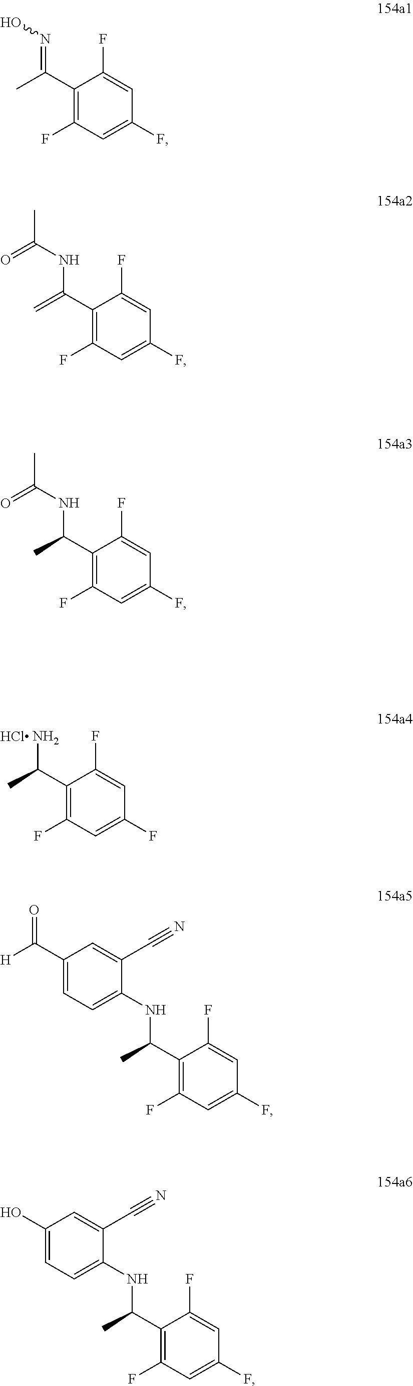

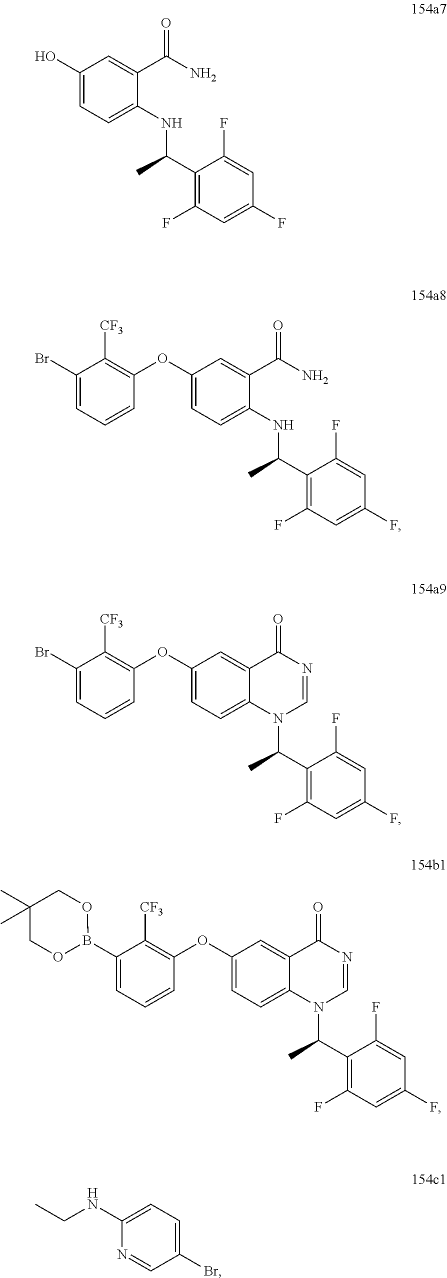

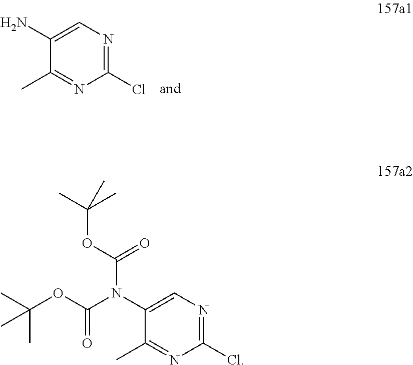

12. A pharmaceutical composition comprising a therapeutically effective amount of a compound of formula (I) according to claim 1 or a pharmaceutically acceptable salt thereof; and one or more pharmaceutically acceptable carriers.

13. The pharmaceutical composition according to claim 12, additionally comprising at least one other antiviral agent.

14. A method for treating hepatitis C viral infection in a mammal comprising administering to said mammal a therapeutically effective amount of a compound of formula (I) according to claim 1, or a pharmaceutically acceptable salt thereof.
